(19) United States
(12) Patent Application Publication
(10) Pub. No.: US 2024/0254199 A1
(43) Pub. Date: Aug. 1, 2024

(54) MODIFIED MAMMALIAN CELLS

(71) Applicants: Genentech, Inc., South San Francisco, CA (US); Hoffmann-La Roche Inc., Little Falls, NJ (US)

(72) Inventors: Amy Shen, South San Francisco, CA (US); Inn Huam Yuk, South San Francisco, CA (US); Gavin Christian Barnard, South San Francisco, CA (US); Shahram Misaghi, South San Francisco, CA (US); Simon Auslaender, Penzberg (DE); Niels Bauer, Penzberg (DE); Benedikt Oswald, Penzberg (DE)

(73) Assignees: Genentech, Inc., South San Francisco, CA (US); Hoffmann-La Roche Inc., Little Falls, NJ (US)

(21) Appl. No.: 18/489,274

(22) Filed: Oct. 18, 2023

Related U.S. Application Data
(63) Continuation of application No. PCT/US2022/025282, filed on Apr. 19, 2022.
(60) Provisional application No. 63/220,181, filed on Jul. 9, 2021.
(60) Provisional application No. 63/220,124, filed on Jul. 9, 2021.
(60) Provisional application No. 63/176,846, filed on Apr. 19, 2021.

(51) Int. Cl. C07K 16/00; C12N 5/00; C12N 9/22; C12N 15/11; C12N 15/90; C12P 21/00

(52) U.S. Cl. CPC C07K 16/00; C12N 5/00; C12N 9/22; C12N 15/11; C12N 15/907; C12P 21/00; C12N 2310/20; C12N 2800/80

(57) ABSTRACT

The present disclosure relates to mammalian cells (e.g., Chinese Hamster Ovary (CHO) cells) that are modified to reduce or eliminate the expression of certain mammalian cell endogenous products (e.g., host cell proteins and virus-like particles), and methods of using such cells in the production of a recombinant product of interest, e.g., a recombinant protein, a recombinant viral particle, or a recombinant viral vector. These modifications were specifically chosen to generate engineered mammalian host cells with desired traits in several key areas, including improved cell culture performance (e.g., higher viability and product titers), improved product quality (e.g., more consistent and favorable glycosylation; more stable drug product), and decreased burden on purification for removing problematic or undesired endogenous host cell products (e.g., hydrolytic host cell proteins and virus-like particles) during biomanufacturing.

### CROSS-REFERENCE TO RELATED APPLICATIONS

This application is a continuation of International Patent Application No. PCT/US2022/025282 filed Apr. 19, 2022, which claims priority to U.S. Provisional Application No. 63/176,846 filed Apr. 19, 2021, U.S. Provisional Application No. 63/220,124 filed on Jul. 9, 2021, U.S. Provisional Application No. 63/220,181 filed Jul. 9, 2021, the contents of each of which are incorporated by reference in their entirety, and to each of which priority is claimed.

### SEQUENCE LISTING

The instant application contains a Sequence Listing which has been submitted electronically in XML format and is hereby incorporated by reference in its entirety. Said sequence listing copy, created on Apr. 2, 2024, is named 00B206_1375SL.xml and is 112, 984 bytes in size.

### 1. FIELD OF INVENTION

The present disclosure relates to mammalian cells (e.g., Chinese Hamster Ovary (CHO) cells) that are modified to reduce or eliminate the expression of certain mammalian cell endogenous products (e.g., host cell proteins and virus-like particles), and methods of using such cells in the production of a recombinant product of interest (e.g., a recombinant protein, a recombinant viral particle, or a recombinant viral vector). These modifications were specifically chosen to generate engineered mammalian host cells with desired traits in several key areas, including improved cell culture performance (e.g., higher viability and product titers), improved product quality (e.g., more consistent and favorable glycosylation; more stable drug product), and decreased burden on purification for removing problematic or undesired endogenous host cell products (e.g., hydrolytic host cell proteins and virus-like particles) during biomanufacturing.

### 2. BACKGROUND

Due to the rapid advancement in cell biology and immunology, there has been an increasing demand to develop novel therapeutic recombinant proteins for a variety of diseases including cancer, cardiovascular diseases and metabolic diseases. These biopharmaceutical candidates are commonly manufactured by commercial cell lines capable of expressing the products of interest. For example, Chinese hamster ovary (CHO) cells have been widely adapted to produce monoclonal antibodies.

Expression of certain proteins by mammalian cells are detrimental for cell culture performance (e.g., proteins that promote apoptosis and hence decrease culture viability and productivity). Certain glycosylation enzymes that are not typically expressed in humans may however be expressed in non-human mammalian cells; therefore, the use of such non-human mammalian cells can give rise to non-human glycosylation patterns in the recombinant product. Moreover, mammalian cells, including CHO cells, express many proteins that are not essential for cell growth, survival, and/or productivity. Expression of these mammalian cell proteins, however, consumes considerable cellular energy and DNA/protein building blocks. Reducing or eliminating the expression of such proteins can render cell growth more efficient. In addition, in contexts where the cell is used for production of a recombinant product of interest (e.g., a recombinant protein), some of these endogenous proteins can co-purify with the recombinant product of interest, leading to increased costs associated with additional purification process improvements and/or decreased shelf-life of the resulting product. For example, certain residual mammalian cell proteins that co-purify with the product of interest can degrade polysorbate used as a surfactant in the final drug product, and lead to particle formation (Dixit et al., J Pharm Sci, 2016, Volume 105, Issue 5, Pages 1657-1666). Likewise, expression of endogenous retrovirus-like particles (RVLPs) by mammalian cells are undesirable, and considerable burden is placed on downstream processing to demonstrate adequate removal of RVLPs in biotherapeutic manufacturing processes.

Accordingly, there is a need in the art for more efficient methods, mammalian cells, and compositions for producing a recombinant product of interest (e.g., a recombinant protein, a recombinant viral particle, or recombinant viral vector), where the modified mammalian cells expressing the recombinant product of interest exhibit improved attributes relevant to mammalian cell viability, expression, and product quality, as well as facilitating downstream purification of the product of interest. Such improved mammalian cells can be achieved by applying carefully-selected modifications to the genome of the mammalian host cells (i.e., cell line engineering).

### 3. SUMMARY

In certain embodiments, the present disclosure is directed to a modified mammalian cell, wherein the cell is modified to reduce or eliminate the expression of one or more endogenous products relative to the expression of the endogenous products in an unmodified cell, wherein the one or more endogenous products: promotes apoptosis of the modified cell during cell culture; promotes clumping and/or aggregation of the modified cell during cell culture; is not essential for the growth, survival, and/or productivity of the modified cell during cell culture; promotes non-human glycosylation patterns in a recombinant protein product produced by the modified cell during cell culture; can co-purify with the product of interest produced by the modified cell during cell culture; and/or requires removal by purification for product quality and/or safety reasons.

In certain embodiments, the present disclosure is directed to a modified mammalian cell, wherein the cell is modified to reduce or eliminate the expression of one or more endogenous products relative to the expression of the endogenous products in an unmodified cell, wherein the one or more endogenous products is selected from endogenous virus-like particles such as retrovirus-like particles (RVLPs), e.g., via a reduction or elimination of RVLP group antigen (GAG) expression, and/or one or more of the endogenous protein group consisting of: BCL2 Associated X, Apoptosis Regulator (BAX); BCL2 Antagonist/Killer 1 (BAK); Intercellular Adhesion Molecule 1 (ICAM-1); Protein Kinase R-like ER Kinase (PERK); Sirtuin 1 (SIRT-1); MYC Proto-Oncogene, BHLH Transcription Factor (MYC); Glycoprotein Alpha-Galactosyltransferase 1 (GGTA1); Cytidine Monophosphate-N-Acetylneuraminic Acid Hydroxylase (CMAH); Lipoprotein lipase (LPL); Phospholipase A2 group XV (LPLA2); Palmitoyl-protein thioesterase 1 (PPT1); Branched Chain Keto Acid Dehydrogenase El alpha subunit (BCKDHA); Branched Chain Keto Acid Dehydrogenase El beta subunit (BCKDHB); and Lipase A (Lysosomal acid lipase/cholesteryl ester hydrolase, Lipase) (LIPA).

In certain embodiments, the present disclosure is directed to a modified cell of wherein the expression of RVLPs is reduced or eliminated, e.g., via a reduction or elimination of RVLP group antigen (GAG) expression.

In certain embodiments, the present disclosure is directed to a modified cell of wherein the expression of:
a) BAX; BAK; ICAM-1; GGTA1; CMAH; LPL; LPLA2; and PPT1;
b) BAX; BAK; ICAM-1; SIRT-1; GGTA1; CMAH; LPL, LPLA2; and PPT1;
c) BAX; BAK; ICAM-1; SIRT-1; MYC; GGTA1; CMAH; LPL; LPLA2; and PPT1;
d) BAX; BAK; ICAM-1; SIRT-1; MYC; GGTA1; CMAH; LPL, LPLA2; PPT1; and LIPA;
e) BAX; BAK; ICAM-1; GGTA1; CMAH; LPLA2; PPT1; and LIPA;
f) BAX; BAK; ICAM-1; SIRT-1; GGTA1; CMAH; LPLA2; PPT1; and LIPA;
g) BAX; BAK; ICAM-1; SIRT-1; MYC; GGTA1; CMAH; LPLA2; PPT1; and LIPA;
h) BAX; BAK; ICAM-1; LPL; LPLA2; and PPT1;
i) BAX; BAK; ICAM-1; SIRT-1; LPL, LPLA2; and PPT1;
j) BAX; BAK; ICAM-1; SIRT-1; MYC; LPL; LPLA2; and PPT1;
k) BAX; BAK; ICAM-1; SIRT-1; MYC; LPL; LPLA2; PPT1; and LIPA;
l) BAX; BAK; ICAM-1; SIRT-1; and MYC;
m) BAX; BAK; ICAM-1; PERK; SIRT-1; and MYC;
n) BAX; BAK; ICAM-1; SIRT-1; PERK; MYC; GGTA1; CMAH; LPL, LPLA2; PPT1; and LIPA;
o) BAX; BAK; LPL; LPLA2; GGTA1; and CMAH;
p) BAX; BAK; PERK; LPL; LPLA2; GGTA1; and CMAH;
q) BAX; BAK; MYC; LPL; LPLA2; GGTA1; and CMAH;
r) BAX; BAK; MYC; PERK; LPL; LPLA2; GGTA1; and CMAH;
s) BAX; BAK; LPL; LPLA2; GGTA1; CMAH; and PPT1;
t) BAX; BAK; PERK; LPL; LPLA2; GGTA1; CMAH; and PPT1;
u) BAX; BAK; MYC; LPL; LPLA2; GGTA1; CMAH; and PPT1;
v) BAX; BAK; MYC; PERK; LPL; LPLA2; GGTA1; CMAH; and PPT1;
w) BAX; BAK; ICAM-1; and SIRT-1;
x) BAX; BAK; and ICAM-1;
y) BAX; BAK; BCKDHA; ICAM-1; GGTA1; CMAH; LPL; LPLA2; and PPT1;
z) BAX; BAK; BCKDHA; ICAM-1; SIRT-1; GGTA1; CMAH; LPL; LPLA2; and PPT1;
aa) BAX; BAK; BCKDHA; ICAM-1; SIRT-1; MYC; GGTA1; CMAH; LPL; LPLA2; and PPT1;
bb) BAX; BAK; BCKDHA; ICAM-1; SIRT-1; MYC; GGTA1; CMAH; LPL, LPLA2; PPT1; and LIPA;
cc) BAX; BAK; BCKDHA; ICAM-1; GGTA1; CMAH; LPLA2; PPT1; and LIPA;
dd)BAX; BAK; BCKDHA; ICAM-1; SIRT-1; GGTA1; CMAH; LPLA2; PPT1; and LIPA;
ee) BAX; BAK; BCKDHA; ICAM-1; SIRT-1; MYC; GGTA1; CMAH; LPLA2; PPT1; and LIPA;
ff) BAX; BAK; BCKDHA; ICAM-1; LPL; LPLA2; and PPT1;
gg)BAX; BAK; BCKDHA; ICAM-1; SIRT-1; LPL, LPLA2; and PPT1;
hh)BAX; BAK; BCKDHA; ICAM-1; SIRT-1; MYC; LPL; LPLA2; and PPT1;
ii) BAX; BAK; BCKDHA; ICAM-1; SIRT-1; MYC; LPL; LPLA2; PPT1; and LIPA;
jj) BAX; BAK; BCKDHA; ICAM-1; SIRT-1; and MYC;
kk)BAX; BAK; BCKDHA; ICAM-1; PERK; SIRT-1; and MYC;
ll) BAX; BAK; BCKDHA; ICAM-1; PERK; SIRT-1; MYC; GGTA1; CMAH; LPL, LPLA2; PPT1; and LIPA;
mm) BAX; BAK; BCKDHA; LPL; LPLA2; GGTA1; and CMAH;
nn)BAX; BAK; BCKDHA; PERK; LPL; LPLA2; GGTA1; and CMAH;
oo)BAX; BAK; BCKDHA; MYC; LPL; LPLA2; GGTA1; and CMAH;
pp)BAX; BAK; BCKDHA; MYC; PERK; LPL; LPLA2; GGTA1; and CMAH;
qq)BAX; BAK; BCKDHA; LPL; LPLA2; GGTA1; CMAH; and PPT1;
rr) BAX; BAK; BCKDHA; PERK; LPL; LPLA2; GGTA1; CMAH; and PPT1;
ss) BAX; BAK; BCKDHA; MYC; LPL; LPLA2; GGTA1; CMAH; and PPT1;
tt) BAX; BAK; BCKDHA; MYC; PERK; LPL; LPLA2; GGTA1; CMAH; and PPT1;
uu)BAX; BAK; BCKDHA; ICAM-1; and SIRT-1;
vv)BAX; BAK; BCKDHA; and ICAM-1;
ww) BAX; BAK; BCKDHB; ICAM-1; GGTA1; CMAH; LPL; LPLA2; and PPT1;
xx)BAX; BAK; BCKDHB; ICAM-1; SIRT-1; GGTA1; CMAH; LPL; LPLA2; and PPT1;
yy)BAX; BAK; BCKDHB; ICAM-1; SIRT-1; MYC; GGTA1; CMAH; LPL; LPLA2; and PPT1;
zz) BAX; BAK; BCKDHB; ICAM-1; SIRT-1; MYC; GGTA1; CMAH; LPL, LPLA2; PPT1; and LIPA;
aaa) BAX; BAK; BCKDHB; ICAM-1; GGTA1; CMAH; LPLA2; PPT1; and LIPA;
bbb) BAX; BAK; BCKDHB; ICAM-1; SIRT-1; GGTAT; CMAH; LPLA2; PPT1; and LIPA;
ccc) BAX; BAK; BCKDHB; ICAM-1; SIRT-1; MYC; GGTA1; CMAH; LPLA2; PPT1; and LIPA;
ddd) BAX; BAK; BCKDHB; ICAM-1; LPL; LPLA2; and PPT1;
eee) BAX; BAK; BCKDHB; ICAM-1; SIRT-1; LPL, LPLA2; and PPT1;
fff) BAX; BAK; BCKDHB; ICAM-1; SIRT-1; MYC; LPL; LPLA2; and PPT1;
ggg) BAX; BAK; BCKDHB; ICAM-1; SIRT-1; MYC; LPL; LPLA2; PPT1; and LIPA;
hhh) BAX; BAK; BCKDHB; ICAM-1; SIRT-1; and MYC;
iii) BAX; BAK; BCKDHB; ICAM-1; PERK; SIRT-1; and MYC;
jjj) BAX; BAK; BCKDHB; ICAM-1; PERK; SIRT-1; MYC; GGTAT; CMAH; LPL, LPLA2; PPT1; and LIPA;
kkk) BAX; BAK; BCKDHB; LPL; LPLA2; GGTA1; and CMAH;
lll) BAX; BAK; BCKDHB; PERK; LPL; LPLA2; GGTA1; and CMAH;
mmm) BAX; BAK; BCKDHB; MYC; LPL; LPLA2; GGTA1; and CMAH;
nnn) BAX; BAK; BCKDHB; MYC; PERK; LPL; LPLA2; GGTAT; and CMAH;
ooo) BAX; BAK; BCKDHB; LPL; LPLA2; GGTA1; CMAH; and PPT1;
ppp) BAX; BAK; BCKDHB; PERK; LPL; LPLA2; GGTA1; CMAH; and PPT1;
qqq) BAX; BAK; BCKDHB; MYC; LPL; LPLA2; GGTA1; CMAH; and PPT1;
rrr) BAX; BAK; BCKDHB; MYC; PERK; LPL; LPLA2; GGTA1; CMAH; and PPT1;
sss) BAX; BAK; BCKDHB; ICAM-1; and SIRT-1;
ttt) BAX; BAK; BCKDHB; and ICAM-1;
uuu) BAX; BAK; BCKDHA; BCKDHB; ICAM-1; GGTA1; CMAH; LPL; LPLA2; and PPT1;
vvv) BAX; BAK; BCKDHA; BCKDHB; ICAM-1; SIRT-1; GGTA1; CMAH; LPL; LPLA2; and PPT1;
www) BAX; BAK; BCKDHA; BCKDHB; ICAM-1; SIRT-1; MYC; GGTA1; CMAH; LPL; LPLA2; and PPT1;
xxx) BAX; BAK; BCKDHA; BCKDHB; ICAM-1; SIRT-1; MYC; GGTA1; CMAH; LPL, LPLA2; PPT1; and LIPA;
yyy) BAX; BAK; BCKDHA; BCKDHB; ICAM-1; GGTA1; CMAH; LPLA2; PPT1; and LIPA;
zzz) BAX; BAK; BCKDHA; BCKDHB; ICAM-1; SIRT-1; GGTA1; CMAH; LPLA2; PPT1; and LIPA;
aaaa) BAX; BAK; BCKDHA; BCKDHB; ICAM-1; SIRT-1; MYC; GGTA1; CMAH; LPLA2; PPT1; and LIPA;
bbbb) BAX; BAK; BCKDHA; BCKDHB; ICAM-1; LPL; LPLA2; and PPT1;
cccc) BAX; BAK; BCKDHA; BCKDHB; ICAM-1; SIRT-1; LPL, LPLA2; and PPT1;
dddd) BAX; BAK; BCKDHA; BCKDHB; ICAM-1; SIRT-1; MYC; LPL; LPLA2; and PPT1;
eeee) BAX; BAK; BCKDHA; BCKDHB; ICAM-1; SIRT-1; MYC; LPL; LPLA2; PPT1; and LIPA;
ffff) BAX; BAK; BCKDHA; BCKDHB; ICAM-1; SIRT-1; and MYC;
gggg) BAX; BAK; BCKDHA; BCKDHB; ICAM-1; PERK; SIRT-1; and MYC;
hhhh) BAX; BAK; BCKDHA; BCKDHB; ICAM-1; PERK; SIRT-1; MYC; GGTA1; CMAH; LPL, LPLA2; PPT1; and LIPA;
iiii)BAX; BAK; BCKDHA; BCKDHB; LPL; LPLA2; GGTA1; and CMAH;
jjjj)BAX; BAK; BCKDHA; BCKDHB; PERK; LPL; LPLA2; GGTA1; and CMAH;
kkkk) BAX; BAK; BCKDHA; BCKDHB; MYC; LPL; LPLA2; GGTA1; and CMAH;
llll)BAX; BAK; BCKDHA; BCKDHB; MYC; PERK; LPL; LPLA2; GGTA1; and CMAH;
mmmm) BAX; BAK; BCKDHA; BCKDHB; LPL; LPLA2; GGTA1; CMAH; and PPT1;
nnnn) BAX; BAK; BCKDHA; BCKDHB; PERK; LPL; LPLA2; GGTA1; CMAH; and PPT1;
oooo) BAX; BAK; BCKDHA; BCKDHB; MYC; LPL; LPLA2; GGTA1; CMAH; and PPT1;
pppp) BAX; BAK; BCKDHA; BCKDHB; MYC; PERK; LPL; LPLA2; GGTA1; CMAH; and PPT1;
qqqq) BAX; BAK; BCKDHA; BCKDHB; ICAM-1; and SIRT-1; or rrrr) BAX; BAK; BCKDHA; BCKDHB; and ICAM-1. is reduced or eliminated.

In certain embodiments, the present disclosure is directed to a modified cell wherein the expression of
a) GAG; BAX; BAK; ICAM-1; GGTA1; CMAH; LPL; LPLA2; and PPT1;
b) GAG; BAX; BAK; ICAM-1; SIRT-1; GGTA1; CMAH; LPL, LPLA2; and PPT1;
c) GAG; BAX; BAK; ICAM-1; SIRT-1; MYC; GGTA1; CMAH; LPL; LPLA2; and PPT1;
d) GAG; BAX; BAK; ICAM-1; SIRT-1; MYC; GGTA1; CMAH; LPL, LPLA2; PPT1; and LIPA;
e) GAG; BAX; BAK; ICAM-1; GGTA1; CMAH; LPLA2; PPT1; and LIPA;
f) GAG; BAX; BAK; ICAM-1; SIRT-1; GGTA1; CMAH; LPLA2; PPT1; and LIPA;
g) GAG; BAX; BAK; ICAM-1; SIRT-1; MYC; GGTA1; CMAH; LPLA2; PPT1; and LIPA;
h) GAG; BAX; BAK; ICAM-1; LPL; LPLA2; and PPT1;
i) GAG; BAX; BAK; ICAM-1; SIRT-1; LPL, LPLA2; and PPT1;
j) GAG; BAX; BAK; ICAM-1; SIRT-1; MYC; LPL; LPLA2; and PPT1;
k) GAG; BAX; BAK; ICAM-1; SIRT-1; MYC; LPL; LPLA2; PPT1; and LIPA;
l) GAG; BAX; BAK; ICAM-1; SIRT-1; and MYC;
m) GAG; BAX; BAK; ICAM-1; PERK; SIRT-1; and MYC;
n) GAG; BAX; BAK; ICAM-1; PERK; SIRT-1; MYC; GGTA1; CMAH; LPL, LPLA2; PPT1; and LIPA;
o) GAG; BAX; BAK; LPL; LPLA2; GGTA1; and CMAH;
p) GAG; BAX; BAK; PERK; LPL; LPLA2; GGTA1; and CMAH;
q) GAG; BAX; BAK; MYC; LPL; LPLA2; GGTA1; and CMAH;
r) GAG; BAX; BAK; MYC; PERK; LPL; LPLA2; GGTA1; and CMAH;
s) GAG; BAX; BAK; LPL; LPLA2; GGTA1; CMAH; and PPT1;
t) GAG; BAX; BAK; PERK; LPL; LPLA2; GGTA1; CMAH; and PPT1;
u) GAG; BAX; BAK; MYC; LPL; LPLA2; GGTA1; CMAH; and PPT1;
v) GAG; BAX; BAK; MYC; PERK; LPL; LPLA2; GGTA1; CMAH; and PPT1;
w) GAG; BAX; BAK; ICAM-1; and SIRT-1;
x) GAG; BAX; BAK; and ICAM-1;
y) BAX; BAK; BCKDHA; ICAM-1; GGTA1; CMAH; LPL; LPLA2; and PPT1;
z) BAX; BAK; BCKDHA; ICAM-1; SIRT-1; GGTA1; CMAH; LPL; LPLA2; and PPT1;
aa) BAX; BAK; BCKDHA; ICAM-1; SIRT-1; MYC; GGTA1; CMAH; LPL; LPLA2; and PPT1;
bb) BAX; BAK; BCKDHA; ICAM-1; SIRT-1; MYC; GGTA1; CMAH; LPL, LPLA2; PPT1; and LIPA;
cc) BAX; BAK; BCKDHA; ICAM-1; GGTA1; CMAH; LPLA2; PPT1; and LIPA;
dd)BAX; BAK; BCKDHA; ICAM-1; SIRT-1; GGTA1; CMAH; LPLA2; PPT1; and LIPA;
ee) BAX; BAK; BCKDHA; ICAM-1; SIRT-1; MYC; GGTA1; CMAH; LPLA2; PPT1; and LIPA;
ff) BAX; BAK; BCKDHA; ICAM-1; LPL; LPLA2; and PPT1;
gg)BAX; BAK; BCKDHA; ICAM-1; SIRT-1; LPL, LPLA2; and PPT1;
hh)BAX; BAK; BCKDHA; ICAM-1; SIRT-1; MYC; LPL; LPLA2; and PPT1;
ii) BAX; BAK; BCKDHA; ICAM-1; SIRT-1; MYC; LPL; LPLA2; PPT1; and LIPA;
jj) BAX; BAK; BCKDHA; ICAM-1; SIRT-1; and MYC;
kk)BAX; BAK; BCKDHA; ICAM-1; PERK; SIRT-1; and MYC;
ll) BAX; BAK; BCKDHA; ICAM-1; PERK; SIRT-1; MYC; GGTA1; CMAH; LPL, LPLA2; PPT1; and LIPA;
mm) BAX; BAK; BCKDHA; LPL; LPLA2; GGTA1; and CMAH;
nn)BAX; BAK; BCKDHA; PERK; LPL; LPLA2; GGTA1; and CMAH;
oo)BAX; BAK; BCKDHA; MYC; LPL; LPLA2; GGTA1; and CMAH;
pp)BAX; BAK; BCKDHA; MYC; PERK; LPL; LPLA2; GGTA1; and CMAH;
qq)BAX; BAK; BCKDHA; LPL; LPLA2; GGTA1; CMAH; and PPT1;
rr) BAX; BAK; BCKDHA; PERK; LPL; LPLA2; GGTA1; CMAH; and PPT1;
ss) BAX; BAK; BCKDHA; MYC; LPL; LPLA2; GGTA1; CMAH; and PPT1;
tt) BAX; BAK; BCKDHA; MYC; PERK; LPL; LPLA2; GGTA1; CMAH; and PPT1;
uu)BAX; BAK; BCKDHA; ICAM-1; and SIRT-1;
vv)BAX; BAK; BCKDHA; and ICAM-1;
ww) BAX; BAK; BCKDHB; ICAM-1; GGTA1; CMAH; LPL; LPLA2; and PPT1;
xx)BAX; BAK; BCKDHB; ICAM-1; SIRT-1; GGTA1; CMAH; LPL; LPLA2; and PPT1;
yy)BAX; BAK; BCKDHB; ICAM-1; SIRT-1; MYC; GGTA1; CMAH; LPL; LPLA2; and PPT1;
zz) BAX; BAK; BCKDHB; ICAM-1; SIRT-1; MYC; GGTA1; CMAH; LPL, LPLA2; PPT1; and LIPA;
aaa) BAX; BAK; BCKDHB; ICAM-1; GGTA1; CMAH; LPLA2; PPT1; and LIPA;
bbb) BAX; BAK; BCKDHB; ICAM-1; SIRT-1; GGTA1; CMAH; LPLA2; PPT1; and LIPA;
ccc) BAX; BAK; BCKDHB; ICAM-1; SIRT-1; MYC; GGTA1; CMAH; LPLA2; PPT1; and LIPA;
ddd) BAX; BAK; BCKDHB; ICAM-1; LPL; LPLA2; and PPT1;
eee) BAX; BAK; BCKDHB; ICAM-1; SIRT-1; LPL, LPLA2; and PPT1;
fff) BAX; BAK; BCKDHB; ICAM-1; SIRT-1; MYC; LPL; LPLA2; and PPT1;
ggg) BAX; BAK; BCKDHB; ICAM-1; SIRT-1; MYC; LPL; LPLA2; PPT1; and LIPA;
hhh) BAX; BAK; BCKDHB; ICAM-1; SIRT-1; and MYC;
iii) BAX; BAK; BCKDHB; ICAM-1; PERK; SIRT-1; and MYC;
jjj) BAX; BAK; BCKDHB; ICAM-1; PERK; SIRT-1; MYC; GGTA1; CMAH; LPL, LPLA2; PPT1; and LIPA;
kkk) BAX; BAK; BCKDHB; LPL; LPLA2; GGTA1; and CMAH;
lll) BAX; BAK; BCKDHB; PERK; LPL; LPLA2; GGTA1; and CMAH;
mmm) BAX; BAK; BCKDHB; MYC; LPL; LPLA2; GGTA1; and CMAH;
nnn) BAX; BAK; BCKDHB; MYC; PERK; LPL; LPLA2; GGTA1; and CMAH;
ooo) BAX; BAK; BCKDHB; LPL; LPLA2; GGTA1; CMAH; and PPT1;
ppp) BAX; BAK; BCKDHB; PERK; LPL; LPLA2; GGTA1; CMAH; and PPT1;
qqq) BAX; BAK; BCKDHB; MYC; LPL; LPLA2; GGTA1; CMAH; and PPT1;
rrr) BAX; BAK; BCKDHB; MYC; PERK; LPL; LPLA2; GGTA1; CMAH; and PPT1;
sss) BAX; BAK; BCKDHB; ICAM-1; and SIRT-1;
ttt) BAX; BAK; BCKDHB; and ICAM-1;
uuu) BAX; BAK; BCKDHA; BCKDHB; ICAM-1; GGTA1; CMAH; LPL; LPLA2; and PPT1;
vvv) BAX; BAK; BCKDHA; BCKDHB; ICAM-1; SIRT-1; GGTA1; CMAH; LPL; LPLA2; and PPT1;
www) BAX; BAK; BCKDHA; BCKDHB; ICAM-1; SIRT-1; MYC; GGTA1; CMAH; LPL; LPLA2; and PPT1;
xxx) BAX; BAK; BCKDHA; BCKDHB; ICAM-1; SIRT-1; MYC; GGTA1; CMAH; LPL, LPLA2; PPT1; and LIPA;
yyy) BAX; BAK; BCKDHA; BCKDHB; ICAM-1; GGTA1; CMAH; LPLA2; PPT1; and LIPA;
zzz) BAX; BAK; BCKDHA; BCKDHB; ICAM-1; SIRT-1; GGTA1; CMAH; LPLA2; PPT1; and LIPA;
aaaa) BAX; BAK; BCKDHA; BCKDHB; ICAM-1; SIRT-1; MYC; GGTA1; CMAH; LPLA2; PPT1; and LIPA;
bbbb) BAX; BAK; BCKDHA; BCKDHB; ICAM-1; LPL; LPLA2; and PPT1;
cccc) BAX; BAK; BCKDHA; BCKDHB; ICAM-1; SIRT-1; LPL, LPLA2; and PPT1;
dddd) BAX; BAK; BCKDHA; BCKDHB; ICAM-1; SIRT-1; MYC; LPL; LPLA2; and PPT1;
eeee) BAX; BAK; BCKDHA; BCKDHB; ICAM-1; SIRT-1; MYC; LPL; LPLA2; PPT1; and LIPA;
ffff) BAX; BAK; BCKDHA; BCKDHB; ICAM-1; SIRT-1; and MYC;
gggg) BAX; BAK; BCKDHA; BCKDHB; ICAM-1; PERK; SIRT-1; and MYC;
hhhh) BAX; BAK; BCKDHA; BCKDHB; ICAM-1; PERK; SIRT-1; MYC; GGTA1; CMAH; LPL, LPLA2; PPT1; and LIPA;
iiii)BAX; BAK; BCKDHA; BCKDHB; LPL; LPLA2; GGTA1; and CMAH;
jjjj)BAX; BAK; BCKDHA; BCKDHB; PERK; LPL; LPLA2; GGTA1; and CMAH;
kkkk) BAX; BAK; BCKDHA; BCKDHB; MYC; LPL; LPLA2; GGTAT; and CMAH;
llll)BAX; BAK; BCKDHA; BCKDHB; MYC; PERK; LPL; LPLA2; GGTA1; and CMAH;
mmmm) BAX; BAK; BCKDHA; BCKDHB; LPL; LPLA2; GGTAT; CMAH; and PPT1;
nnnn) BAX; BAK; BCKDHA; BCKDHB; PERK; LPL; LPLA2; GGTA1; CMAH; and PPT1;
oooo) BAX; BAK; BCKDHA; BCKDHB; MYC; LPL; LPLA2; GGTAT; CMAH; and PPT1;
pppp) BAX; BAK; BCKDHA; BCKDHB; MYC; PERK; LPL; LPLA2; GGTAT; CMAH; and PPT1;
qqqq) BAX; BAK; BCKDHA; BCKDHB; ICAM-1; and SIRT-1; or rrrr) BAX; BAK; BCKDHA; BCKDHB; and ICAM-1. is reduced or eliminated.

In certain embodiments, the present disclosure is directed to the above-described modified cells, where the one or more endogenous products have no detectable expression.

In certain embodiments, the present disclosure is directed to the above-described modified cells, where the modified cells are transfected to express a recombinant product of interest. In certain embodiments, the present disclosure is directed to the above-described modified cells, where the recombinant product of interest comprises a recombinant viral vector.

In certain embodiments, the present disclosure is directed to the above-described modified cells, where the recombinant product of interest comprises a recombinant viral particle. In certain embodiments, the present disclosure is directed to the above-described modified cells, where the recombinant product of interest comprises a recombinant protein. In certain embodiments, the present disclosure is directed to the above-described modified cells, where the recombinant protein is antibody or an antigen-binding fragment thereof. In certain embodiments, the present disclosure is directed to the above-described modified cells, where the antibody is a multispecific antibody or an antigen-binding fragment thereof. In certain embodiments, the present disclosure is directed to the above-described modified cells, where the antibody consists of a single heavy chain sequence and a single light chain sequence or antigen-binding fragments thereof. In certain embodiments, the present disclosure is directed to the above-described modified cells, where the antibody is a chimeric antibody, a human antibody or a humanized antibody. In certain embodiments, the present disclosure is directed to the above described modified cells, where the antibody is a monoclonal antibody. In certain embodiments, the present disclosure is directed to the above-described modified cells, where the exogenous nucleic acid sequence is integrated in the cellular genome of the mammalian cell at one or more targeted locations.

In certain embodiments, the present disclosure is directed to the above-described modified cells, where the modified cell does not express detectable BAX; BAK; ICAM-1; SIRT-1; PERK; MYC; GGTA1; CMAH; LPLA2; PPT1; BCKDHA; BCKDHB; LPL; and/or LIPA. In certain embodiments, the present disclosure is directed to the above-described modified cells, where the modified cell expresses decreased levels of GAG; BAX; BAK; ICAM-1; PERK; SIRT-1; MYC; GGTAT; CMAH; LPLA2; PPT1; BCKDHA; BCKDHB; LPL; and/or LIPA.

In certain embodiments, the present disclosure is directed to the above-described modified cells, where the modified cell is a modified mammalian cell. In certain embodiments, the modified cell is a modified CHO cell. In another embodiment, the modified cell is a modified HEK 293, HEK-293T, BHK, A549 or HeLa cell.

In certain embodiments, the present disclosure is directed to a composition comprising the above-described modified cells.

In certain embodiments, the present disclosure is directed to a method of producing a recombinant product of interest comprising culturing a modified mammalian cell expressing the recombinant product of interest, wherein the modified cells expressing the recombinant product of interest exhibit reduced or eliminated expression of one or more of the following GAG; BAX; BAK; ICAM-1; PERK; SIRT-1; MYC; GGTAT; CMAH; LPLA2; BCKDHA; BCKDHB; PPT1; LPL; and/or LIPA.

In certain embodiments, the present disclosure is directed to a method of culturing a population of mammalian cells expressing a recombinant product of interest, wherein the modified cells expressing the recombinant product of interest exhibit reduced or eliminated expression of one or more of the following GAG; BAX; BAK; ICAM-1; PERK; SIRT-1; MYC; GGTA1; CMAH; LPLA2; PPT1; BCKDHA; BCKDHB; LPL; and/or LIPA.

In certain embodiments, the present disclosure is directed to a method culturing a population of modified mammalian cells expressing a recombinant product of interest or a method of producing a recombinant product of interest comprising culturing a population of mammalian cells expressing a recombinant product of interest, wherein modified cells expressing the recombinant product of interest exhibit reduced or eliminated expression of: a) BAX; BAK; ICAM-1; GGTA1; CMAH; LPL; LPLA2; and PPT1;
b) BAX; BAK; ICAM-1; SIRT-1; GGTA1; CMAH; LPL; LPLA2; and PPT1;
c) BAX; BAK; ICAM-1; SIRT-1; MYC; GGTA1; CMAH; LPL; LPLA2; and PPT1;
d) BAX; BAK; ICAM-1; SIRT-1; MYC; GGTA1; CMAH; LPL, LPLA2; PPT1; and LIPA;
e) BAX; BAK; ICAM-1; GGTA1; CMAH; LPLA2; PPT1; and LIPA;
f) BAX; BAK; ICAM-1; SIRT-1; GGTA1; CMAH; LPLA2; PPT1; and LIPA;
g) BAX; BAK; ICAM-1; SIRT-1; MYC; GGTA1; CMAH; LPLA2; PPT1; and LIPA;
h) BAX; BAK; ICAM-1; LPL; LPLA2; and PPT1;
i) BAX; BAK; ICAM-1; SIRT-1; LPL, LPLA2; and PPT1;
j) BAX; BAK; ICAM-1; SIRT-1; MYC; LPL; LPLA2; and PPT1;
k) BAX; BAK; ICAM-1; SIRT-1; MYC; LPL; LPLA2; PPT1; and LIPA;
l) BAX; BAK; ICAM-1; SIRT-1; and MYC;
m) BAX; BAK; ICAM-1; PERK; SIRT-1; and MYC;
n) BAX; BAK; ICAM-1; PERK; SIRT-1; MYC; GGTAT; CMAH; LPL, LPLA2; PPT1; and LIPA;
o) BAX; BAK; LPL; LPLA2; GGTA1; and CMAH;
p) BAX; BAK; PERK; LPL; LPLA2; GGTAT; and CMAH;
q) BAX; BAK; MYC; LPL; LPLA2; GGTA1; and CMAH;
r) BAX; BAK; MYC; PERK; LPL; LPLA2; GGTAT; and CMAH;
s) BAX; BAK; LPL; LPLA2; GGTA1; CMAH; and PPT1;
t) BAX; BAK; PERK; LPL; LPLA2; GGTAT; CMAH; and PPT1;
u) BAX; BAK; MYC; LPL; LPLA2; GGTA1; CMAH; and PPT1;
v) BAX; BAK; MYC; PERK; LPL; LPLA2; GGTAT; CMAH; and PPT1;
w) BAX; BAK; ICAM-1; and SIRT-1;
x) BAX; BAK; and ICAM-1;
y) BAX; BAK; BCKDHA; ICAM-1; GGTA1; CMAH; LPL; LPLA2; and PPT1;
z) BAX; BAK; BCKDHA; ICAM-1; SIRT-1; GGTA1; CMAH; LPL; LPLA2; and PPT1;
aa) BAX; BAK; BCKDHA; ICAM-1; SIRT-1; MYC; GGTA1; CMAH; LPL; LPLA2; and PPT1;
bb)BAX; BAK; BCKDHA; ICAM-1; SIRT-1; MYC; GGTA1; CMAH; LPL, LPLA2; PPT1; and LIPA;
cc) BAX; BAK; BCKDHA; ICAM-1; GGTA1; CMAH; LPLA2; PPT1; and LIPA;
dd)BAX; BAK; BCKDHA; ICAM-1; SIRT-1; GGTA1; CMAH; LPLA2; PPT1; and LIPA;
ee) BAX; BAK; BCKDHA; ICAM-1; SIRT-1; MYC; GGTA1; CMAH; LPLA2; PPT1; and LIPA;
ff) BAX; BAK; BCKDHA; ICAM-1; LPL; LPLA2; and PPT1;
gg)BAX; BAK; BCKDHA; ICAM-1; SIRT-1; LPL, LPLA2; and PPT1;
hh) BAX; BAK; BCKDHA; ICAM-1; SIRT-1; MYC; LPL; LPLA2; and PPT1;
ii) BAX; BAK; BCKDHA; ICAM-1; SIRT-1; MYC; LPL; LPLA2; PPT1; and LIPA;
jj) BAX; BAK; BCKDHA; ICAM-1; SIRT-1; and MYC;
kk) BAX; BAK; BCKDHA; ICAM-1; PERK; SIRT-1; and MYC;
ll) BAX; BAK; BCKDHA; ICAM-1; PERK; SIRT-1; MYC; GGTA1; CMAH; LPL, LPLA2; PPT1; and LIPA;
mm) BAX; BAK; BCKDHA; LPL; LPLA2; GGTA1; and CMAH;
nn)BAX; BAK; BCKDHA; PERK; LPL; LPLA2; GGTA1; and CMAH;
oo)BAX; BAK; BCKDHA; MYC; LPL; LPLA2; GGTA1; and CMAH;
pp)BAX; BAK; BCKDHA; MYC; PERK; LPL; LPLA2; GGTA1; and CMAH;
qq)BAX; BAK; BCKDHA; LPL; LPLA2; GGTA1; CMAH; and PPT1;
rr) BAX; BAK; BCKDHA; PERK; LPL; LPLA2; GGTA1; CMAH; and PPT1;
ss) BAX; BAK; BCKDHA; MYC; LPL; LPLA2; GGTA1; CMAH; and PPT1;
tt) BAX; BAK; BCKDHA; MYC; PERK; LPL; LPLA2; GGTA1; CMAH; and PPT1;
uu) BAX; BAK; BCKDHA; ICAM-1; and SIRT-1;
vv) BAX; BAK; BCKDHA; and ICAM-1;
ww) BAX; BAK; BCKDHB; ICAM-1; GGTA1; CMAH; LPL; LPLA2; and PPT1;
xx) BAX; BAK; BCKDHB; ICAM-1; SIRT-1; GGTA1; CMAH; LPL; LPLA2; and PPT1;
yy) BAX; BAK; BCKDHB; ICAM-1; SIRT-1; MYC; GGTA1; CMAH; LPL; LPLA2; and PPT1;
zz) BAX; BAK; BCKDHB; ICAM-1; SIRT-1; MYC; GGTA1; CMAH; LPL, LPLA2; PPT1; and LIPA;
aaa) BAX; BAK; BCKDHB; ICAM-1; GGTA1; CMAH; LPLA2; PPT1; and LIPA;
bbb) BAX; BAK; BCKDHB; ICAM-1; SIRT-1; GGTA1; CMAH; LPLA2; PPT1; and LIPA;
ccc) BAX; BAK; BCKDHB; ICAM-1; SIRT-1; MYC; GGTA1; CMAH; LPLA2; PPT1; and LIPA;
ddd) BAX; BAK; BCKDHB; ICAM-1; LPL; LPLA2; and PPT1;
eee) BAX; BAK; BCKDHB; ICAM-1; SIRT-1; LPL, LPLA2; and PPT1;
fff) BAX; BAK; BCKDHB; ICAM-1; SIRT-1; MYC; LPL; LPLA2; and PPT1;
ggg) BAX; BAK; BCKDHB; ICAM-1; SIRT-1; MYC; LPL; LPLA2; PPT1; and LIPA;
hhh) BAX; BAK; BCKDHB; ICAM-1; SIRT-1; and MYC;
iii) BAX; BAK; BCKDHB; ICAM-1; PERK; SIRT-1; and MYC;
jjj) BAX; BAK; BCKDHB; ICAM-1; PERK; SIRT-1; MYC; GGTA1; CMAH; LPL, LPLA2; PPT1; and LIPA;
kkk) BAX; BAK; BCKDHB; LPL; LPLA2; GGTA1; and CMAH;
lll) BAX; BAK; BCKDHB; PERK; LPL; LPLA2; GGTA1; and CMAH;
mmm) BAX; BAK; BCKDHB; MYC; LPL; LPLA2; GGTA1; and CMAH;
nnn) BAX; BAK; BCKDHB; MYC; PERK; LPL; LPLA2; GGTA1; and CMAH;
ooo) BAX; BAK; BCKDHB; LPL; LPLA2; GGTA1; CMAH; and PPT1;
ppp) BAX; BAK; BCKDHB; PERK; LPL; LPLA2; GGTA1; CMAH; and PPT1;
qqq) BAX; BAK; BCKDHB; MYC; LPL; LPLA2; GGTA1; CMAH; and PPT1;
rrr) BAX; BAK; BCKDHB; MYC; PERK; LPL; LPLA2; GGTA1; CMAH; and PPT1;
sss) BAX; BAK; BCKDHB; ICAM-1; and SIRT-1;
ttt) BAX; BAK; BCKDHB; and ICAM-1;
uuu) BAX; BAK; BCKDHA; BCKDHB; ICAM-1; GGTA1; CMAH; LPL; LPLA2; and PPT1;
vvv) BAX; BAK; BCKDHA; BCKDHB; ICAM-1; SIRT-1; GGTA1; CMAH; LPL; LPLA2; and PPT1;
www) BAX; BAK; BCKDHA; BCKDHB; ICAM-1; SIRT-1; MYC; GGTA1; CMAH; LPL; LPLA2; and PPT1;
xxx) BAX; BAK; BCKDHA; BCKDHB; ICAM-1; SIRT-1; MYC; GGTA1; CMAH; LPL, LPLA2; PPT1; and LIPA;
yyy) BAX; BAK; BCKDHA; BCKDHB; ICAM-1; GGTA1; CMAH; LPLA2; PPT1; and LIPA;
zzz) BAX; BAK; BCKDHA; BCKDHB; ICAM-1; SIRT-1; GGTA1; CMAH; LPLA2; PPT1; and LIPA;
aaaa) BAX; BAK; BCKDHA; BCKDHB; ICAM-1; SIRT-1; MYC; GGTA1; CMAH; LPLA2; PPT1; and LIPA;
bbbb) BAX; BAK; BCKDHA; BCKDHB; ICAM-1; LPL; LPLA2; and PPT1;
cccc) BAX; BAK; BCKDHA; BCKDHB; ICAM-1; SIRT-1; LPL, LPLA2; and PPT1;
dddd) BAX; BAK; BCKDHA; BCKDHB; ICAM-1; SIRT-1; MYC; LPL; LPLA2; and PPT1;
eeee) BAX; BAK; BCKDHA; BCKDHB; ICAM-1; SIRT-1; MYC; LPL; LPLA2; PPT1; and LIPA;
ffff) BAX; BAK; BCKDHA; BCKDHB; ICAM-1; SIRT-1; and MYC;
gggg) BAX; BAK; BCKDHA; BCKDHB; ICAM-1; PERK; SIRT-1; and MYC;
hhhh) BAX; BAK; BCKDHA; BCKDHB; ICAM-1; PERK; SIRT-1; MYC; GGTA1; CMAH; LPL, LPLA2; PPT1; and LIPA;
iiii)BAX; BAK; BCKDHA; BCKDHB; LPL; LPLA2; GGTA1; and CMAH;
jjjj)BAX; BAK; BCKDHA; BCKDHB; PERK; LPL; LPLA2; GGTA1; and CMAH;
kkkk) BAX; BAK; BCKDHA; BCKDHB; MYC; LPL; LPLA2; GGTA1; and CMAH;
llll)BAX; BAK; BCKDHA; BCKDHB; MYC; PERK; LPL; LPLA2; GGTA1; and CMAH;
mmmm) BAX; BAK; BCKDHA; BCKDHB; LPL; LPLA2; GGTA1; CMAH; and PPT1;
nnnn) BAX; BAK; BCKDHA; BCKDHB; PERK; LPL; LPLA2; GGTA1; CMAH; and PPT1;
oooo) BAX; BAK; BCKDHA; BCKDHB; MYC; LPL; LPLA2; GGTAT; CMAH; and PPT1;
pppp) BAX; BAK; BCKDHA; BCKDHB; MYC; PERK; LPL; LPLA2; GGTA1; CMAH; and PPT1;
qqqq) BAX; BAK; BCKDHA; BCKDHB; ICAM-1; and SIRT-1; or rrrr) BAX; BAK; BCKDHA; BCKDHB; and ICAM-1.

In certain embodiments, the present disclosure is directed to a method culturing a population of modified mammalian cells expressing a recombinant product of interest or a method of producing a recombinant product of interest comprising culturing a population of mammalian cells expressing a recombinant product of interest, wherein modified cells expressing the recombinant product of interest exhibit reduced or eliminated expression of: a) GAG; BAX; BAK; ICAM-1; GGTA1; CMAH; LPL; LPLA2; and PPT1;
b) GAG; BAX; BAK; ICAM-1; SIRT-1; GGTA1; CMAH; LPL; LPLA2; and PPT1;
c) GAG; BAX; BAK; ICAM-1; SIRT-1; MYC; GGTA1; CMAH; LPL; LPLA2; and PPT1;
d) GAG; BAX; BAK; ICAM-1; SIRT-1; MYC; GGTAT; CMAH; LPL, LPLA2; PPT1; and LIPA;
e) GAG; BAX; BAK; ICAM-1; GGTA1; CMAH; LPLA2; PPT1; and LIPA;
f) GAG; BAX; BAK; ICAM-1; SIRT-1; GGTA1; CMAH; LPLA2; PPT1; and LIPA;
g) GAG; BAX; BAK; ICAM-1; SIRT-1; MYC; GGTA1; CMAH; LPLA2; PPT1; and LIPA;
h) GAG; BAX; BAK; ICAM-1; LPL; LPLA2; and PPT1;
i) GAG; BAX; BAK; ICAM-1; SIRT-1; LPL, LPLA2; and PPT1;
j) GAG; BAX; BAK; ICAM-1; SIRT-1; MYC; LPL; LPLA2; and PPT1;
k) GAG; BAX; BAK; ICAM-1; SIRT-1; MYC; LPL; LPLA2; PPT1; and LIPA;
l) GAG; BAX; BAK; ICAM-1; SIRT-1; and MYC;
m) GAG; BAX; BAK; ICAM-1; PERK; SIRT-1; and MYC;
n) GAG; BAX; BAK; ICAM-1; PERK; SIRT-1; MYC; GGTA1; CMAH; LPL, LPLA2; PPT1; and LIPA;
o) BAX; BAK; LPL; LPLA2; GGTA1; and CMAH;
p) BAX; BAK; PERK; LPL; LPLA2; GGTAT; and CMAH;
q) BAX; BAK; MYC; LPL; LPLA2; GGTA1; and CMAH;
r) BAX; BAK; MYC; PERK; LPL; LPLA2; GGTAT; and CMAH;
s) GAG; BAX; BAK; LPL; LPLA2; GGTA1; CMAH; and PPT1;
t) GAG; BAX; BAK; PERK; LPL; LPLA2; GGTA1; CMAH; and PPT1;
u) GAG; BAX; BAK; MYC; LPL; LPLA2; GGTA1; CMAH; and PPT1;
v) GAG; BAX; BAK; MYC; PERK; LPL; LPLA2; GGTAT; CMAH; and PPT1;
w) GAG; BAX; BAK; ICAM-1; and SIRT-1;
x) GAG; BAX; BAK; and ICAM-1;
y) BAX; BAK; BCKDHA; ICAM-1; GGTA1; CMAH; LPL; LPLA2; and PPT1;
z) BAX; BAK; BCKDHA; ICAM-1; SIRT-1; GGTA1; CMAH; LPL; LPLA2; and PPT1;
aa) BAX; BAK; BCKDHA; ICAM-1; SIRT-1; MYC; GGTA1; CMAH; LPL; LPLA2; and PPT1;
bb)BAX; BAK; BCKDHA; ICAM-1; SIRT-1; MYC; GGTA1; CMAH; LPL, LPLA2; PPT1; and LIPA;
cc) BAX; BAK; BCKDHA; ICAM-1; GGTA1; CMAH; LPLA2; PPT1; and LIPA;
dd)BAX; BAK; BCKDHA; ICAM-1; SIRT-1; GGTA1; CMAH; LPLA2; PPT1; and LIPA;
ee) BAX; BAK; BCKDHA; ICAM-1; SIRT-1; MYC; GGTA1; CMAH; LPLA2; PPT1; and LIPA;
ff) BAX; BAK; BCKDHA; ICAM-1; LPL; LPLA2; and PPT1;
gg)BAX; BAK; BCKDHA; ICAM-1; SIRT-1; LPL, LPLA2; and PPT1;
hh) BAX; BAK; BCKDHA; ICAM-1; SIRT-1; MYC; LPL; LPLA2; and PPT1;
ii) BAX; BAK; BCKDHA; ICAM-1; SIRT-1; MYC; LPL; LPLA2; PPT1; and LIPA;
jj) BAX; BAK; BCKDHA; ICAM-1; SIRT-1; and MYC;
kk) BAX; BAK; BCKDHA; ICAM-1; PERK; SIRT-1; and MYC;
ll) BAX; BAK; BCKDHA; ICAM-1; PERK; SIRT-1; MYC; GGTAT; CMAH; LPL, LPLA2; PPT1; and LIPA;
mm) BAX; BAK; BCKDHA; LPL; LPLA2; GGTA1; and CMAH;
nn)BAX; BAK; BCKDHA; PERK; LPL; LPLA2; GGTA1; and CMAH;
oo)BAX; BAK; BCKDHA; MYC; LPL; LPLA2; GGTAT; and CMAH;
pp)BAX; BAK; BCKDHA; MYC; PERK; LPL; LPLA2; GGTA1; and CMAH;
qq)BAX; BAK; BCKDHA; LPL; LPLA2; GGTA1; CMAH; and PPT1;
rr) BAX; BAK; BCKDHA; PERK; LPL; LPLA2; GGTA1; CMAH; and PPT1;
ss) BAX; BAK; BCKDHA; MYC; LPL; LPLA2; GGTA1; CMAH; and PPT1;
tt) BAX; BAK; BCKDHA; MYC; PERK; LPL; LPLA2; GGTA1; CMAH; and PPT1;
uu) BAX; BAK; BCKDHA; ICAM-1; and SIRT-1;
vv) BAX; BAK; BCKDHA; and ICAM-1;
ww) BAX; BAK; BCKDHB; ICAM-1; GGTA1; CMAH; LPL; LPLA2; and PPT1;
xx) BAX; BAK; BCKDHB; ICAM-1; SIRT-1; GGTA1; CMAH; LPL; LPLA2; and PPT1;
yy) BAX; BAK; BCKDHB; ICAM-1; SIRT-1; MYC; GGTA1; CMAH; LPL; LPLA2; and PPT1;
zz) BAX; BAK; BCKDHB; ICAM-1; SIRT-1; MYC; GGTA1; CMAH; LPL, LPLA2; PPT1; and LIPA;
aaa) BAX; BAK; BCKDHB; ICAM-1; GGTA1; CMAH; LPLA2; PPT1; and LIPA;
bbb) BAX; BAK; BCKDHB; ICAM-1; SIRT-1; GGTA1; CMAH; LPLA2; PPT1; and LIPA;
ccc) BAX; BAK; BCKDHB; ICAM-1; SIRT-1; MYC; GGTA1; CMAH; LPLA2; PPT1; and LIPA;
ddd) BAX; BAK; BCKDHB; ICAM-1; LPL; LPLA2; and PPT1;
eee) BAX; BAK; BCKDHB; ICAM-1; SIRT-1; LPL, LPLA2; and PPT1;
fff) BAX; BAK; BCKDHB; ICAM-1; SIRT-1; MYC; LPL; LPLA2; and PPT1;
ggg) BAX; BAK; BCKDHB; ICAM-1; SIRT-1; MYC; LPL; LPLA2; PPT1; and LIPA;
hhh) BAX; BAK; BCKDHB; ICAM-1; SIRT-1; and MYC;
iii) BAX; BAK; BCKDHB; ICAM-1; PERK; SIRT-1; and MYC;
jjj) BAX; BAK; BCKDHB; ICAM-1; PERK; SIRT-1; MYC; GGTA1; CMAH; LPL, LPLA2; PPT1; and LIPA;
kkk) BAX; BAK; BCKDHB; LPL; LPLA2; GGTA1; and CMAH;
lll) BAX; BAK; BCKDHB; PERK; LPL; LPLA2; GGTA1; and CMAH;
mmm) BAX; BAK; BCKDHB; MYC; LPL; LPLA2; GGTA1; and CMAH;
nnn) BAX; BAK; BCKDHB; MYC; PERK; LPL; LPLA2; GGTA1; and CMAH;
ooo) BAX; BAK; BCKDHB; LPL; LPLA2; GGTA1; CMAH; and PPT1;
ppp) BAX; BAK; BCKDHB; PERK; LPL; LPLA2; GGTA1; CMAH; and PPT1;
qqq) BAX; BAK; BCKDHB; MYC; LPL; LPLA2; GGTA1; CMAH; and PPT1;
rrr) BAX; BAK; BCKDHB; MYC; PERK; LPL; LPLA2; GGTA1; CMAH; and PPT1;
sss) BAX; BAK; BCKDHB; ICAM-1; and SIRT-1;
ttt) BAX; BAK; BCKDHB; and ICAM-1;
uuu) BAX; BAK; BCKDHA; BCKDHB; ICAM-1; GGTA1; CMAH; LPL; LPLA2; and PPT1;
vvv) BAX; BAK; BCKDHA; BCKDHB; ICAM-1; SIRT-1; GGTA1; CMAH; LPL; LPLA2; and PPT1;
www) BAX; BAK; BCKDHA; BCKDHB; ICAM-1; SIRT-1; MYC; GGTA1; CMAH; LPL; LPLA2; and PPT1;
xxx) BAX; BAK; BCKDHA; BCKDHB; ICAM-1; SIRT-1; MYC; GGTA1; CMAH; LPL, LPLA2; PPT1; and LIPA;
yyy) BAX; BAK; BCKDHA; BCKDHB; ICAM-1; GGTA1; CMAH; LPLA2; PPT1; and LIPA;
zzz) BAX; BAK; BCKDHA; BCKDHB; ICAM-1; SIRT-1; GGTA1; CMAH; LPLA2; PPT1; and LIPA;
aaaa) BAX; BAK; BCKDHA; BCKDHB; ICAM-1; SIRT-1; MYC; GGTA1; CMAH; LPLA2; PPT1; and LIPA;
bbbb) BAX; BAK; BCKDHA; BCKDHB; ICAM-1; LPL; LPLA2; and PPT1;
cccc) BAX; BAK; BCKDHA; BCKDHB; ICAM-1; SIRT-1; LPL, LPLA2; and PPT1;
dddd) BAX; BAK; BCKDHA; BCKDHB; ICAM-1; SIRT-1; MYC; LPL; LPLA2; and PPT1;
eeee) BAX; BAK; BCKDHA; BCKDHB; ICAM-1; SIRT-1; MYC; LPL; LPLA2; PPT1; and LIPA;
ffff) BAX; BAK; BCKDHA; BCKDHB; ICAM-1; SIRT-1; and MYC;
gggg) BAX; BAK; BCKDHA; BCKDHB; ICAM-1; PERK; SIRT-1; and MYC;
hhhh) BAX; BAK; BCKDHA; BCKDHB; ICAM-1; PERK; SIRT-1; MYC; GGTA1; CMAH; LPL, LPLA2; PPT1; and LIPA;
iiii)BAX; BAK; BCKDHA; BCKDHB; LPL; LPLA2; GGTA1; and CMAH;
jjjj)BAX; BAK; BCKDHA; BCKDHB; PERK; LPL; LPLA2; GGTA1; and CMAH;
kkkk) BAX; BAK; BCKDHA; BCKDHB; MYC; LPL; LPLA2; GGTAT; and CMAH;
llll)BAX; BAK; BCKDHA; BCKDHB; MYC; PERK; LPL; LPLA2; GGTA1; and CMAH;
mmmm) BAX; BAK; BCKDHA; BCKDHB; LPL; LPLA2; GGTA1; CMAH; and PPT1;
nnnn) BAX; BAK; BCKDHA; BCKDHB; PERK; LPL; LPLA2; GGTA1; CMAH; and PPT1;
oooo) BAX; BAK; BCKDHA; BCKDHB; MYC; LPL; LPLA2; GGTAT; CMAH; and PPT1;
pppp) BAX; BAK; BCKDHA; BCKDHB; MYC; PERK; LPL; LPLA2; GGTA1; CMAH; and PPT1;
qqqq) BAX; BAK; BCKDHA; BCKDHB; ICAM-1; and SIRT-1; or rrrr) BAX; BAK; BCKDHA; BCKDHB; and ICAM-1.

In certain embodiments, the present disclosure is directed to the above-described methods for culturing a population of modified mammalian cells expressing a recombinant product of interest or producing a recombinant product of interest comprising culturing a population of mammalian cells expressing a recombinant product of interest, wherein the recombinant product of interest is encoded by a nucleic acid sequence. In certain embodiments, the nucleic acid sequence is integrated in the cellular genome of the modified cells at one or more targeted locations. In certain embodiments, the recombinant product of interest expressed by the modified cells is encoded by a nucleic acid sequence that is randomly integrated in the cellular genome of the mammalian cells. In certain embodiments, the recombinant product of interest comprises a recombinant viral vector. In certain embodiments, the recombinant product of interest comprises a recombinant viral particle. In certain embodiments, the recombinant product of interest comprises a recombinant protein. In certain embodiments, the recombinant protein is an antibody or an antigen-binding fragment thereof. In certain embodiments, the antibody is a multispecific antibody or an antigen-binding fragment thereof. In certain embodiments, the antibody consists of a single heavy chain sequence and a single light chain sequence or antigen-binding fragments thereof. In certain embodiments, the antibody is a chimeric antibody, a human antibody or a humanized antibody. In certain embodiments, the antibody is a monoclonal antibody. In certain embodiments, the methods comprise purifying the recombinant product of interest, harvesting the product of interest, and/or formulating the product of interest. In certain embodiments, the modified cell is a modified CHO cell. In certain embodiments, the modified cell is a modified HEK 293, HEK 293T, BHK, A549, or HeLa cell

In certain embodiments, the subject matter of the present disclosure is directed to compositions comprising a modified mammalian cell as described herein.

In certain embodiments, the subject matter of the present disclosure is directed to methods of producing a recombinant product of interest comprising: i) culturing a modified mammalian cell comprising an exogenous nucleic acid encoding a recombinant product of interest as described herein; ii) recovering the recombinant product of interest from a cultivation medium or the modified mammalian cells, wherein the modified cells expressing the recombinant product of interest exhibit reduced or eliminated expression of one or more of the following GAG; BAX; BAK; ICAM-1; SIRT-1; PERK; MYC; GGTA1; CMAH; LPLA2; PPT1; BCKDHA; BCKDHB; LPL; and/or LIPA.

In certain embodiments, the present disclosure is directed to methods for producing a modified mammalian cell, comprising: applying a nuclease-assisted and/or nucleic acid targeting at least one endogenous gene selected from the group of genes consisting GAG; BAX; BAK; ICAM-1; PERK; SIRT-1; MYC; GGTA1; CMAH; LPLA2; PPT1; BCKDHA; BCKDHB; LPL; and LIPA, in the mammalian cell to reduce or eliminate the expression of said endogenous gene, and selecting the modified mammalian cell wherein the expression of said endogenous gene has been reduced or eliminated as compared to an unmodified mammalian cell. In certain embodiments, the nuclease-assisted gene targeting system is selected from the group consisting of CRISPR/Cas9, CRISPR/Cpf1, zinc-finger nuclease, TALEN or meganuclease.

In certain embodiments, the modification to the modified mammalian cells described herein is performed before the introduction of the exogenous nucleic acid encoding the recombinant product of interest, or after the introduction of the exogenous nucleic acid encoding the recombinant product of interest.

In certain embodiments, the reduction of gene expression in the modified mammalian cells of the present disclosure is mediated by RNA silencing. In certain embodiments, the RNA silencing is selected from the group consisting of siRNA gene targeting and knockdown, shRNA gene targeting and knockdown, and miRNA gene targeting and knockdown.

In certain embodiments, the modified cells expressing a recombinant product of interest exhibit reduced or eliminated expression of:
a) BAX; BAK; ICAM-1; GGTA1; CMAH; LPL; LPLA2; and PPT1;
b) BAX; BAK; ICAM-1; SIRT-1; GGTA1; CMAH; LPL; LPLA2; and PPT1;
c) BAX; BAK; ICAM-1; SIRT-1; MYC; GGTA1; CMAH; LPL; LPLA2; and PPT1;
d) BAX; BAK; ICAM-1; SIRT-1; MYC; GGTA1; CMAH; LPL, LPLA2; PPT1; and LIPA;
e) BAX; BAK; ICAM-1; GGTA1; CMAH; LPLA2; PPT1; and LIPA;
f) BAX; BAK; ICAM-1; SIRT-1; GGTA1; CMAH; LPLA2; PPT1; and LIPA;
g) BAX; BAK; ICAM-1; SIRT-1; MYC; GGTA1; CMAH; LPLA2; PPT1; and LIPA;
h) BAX; BAK; ICAM-1; LPL; LPLA2; and PPT1;
i) BAX; BAK; ICAM-1; SIRT-1; LPL, LPLA2; and PPT1;
j) BAX; BAK; ICAM-1; SIRT-1; MYC; LPL; LPLA2; and PPT1;
k) BAX; BAK; ICAM-1; SIRT-1; MYC; LPL; LPLA2; PPT1; and LIPA;
l) BAX; BAK; ICAM-1; SIRT-1; and MYC;
m) BAX; BAK; ICAM-1; PERK; SIRT-1; and MYC;
n) BAX; BAK; ICAM-1; SIRT-1; PERK; MYC; GGTA1; CMAH; LPL, LPLA2; PPT1; and LIPA;
o) BAX; BAK; LPL; LPLA2; GGTA1; and CMAH;
p) BAX; BAK; PERK; LPL; LPLA2; GGTA1; and CMAH;
q) BAX; BAK; MYC; LPL; LPLA2; GGTA1; and CMAH;
r) BAX; BAK; MYC; PERK; LPL; LPLA2; GGTA1; and CMAH;
s) BAX; BAK; LPL; LPLA2; GGTA1; CMAH; and PPT1;
t) BAX; BAK; PERK; LPL; LPLA2; GGTA1; CMAH; and PPT1;
u) BAX; BAK; MYC; LPL; LPLA2; GGTA1; CMAH; and PPT1;
v) BAX; BAK; MYC; PERK; LPL; LPLA2; GGTA1; CMAH; and PPT1;
w) BAX; BAK; ICAM-1; and SIRT-1;
x) BAX; BAK; and ICAM-1
y) BAX; BAK; BCKDHA; ICAM-1; GGTA1; CMAH; LPL; LPLA2; and PPT1;
z) BAX; BAK; BCKDHA; ICAM-1; SIRT-1; GGTA1; CMAH; LPL; LPLA2; and PPT1;
aa) BAX; BAK; BCKDHA; ICAM-1; SIRT-1; MYC; GGTA1; CMAH; LPL; LPLA2; and PPT1;
bb) BAX; BAK; BCKDHA; ICAM-1; SIRT-1; MYC; GGTA1; CMAH; LPL, LPLA2; PPT1; and LIPA;
cc) BAX; BAK; BCKDHA; ICAM-1; GGTAT; CMAH; LPLA2; PPT1; and LIPA;
dd)BAX; BAK; BCKDHA; ICAM-1; SIRT-1; GGTAT; CMAH; LPLA2; PPT1; and LIPA;
ee) BAX; BAK; BCKDHA; ICAM-1; SIRT-1; MYC; GGTA1; CMAH; LPLA2; PPT1; and LIPA;
ff) BAX; BAK; BCKDHA; ICAM-1; LPL; LPLA2; and PPT1;
gg)BAX; BAK; BCKDHA; ICAM-1; SIRT-1; LPL, LPLA2; and PPT1;
hh)BAX; BAK; BCKDHA; ICAM-1; SIRT-1; MYC; LPL; LPLA2; and PPT1;
ii) BAX; BAK; BCKDHA; ICAM-1; SIRT-1; MYC; LPL; LPLA2; PPT1; and LIPA;
jj) BAX; BAK; BCKDHA; ICAM-1; SIRT-1; and MYC;
kk)BAX; BAK; BCKDHA; ICAM-1; PERK; SIRT-1; and MYC;
ll) BAX; BAK; BCKDHA; ICAM-1; PERK; SIRT-1; MYC; GGTA1; CMAH; LPL, LPLA2; PPT1; and LIPA;
mm) BAX; BAK; BCKDHA; LPL; LPLA2; GGTA1; and CMAH;
nn)BAX; BAK; BCKDHA; PERK; LPL; LPLA2; GGTA1; and CMAH;
oo)BAX; BAK; BCKDHA; MYC; LPL; LPLA2; GGTA1; and CMAH;
pp)BAX; BAK; BCKDHA; MYC; PERK; LPL; LPLA2; GGTA1; and CMAH;
qq)BAX; BAK; BCKDHA; LPL; LPLA2; GGTAT; CMAH; and PPT1;
rr) BAX; BAK; BCKDHA; PERK; LPL; LPLA2; GGTA1; CMAH; and PPT1;
ss) BAX; BAK; BCKDHA; MYC; LPL; LPLA2; GGTA1; CMAH; and PPT1;
tt) BAX; BAK; BCKDHA; MYC; PERK; LPL; LPLA2; GGTA1; CMAH; and PPT1;
uu)BAX; BAK; BCKDHA; ICAM-1; and SIRT-1;
vv)BAX; BAK; BCKDHA; and ICAM-1;
ww) BAX; BAK; BCKDHB; ICAM-1; GGTA1; CMAH; LPL; LPLA2; and PPT1;
xx)BAX; BAK; BCKDHB; ICAM-1; SIRT-1; GGTA1; CMAH; LPL; LPLA2; and PPT1;
yy)BAX; BAK; BCKDHB; ICAM-1; SIRT-1; MYC; GGTA1; CMAH; LPL; LPLA2; and PPT1;
zz) BAX; BAK; BCKDHB; ICAM-1; SIRT-1; MYC; GGTA1; CMAH; LPL, LPLA2; PPT1; and LIPA;
aaa) BAX; BAK; BCKDHB; ICAM-1; GGTA1; CMAH; LPLA2; PPT1; and LIPA;
bbb) BAX; BAK; BCKDHB; ICAM-1; SIRT-1; GGTA1; CMAH; LPLA2; PPT1; and LIPA;
ccc) BAX; BAK; BCKDHB; ICAM-1; SIRT-1; MYC; GGTA1; CMAH; LPLA2; PPT1; and LIPA;
ddd) BAX; BAK; BCKDHB; ICAM-1; LPL; LPLA2; and PPT1;
eee) BAX; BAK; BCKDHB; ICAM-1; SIRT-1; LPL, LPLA2; and PPT1;
fff) BAX; BAK; BCKDHB; ICAM-1; SIRT-1; MYC; LPL; LPLA2; and PPT1;
ggg) BAX; BAK; BCKDHB; ICAM-1; SIRT-1; MYC; LPL; LPLA2; PPT1; and LIPA;
hhh) BAX; BAK; BCKDHB; ICAM-1; SIRT-1; and MYC;
iii) BAX; BAK; BCKDHB; ICAM-1; PERK; SIRT-1; and MYC;
jjj) BAX; BAK; BCKDHB; ICAM-1; PERK; SIRT-1; MYC; GGTA1; CMAH; LPL, LPLA2; PPT1; and LIPA;
kkk) BAX; BAK; BCKDHB; LPL; LPLA2; GGTA1; and CMAH;
lll) BAX; BAK; BCKDHB; PERK; LPL; LPLA2; GGTA1; and CMAH;
mmm) BAX; BAK; BCKDHB; MYC; LPL; LPLA2; GGTA1; and CMAH;
nnn) BAX; BAK; BCKDHB; MYC; PERK; LPL; LPLA2; GGTA1; and CMAH;
ooo) BAX; BAK; BCKDHB; LPL; LPLA2; GGTA1; CMAH; and PPT1;
ppp) BAX; BAK; BCKDHB; PERK; LPL; LPLA2; GGTA1; CMAH; and PPT1;
qqq) BAX; BAK; BCKDHB; MYC; LPL; LPLA2; GGTA1; CMAH; and PPT1;
rrr) BAX; BAK; BCKDHB; MYC; PERK; LPL; LPLA2; GGTA1; CMAH; and PPT1;
sss) BAX; BAK; BCKDHB; ICAM-1; and SIRT-1;
ttt) BAX; BAK; BCKDHB; and ICAM-1;
uuu) BAX; BAK; BCKDHA; BCKDHB; ICAM-1; GGTA1; CMAH; LPL; LPLA2; and PPT1;
vvv) BAX; BAK; BCKDHA; BCKDHB; ICAM-1; SIRT-1; GGTA1; CMAH; LPL; LPLA2; and PPT1;
www) BAX; BAK; BCKDHA; BCKDHB; ICAM-1; SIRT-1; MYC; GGTA1; CMAH; LPL; LPLA2; and PPT1;
xxx) BAX; BAK; BCKDHA; BCKDHB; ICAM-1; SIRT-1; MYC; GGTA1; CMAH; LPL, LPLA2; PPT1; and LIPA;
yyy) BAX; BAK; BCKDHA; BCKDHB; ICAM-1; GGTA1; CMAH; LPLA2; PPT1; and LIPA;
zzz) BAX; BAK; BCKDHA; BCKDHB; ICAM-1; SIRT-1; GGTA1; CMAH; LPLA2; PPT1; and LIPA;
aaaa) BAX; BAK; BCKDHA; BCKDHB; ICAM-1; SIRT-1; MYC; GGTA1; CMAH; LPLA2; PPT1; and LIPA;
bbbb) BAX; BAK; BCKDHA; BCKDHB; ICAM-1; LPL; LPLA2; and PPT1;
cccc) BAX; BAK; BCKDHA; BCKDHB; ICAM-1; SIRT-1; LPL, LPLA2; and PPT1;
dddd) BAX; BAK; BCKDHA; BCKDHB; ICAM-1; SIRT-1; MYC; LPL; LPLA2; and PPT1;
eeee) BAX; BAK; BCKDHA; BCKDHB; ICAM-1; SIRT-1; MYC; LPL; LPLA2; PPT1; and LIPA;
ffff) BAX; BAK; BCKDHA; BCKDHB; ICAM-1; SIRT-1; and MYC;
gggg) BAX; BAK; BCKDHA; BCKDHB; ICAM-1; PERK; SIRT-1; and MYC;
hhhh) BAX; BAK; BCKDHA; BCKDHB; ICAM-1; PERK; SIRT-1; MYC; GGTA1; CMAH; LPL, LPLA2; PPT1; and LIPA;
iiii)BAX; BAK; BCKDHA; BCKDHB; LPL; LPLA2; GGTA1; and CMAH;
jjjj)BAX; BAK; BCKDHA; BCKDHB; PERK; LPL; LPLA2; GGTA1; and CMAH;
kkkk) BAX; BAK; BCKDHA; BCKDHB; MYC; LPL; LPLA2; GGTA1; and CMAH;
llll)BAX; BAK; BCKDHA; BCKDHB; MYC; PERK; LPL; LPLA2; GGTA1; and CMAH;
mmmm) BAX; BAK; BCKDHA; BCKDHB; LPL; LPLA2; GGTA1; CMAH; and PPT1;
nnnn) BAX; BAK; BCKDHA; BCKDHB; PERK; LPL; LPLA2; GGTA1; CMAH; and PPT1;
oooo) BAX; BAK; BCKDHA; BCKDHB; MYC; LPL; LPLA2; GGTA1; CMAH; and PPT1;
pppp) BAX; BAK; BCKDHA; BCKDHB; MYC; PERK; LPL; LPLA2; GGTA1; CMAH; and PPT1;
qqqq) BAX; BAK; BCKDHA; BCKDHB; ICAM-1; and SIRT-1; or rrrr) BAX; BAK; BCKDHA; BCKDHB; and ICAM-1.

In certain embodiments, the modified cells expressing the recombinant product of interest exhibit reduced or eliminated expression of:
a) GAG; BAX; BAK; ICAM-1; GGTA1; CMAH; LPL; LPLA2; and PPT1;
b) GAG; BAX; BAK; ICAM-1; SIRT-1; GGTA1; CMAH; LPL; LPLA2; and PPT1;
c) GAG; BAX; BAK; ICAM-1; SIRT-1; MYC; GGTA1; CMAH; LPL; LPLA2; and PPT1;
d) GAG; BAX; BAK; ICAM-1; SIRT-1; MYC; GGTA1; CMAH; LPL, LPLA2; PPT1; and LIPA;
e) GAG; BAX; BAK; ICAM-1; GGTA1; CMAH; LPLA2; PPT1; and LIPA;
f) GAG; BAX; BAK; ICAM-1; SIRT-1; GGTA1; CMAH; LPLA2; PPT1; and LIPA;
g) GAG; BAX; BAK; ICAM-1; SIRT-1; MYC; GGTA1; CMAH; LPLA2; PPT1; and LIPA;
h) GAG; BAX; BAK; ICAM-1; LPL; LPLA2; and PPT1;
i) GAG; BAX; BAK; ICAM-1; SIRT-1; LPL, LPLA2; and PPT1;
j) GAG; BAX; BAK; ICAM-1; SIRT-1; MYC; LPL; LPLA2; and PPT1;
k) GAG; BAX; BAK; ICAM-1; SIRT-1; MYC; LPL; LPLA2; PPT1; and LIPA;
l) GAG; BAX; BAK; ICAM-1; SIRT-1; and MYC;
m) GAG; BAX; BAK; ICAM-1; PERK; SIRT-1; and MYC;
n) GAG; BAX; BAK; ICAM-1; PERK; SIRT-1; MYC; GGTA1; CMAH; LPL, LPLA2; PPT1; and LIPA;
o) GAG; BAX; BAK; LPL; LPLA2; GGTA1; and CMAH;
p) GAG; BAX; BAK; PERK; LPL; LPLA2; GGTA1; and CMAH;
q) GAG; BAX; BAK; MYC; LPL; LPLA2; GGTA1; and CMAH;
r) GAG; BAX; BAK; MYC; PERK; LPL; LPLA2; GGTA1; and CMAH;
s) GAG; BAX; BAK; LPL; LPLA2; GGTA1; CMAH; and PPT1;
t) GAG; BAX; BAK; PERK; LPL; LPLA2; GGTA1; CMAH; and PPT1;
u) GAG; BAX; BAK; MYC; LPL; LPLA2; GGTA1; CMAH; and PPT1;
v) GAG; BAX; BAK; MYC; PERK; LPL; LPLA2; GGTA1; CMAH; and PPT1;
w) GAG; BAX; BAK; ICAM-1; and SIRT-1;
x) GAG; BAX; BAK; and ICAM-1;
y) BAX; BAK; BCKDHA; ICAM-1; GGTA1; CMAH; LPL; LPLA2; and PPT1;
z) BAX; BAK; BCKDHA; ICAM-1; SIRT-1; GGTA1; CMAH; LPL; LPLA2; and PPT1;
aa) BAX; BAK; BCKDHA; ICAM-1; SIRT-1; MYC; GGTA1; CMAH; LPL; LPLA2; and PPT1;
bb) BAX; BAK; BCKDHA; ICAM-1; SIRT-1; MYC; GGTA1; CMAH; LPL, LPLA2; PPT1; and LIPA;
cc) BAX; BAK; BCKDHA; ICAM-1; GGTA1; CMAH; LPLA2; PPT1; and LIPA;
dd)BAX; BAK; BCKDHA; ICAM-1; SIRT-1; GGTA1; CMAH; LPLA2; PPT1; and LIPA;
ee) BAX; BAK; BCKDHA; ICAM-1; SIRT-1; MYC; GGTA1; CMAH; LPLA2; PPT1; and LIPA;
ff) BAX; BAK; BCKDHA; ICAM-1; LPL; LPLA2; and PPT1;
gg)BAX; BAK; BCKDHA; ICAM-1; SIRT-1; LPL, LPLA2; and PPT1;
hh)BAX; BAK; BCKDHA; ICAM-1; SIRT-1; MYC; LPL; LPLA2; and PPT1;
ii) BAX; BAK; BCKDHA; ICAM-1; SIRT-1; MYC; LPL; LPLA2; PPT1; and LIPA;
jj) BAX; BAK; BCKDHA; ICAM-1; SIRT-1; and MYC;
kk)BAX; BAK; BCKDHA; ICAM-1; PERK; SIRT-1; and MYC;
ll) BAX; BAK; BCKDHA; ICAM-1; PERK; SIRT-1; MYC; GGTA1; CMAH; LPL, LPLA2; PPT1; and LIPA;
mm) BAX; BAK; BCKDHA; LPL; LPLA2; GGTA1; and CMAH;
nn)BAX; BAK; BCKDHA; PERK; LPL; LPLA2; GGTA1; and CMAH;
oo)BAX; BAK; BCKDHA; MYC; LPL; LPLA2; GGTA1; and CMAH;
pp)BAX; BAK; BCKDHA; MYC; PERK; LPL; LPLA2; GGTA1; and CMAH;
qq)BAX; BAK; BCKDHA; LPL; LPLA2; GGTA1; CMAH; and PPT1;
rr) BAX; BAK; BCKDHA; PERK; LPL; LPLA2; GGTA1; CMAH; and PPT1;
ss) BAX; BAK; BCKDHA; MYC; LPL; LPLA2; GGTA1; CMAH; and PPT1;
tt) BAX; BAK; BCKDHA; MYC; PERK; LPL; LPLA2; GGTA1; CMAH; and PPT1;
uu)BAX; BAK; BCKDHA; ICAM-1; and SIRT-1;
vv)BAX; BAK; BCKDHA; and ICAM-1;
ww) BAX; BAK; BCKDHB; ICAM-1; GGTA1; CMAH; LPL; LPLA2; and PPT1;
xx)BAX; BAK; BCKDHB; ICAM-1; SIRT-1; GGTA1; CMAH; LPL; LPLA2; and PPT1;
yy)BAX; BAK; BCKDHB; ICAM-1; SIRT-1; MYC; GGTA1; CMAH; LPL; LPLA2; and PPT1;
zz) BAX; BAK; BCKDHB; ICAM-1; SIRT-1; MYC; GGTA1; CMAH; LPL, LPLA2; PPT1; and LIPA;
aaa) BAX; BAK; BCKDHB; ICAM-1; GGTA1; CMAH; LPLA2; PPT1; and LIPA;
bbb) BAX; BAK; BCKDHB; ICAM-1; SIRT-1; GGTA1; CMAH; LPLA2; PPT1; and LIPA;
ccc) BAX; BAK; BCKDHB; ICAM-1; SIRT-1; MYC; GGTA1; CMAH; LPLA2; PPT1; and LIPA;
ddd) BAX; BAK; BCKDHB; ICAM-1; LPL; LPLA2; and PPT1;
eee) BAX; BAK; BCKDHB; ICAM-1; SIRT-1; LPL, LPLA2; and PPT1;
fff) BAX; BAK; BCKDHB; ICAM-1; SIRT-1; MYC; LPL; LPLA2; and PPT1;
ggg) BAX; BAK; BCKDHB; ICAM-1; SIRT-1; MYC; LPL; LPLA2; PPT1; and LIPA;
hhh) BAX; BAK; BCKDHB; ICAM-1; SIRT-1; and MYC;
iii) BAX; BAK; BCKDHB; ICAM-1; PERK; SIRT-1; and MYC;
jjj) BAX; BAK; BCKDHB; ICAM-1; PERK; SIRT-1; MYC; GGTA1; CMAH; LPL, LPLA2; PPT1; and LIPA;
kkk) BAX; BAK; BCKDHB; LPL; LPLA2; GGTA1; and CMAH;
lll) BAX; BAK; BCKDHB; PERK; LPL; LPLA2; GGTA1; and CMAH;
mmm) BAX; BAK; BCKDHB; MYC; LPL; LPLA2; GGTA1; and CMAH;
nnn) BAX; BAK; BCKDHB; MYC; PERK; LPL; LPLA2; GGTA1; and CMAH;
ooo) BAX; BAK; BCKDHB; LPL; LPLA2; GGTA1; CMAH; and PPT1;
ppp) BAX; BAK; BCKDHB; PERK; LPL; LPLA2; GGTA1; CMAH; and PPT1;
qqq) BAX; BAK; BCKDHB; MYC; LPL; LPLA2; GGTA1; CMAH; and PPT1;
rrr) BAX; BAK; BCKDHB; MYC; PERK; LPL; LPLA2; GGTA1; CMAH; and PPT1;
sss) BAX; BAK; BCKDHB; ICAM-1; and SIRT-1;
ttt) BAX; BAK; BCKDHB; and ICAM-1;
uuu) BAX; BAK; BCKDHA; BCKDHB; ICAM-1; GGTA1; CMAH; LPL; LPLA2; and PPT1;
vvv) BAX; BAK; BCKDHA; BCKDHB; ICAM-1; SIRT-1; GGTA1; CMAH; LPL; LPLA2; and PPT1;
www) BAX; BAK; BCKDHA; BCKDHB; ICAM-1; SIRT-1; MYC; GGTA1; CMAH; LPL; LPLA2; and PPT1;
xxx) BAX; BAK; BCKDHA; BCKDHB; ICAM-1; SIRT-1; MYC; GGTA1; CMAH; LPL, LPLA2; PPT1; and LIPA;
yyy) BAX; BAK; BCKDHA; BCKDHB; ICAM-1; GGTA1; CMAH; LPLA2; PPT1; and LIPA;
zzz) BAX; BAK; BCKDHA; BCKDHB; ICAM-1; SIRT-1; GGTA1; CMAH; LPLA2; PPT1; and LIPA;
aaaa) BAX; BAK; BCKDHA; BCKDHB; ICAM-1; SIRT-1; MYC; GGTA1; CMAH; LPLA2; PPT1; and LIPA;
bbbb) BAX; BAK; BCKDHA; BCKDHB; ICAM-1; LPL; LPLA2; and PPT1;
cccc) BAX; BAK; BCKDHA; BCKDHB; ICAM-1; SIRT-1; LPL, LPLA2; and PPT1;
dddd) BAX; BAK; BCKDHA; BCKDHB; ICAM-1; SIRT-1; MYC; LPL; LPLA2; and PPT1;
eeee) BAX; BAK; BCKDHA; BCKDHB; ICAM-1; SIRT-1; MYC; LPL; LPLA2; PPT1; and LIPA;
ffff) BAX; BAK; BCKDHA; BCKDHB; ICAM-1; SIRT-1; and MYC;
gggg) BAX; BAK; BCKDHA; BCKDHB; ICAM-1; PERK; SIRT-1; and MYC;
hhhh) BAX; BAK; BCKDHA; BCKDHB; ICAM-1; PERK; SIRT-1; MYC; GGTA1; CMAH; LPL, LPLA2; PPT1; and LIPA;
iiii)BAX; BAK; BCKDHA; BCKDHB; LPL; LPLA2; GGTA1; and CMAH;
jjjj)BAX; BAK; BCKDHA; BCKDHB; PERK; LPL; LPLA2; GGTA1; and CMAH;
kkkk) BAX; BAK; BCKDHA; BCKDHB; MYC; LPL; LPLA2; GGTA1; and CMAH;
llll)BAX; BAK; BCKDHA; BCKDHB; MYC; PERK; LPL; LPLA2; GGTA1; and CMAH;
mmmm) BAX; BAK; BCKDHA; BCKDHB; LPL; LPLA2; GGTA1; CMAH; and PPT1;
nnnn) BAX; BAK; BCKDHA; BCKDHB; PERK; LPL; LPLA2; GGTA1; CMAH; and PPT1;
oooo) BAX; BAK; BCKDHA; BCKDHB; MYC; LPL; LPLA2; GGTA1; CMAH; and PPT1;
pppp) BAX; BAK; BCKDHA; BCKDHB; MYC; PERK; LPL; LPLA2; GGTA1; CMAH; and PPT1;
qqqq) BAX; BAK; BCKDHA; BCKDHB; ICAM-1; and SIRT-1; or rrrr) BAX; BAK; BCKDHA; BCKDHB; and ICAM-1.

In certain embodiments of the above-described methods for expressing a recombinant product of interest, the recombinant product of interest is encoded by a nucleic acid sequence.

In certain embodiments, the nucleic acid sequence encoding the recombinant product of interest is integrated in the cellular genome of the modified cells at one or more targeted locations. In certain embodiments, the nucleic acid sequence encoding the recombinant product of interest is randomly integrated in the cellular genome of the mammalian cells. In certain embodiments, the nucleic acid sequence encoding the recombinant product of interest is integrated into the cellular genome of a mammalian cell by means of a transposase-mediated gene integration (using, for e.g., Lonza's GS piggyBac transposase system, ATUM's Leap-In transposase system, or Probiogen's DirectedLuck transposase with epigenetic targeting).

In certain embodiments, the recombinant product of interest comprises a viral vector. In certain embodiments, the recombinant product of interest comprises a viral particle. In certain embodiments, the recombinant product of interest comprises a recombinant protein. In certain embodiments, the recombinant protein is an antibody or an antigen-binding fragment thereof. In certain embodiments, antibody is a multispecific antibody or an antigen-binding fragment thereof. In certain embodiments, the antibody consists of a single heavy chain sequence and a single light chain sequence or antigen-binding fragments thereof. In certain embodiments, the antibody is a chimeric antibody, a human antibody or a humanized antibody. In certain embodiments, the antibody is a monoclonal antibody.

In certain embodiments, the subject matter of the present disclosure comprises purifying the product of interest expressed by a modified mammalian cell as disclosed herein, harvesting the product of interest, and/or formulating the product of interest.

### 4. BRIEF DESCRIPTION OF THE DRAWINGS

FIG. 1. CRISPR/Cas9 multiplex knockout (KO) method achieves high-efficiency knockouts (confirmed by LC-MS/MS). A schematic displaying the multiplex gene editing approach is illustrated. Individual gRNAs were first screened for each knockout target. The most potent gRNAs were multiplexed with Cas9 protein and transfected into cells sequentially to generate a highly edited pool of cells (>75% indel frequency). Percent indel was measured at the pool stage of each target to determine the probability of clones with all target genes knocked out. After single cell cloning (SCC), clones were analyzed and screened via PCR and Sanger sequencing to identify those with all targets knocked out. Top clones were selected to initiate production cultures to characterize their growth profiles. At the end of the production culture, the harvested cell culture fluid (HCCF) was submitted for LC-MS/MS for verification of knockouts at the protein level. The top knockout hosts were selected for cryopreservation to create cell banks.

FIGS. 2A-2B. Screening process and indel analysis for detecting knockout efficiencies. Sanger traces generated from workflow illustrated in FIG. 1 were analyzed using ICE software (Synthego) to determine editing efficiency.

FIG. 3. CRISPR/Cas9 multiplex knockout method achieves high-efficiency knockouts. Comparison of KO efficiency for each gene in the 10× transfected CHO pools. The 10× transfected pools were generated by transfecting 2× KO cells (BAX/BAK double KO cells). The KO targets for the 10× KO cells were BAX, BAK, SIRT-1, MYC, ICAM-1, LPLA2, LPL, PPT1, CMAH, and GGTA1.

FIG. 4. CRISPR/Cas9 multiplex knockout method achieves high-efficiency knockouts. Comparison of KO efficiency for each gene in the 6× KO CHO host. The percentage of KO measured in targeted pool by ICE. The indel percentage for the Bax and Bak1 genes were determined by Western blot analysis to be 100%. The indel percentage of the remaining genes were determined by genomic DNA sequencing analysis. The KO targets for 6× KO host were BAX, BAK, LPLA2 (also referred to as PLA2G7), LPL (also referred to as LPL1), CMAH, and GGTA1.

FIGS. 5A-5F. Key measurements and parameters for 6× knockout cells expressing mAb-M and mAb-N. Wild-type (WT) control and 6× knockout (KO) CHO cells were transfected with vectors expressing mAb-M and mAb-N and recovered pools were used to set up production run in 2-L bioreactor vessels. The 2-L bioreactor cultures of WT and 6× KO CHO pools were analyzed for (5A) mAb titer, (5B) cell-specific productivity (Qp), (5C) integrated viable cell count (IVCC), (5D) viable cell counts, and (5E) viability. The harvested materials from the 2-L bioreactor cultures for WT and 6× KO CHO pools were also analyzed for (5F) product quality in terms of % aggregates, charge distribution, alpha-Gal, and NGNA (N-Glycolylneuraminic acid) levels. The WT CHO control is the parental host with no gene knockouts. The WT-N production bioreactor run was stopped on day 12 because of a large decrease in viability. The KO targets for 6× CHO pools were BAX, BAK, LPLA2 (also referred to as LPA2G7), LPL, CMAH, and GGTA1. NGNA Method: The levels of N-glycolylneuraminic acid (NGNA) containing glycans were determined by hydrophilic interaction liquid chromatography-mass spectrometry (HILIC-MS). In this analysis, glycans were enzymatically released from the protein by treatment with PNGase F and then fluorescently labeled with a procaine-based IPC fluorophore (InstantPC, Agilent Technologies) prior to their separation by hydrophilic interaction liquid chromatography. Relative quantification of the labeled glycans was accomplished by integration of the glycan fluorescence signals and identification of the separated glycans was determined by mass spectrometry. Alpha-Gal Method: The levels of alpha-Gal containing glycans were determined by hydrophilic interaction liquid chromatography-mass spectrometry (HILIC-MS) analysis of sialidase treated glycans. In this analysis, glycans were first treated with sialidase to remove sialic acids and then enzymatically released from the protein by treatment with PNGase F. The release glycans were subsequently labeled with a procaine-based IPC fluorophore (InstantPC, Agilent Technologies) prior to their separation by hydrophilic interaction liquid chromatography. Relative quantification of the labeled glycans was accomplished by integration of the glycan fluorescence signals and identification of the separated glycans was determined by mass spectrometry.

FIG. 6. Comparison of KO efficiency for each gene in each of three 6× KO clonal CHO hosts. The indel percentage for the Bax and Bak1 genes were determined by Western blot analysis. The indel percentage of the remaining genes were determined by genomic DNA sequencing analysis. The KO targets for the 6× KO hosts were BAX, BAK, LPLA2 (also referred to as PLA2G7), LPL, CMAH, and GGTA1.

FIGS. 7A-7F. Key measurements and parameters for 6× knockout clonal CHO hosts expressing mAb-M. Wild-type (WT) control and 6× KO clonal CHO hosts were transfected with vectors expressing mAb-M and the recovered pools were used to set up the bioreactor production cultures in AMBR15 vessels. The bioreactor cultures were analyzed for (7A) titer, (7B) specific productivity (Qp), (7C) integrated viable cell count (IVCC), (7D) viable cell counts, and (7E) viability. The harvested materials from the bioreactor cultures for WT and 6× KO cells were also analyzed for (7F) product quality in terms of % aggregates and charge distribution. The KO targets for 6× KO host were BAX, BAK, LPLA2 (also referred to as PLA2G7), LPL, CMAH, and GGTA1.

FIG. 8. Key measurements and parameters for 6× knockout clonal CHO hosts expressing mAb-N. Wild-type (WT) control and 6× KO clonal CHO hosts were transfected with vectors expressing mAb-N and the recovered pools were used to set up bioreactor production cultures in AMBR15 vessels. The WT and 6× KO bioreactor cultures were analyzed at harvest for titer, specific productivity (Qp), % viability, viable cell count (VCC), integrated viable cell count (IVCC), and glycoform levels for alpha-Gal and NGNA (N-Glycolylneuraminic acid). The KO targets for 6× KO clonal host were BAX, BAK, LPLA2 (also referred to as PLA2G7), LPL, CMAH, and GGTA.

FIG. 9. CRISPR/Cas9 multiplex knockout method achieves high-efficiency knockouts. Comparison of KO efficiency for each gene in the 9× and 10× KO CHO hosts. The KO targets for 9× KO host were BAX, BAK, SIRT-1, ICAM-1, LPLA2, LPL, PPT1, CMAH, and GGTA1; the KO targets for 10× KO host were BAX, BAK, SIRT-1, MYC, ICAM-1, LPLA2, LPL, PPT1, CMAH, and GGTA1. The indel percentage for the Bax and Bak1 genes were determined by Western blot analysis. The indel percentage of the remaining genes were determined by genomic DNA sequencing analysis. Three different pools for the 9× KO CHO host and two different pools for the 10× KO CHO host were assessed for indel percentage. The 10× KO hosts differed from the 9× KO hosts in the use of Myc as a KO target.

FIGS. 10A-10F. Key measurements and parameters for 9× and 10× CHO hosts expressing mAb-H. Titer (10A), specific productivity (Qp) (10B), integrated viable cell count (IVCC) (10C) viable cell counts for days 0, 7, 10 and 12 (10D), viability (10E), and product quality analysis measuring % aggregates and charge variant (10F) for the 9× and 10× KO CHO pools for mAb-H. The KO targets for 9× KO were BAX, BAK, SIRT-1, ICAM-1, LPLA2, LPL, PPT1, CMAH, and GGTA1; the KO targets for 10× KO were BAX, BAK, SIRT-1, MYC, ICAM-1, LPLA2, LPL, PPT1, CMAH, and GGTA1. The wild-type (WT) CHO pool was the parental host with no gene knockouts. Three different pools for the 9× KO host and two different pools for the 10× KO host were assessed in fed-batch production cultures.

FIGS. 11A-11E. Key measurements and parameters for 9× and bOx CHO hosts expressing mAb-I. Wild-type (WT) control, 9× KO, and 10× KO hosts transfected to express mAb-I were cultured in AMBR15 bioreactors for 14 days. The CHO cultures were analyzed for (11A) titer, (11B) specific productivity (Qp), (1IC) integrated viable cell count (IVCC), (1 ID) viability, and (11 E) product quality in terms of % aggregates, charge distribution, and alpha-Gal levels. The KO targets for 9× KO were BAX, BAK, SIRT-1, ICAM-1, LPLA2, LPL, PPT1, CMAH, and GGTA1; the KO targets for 10× KO were BAX, BAK, SIRT-1, MYC, ICAM-1, LPLA2, LPL, PPT1, CMAH, and GGTA1.

FIG. 12. Key measurements and parameters for 10× knockout CHO clones expressing mAb-H. Wild-type (WT) and 10× KO clones expressing mAb-H were assessed for cell culture performance and product quality. WT and 10× KO CHO pools expressing mAb-H were single cell cloned and then screened to select the top mAb-expressing clones from each arm. The selected clones were evaluated over 14 days in AMBR15 production bioreactor cultures for titer, specific productivity (Qp), integrated viable cell count (IVCC), % viability, charge distribution, and % aggregates. The KO targets for 10× KO were BAX, BAK, SIRT-1, MYC, ICAM-1, LPLA2, LPL, PPT1, CMAH, and GGTA1.

FIG. 13. CRISPR/Cas9 multiplex knockout method achieves high-efficiency knockouts. Comparison of KO efficiency for each gene in each of four 8× KO clonal hosts. The indel percentage for the Bax and Bak1 genes were determined by Western blot analysis. The indel percentage of the remaining genes were determined by genomic DNA sequencing analysis. The KO targets for 8× KO clonal hosts were BAX, BAK, LPLA2 (also referred to as PLA2G7), LPL, CMAH, GGTA1, BCKDHA and BCKDHB.

FIG. 14. Key measurements and parameters for 8× clonal CHO hosts expressing mAb-N. Wild-type (WT) and four 8× KO CHO pools expressing mAb-N were assessed for cell culture performance and product quality. WT and 8× KO clonal CHO hosts were transfected to express mAb-N and the recovered pools were evaluated over 14 days in AMBR15 production bioreactor cultures for titer, specific productivity (Qp), viability, viable cell count (VCC), and integrated viable cell count (IVCC). The KO targets for 8× KO clonal hosts were BAX, BAK, LPLA2 (also referred to as PLA2G7), LPL, CMAH, GGTA1, BCKDHA and BCKDHB.

FIGS. 15A-15B. Key measurements and parameters for Penta (5×), 9× and 10× CHO cell lines expressing mAb-O and mAb-P. Wild-type (WT) and Penta (5×), 9×, and 10× KO pools expressing mAb-O or mAb-P were assessed for cell culture performance and product quality impact. WT and Penta (5×), 9×, and 10× KO hosts were transfected and the recovered pools were evaluated over—12 days in AMBR250 production bioreactor cultures. The KO targets for Penta (5×) KO were BAX, BAK, SIRT-1, MYC, and ICAM-1; the KO targets for 9× KO were BAX, BAK, SIRT-1, ICAM-1, LPLA2, LPL, PPT1, CMAH, and GGTA1; the KO targets for 10× KO were BAX, BAK, SIRT-1, MYC, ICAM-1, LPLA2, LPL, PPT1, CMAH, and GGTA1. (15A) Viability profiles for mAb-O (top panel) and mAb-P (bottom panel) are shown for the production bioreactor cultures. (15B) The bioreactor cultures were harvested on day 12 and the culture supernatants were purified through affinity chromatography followed by two polishing chromatography steps. The resulting purified materials (after three chromatography operations) were then analyzed for host cell protein (HCP) content by HCP ELISA. The purified materials were also analyzed for their levels of polysorbate-degrading residual enzymes by measuring the rates of polysorbate degradation as represented by specific FAR (fatty acid release) rates. The specific FAR rates indicate the residual level of enzymatic HCPs in the purified materials that hydrolytically degrade polysorbate. Higher specific FAR rates indicate higher risks for polysorbate degradation and associated free fatty acid particle formation in the drug product.

FIGS. 16A-16D. Fluorescence in situ hybridization (FISH) analysis of four different CHO cell lines (FIG. 16A to FIG. 16D). Two of the cell lines were CHO host cell lines (one is derived from CHO-K1 and one that is a targeted integration (TI) cell line) and two of the cell lines were CHO recombinant cell lines (generated from transfection of TI host) that produce a recombinant monoclonal antibody. A probe for retrovirus-like particle (RVLP) was used to seek RVLP signal on CHO chromosomes. A strong RVLP signal (as indicated by arrow with line) on one chromosome and several weak signals on various other chromosomes (as indicated by arrowhead without line) were observed for all four tested CHO cell lines.

FIG. 17. RVLP DNA copy number analysis of two CHO host cell lines. A plasmid specific for RVLP was used as the standard (1 uL of the DNA standard was equivalent to 1.8×10⁸ copies). This plasmid used the same sequence as the RVLP probe for FISH analysis.

FIG. 18. Design of guide RNA (gRNA) construct for disrupting RVLP expression in CHO cells. Different guide RNAs for matrix (gMax) and capsid (gCap) of the RVLPs were designed with the aim to eliminate functional GAG protein production.

FIGS. 19A-19G. PDGFRa is down-regulated by UPR activation. FIG. 19A and FIG. 19B depict that PDGFRa protein levels and mRNA levels, respectively, were downregulated when mAb1-expressing CHO cells are grown at pH 7.07. FIG. 19C depicts western blot analysis of two mAb1-expressing host cell lines, CHO DG44 and CHO-K1, treated with chemical UPR inducers: tunicamycin and DTT. FIG. 19D depicts qPCR analysis of PDGFRa mRNA levels in both host cell lines of FIG. 19C treated with tunicamycin and DTT. FIG. 19E depicts western blot analysis of mAb1-expressing CHO-K1 cells treated with tunicamycin to activate the UPR in the presence of UPR pathway-specific inhibitors. RT-PCR panel for XBP-1 shows IRE1alpha RNase activation. FIG. 19F depicts qPCR analysis of PDGFRa mRNA levels in CHO-K1 cells treated with tunicamycin in the presence of UPR pathway-specific inhibitors. FIG. 19G depicts western blot analysis of WT and PERK KO empty host CHO-K1 (Clone 9) cell lines treated with tunicamycin and PERK inhibitor.

FIGS. 20A-20E. FIG. 20A depicts western blot analysis of mAb1-expressing CHO-K1 cells treated with thapsigargin to activate the UPR in the presence of different UPR pathway-specific inhibitors. RT-PCR panel of XBP-1 shows IRE1alpha RNase activation.

FIG. 20B depicts western blot analysis of empty host CHO-K1 cells treated with Tunicamycin to activate the UPR in the presence of different UPR pathway-specific inhibitors.

FIG. 20C depicts qPCR analysis of PDGFRa mRNA levels in CHO-K1 cells treated with thapsigargin in the presence of different UPR pathway-specific inhibitors. FIG. 20D depicts western blot analysis of Cas9-sgRNAs against the PERK gene with a sgRNA against luciferase as control. FIG. 20E depicts western blot analysis of empty host CHO-K1 single cell clones after using Cas9 to knockout PERK. Clone 9 was used in FIG. 19G.

FIG. 21A-21D. PDGFRa signaling is important for cell growth, e.g., CHO cell growth, and growth factor signaling is intact after PDGFRa inhibition. FIG. 21A is a schematic of PDGFRa and insulin receptor (IR) signaling upstream of protein synthesis, cell cycle progression and cell proliferation. Bolder arrows indicate stronger activation by respective receptors. FIG. 21B depicts empty CHO-K1 host cells VCC and % viability after 4 days in the presence or absence of PDGFRa inhibitor and/or insulin in the seed train media.

FIG. 21C depicts western blot analysis of empty host CHO-K1 cells (of FIG. 21B) after 4 days in the presence or absence of PDGFRa inhibitor and/or insulin in the seed train media.

FIG. 21D depicts Day 12 relative IVCC, % viability, relative titer and relative Qp of mAb2-expressing CHO-K1 cells in the presence of PDGFRa inhibitor and/or insulin during production.

FIGS. 22A-22D. FIG. 22A depicts empty host CHO-K1 cells viable cell count (VCC) and % viability after 4 days in increasing PDGFRa inhibitor concentrations in the seed train media. FIG. 22B depicts western blot analysis of empty host CHO-K1 cells after 4 days in increasing PDGFRa inhibitor concentrations in the seed train media. FIG. 22C depicts western blot analysis mAb2-expressing CHO-K1 cells in production in the presence or absence of PERK inhibitor at 10 μM concentration. FIG. 22D depicts qPCR analysis of downstream targets of PERK branch of UPR, CHOP and GADD34, during production for mAb2-expressing CHO-K1 cells in the presence or absence of PERK inhibitor.

FIGS. 23A-23C. PDGFRa levels are stabilized during production in PERK KO cell lines. FIG. 23A depicts western blot analysis of mAb2-expressing CHO-K1 single cell clones after using CRISPR-Cas9 to knockout PERK. FIG. 23B depicts Day 14 relative IVCC, % viability, relative titer and relative Qp of mAb2-expressing CHO-K1 PERK KO cells.

FIG. 23C depicts western blot analysis of production for mAb2-expressing CHO-K1 WT and PERK KO cells.

FIGS. 24A-24E. PERK and Bax/Bak TKOs synergistically increase bioprocess outcomes. FIG. 24A depicts western blot analysis of mAb3-expressing CHO-K1 single cell clones in seed train after using Cas9 to knockout PERK. Overall titer, depicted in FIG. 24B, and relative Qp, depicted in FIG. 24C, of various mAb3-expressing CHO-K1 hosts across different bioprocesses: lean production media, rich production media and intensified process using rich production media. FIG. 24D depicts western blot analysis of various mAb3-expressing CHO-K1 hosts in rich production media. FIG. 24E depicts qPCR analysis of heavy chain and light chain mRNA levels in lean production media and rich production media.

FIGS. 25A-25B. FIG. 25A depicts bioprocess outcomes for a 6-day production of mAb3-expressing pools in either a Bax/Bak DKO background or a PERK/Bax/Bak TKO background showing relative titer, Qp and IVCC. FIG. 25B depicts bioprocess outcomes for a 14-day production of Fab1-expressing pools in either a WT, PERK KO, Bax/Bak DKO or PERK/Bax/Bak TKO background showing relative titer, Qp and IVCC.

FIG. 26 depicts the time-dependent titer of mAb-Q in different hosts: (1)=BAX/BAK-knock-out; (2)=ICAM-1-knock-out; (3)=control, no-knock-out; (4)=MYC-knock-out; (5)=BAX/BAK/ICAM-1/MYC/SIRT-1-knock-out (Penta-KO); (6)=SIRT-1-knock-out.

FIG. 27 depicts the time-dependent titer of mAb-R in different hosts: (1)=BAX/BAK-knock-out; (2)=ICAM-1-knock-out; (3)=control, no-knock-out; (4)=MYC-knock-out; (5)=BAX/BAK/ICAM-1/MYC/SIRT-1-knock-out (Penta-KO); (6)=SIRT-1-knock-out.

FIG. 28 depicts the increase of average cell diameter over cultivation time for a cell expressing mAb-R: (1)=control, no-knock-out; (2)=MYC-knock-out; (3)=BAX/BAK/ICAM-1/MYC/SIRT-1-knock-out (Penta-KO).

### 5. DETAILED DESCRIPTION

The present disclosure relates to mammalian cells (e.g., Chinese Hamster Ovary (CHO) cells) that are modified to reduce or eliminate the expression of certain mammalian cell endogenous products (e.g., host cell proteins and virus-like particles), and methods of using such cells in the production of a recombinant product of interest (e.g., a recombinant protein, a recombinant viral particle, or a recombinant viral vector). These modifications were specifically chosen to generate engineered mammalian host cells with desired traits in several key areas, including improved cell culture performance (e.g., higher viability and product titers), improved product quality (e.g., more consistent and favorable glycosylation; more stable drug product), and decreased burden on purification for removing problematic or undesired endogenous host cell products (e.g., hydrolytic host cell proteins and virus-like particles) during biomanufacturing.

For clarity, but not by way of limitation, the detailed description of the presently disclosed subject matter is divided into the following subsections:
5.1 Definitions;
5.2 Reduced or Eliminated Expression of Endogenous Products;
5.3 Mammalian Cells Comprising Gene-Specific Modifications;
5.4 Cell Culture Methods; and
5.5 Production of a Recombinant Product of Interest

### 5.1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one” and “one or more than one.”

The terms “comprise(s),” “include(s),” “having,” “has,” “can,” “contain(s)” and variants thereof, as used herein, are intended to be open-ended transitional phrases, terms or words that do not preclude the possibility of additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. The terms “cell culture medium” and “culture medium” refer to a nutrient solution used for growing mammalian cells that typically provides at least one component from one or more of the following categories:
1) an energy source, usually in the form of a carbohydrate such as glucose;
2) all essential amino acids, and usually the basic set of twenty amino acids plus cysteine;
3) vitamins and/or other organic compounds required at low concentrations;
4) free fatty acids; and
5) trace elements, where trace elements are defined as inorganic compounds or naturally occurring elements that are typically required at very low concentrations, usually in the micromolar range.

The nutrient solution can optionally be supplemented with one or more components from any of the following categories:
1) hormones and other growth factors as, for example, insulin, transferrin, and epidermal growth factor;
2) salts and buffers as, for example, calcium, magnesium, and phosphate;
3) nucleosides and bases such as, for example, adenosine, thymidine, and hypoxanthine; and
4) protein and tissue hydrolysates.

“Culturing” a cell refers to contacting a cell with a cell culture medium under conditions suitable to the survival and/or growth and/or proliferation of the cell.

“Batch culture” refers to a culture in which all components for cell culturing (including the cells and all culture nutrients) are supplied to the culturing bioreactor at the start of the culturing process.

“Fed-batch cell culture,” as used herein refers to a batch culture wherein the cells and culture medium are supplied to the culturing bioreactor initially, and additional culture nutrients are fed, continuously or in discrete increments, to the culture during the culturing process, with or without periodic cell and/or product harvest before termination of culture.

“Perfusion culture,” sometimes referred to as continuous culture, is a culture by which the cells are restrained in the culture by, e.g., filtration, encapsulation, anchoring to microcarriers, etc., and the culture medium is continuously, step-wise or intermittently introduced (or any combination of these) and removed from the culturing bioreactor.

As used herein, the term “cell,” refers to animal cells, mammalian cells, cultured cells, host cells, recombinant cells and recombinant host cells. Such cells are generally cell lines obtained or derived from mammalian tissues which are able to grow and survive when placed in media containing appropriate nutrients and/or growth factors.

The terms “host cell,” “host cell line” and “host cell culture” are used interchangeably and refer to cells and their progeny into which exogenous nucleic acid can be subsequently introduced to create recombinant cells. These host cells may also have been modified (i.e., engineered) to alter or delete the expression of certain endogenous host cell products (e.g., endogenous virus-like particles or endogenous host cell proteins). Host cells include “transformants” and “transformed cells,” which include the primary transformed cell and progeny derived therefrom without regard to the number of passages. Progeny does not need to be completely identical in nucleic acid content to a parent cell, but can contain mutations. Mutant progeny that have the same function or biological activity as screened or selected for in the originally transformed cell are included herein. The introduction of exogenous nucleic acid (e.g., by transfection) to these host cells would create recombinant cells that are derived from the original “host cell,” “host cell line” or “host cell line”. The terms “host cell,” “host cell line” and “host cell culture” may also refer to such recombinant cells and their progeny.

The terms “recombinant cell”, “recombinant cell line” and “recombinant cell culture” are used interchangeably and refer to cells and their progeny into which exogenous nucleic acid has been introduced to enable the expression of recombinant product of interest. The recombinant product expressed by such cells may be a recombinant protein, a recombinant viral particle, or a recombinant viral vector.

The term “mammalian host cell” or “mammalian cell” refers to cell lines derived from mammals that are capable of growth and survival when placed in either monolayer culture or in suspension culture in a medium containing the appropriate nutrients and growth factors. The necessary growth factors for a particular cell line are readily determined empirically without undue experimentation, as described for example in Mammalian Cell Culture (Mather, J. P. ed., Plenum Press, N.Y. 1984), and Barnes and Sato, (1980) Cell, 22:649. Typically, the cells are capable of expressing and secreting large quantities of a particular protein, e.g., glycoprotein, of interest into the culture medium. Examples of suitable mammalian host cells within the context of the present disclosure can include Chinese hamster ovary cells/-DHFR (CHO, Urlaub and Chasm, Proc. Natl. Acad. Sci. USA, 77:4216 1980); dp12.CHO cells (EP 307,247 published 15 Mar. 1989); CHO-K1 (ATCC, CCL-61); monkey kidney CVI line transformed by SV40 (COS-7, ATCC CRL 1651); human embryonic kidney line (293 or 293 cells subcloned for growth in suspension culture, Graham et al., J. Gen Virol., 36:59 1977); baby hamster kidney cells (BHK, ATCC CCL 10); mouse sertoli cells (TM4, Mather, Biol. Reprod., 23:243-251 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 1982); MRC 5 cells; FS4 cells; and a human hepatoma line (Hep G2). In certain embodiments, the mammalian cells include Chinese hamster ovary cells/-DHFR (CHO, Urlaub and Chasm, Proc. Natl. Acad. Sci. USA, 77:4216 1980); dp12.CHO cells (EP 307,247 published 15 Mar. 1989).

“Growth phase” of the cell culture refers to the period of exponential cell growth (the log phase) where cells are generally rapidly dividing. The duration of time for which the cells are maintained at growth phase can vary based on the cell-type, the rate of growth of cells and/or the culture conditions, for example. In certain embodiments, during this phase, cells are cultured for a period of time, usually between 1-4 days, and under such conditions that cell growth is maximized. The determination of the growth cycle for the host cell can be determined for the particular host cell envisioned without undue experimentation. “Period of time and under such conditions that cell growth is maximized” and the like, refer to those culture conditions that, for a particular cell line, are determined to be optimal for cell growth and division. In certain embodiments, during the growth phase, cells are cultured in nutrient medium containing the necessary additives generally at about 30°-40° C. in a humidified, controlled atmosphere, such that optimal growth is achieved for the particular cell line. In certain embodiments, cells are maintained in the growth phase for a period of about between one and four days, usually between two to three days.

“Production phase” of the cell culture refers to the period of time during which cell growth is/has plateaued. The logarithmic cell growth typically decreases before or during this phase and protein production takes over. During the production phase, logarithmic cell growth has ended, and protein production is primary. During this period of time the medium is generally supplemented to support continued protein production and to achieve the desired glycoprotein product. Fed-batch and/or perfusion cell culture processes supplement the cell culture medium or provide fresh medium during this phase to achieve and/or maintain desired cell density, viability and/or recombinant protein product titer. A production phase can be conducted at large scale.

The term “activity” as used herein with respect to activity of a protein refers to any activity of a protein including, but not limited to, enzymatic activity, ligand binding, drug transport, ion transport, protein localization, receptor binding, and/or structural activity. Such activity can be modulated, e.g., reduced or eliminated, by reducing or eliminating the expression of the protein, thereby reducing or eliminating the presence of the protein. Such activity can also be modulated, e.g., reduced or eliminated, by altering the nucleic acid sequence encoding the protein such that the resulting modified protein exhibits reduced or eliminated activity relative to a wild type protein.

The term “expression” or “expresses” are used herein to refer to transcription and translation occurring within a host cell. The level of expression of a product gene in a host cell can be determined on the basis of either the amount of corresponding mRNA that is present in the cell or the amount of the protein encoded by the product gene that is produced by the cell.

For example, mRNA transcribed from a product gene is desirably quantitated by northern hybridization. Sambrook et al., Molecular Cloning: A Laboratory Manual, pp. 7.3-7.57 (Cold Spring Harbor Laboratory Press, 1989). Protein encoded by a product gene can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989). When reference is made to reduction and/or elimination of the expression of one or more endogenous products relative to the expression of the endogenous product(s) in an unmodified cell, such reductions and/or eliminations of expression encompass reductions and/or eliminations of the active endogenous product, notwithstanding the presence of mRNA encoding all or a portion of the endogenous product or the presence of endogenous product translated from such mRNA.

As used herein, “polypeptide” refers generally to peptides and proteins having more than about ten amino acids. The polypeptides can be homologous to the host cell, or preferably, can be exogenous, meaning that they are heterologous, i.e., foreign, to the host cell being utilized, such as a human protein produced by a Chinese hamster ovary cell, or a yeast polypeptide produced by a mammalian cell. In certain embodiments, mammalian polypeptides (polypeptides that were originally derived from a mammalian organism) are used, more preferably those which are directly secreted into the medium.

The term “protein” is meant to refer to a sequence of amino acids for which the chain length is sufficient to produce the higher levels of tertiary and/or quaternary structure. This is to distinguish from “peptides” or other small molecular weight drugs that do not have such structure. Typically, the protein herein will have a molecular weight of at least about 15-20 kD, preferably at least about 20 kD. Examples of proteins encompassed within the definition herein include host cell proteins as well as all mammalian proteins, in particular, therapeutic and diagnostic proteins, such as therapeutic and diagnostic antibodies, and, in general proteins that contain one or more disulfide bonds, including multi-chain polypeptides comprising one or more inter- and/or intrachain disulfide bonds.

The term “antibody” is used herein in the broadest sense and encompasses various antibody structures including, but not limited to, monoclonal antibodies, polyclonal antibodies, monospecific antibodies (e.g., antibodies consisting of a single heavy chain sequence and a single light chain sequence, including multimers of such pairings), multispecific antibodies (e.g., bispecific antibodies) and antibody fragments so long as they exhibit the desired antigen-binding activity.

An “antibody fragment,” “antigen-binding portion” of an antibody (or simply “antibody portion”) or “antigen-binding fragment” of an antibody, as used herein, refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG and IgM, and several of these can be further divided into subclasses (isotypes), e.g., IgG1, IgG2, IgG3, IgG4, IgA1, and IgA2. In certain embodiments, the antibody is of the IgG1 isotype. In certain embodiments, the antibody is of the IgG2 isotype. The heavy chain constant domains that correspond to the different classes of immunoglobulins are called α, δ, ε, γ and μ, respectively. The light chain of an antibody can be assigned to one of two types, called kappa (κ) and lambda (λ), based on the amino acid sequence of its constant domain.

The term “titer” as used herein refers to the total amount of recombinantly expressed antibody produced by a cell culture divided by a given amount of medium volume. Titer is typically expressed in units of milligrams of antibody per milliliter or liter of medium (mg/ml or mg/L). In certain embodiments, titer is expressed in grams of antibody per liter of medium (g/L). Titer can be expressed or assessed in terms of a relative measurement, such as a percentage increase in titer as compared obtaining the protein product under different culture conditions.

The term “nucleic acid,” “nucleic acid molecule” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the disclosure in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see, e.g., Stadler et al, Nature Medicine 2017, published online 12 Jun. 2017, doi:10.1038/nm.4356 or EP 2 101 823 B1).

As used herein, the term “vector” refers to a nucleic acid molecule capable of transporting another nucleic acid to which it has been linked.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally can comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “immunoconjugate” is an antibody conjugated to one or more heterologous molecule(s), including but not limited to a cytotoxic agent.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the presently disclosed subject matter can be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6th ed., W. H. Freeman and Co., page 91 (2007).) A single VH or VL domain can be sufficient to confer antigen-binding specificity. Furthermore, antibodies that bind a particular antigen can be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

As used herein, the term “cell density” refers to the number of cells in a given volume of medium. In certain embodiments, a high cell density is desirable in that it can lead to higher protein productivity. Cell density can be monitored by any technique known in the art, including, but not limited to, extracting samples from a culture and analyzing the cells under a microscope, using a commercially available cell counting device or by using a commercially available suitable probe introduced into the bioreactor itself (or into a loop through which the medium and suspended cells are passed and then returned to the bioreactor).

As used herein “retrovirus-like particles” (RVLPs) refers to endogenous products produced by mammalian cells that resemble viral particles, but which, without being bound by theory, are believed to be the result if expression of endogenous retroviral genes. RVLPs are described in the art, e.g., in Duroy et al., Biotechnology and Bioengineering, 117(2); 446-485 (2020), which is incorporated herein by reference in its entirety. RVLPs can be composed of a plurality of proteins, thus the methods and compositions described herein relate to the reduction or elimination of an RVLP in its entirety or any component of an RVLP, e.g., the RVLP group antigen (“GAG”).

As used herein, the term “recombinant protein” refers generally to peptides and proteins, including antibodies, that are encoded by a nucleic acid that is “heterologous,” i.e., foreign to the host cell being utilized, such as a nucleic acid encoding a human antibody that is introduced into a non-human host cell.

As used herein, the term “recombinant viral particle” refers generally to virus particles that may occur naturally or be produced by recombining exogenous nucleic acid for use in vaccine production.

As used herein, the term “recombinant viral vector” refers generally to viral vectors that have been modified to express exogenous viral elements, e.g., for use in gene therapy, including but not limited to recombinant vectors based on adeno-associated virus (AAV), herpes simplex virus (HSV), retrovirus, poxvirus, lentivirus.

### 5.2. Reduced or Eliminated Expression of Endogenous Products

In certain embodiments, the present disclosure relates to modified mammalian cells, e.g., CHO cells, where the expression of one or more mammalian cell endogenous products (e.g., host cell proteins and virus-like particles), is reduced or eliminated. For example, but not by way of limitation, methods for reducing or eliminating endogenous product expression in a mammalian cell include: (1) modification of a gene coding for the endogenous product or component thereof, e.g., by introducing a deletion, insertion, substitution, or combination thereof into the gene; (2) reducing or eliminating the transcription and/or stability of the mRNA encoding the endogenous product or a component thereof; and (3) reducing or eliminating the translation of the mRNA encoding the endogenous product or a component thereof. In certain embodiments, the reduction or elimination of protein expression is obtained by targeted genome editing. For example, CRISPR/Cas9-based genome editing can be employed to modify one or more target genes, resulting in the reduction or elimination of expression of the gene (or genes) targeted for editing.

In certain embodiments, one or more of the mammalian cell endogenous products targeted for reduced or eliminated expression are selected based on their role in promoting apoptosis. As apoptosis can decrease culture viability and productivity, reducing or eliminating expression of such proteins can positively impact culture viability and productivity. For example, but not by way of limitation, the mammalian cell protein selected based on its role in promoting apoptosis is BCL2 Associated X, Apoptosis Regulator (BAX) or BCL2 Antagonist/Killer 1 (BAK). In certain embodiments, the mammalian cells of the present disclosure exhibit reduced or eliminated expression of BAX. In certain embodiments, the mammalian cells of the present disclosure exhibit reduced or eliminated expression of BAK. In certain embodiments, the mammalian cells of the present disclosure exhibit reduced or eliminated expression of BAX and BAK.

In certain embodiments, the mammalian cell endogenous product targeted for reduced or eliminated expression is selected based on its role in promoting clumping and/or aggregation during cell culture. When mammalian cells are used for production of a recombinant product of interest, such clumping and/or aggregation during cell culture can lead to reduced product titers due to the negative impact of clumping and/or aggregation on mammalian cell viability. For example, but not by way of limitation, the mammalian cell endogenous product selected based on its role in promoting clumping and/or aggregation during cell culture is Intercellular Adhesion Molecule 1 (ICAM-1). In certain embodiments, the mammalian cells of the present disclosure exhibit reduced or eliminated expression of ICAM-1.

In certain embodiments, the mammalian cell endogenous product that is targeted for reduced or eliminated expression is an endogenous product that is selected based on its role in regulating the unfolded protein response (UPR). For example, but not by way of limitation, the cellular product selected based on its role in regulating the UPR is inositol-requiring enzyme 1 (IRE1), protein kinase R-like ER kinase (PERK) or activating transcription factor 6 (ATF6). In certain embodiments, the modified cells of the present disclosure exhibit reduced or eliminated expression of PERK. In certain embodiments, PERK, as used herein, refers to a eukaryotic PERK cellular protein, e.g., the CHO PERK cellular protein (Gene ID: 100765343; GenBank: EGW03658.1; and isoforms NCBI Reference Sequence: XP_027285344.2 and NCBI Reference Sequence: XP_016831844.1), and functional variants thereof. In certain embodiments, functional variants of PERK, as used herein encompass PERK sequence variants having 50%, 55%, 60%, 65%, 70%, 75%, 80%, 85%, 90%, 91%, 92%, 93%, 94%, 95%, 96%, 97%, 98%, 99% identity to the wild type PERK sequence of the modified cell used for the production of a recombinant product of interest.

In certain embodiments, one or more of the mammalian cell endogenous products targeted for reduced or eliminated expression are selected based on their role in promoting inefficient cell growth. Mammalian cells express many endogenous products that are not essential for cell growth, survival, and/or productivity. Because expression of these endogenous products consumes considerable cellular energy and DNA/protein building blocks, reducing or eliminating the expression of such endogenous products can render cell growth more efficient and, in the case of cells used to produce a recombinant product of interest, those cellular resources can be diverted to achieve higher productivity of the recombinant product of interest. For example, but not by way of limitation, the mammalian cell endogenous product selected based on its role in promoting efficient cell growth and higher productivity of a recombinant product of interest is BAX, BAK, ICAM-1, PERK, Sirtuin 1 (SIRT-1) or MYC Proto-Oncogene, BHLH Transcription Factor (MYC). In certain embodiments, the mammalian cells of the present disclosure exhibit reduced or eliminated expression of SIRT-1. In certain embodiments, the mammalian cells of the present disclosure exhibit reduced or eliminated expression of PERK. In certain embodiments, the mammalian cells of the present disclosure exhibit reduced or eliminated expression of MYC. In certain embodiments, the mammalian cells of the present disclosure exhibit reduced or eliminated expression of SIRT-1 and MYC. In certain embodiments, the mammalian cells of the present disclosure exhibit reduced or eliminated expression of BAX and MYC. In certain embodiments, the mammalian cells of the present disclosure exhibit reduced or eliminated expression of BAK and MYC. In certain embodiments, the mammalian cells of the present disclosure exhibit reduced or eliminated expression of ICAM-1 and MYC. In certain embodiments, the mammalian cells of the present disclosure exhibit reduced or eliminated expression of BAX and SIRT-1. In certain embodiments, the mammalian cells of the present disclosure exhibit reduced or eliminated expression of BAK and SIRT-1. In certain embodiments, the mammalian cells of the present disclosure exhibit reduced or eliminated expression of ICAM-1 and SIRT-1. In certain embodiments, the mammalian cells of the present disclosure exhibit reduced or eliminated expression of BAX, SIRT-1, and MYC. In certain embodiments, the mammalian cells of the present disclosure exhibit reduced or eliminated expression of BAK, SIRT-1, and MYC. In certain embodiments, the mammalian cells of the present disclosure exhibit reduced or eliminated expression of ICAM-1, SIRT-1, and MYC. In certain embodiments, the mammalian cells of the present disclosure exhibit reduced or eliminated expression of BAX, BAK, SIRT-1, and MYC. In certain embodiments, the mammalian cells of the present disclosure exhibit reduced or eliminated expression of BAX, ICAM-1, SIRT-1, and MYC. In certain embodiments, the mammalian cells of the present disclosure exhibit reduced or eliminated expression of BAK, ICAM-1, SIRT-1, and MYC. In certain embodiments, the mammalian cells of the present disclosure exhibit reduced or eliminated expression of BAX, BAK, MYC, SIRT-1, and ICAM. In certain embodiments, the mammalian cells of the present disclosure exhibit reduced or eliminated expression of BAX, BAK, ICAM-1, PERK, SIRT-1, and/or MYC.

In certain embodiments, the mammalian cell endogenous product that is targeted for reduced or eliminated expression is an endogenous product that can promote non-human glycosylation patterns in a recombinant protein product, e.g., when the cell is used for recombinant protein production. Such non-human glycosylation patterns can include the addition of Galactose-α-1,3-galactose (αGAL) and/or N-glycolylneuraminic acid (NGNA). For example, but not by way of limitation, the mammalian cell protein selected based on its role in promoting non-human glycosylation patterns is Glycoprotein Alpha-Galactosyltransferase 1 (GGTA1), which promotes αGAL addition, or Cytidine Monophosphate-N-Acetylneuraminic Acid Hydroxylase (CMAH), which promotes NGNA addition. In certain embodiments, the mammalian cells of the present disclosure exhibit reduced or eliminated expression of GGTA1. In certain embodiments, the mammalian cells of the present disclosure exhibit reduced or eliminated expression of CMAH. In certain embodiments, the mammalian cells of the present disclosure exhibit reduced or eliminated expression of GGTA1 and CMAH.

In certain embodiments, the mammalian cell endogenous product that is targeted for reduced or eliminated expression is an endogenous product that promotes the catabolism of branched chain amino acids (BCAAs). While branched chain amino acids, e.g., leucine, isoleucine, and valine, are essential amino acids and are thus generally included in chemically defined media employed in mammalian cell culture, the catabolism of BCAAs can lead to toxic intermediates and metabolites that decrease cell growth, productivity and product quality. For example, the mammalian cell protein selected based on its role in promoting BCAA catabolism is Branched chain keto acid dehydrogenase El alpha subunit (BCKDHA) or Branched-chain alpha-keto acid dehydrogenase El beta subunit (BCKDHB).

In contexts where the cell is used for production of a recombinant product of interest (e.g., a recombinant protein, a recombinant viral particle, or recombinant viral vector), certain mammalian cell endogenous products can co-purify with the product of interest, leading to increased costs associated with additional purification processes and/or decreased shelf-life of the resulting recombinant product. For example, certain endogenous virus-like particles from mammalian cells (e.g., RVLPs in CHO cells) that are produced during biotherapeutic manufacturing need to be removed to sufficiently low levels by the purification process to ensure patient safety. For example, certain residual host cell proteins that co-purify with the recombinant product of interest can degrade polysorbate used as a surfactant in the final drug product, and lead to particle formation. For example, but not by way of limitation, the mammalian cell endogenous host cell proteins targeted for reduced or eliminated expression based on its potential to co-purify with the recombinant product of interest and degrade polysorbate used as a surfactant in the final drug product include Lipoprotein lipase (LPL) which is also referred to as LPL1; Phospholipase A2 group (LPLA2) which is also referred to as PLA2G7; Palmitoyl-protein thioesterase 1 (PPT1); or Lipase A (Lysosomal acid lipase/cholesteryl ester hydrolase, Lipase) (LIPA). In certain embodiments, the mammalian cells of the present disclosure exhibit reduced or eliminated expression of PPT1. In certain embodiments, the mammalian cells of the present disclosure exhibit reduced or eliminated expression of PPT1 and LPL. In certain embodiments, the mammalian cells of the present disclosure exhibit reduced or eliminated expression of LPLA2. In certain embodiments, the mammalian cells of the present disclosure exhibit reduced or eliminated expression of PPT1 and LIPA. In certain embodiments, the mammalian cells of the present disclosure exhibit reduced or eliminated expression of PPT1, LPL and LPLA2. In certain embodiments, the mammalian cells of the present disclosure exhibit reduced or eliminated expression of PPT1, LPL and LIPA. In certain embodiments, the mammalian cells of the present disclosure exhibit reduced or eliminated expression of PPT1, LIPA and LPLA2. In certain embodiments, the mammalian cells of the present disclosure exhibit reduced or eliminated expression of PPT1, LPL, LIPA, and LPLA2.

In certain embodiments, the host cells of the present disclosure exhibit reduced or eliminated expression of one or more endogenous products in order to facilitate purification of a recombinant product of interest by reducing the overall amount of host cell endogenous product produced during cell culture. Such reduction in overall host cell endogenous product production can reduce the burden on the chromatographic and other materials and systems employed in the purification process, thereby reducing the overall cost of purification and increasing purification process efficiency. For example, but not by way of limitation, the host cell endogenous product targeted for reduced or eliminated expression based on the overall amount of the endogenous product produced during cell culture is selected from the following endogenous product: a RVLP group antigen (GAG); MYC Proto-Oncogene, BHLH Transcription Factor (MYC); BCL2 Associated X, Apoptosis Regulator (BAX); BCL2 Antagonist/Killer 1 (BAK); Intercellular Adhesion Molecule 1 (ICAM-1); Protein Kinase R-like ER Kinase (PERK); Sirtuin 1 (SIRT-1); Glycoprotein Alpha-Galactosyltransferase 1 (GGTA1); Cytidine Monophosphate-N-Acetylneuraminic Acid Hydroxylase (CMAH); Lipoprotein lipase (LPL); Phospholipase A2 group (LPLA2); Palmitoyl-protein thioesterase 1 (PPT1); Branched Chain Keto Acid Dehydrogenase El alpha subunit (BCKDHA); Branched Chain Keto Acid Dehydrogenase El beta subunit (BCKDHB); and Lipase A (Lysosomal acid lipase/cholesteryl ester hydrolase, Lipase) (LIPA).

In certain embodiments, the host cells of the present disclosure exhibit reduced or eliminated expression of the following endogenous products: BAX; BAK; ICAM-1; GGTA1; CMAH; LPL; LPLA2; and PPT1.

In certain embodiments, the host cells of the present disclosure exhibit reduced or eliminated expression of the following endogenous products: BAX; BAK; ICAM-1; SIRT-1; GGTA1; CMAH; LPL; LPLA2; and PPT1.

In certain embodiments, the host cells of the present disclosure exhibit reduced or eliminated expression of the following endogenous products: BAX; BAK; ICAM-1; SIRT-1; MYC; GGTA1; CMAH; LPL; LPLA2; and PPT1.

In certain embodiments, the host cells of the present disclosure exhibit reduced or eliminated expression of the following endogenous products: BAX; BAK; ICAM-1; SIRT-1; MYC; GGTA1; CMAH; LPL, LPLA2; PPT1; and LIPA.

In certain embodiments, the host cells of the present disclosure exhibit reduced or eliminated expression of the following endogenous products: BAX; BAK; ICAM-1; GGTA1; CMAH; LPLA2; PPT1; and LIPA.

In certain embodiments, the host cells of the present disclosure exhibit reduced or eliminated expression of the following endogenous products: BAX; BAK; ICAM-1; SIRT-1; GGTA1; CMAH; LPLA2; PPT1; and LIPA.

In certain embodiments, the host cells of the present disclosure exhibit reduced or eliminated expression of the following endogenous products: BAX; BAK; ICAM-1; SIRT-1; MYC; GGTA1; CMAH; LPLA2; PPT1; and LIPA.

In certain embodiments, the host cells of the present disclosure exhibit reduced or eliminated expression of the following endogenous products: BAX; BAK; ICAM-1; LPL; LPLA2; and PPT1.

In certain embodiments, the host cells of the present disclosure exhibit reduced or eliminated expression of the following endogenous products: BAX; BAK; ICAM-1; SIRT-1; LPL, LPLA2; and PPT1.

In certain embodiments, the host cells of the present disclosure exhibit reduced or eliminated expression of the following endogenous products: BAX; BAK; ICAM-1; SIRT-1; MYC; LPL; LPLA2; and PPT1.

In certain embodiments, the host cells of the present disclosure exhibit reduced or eliminated expression of the following endogenous products: BAX; BAK; ICAM-1; SIRT-1; MYC; LPL; LPLA2; PPT1; and LIPA.

In certain embodiments, the host cells of the present disclosure exhibit reduced or eliminated expression of the following endogenous products: BAX; BAK; ICAM-1; PERK; SIRT-1; and MYC.

In certain embodiments, the host cells of the present disclosure exhibit reduced or eliminated expression of the following endogenous products: MYC; BAX; BAK; ICAM-1; PERK; SIRT-1; GGTA1; CMAH; LPL; LPLA2; PPT1 and LIPA.

In certain embodiments, the host cells of the present disclosure exhibit reduced or eliminated expression of the following endogenous products: BAX; BAK; and PERK.

In certain embodiments, the host cells of the present disclosure exhibit reduced or eliminated expression of the following endogenous products: BAX; BAK; MYC; SIRT-1; and ICAM.

In certain embodiments, the host cells of the present disclosure exhibit reduced or eliminated expression of the following endogenous products: BAX; BAK; LPL; LPLA2; GGTA1; and CMAH.

In certain embodiments, the host cells of the present disclosure exhibit reduced or eliminated expression of the following endogenous products: BAX; BAK; PERK; LPL; LPLA2; GGTA1; and CMAH.

In certain embodiments, the host cells of the present disclosure exhibit reduced or eliminated expression of the following endogenous products: BAX; BAK; MYC; LPL; LPLA2; GGTA1; and CMAH.

In certain embodiments, the host cells of the present disclosure exhibit reduced or eliminated expression of the following endogenous products: BAX; BAK; MYC; PERK; LPL; LPLA2; GGTA1; and CMAH.

In certain embodiments, the host cells of the present disclosure exhibit reduced or eliminated expression of the following endogenous products: BAX; BAK; LPL; LPLA2; GGTA1; CMAH; and PPT1.

In certain embodiments, the host cells of the present disclosure exhibit reduced or eliminated expression of the following endogenous products: BAX; BAK; PERK; LPL; LPLA2; GGTA1; CMAH; and PPT1.

In certain embodiments, the host cells of the present disclosure exhibit reduced or eliminated expression of the following endogenous products: BAX; BAK; MYC; LPL; LPLA2; GGTA1; CMAH; and PPT1.

In certain embodiments, the host cells of the present disclosure exhibit reduced or eliminated expression of the following endogenous products: BAX; BAK; MYC; PERK; LPL; LPLA2; GGTA1; CMAH; and PPT1.

In certain embodiments, the host cells of the present disclosure exhibit reduced or eliminated expression of the following endogenous products: BAX; BAK; BCKDHA; ICAM-1; GGTAT; CMAH; LPL; LPLA2; and PPT1.

In certain embodiments, the host cells of the present disclosure exhibit reduced or eliminated expression of the following endogenous products: BAX; BAK; BCKDHA; ICAM-1; SIRT-1; GGTA1; CMAH; LPL; LPLA2; and PPT1.

In certain embodiments, the host cells of the present disclosure exhibit reduced or eliminated expression of the following endogenous products: BAX; BAK; BCKDHA; ICAM-1; SIRT-1; MYC; GGTA1; CMAH; LPL; LPLA2; and PPT1.

In certain embodiments, the host cells of the present disclosure exhibit reduced or eliminated expression of the following endogenous products: BAX; BAK; BCKDHA; ICAM-1; SIRT-1; MYC; GGTA1; CMAH; LPL, LPLA2; PPT1; and LIPA.

In certain embodiments, the host cells of the present disclosure exhibit reduced or eliminated expression of the following endogenous products: BAX; BAK; BCKDHA; ICAM-1; GGTAT; CMAH; LPLA2; PPT1; and LIPA.

In certain embodiments, the host cells of the present disclosure exhibit reduced or eliminated expression of the following endogenous products: BAX; BAK; BCKDHA; ICAM-1; SIRT-1; GGTA1; CMAH; LPLA2; PPT1; and LIPA.

In certain embodiments, the host cells of the present disclosure exhibit reduced or eliminated expression of the following endogenous products: BAX; BAK; BCKDHA; ICAM-1; SIRT-1; MYC; GGTA1; CMAH; LPLA2; PPT1; and LIPA.

In certain embodiments, the host cells of the present disclosure exhibit reduced or eliminated expression of the following endogenous products: BAX; BAK; BCKDHA; ICAM-1; LPL; LPLA2; and PPT1.

In certain embodiments, the host cells of the present disclosure exhibit reduced or eliminated expression of the following endogenous products: BAX; BAK; BCKDHA; ICAM-1; SIRT-1; LPL, LPLA2; and PPT1.

In certain embodiments, the host cells of the present disclosure exhibit reduced or eliminated expression of the following endogenous products: BAX; BAK; BCKDHA; ICAM-1; SIRT-1; MYC; LPL; LPLA2; and PPT1.

In certain embodiments, the host cells of the present disclosure exhibit reduced or eliminated expression of the following endogenous products: BAX; BAK; BCKDHA; ICAM-1; SIRT-1; MYC; LPL; LPLA2; PPT1; and LIPA.

In certain embodiments, the host cells of the present disclosure exhibit reduced or eliminated expression of the following endogenous products: BAX; BAK; BCKDHA; ICAM-1; PERK; SIRT-1; and MYC.

In certain embodiments, the host cells of the present disclosure exhibit reduced or eliminated expression of the following endogenous products: MYC; BAX; BAK; BCKDHA; ICAM-1; PERK; SIRT-1; GGTA1; CMAH; LPL; LPLA2; PPT1 and LIPA.

In certain embodiments, the host cells of the present disclosure exhibit reduced or eliminated expression of the following endogenous products: BAX; BAK; BCKDHA; and PERK.

In certain embodiments, the host cells of the present disclosure exhibit reduced or eliminated expression of the following endogenous products: BAX; BAK; BCKDHA; MYC; SIRT-1; and ICAM.

In certain embodiments, the host cells of the present disclosure exhibit reduced or eliminated expression of the following endogenous products: BAX; BAK; BCKDHA; LPL; LPLA2; GGTA1; and CMAH.

In certain embodiments, the host cells of the present disclosure exhibit reduced or eliminated expression of the following endogenous products: BAX; BAK; BCKDHA; PERK; LPL; LPLA2; GGTA1; and CMAH.

In certain embodiments, the host cells of the present disclosure exhibit reduced or eliminated expression of the following endogenous products: BAX; BAK; BCKDHA; MYC; LPL; LPLA2; GGTA1; and CMAH.

In certain embodiments, the host cells of the present disclosure exhibit reduced or eliminated expression of the following endogenous products: BAX; BAK; BCKDHA; MYC; PERK; LPL; LPLA2; GGTA1; and CMAH.

In certain embodiments, the host cells of the present disclosure exhibit reduced or eliminated expression of the following endogenous products: BAX; BAK; BCKDHA; LPL; LPLA2; GGTA1; CMAH; and PPT1.

In certain embodiments, the host cells of the present disclosure exhibit reduced or eliminated expression of the following endogenous products: BAX; BAK; BCKDHA; PERK; LPL; LPLA2; GGTAT; CMAH; and PPT1.

In certain embodiments, the host cells of the present disclosure exhibit reduced or eliminated expression of the following endogenous products: BAX; BAK; BCKDHA; MYC; LPL; LPLA2; GGTAT; CMAH; and PPT1.

In certain embodiments, the host cells of the present disclosure exhibit reduced or eliminated expression of the following endogenous products: BAX; BAK; BCKDHA; MYC; PERK; LPL; LPLA2; GGTA1; CMAH; and PPT1.

In certain embodiments, the host cells of the present disclosure exhibit reduced or eliminated expression of the following endogenous products: BAX; BAK; BCKDHB; MYC; PERK; LPL; LPLA2; GGTA1; CMAH; and PPT1.

In certain embodiments, the host cells of the present disclosure exhibit reduced or eliminated expression of the following endogenous products: BAX; BAK; BCKDHB; ICAM-1; GGTAT; CMAH; LPL; LPLA2; and PPT1.

In certain embodiments, the host cells of the present disclosure exhibit reduced or eliminated expression of the following endogenous products: BAX; BAK; BCKDHB; ICAM-1; SIRT-1; GGTA1; CMAH; LPL; LPLA2; and PPT1.

In certain embodiments, the host cells of the present disclosure exhibit reduced or eliminated expression of the following endogenous products: BAX; BAK; BCKDHB; ICAM-1; SIRT-1; MYC; GGTA1; CMAH; LPL; LPLA2; and PPT1.

In certain embodiments, the host cells of the present disclosure exhibit reduced or eliminated expression of the following endogenous products: BAX; BAK; BCKDHB; ICAM-1; SIRT-1; MYC; GGTA1; CMAH; LPL, LPLA2; PPT1; and LIPA.

In certain embodiments, the host cells of the present disclosure exhibit reduced or eliminated expression of the following endogenous products: BAX; BAK; BCKDHB; ICAM-1; GGTAT; CMAH; LPLA2; PPT1; and LIPA.

In certain embodiments, the host cells of the present disclosure exhibit reduced or eliminated expression of the following endogenous products: BAX; BAK; BCKDHB; ICAM-1; SIRT-1; GGTA1; CMAH; LPLA2; PPT1; and LIPA.

In certain embodiments, the host cells of the present disclosure exhibit reduced or eliminated expression of the following endogenous products: BAX; BAK; BCKDHB; ICAM-1; SIRT-1; MYC; GGTA1; CMAH; LPLA2; PPT1; and LIPA.

In certain embodiments, the host cells of the present disclosure exhibit reduced or eliminated expression of the following endogenous products: BAX; BAK; BCKDHB; ICAM-1; LPL; LPLA2; and PPT1.

In certain embodiments, the host cells of the present disclosure exhibit reduced or eliminated expression of the following endogenous products: BAX; BAK; BCKDHB; ICAM-1; SIRT-1; LPL, LPLA2; and PPT1.

In certain embodiments, the host cells of the present disclosure exhibit reduced or eliminated expression of the following endogenous products: BAX; BAK; BCKDHB; ICAM-1; SIRT-1; MYC; LPL; LPLA2; and PPT1.

In certain embodiments, the host cells of the present disclosure exhibit reduced or eliminated expression of the following endogenous products: BAX; BAK; BCKDHB; ICAM-1; SIRT-1; MYC; LPL; LPLA2; PPT1; and LIPA.

In certain embodiments, the host cells of the present disclosure exhibit reduced or eliminated expression of the following endogenous products: BAX; BAK; BCKDHB; ICAM-1; PERK; SIRT-1; and MYC.

In certain embodiments, the host cells of the present disclosure exhibit reduced or eliminated expression of the following endogenous products: MYC; BAX; BAK; BCKDHB; ICAM-1; PERK; SIRT-1; GGTA1; CMAH; LPL; LPLA2; PPT1 and LIPA.

In certain embodiments, the host cells of the present disclosure exhibit reduced or eliminated expression of the following endogenous products: BAX; BAK; BCKDHB; and PERK.

In certain embodiments, the host cells of the present disclosure exhibit reduced or eliminated expression of the following endogenous products: BAX; BAK; BCKDHB; MYC; SIRT-1; and ICAM.

In certain embodiments, the host cells of the present disclosure exhibit reduced or eliminated expression of the following endogenous products: BAX; BAK; BCKDHB; LPL; LPLA2; GGTA1; and CMAH.

In certain embodiments, the host cells of the present disclosure exhibit reduced or eliminated expression of the following endogenous products: BAX; BAK; BCKDHB; PERK; LPL; LPLA2; GGTA1; and CMAH.

In certain embodiments, the host cells of the present disclosure exhibit reduced or eliminated expression of the following endogenous products: BAX; BAK; BCKDHB; MYC; LPL; LPLA2; GGTA1; and CMAH.

In certain embodiments, the host cells of the present disclosure exhibit reduced or eliminated expression of the following endogenous products: BAX; BAK; BCKDHB; MYC; PERK; LPL; LPLA2; GGTA1; and CMAH.

In certain embodiments, the host cells of the present disclosure exhibit reduced or eliminated expression of the following endogenous products: BAX; BAK; BCKDHB; LPL; LPLA2; GGTA1; CMAH; and PPT1.

In certain embodiments, the host cells of the present disclosure exhibit reduced or eliminated expression of the following endogenous products: BAX; BAK; BCKDHB; PERK; LPL; LPLA2; GGTA1; CMAH; and PPT1.

In certain embodiments, the host cells of the present disclosure exhibit reduced or eliminated expression of the following endogenous products: BAX; BAK; BCKDHB; MYC; LPL; LPLA2; GGTA1; CMAH; and PPT1.

In certain embodiments, the host cells of the present disclosure exhibit reduced or eliminated expression of the following endogenous products: BAX; BAK; BCKDHB; MYC; PERK; LPL; LPLA2; GGTA1; CMAH; and PPT1.

In certain embodiments, the host cells of the present disclosure exhibit reduced or eliminated expression of the following endogenous products: BAX; BAK; BCKDHA; BCKDHB; MYC; PERK; LPL; LPLA2; GGTA1; CMAH; and PPT1.

In certain embodiments, the host cells of the present disclosure exhibit reduced or eliminated expression of the following endogenous products: BAX; BAK; BCKDHA; BCKDHB; ICAM-1; GGTA1; CMAH; LPL; LPLA2; and PPT1.

In certain embodiments, the host cells of the present disclosure exhibit reduced or eliminated expression of the following endogenous products: BAX; BAK; BCKDHA; BCKDHB; ICAM-1; SIRT-1; GGTA1; CMAH; LPL; LPLA2; and PPT1.

In certain embodiments, the host cells of the present disclosure exhibit reduced or eliminated expression of the following endogenous products: BAX; BAK; BCKDHA; BCKDHB; ICAM-1; SIRT-1; MYC; GGTA1; CMAH; LPL; LPLA2; and PPT1.

In certain embodiments, the host cells of the present disclosure exhibit reduced or eliminated expression of the following endogenous products: BAX; BAK; BCKDHA; BCKDHB; ICAM-1; SIRT-1; MYC; GGTA1; CMAH; LPL, LPLA2; PPT1; and LIPA.

In certain embodiments, the host cells of the present disclosure exhibit reduced or eliminated expression of the following endogenous products: BAX; BAK; BCKDHA; BCKDHB; ICAM-1; GGTA1; CMAH; LPLA2; PPT1; and LIPA.

In certain embodiments, the host cells of the present disclosure exhibit reduced or eliminated expression of the following endogenous products: BAX; BAK; BCKDHA; BCKDHB; ICAM-1; SIRT-1; GGTA1; CMAH; LPLA2; PPT1; and LIPA.

In certain embodiments, the host cells of the present disclosure exhibit reduced or eliminated expression of the following endogenous products: BAX; BAK; BCKDHA; BCKDHB; ICAM-1; SIRT-1; MYC; GGTA1; CMAH; LPLA2; PPT1; and LIPA.

In certain embodiments, the host cells of the present disclosure exhibit reduced or eliminated expression of the following endogenous products: BAX; BAK; BCKDHA; BCKDHB; ICAM-1; LPL; LPLA2; and PPT1.

In certain embodiments, the host cells of the present disclosure exhibit reduced or eliminated expression of the following endogenous products: BAX; BAK; BCKDHA; BCKDHB; ICAM-1; SIRT-1; LPL, LPLA2; and PPT1.

In certain embodiments, the host cells of the present disclosure exhibit reduced or eliminated expression of the following endogenous products: BAX; BAK; BCKDHA; BCKDHB; ICAM-1; SIRT-1; MYC; LPL; LPLA2; and PPT1.

In certain embodiments, the host cells of the present disclosure exhibit reduced or eliminated expression of the following endogenous products: BAX; BAK; BCKDHA; BCKDHB; ICAM-1; SIRT-1; MYC; LPL; LPLA2; PPT1; and LIPA.

In certain embodiments, the host cells of the present disclosure exhibit reduced or eliminated expression of the following endogenous products: BAX; BAK; BCKDHA; BCKDHB; ICAM-1; PERK; SIRT-1; and MYC.

In certain embodiments, the host cells of the present disclosure exhibit reduced or eliminated expression of the following endogenous products: MYC; BAX; BAK; BCKDHA; BCKDHB; ICAM-1; PERK; SIRT-1; GGTA1; CMAH; LPL; LPLA2; PPT1 and LIPA.

In certain embodiments, the host cells of the present disclosure exhibit reduced or eliminated expression of the following endogenous products: BAX; BAK; BCKDHA; BCKDHB; and PERK.

In certain embodiments, the host cells of the present disclosure exhibit reduced or eliminated expression of the following endogenous products: BAX; BAK; BCKDHA; BCKDHB; MYC; SIRT-1; and ICAM.

In certain embodiments, the host cells of the present disclosure exhibit reduced or eliminated expression of the following endogenous products: BAX; BAK; BCKDHA; BCKDHB; LPL; LPLA2; GGTA1; and CMAH.

In certain embodiments, the host cells of the present disclosure exhibit reduced or eliminated expression of the following endogenous products: BAX; BAK; BCKDHA; BCKDHB; PERK; LPL; LPLA2; GGTA1; and CMAH.

In certain embodiments, the host cells of the present disclosure exhibit reduced or eliminated expression of the following endogenous products: BAX; BAK; BCKDHA; BCKDHB; MYC; LPL; LPLA2; GGTA1; and CMAH.

In certain embodiments, the host cells of the present disclosure exhibit reduced or eliminated expression of the following endogenous products: BAX; BAK; BCKDHA; BCKDHB; MYC; PERK; LPL; LPLA2; GGTA1; and CMAH.

In certain embodiments, the host cells of the present disclosure exhibit reduced or eliminated expression of the following endogenous products: BAX; BAK; BCKDHA; BCKDHB; LPL; LPLA2; GGTA1; CMAH; and PPT1.

In certain embodiments, the host cells of the present disclosure exhibit reduced or eliminated expression of the following endogenous products: BAX; BAK; BCKDHA; BCKDHB; PERK; LPL; LPLA2; GGTA1; CMAH; and PPT1.

In certain embodiments, the host cells of the present disclosure exhibit reduced or eliminated expression of the following endogenous products: BAX; BAK; BCKDHA; BCKDHB; MYC; LPL; LPLA2; GGTA1; CMAH; and PPT1.

In certain embodiments, the host cells of the present disclosure exhibit reduced or eliminated expression of the following endogenous products: BAX; BAK; BCKDHA; BCKDHA; BCKDHB; MYC; PERK; LPL; LPLA2; GGTA1; CMAH; and PPT1.

In certain embodiments, a host cell of the present disclosure is modified to reduce or eliminate the expression of one or more host cell endogenous products relative to the expression of the host cell endogenous products in an unmodified, i.e., “reference”, host cell.

In certain embodiments, the reference host cells are host cells where the expression of one or more particular endogenous product, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide, is not reduced or eliminated. In certain embodiments, a reference host cell is a cell that comprises at least one or both wild-type alleles of the gene(s) coding for GAG component, and/or BAX; BAK; ICAM-1; SIRT-1; MYC; GGTA1; CMAH; LPL; LIPA; LPLA2; BCKDHA; BCKDHB; PPT1; and/or PERK. For example, but not by way of limitation, a reference host cell is a host cell that has both wild-type alleles of the gene(s) coding for GAG and/or BAX; BAK; ICAM-1; SIRT-1; MYC; GGTA1; CMAH; LPL; LIPA; LPLA2; BCKDHA; BCKDHB; PPT1; and/or PERK. In certain embodiments, the reference host cells are WT host cells. In certain embodiments, the modification of reducing or eliminating the expression of one or more host cell endogenous products is performed before the introduction of the exogenous nucleic acid encoding the recombinant product of interest. In certain embodiments, the modification of reducing or eliminating the expression of one or more host cell endogenous products is performed after the introduction of the exogenous nucleic acid encoding the recombinant product of interest.

In certain embodiments, the expression of one or more endogenous products, e.g., GAG and/or a BAX; BAK; ICAM-1; SIRT-1; MYC; GGTA1; CMAH; LPL; LPLA2; BCKDHA; BCKDHB; PPT1; and/or PERK polypeptide, in a cell that has been modified to reduce or eliminate expression of the endogenous product, is less than about 90%, less than about 80%, less than about 70%, less than about 60%, less than about 50%, less than about 40%, less than about 30%, less than about 20%, less than about 10%, less than about 5%, less than about 4%, less than about 3%, less than about 2% or less than about 1% of the corresponding endogenous product expression of a reference cell, e.g., a WT host cell. In certain embodiments, the expression of one or more endogenous products in a cell that has been modified to reduce or eliminate expression of the endogenous products, is less than about 90%, less than about 80%, less than about 70%, less than about 60%, less than about 50%, less than about 40%, less than about 30%, less than about 20%, less than about 10%, less than about 5%, less than about 4%, less than about 3%, less than about 2%, or less than about 1% of the corresponding endogenous product expression of a reference cell, e.g., a WT host cell.

In certain embodiments, the expression of one or more endogenous products, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide, in a host cell that has been modified to reduce or eliminate expression of the endogenous products, is at least about 90%, at least about 80%, at least about 70%, at least about 60%, at least about 50%, at least about 40%, at least about 30%, at least about 20%, at least about 10%, at least about 5%, at least about 4%, at least about 3%, at least about 2% or at least about 1% of the corresponding endogenous product expression of a reference host cell, e.g., a WT host cell. In certain embodiments, the expression of one or more endogenous products in a host cell that has been modified to reduce or eliminate expression of the endogenous product, is at least about 90%, at least about 80%, at least about 70%, at least about 60%, at least about 50%, at least about 40%, at least about 30%, at least about 20%, at least about 10%, at least about 5%, at least about 4%, at least about 3%, at least about 2%, or at least about 1% of the corresponding endogenous product expression of a reference cell, e.g., a WT mammalian cell.

In certain embodiments, the expression of one or more particular endogenous products, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide, in a cell that has been modified to reduce or eliminate expression of the endogenous products, is no more than about 90%, no more than about 80%, no more than about 70%, no more than about 60%, no more than about 50%, no more than about 40%, no more than about 30%, no more than about 20%, no more than about 10%, no more than about 5%, no more than about 4%, no more than about 3%, no more than about 2% or no more than about 1% of the corresponding endogenous product expression of a reference host cell, e.g., a WT host cell. In certain embodiments, the expression of one or more endogenous products, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide, in a cell that has been modified to reduce or eliminate expression of the endogenous products, is no more than about 40% of the corresponding endogenous product expression of a reference cell, e.g., a WT mammalian cell. In certain embodiments, the expression of one or more endogenous products in a cell that has been modified to reduce or eliminate expression of the endogenous products, is no more than about 90%, no more than about 80%, no more than about 70%, no more than about 60%, no more than about 50%, no more than about 40%, no more than about 30%, no more than about 20%, no more than about 10%, no more than about 5%, no more than about 4%, no more than about 3%, no more than about 2% or no more than about 1% of the corresponding endogenous product expression of a reference cell, e.g., a WT host cell.

In certain embodiments, the expression of one or more endogenous products, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide, in a cell that has been modified to reduce or eliminate expression of the endogenous products, is between about 1% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 1% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 1% and about 70%, between about 10% and about 70%, between about 20% and about 70%, between about 30% and about 70%, between about 40% and about 70%, between about 50% and about 70%, between about 60% and about 70%, between about 65% and about 70%, between about 1% and about 60%, between about 10% and about 60%, between about 20% and about 60%, between about 30% and about 60%, between about 40% and about 60%, between about 50% and about 60%, between about 55% and about 60%, between about 1% and about 50%, between about 10% and about 50%, between about 20% and about 50%, between about 30% and about 50%, between about 40% and about 50%, between about 45% and about 50%, between about 1% and about 40%, between about 10% and about 40%, between about 20% and about 40%, between about 30% and about 40%, between about 35% and about 40%, between about 1% and about 30%, between about 10% and about 30%, between about 20% and about 30%, between about 25% and about 30%, between about 1% and about 20%, between about 5% and about 20%, between about 10% and about 20%, between about 15% and about 20%, between about 1% and about 10%, between about 5% and about 10%, between about 5% and about 20%, between about 5% and about 30%, between about 5% and about 40% of the corresponding endogenous products expression of a reference cell, e.g., a WT host cell. In certain embodiments, the expression of one or more endogenous products, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide, in a cell that has been modified to reduce or eliminate expression of the endogenous products, is between about 1% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 1% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 1% and about 70%, between about 10% and about 70%, between about 20% and about 70%, between about 30% and about 70%, between about 40% and about 70%, between about 50% and about 70%, between about 60% and about 70%, between about 65% and about 70%, between about 1% and about 60%, between about 10% and about 60%, between about 20% and about 60%, between about 30% and about 60%, between about 40% and about 60%, between about 50% and about 60%, between about 55% and about 60%, between about 1% and about 50%, between about 10% and about 50%, between about 20% and about 50%, between about 30% and about 50%, between about 40% and about 50%, between about 45% and about 50%, between about 1% and about 40%, between about 10% and about 40%, between about 20% and about 40%, between about 30% and about 40%, between about 35% and about 40%, between about 1% and about 30%, between about 10% and about 30%, between about 20% and about 30%, between about 25% and about 30%, between about 1% and about 20%, between about 5% and about 20%, between about 10% and about 20%, between about 15% and about 20%, between about 1% and about 10%, between about 5% and about 10%, between about 5% and about 20%, between about 5% and about 30%, between about 5% and about 40% of the corresponding endogenous product expression of a reference cell, e.g., a WT host cell.

In certain embodiments, the expression of one or more endogenous products, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide, in a cell that has been modified to reduce or eliminate expression of the endogenous products, is between about 5% and about 40% of the corresponding endogenous product expression of a reference cell, e.g., a WT host cell.

In certain embodiments, the expression level of the one or more endogenous products, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide, in different reference cells (e.g., cells that comprise at least one or both wild-type alleles of the corresponding gene) can vary.

In certain embodiments, a genetic engineering system is employed to reduce or eliminate the expression of one or more particular endogenous product (e.g., GAG and/or a BAX; BAK; ICAM-1; SIRT-1; MYC; GGTA1; CMAH; LPL; LIPA; LPLA2; BCKDHA; BCKDHB; PPT1; and/or PERK expression). Various genetic engineering systems known in the art can be used for the methods disclosed herein. Non-limiting examples of such systems include the CRISPR/Cas system, the zinc-finger nuclease (ZFN) system, the transcription activator-like effector nuclease (TALEN) system and the use of other tools for reducing or eliminating protein expression by gene silencing, such as small interfering RNAs (siRNAs), short hairpin RNA (shRNA), and microRNA (miRNA). Any CRISPR/Cas systems known in the art, including traditional, enhanced or modified Cas systems, as well as other bacterial based genome excising tools such as Cpf-1 can be used with the methods disclosed herein.

In certain embodiments, a portion of one or more genes, e.g., genes coding for a endogenous product such as GAG and/or BAX; BAK; ICAM-1; SIRT-1; MYC; GGTA1; CMAH; LPL; LIPA; LPLA2; BCKDHA; BCKDHB; PPT1; and/or PERK polypeptides, is deleted to reduce or eliminate expression of the corresponding endogenous product in a host cell. In certain embodiments, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85% or at least about 90% of the gene is deleted. In certain embodiments, no more than about 2%, no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85% or no more than about 90% of the gene is deleted. In certain embodiments, between about 2% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 2% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 2% and about 70%, between about 10% and about 70%, between about 20% and about 70%, between about 30% and about 70%, between about 40% and about 70%, between about 50% and about 70%, between about 60% and about 70%, between about 65% and about 70%, between about 2% and about 60%, between about 10% and about 60%, between about 20% and about 60%, between about 30% and about 60%, between about 40% and about 60%, between about 50% and about 60%, between about 55% and about 60%, between about 2% and about 50%, between about 10% and about 50%, between about 20% and about 50%, between about 30% and about 50%, between about 40% and about 50%, between about 45% and about 50%, between about 2% and about 40%, between about 10% and about 40%, between about 20% and about 40%, between about 30% and about 40%, between about 35% and about 40%, between about 2% and about 30%, between about 10% and about 30%, between about 20% and about 30%, between about 25% and about 30%, between about 2% and about 20%, between about 5% and about 20%, between about 10% and about 20%, between about 15% and about 20%, between about 2% and about 10%, between about 5% and about 10%, or between about 2% and about 5% of the gene is deleted.

In certain embodiments, at least one exon of a gene encoding GAG and/or a BAX; BAK; ICAM-1; SIRT-1; MYC; GGTA1; CMAH; LPL; LIPA; LPLA2; BCKDHA; BCKDHB; PPT1; and/or PERK polypeptide is at least partially deleted in a host cell. “Partially deleted,” as used herein, refers to at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, no more than about 2%, no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85%, no more than about 90%, no more than about 95%, between about 2% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 2% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 2% and about 70%, between about 10% and about 70%, between about 20% and about 70%, between about 30% and about 70%, between about 40% and about 70%, between about 50% and about 70%, between about 60% and about 70%, between about 65% and about 70%, between about 2% and about 60%, between about 10% and about 60%, between about 20% and about 60%, between about 30% and about 60%, between about 40% and about 60%, between about 50% and about 60%, between about 55% and about 60%, between about 2% and about 50%, between about 10% and about 50%, between about 20% and about 50%, between about 30% and about 50%, between about 40% and about 50%, between about 45% and about 50%, between about 2% and about 40%, between about 10% and about 40%, between about 20% and about 40%, between about 30% and about 40%, between about 35% and about 40%, between about 2% and about 30%, between about 10% and about 30%, between about 20% and about 30%, between about 25% and about 30%, between about 2% and about 20%, between about 5% and about 20%, between about 10% and about 20%, between about 15% and about 20%, between about 2% and about 10%, between about 5% and about 10%, or between about 2% and about 5% of a region, e.g., of the exon, is deleted.

In certain non-limiting embodiments, a CRISPR/Cas9 system is employed to reduce or eliminate the expression of one or more endogenous products, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide in a host cell. A clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), and trans-activating crRNA (tracrRNA, binds to crRNA and forms an active complex with Cas9). The terms “guide RNA” and “gRNA” refer to any nucleic acid that promotes the specific association (or “targeting”) of an RNA-guided nuclease such as a Cas9 to a target sequence such as a genomic or episomal sequence in a cell. gRNAs can be unimolecular (comprising a single RNA molecule, and referred to alternatively as chimeric) or modular (comprising more than one, and typically two, separate RNA molecules, such as a crRNA and a tracrRNA, which are usually associated with one another, for instance by duplexing).

CRISPR/Cas9 strategies can employ a vector to transfect the mammalian cell. The guide RNA (gRNA) can be designed for each application as this is the sequence that Cas9 uses to identify and directly bind to the target DNA in a mammalian cell. Multiple crRNAs and the tracrRNA can be packaged together to form a single-guide RNA (sgRNA). The sgRNA can be joined together with the Cas9 gene and made into a vector in order to be transfected into mammalian cells.

In certain embodiments, the CRISPR/Cas9 system for use in reducing or eliminating the expression of one or more endogenous products, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide, comprises a Cas9 molecule and one or more gRNAs comprising a targeting domain that is complementary to a target sequence of the gene encoding the endogenous product or a component thereof. In certain embodiments, the target gene is a region of the gene coding for the endogenous product, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide. The target sequence can be any exon or intron region within the gene.

In certain embodiments, the gRNAs are administered to the mammalian cell in a single vector and the Cas9 molecule is administered to the host cell in a second vector. In certain embodiments, the gRNAs and the Cas9 molecule are administered to the host cell in a single vector. Alternatively, each of the gRNAs and Cas9 molecule can be administered by separate vectors. In certain embodiments, the CRISPR/Cas9 system can be delivered to the host cell as a ribonucleoprotein complex (RNP) that comprises a Cas9 protein complexed with one or more gRNAs, e.g., delivered by electroporation (see, e.g., DeWitt et al., Methods 121-122:9-15 (2017) for additional methods of delivering RNPs to a cell). In certain embodiments, administering the CRISPR/Cas9 system to the host cell results in the reduction or elimination of the expression of an endogenous product, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide.

Using CRISPR/Cas9, a particular target gene can be targeted at one, two, three or more different sites. For example, but not by way of limitation, three different sites within the coding sequence can be targeted using three different gRNAs at the same time using multiplexed ribonucleoprotein delivery. In certain embodiments, multiplexed ribonucleoprotein delivery shows higher gene-editing efficacy and specificity compared to the common plasmid based CRISPR/Cas9 editing. In certain embodiments, double-strand breaks at the gene target site(s) induce indel formations. In certain embodiments, e.g., when multiple sites are targeted due to multiplexed gRNA usage, deletions of sequences between the target sites, e.g., intervening exons, result a frameshift of the CDS of the target protein.

In certain embodiments, sequencing of the PCR-amplified gene locus in the modified cell pools will reveal an interruption of the sequencing reaction at the first gRNA site showing successful targeting for the gene. In certain embodiments the cell pools will comprise modification(s) at all targeted genes in at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cells in the pool. In certain embodiments the cell pools will comprise modification(s) at “n-i” of the “n” targeted genes (where “n” is the number of targeted genes) in at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cells in the pool. In certain embodiments the cell pools will comprise modification(s) at “n-2” of the “n” targeted genes in at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cells in the pool. In certain embodiments the cell pools will comprise modification(s) at “n-3” of the “n” targeted genes in at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cells in the pool. In certain embodiments the cell pools will comprise modification(s) at “n-4” of the “n” targeted genes in at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cells in the pool. In certain embodiments the cell pools will comprise modification(s) at one to “n” of the “n” targeted genes in at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cells in the pool.

In certain embodiments, the genetic engineering system is a ZFN system for reducing or eliminating the expression of one or more particular endogenous product in a mammalian cell, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide. The ZFN can act as restriction enzyme, which is generated by combining a zinc finger DNA-binding domain with a DNA-cleavage domain. A zinc finger domain can be engineered to target specific DNA sequences which allows the zinc-finger nuclease to target desired sequences within genomes. The DNA-binding domains of individual ZFNs typically contain a plurality of individual zinc finger repeats and can each recognize a plurality of base pairs. The most common method to generate a new zinc-finger domain is to combine smaller zinc-finger “modules” of known specificity. The most common cleavage domain in ZFNs is the non-specific cleavage domain from the type IIs restriction endonuclease FokI. ZFN modulates the expression of proteins by producing double-strand breaks (DSBs) in the target DNA sequence, which will, in the absence of a homologous template, be repaired by non-homologous end-joining (NHEJ). Such repair can result in deletion or insertion of base-pairs, producing frame-shift and preventing the production of the harmful protein (Durai et al., Nucleic Acids Res.; 33 (18): 5978-90 (2005)). Multiple pairs of ZFNs can also be used to completely remove entire large segments of genomic sequence (Lee et al., Genome Res.; 20 (1): 81-9 (2010)).

In certain embodiments, the genetic engineering system is a TALEN system for reducing or eliminating the expression of one or more particular endogenous product, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide, in a mammalian cell. TALENs are restriction enzymes that can be engineered to cut specific sequences of DNA. TALEN systems operate on a similar principle as ZFNs. TALENs are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome (Boch et al., Nature Biotechnology; 29(2):135-6 (2011)). In certain embodiments, the target gene encodes GAG and/or a BAX; BAK; ICAM-1; SIRT-1; MYC; GGTA1; CMAH; LPL; LIPA; LPLA2; BCKDHA; BCKDHB; PPT1; and/or PERK.

In certain embodiments, the expression of one or more particular endogenous product, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide, can be reduced or eliminated using oligonucleotides that have complementary sequences to corresponding nucleic acids (e.g., mRNA). Non-limiting examples of such oligonucleotides include small interference RNA (siRNA), short hairpin RNA (shRNA), and micro RNA (miRNA). In certain embodiments, such oligonucleotides can be homologous to at least a portion of a GAG component and/or a BAX; BAK; ICAM-1; SIRT-1; MYC; GGTA1; CMAH; LPL; LIPA; LPLA2; BCKDHA; BCKDHB; PPT1; and/or PERK nucleic acid sequence, wherein the homology of the portion relative to the corresponding nucleic acid sequence is at least about 75 or at least about 80 or at least about 85 or at least about 90 or at least about 95 or at least about 98 percent. In certain non-limiting embodiments, the complementary portion can constitute at least 10 nucleotides or at least 15 nucleotides or at least 20 nucleotides or at least 25 nucleotides or at least 30 nucleotides and the antisense nucleic acid, shRNA, mRNA or siRNA molecules can be up to 15 or up to 20 or up to 25 or up to 30 or up to 35 or up to 40 or up to 45 or up to 50 or up to 75 or up to 100 nucleotides in length. Antisense nucleic acid, shRNA, mRNA or siRNA molecules can comprise DNA or atypical or non-naturally occurring residues, for example, but not limited to, phosphorothioate residues.

The genetic engineering systems disclosed herein can be delivered into the mammalian cell using a viral vector, e.g., retroviral vectors such as gamma-retroviral vectors, and lentiviral vectors. Combinations of retroviral vector and an appropriate packaging line are suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art. Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify the mammalian cells disclosed herein. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic engineering of the mammalian cell disclosed herein. For example, a nucleic acid molecule can be introduced into the mammalian cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation and protoplast fusion. Liposomes can also be potentially beneficial for delivery of nucleic acid molecules into a mammalian cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

### 5.3 Mammalian Cells Comprising Gene-Specific Modifications

In one aspect, the present disclosure relates to cells or compositions comprising one or more cells, e.g., mammalian cells, having reduced or eliminated expression of one or more endogenous products. In certain embodiments, the cell has reduced or eliminated expression of GAG and/or a BAX; BAK; ICAM-1; SIRT-1; MYC; GGTA1; CMAH; LPL; LPLA2; BCKDHA; BCKDHB; PPT1; and/or PERK polypeptide.

As used herein, eliminated expression refers to the elimination of the expression of a particular endogenous product, e.g., GAG and/or a BAX; BAK; ICAM-1; SIRT-1; MYC; GGTA1; CMAH; LPL; LPLA2; BCKDHA; BCKDHB; PPT1; and/or PERK polypeptide, in the cell as compared to a reference cell. As used herein, reduced expression refers to a reduction in the expression of an endogenous product, e.g., GAG and/or a BAX; BAK; ICAM-1; SIRT-1; MYC; GGTA1; CMAH; LPL; LPLA2; BCKDHA; BCKDHB; PPT1; and/or PERK polypeptide, in the cell as compared to a reference cell.

Non-limiting examples of cells useful in connection with the subject matter of the present disclosure include CHO cells (e.g., DHFR CHO cells), dp12.CHO cells, CHO-K1 (ATCC, CCL-61), monkey kidney CV1 line transformed by SV40 (e.g., COS-7 ATCC CRL-1651), human embryonic kidney line (e.g., HEK 293 or HEK 293 cells subcloned for growth in suspension culture), baby hamster kidney cells (e.g., BHK, ATCC CCL 10), mouse sertoli cells (e.g. TM4), monkey kidney cells (e.g., CV1 ATCC CCL 70), African green monkey kidney cells (e.g., VERO-76, ATCC CRL-1587), human cervical carcinoma cells (e.g., HELA, ATCC CCL 2), canine kidney cells (e.g., MDCK, ATCC CCL 34), buffalo rat liver cells (e.g., BRL 3A, ATCC CRL 1442), human lung cells (e.g., W138, ATCC CCL 75), human liver cells (e.g., Hep G2, HB 8065), mouse mammary tumor (e.g., MMT 060562, ATCC CCL51), TRI cells, MRC 5 cells, FS4 cells, human hepatoma line (e.g., Hep G2), myeloma cell lines (e.g., Y0, NSO and Sp2/0). In certain embodiments, the cells are CHO cells. Additional non-limiting examples of CHO host cells include CHO K1SV cells, CHO DG44 cells, a CHO DUKXB-11 cells, CHOKIS cells and CHO KIM cells.

In certain embodiments, the cells disclosed herein express a recombinant product of interest. In certain embodiments, the recombinant product of interest is a recombinant protein. In certain embodiments, the recombinant product of interest is a monoclonal antibody. Additional non-limiting examples of recombinant products of interest are provided in Section 5.5.

In certain embodiments, the cells disclosed herein can be used for production of commercially useful amounts of the recombinant product of interest. In certain embodiments, the cells disclosed herein facilitate the production of commercially useful amounts of a recombinant product of interest, at least in part, via inducing a reduced level of degradation of components of the production process, relative to a reference cells, e.g., WT host cells. In certain embodiments, the components of the production process are lipid-containing components. In certain embodiments, the lipid-containing components are detergents. In certain embodiments, the detergent is a polysorbate-containing component. In certain embodiments, the polysorbate-containing component is PS20 (polyoxyethylene (20) sorbitan monolaurate). In certain embodiments, the polysorbate-containing component is PS80 (Polyoxyethylene (80) sorbitan monooleate). In certain embodiments, cells of the present disclosure can reduce degradation of a component of the production process, e.g., PS20, to less than about 90%, less than about 80%, less than about 70%, less than about 60%, less than about 50%, less than about 40%, less than about 30%, less than about 20%, less than about 10%, less than about 5%, less than about 4%, less than about 3%, less than about 2% or less than about 1% of the corresponding PS20 degradation observed with a reference cell, e.g., a WT host cell.

In certain embodiments, the cells disclosed herein can comprise a nucleic acid that encodes a recombinant product of interest. In certain embodiments, the nucleic acid can be present in one or more vectors, e.g., expression vectors. One type of vector is a “plasmid,” which refers to a circular double stranded DNA loop into which additional DNA segments can be ligated. Another type of vector is a viral vector, where additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). Additional non-limiting examples of expression vectors for use in the present disclosure include viral vectors (e.g., replication defective retroviruses, adenoviruses and adeno-associated viruses) that serve equivalent functions.

In certain embodiments, the nucleic acid encoding a recombinant product of interest can be introduced into a host cell, disclosed herein. In certain embodiments, the introduction of a nucleic acid into a cell can be carried out by any method known in the art including, but not limited to, transfection, electroporation, microinjection, infection with a viral or bacteriophage vector containing the nucleic acid sequences, cell fusion, chromosome-mediated gene transfer, microcell-mediated gene transfer, spheroplast fusion, etc. In certain embodiments, the host cell is eukaryotic, e.g., a Chinese Hamster Ovary (CHO) cell or lymphoid cell (e.g., Y0, NSO, Sp20 cell).

In certain embodiments, the nucleic acid encoding a recombinant product of interest can be randomly integrated into a host cell genome (“Random Integration” or “RI”). For example, but not by way of limitation, a nucleic acid encoding a recombinant product of interest can be randomly integrated into the genome of a cell that has also been modified to have reduced or eliminated expression of one or more particular endogenous products, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide.

In certain embodiments, the nucleic acid encoding a recombinant product of interest can be integrated into a host cell genome in a targeted manner (“Targeted Integration” or “TI”, as described in detail herein). For example, but not by way of limitation, a nucleic acid encoding a recombinant product of interest can be integrated in a targeted manner into the genome of a cell that has been modified to have reduced or eliminated expression of one or more particular endogenous product, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide. In certain embodiments, the use of a TI host cell for the introduction of a nucleic acid encoding a recombinant product of interest will provide for robust, stable cell culture performance and lower risk of sequence variants in the resulting recombinant product of interest. TI host cells and strategies for the use of the same are described in detail in U.S. Patent Application Publication No. US20210002669, the contents of which are incorporated by reference in their entirety.

In certain embodiments employing targeted integration, the exogenous nucleotide sequence is integrated at a site within a specific locus of the genome of a TI host cell. In certain embodiments, the locus into which the exogenous nucleotide sequence is integrated is at least about 50%, at least about 60%, at least about 70%, at least about 80%, at least about 90%, at least about 95%, at least about 99%, or at least about 99.9% homologous to a sequence selected from Contigs NW_006874047.1, NW_006884592.1, NW_006881296.1, NW_003616412.1, NW 003615063.1, NW 006882936.1, and NW 003615411.1.

In certain embodiments, the nucleotide sequence immediately 5′ of the integrated exogenous sequence is selected from the group consisting of nucleotides 41190-45269 of NW_006874047.1, nucleotides 63590-207911 of NW_006884592.1, nucleotides 253831-491909 of NW 006881296.1, nucleotides 69303-79768 of NW 003616412.1, nucleotides 293481-315265 of NW_003615063.1, nucleotides 2650443-2662054 of NW_006882936.1, or nucleotides 82214-97705 of NW_003615411.1 and sequences at least 50% homologous thereto. In certain embodiments, the nucleotide sequence immediately 5′ of the integrated exogenous sequence are at least about 50%, at least about 60%, at least about 70%, at least about 80%, at least about 90%, at least about 95%, at least about 99%, or at least about 99.9% homologous to nucleotides 41190-45269 of NW_006874047.1, nucleotides 63590-207911 of NW_006884592.1, nucleotides 253831-491909 of NW_006881296.1, nucleotides 69303-79768 of NW 003616412.1, nucleotides 293481-315265 of NW 003615063.1, nucleotides 2650443-2662054 of NW_006882936.1, or nucleotides 82214-97705 of NW_003615411.1.

In certain embodiments, the nucleotide sequence immediately 3′ of the integrated exogenous sequence is selected from the group consisting of nucleotides 45270-45490 of NW 006874047.1, nucleotides 207912-792374 of NW 006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, or nucleotides 97706-105117 of NW_003615411.1 and sequences at least 50% homologous thereto. In certain embodiments, the nucleotide sequence immediately 3′ of the integrated exogenous sequence is at least about 50%, at least about 60%, at least about 70%, at least about 80%, at least about 90%, at least about 95%, at least about 99%, or at least about 99.9% homologous to nucleotides 45270-45490 of NW_006874047.1, nucleotides 207912-792374 of NW 006884592.1, nucleotides 491910-667813 of NW 006881296.1, nucleotides 79769-100059 of NW_003616412.1, nucleotides 315266-362442 of NW_003615063.1, nucleotides 2662055-2701768 of NW_006882936.1, or nucleotides 97706-105117 of NW_003615411.1.

In certain embodiments, the integrated exogenous sequence is flanked 5′ by a nucleotide sequence selected from the group consisting of nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, and nucleotides 82214-97705 of NW_003615411.1.and sequences at least 50% homologous thereto. In certain embodiments, the integrated exogenous sequence is flanked 3′ by a nucleotide sequence selected from the group consisting of nucleotides 45270-45490 of NW_006874047.1, nucleotides 207912-792374 of NW_006884592.1, nucleotides 491910-667813 of NW 006881296.1, nucleotides 79769-100059 of NW 003616412.1, nucleotides 315266-362442 of NW_003615063.1, nucleotides 2662055-2701768 of NW_006882936.1, and nucleotides 97706-105117 of NW_003615411.1 and sequences at least 50% homologous thereto. In certain embodiments, the nucleotide sequence flanking 5′ of the integrated exogenous nucleotide sequence is at least about 50%, at least about 60%, at least about 70%, at least about 80%, at least about 90%, at least about 95%, at least about 99%, or at least about 99.9% homologous to nucleotides 41190-45269 of NW_006874047.1, nucleotides 63590-207911 of NW_006884592.1, nucleotides 253831-491909 of NW_006881296.1, nucleotides 69303-79768 of NW 003616412.1, nucleotides 293481-315265 of NW 003615063.1, nucleotides 2650443-2662054 of NW_006882936.1, and nucleotides 82214-97705 of NW_003615411.1. In certain embodiments, the nucleotide sequence flanking 3′ of the integrated exogenous nucleotide sequence is at least about 50%, at least about 60%, at least about 70%, at least about 80%, at least about 90%, at least about 95%, at least about 99%, or at least about 99.9% homologous to nucleotides 45270-45490 of NW_006874047.1, nucleotides 207912-792374 of NW_006884592.1, nucleotides 491910-667813 of NW_006881296.1, nucleotides 79769-100059 of NW_003616412.1, nucleotides 315266-362442 of NW 003615063.1, nucleotides 2662055-2701768 of NW 006882936.1, and nucleotides 97706-105117 of NW_003615411.1.

In certain embodiments, the integrated exogenous nucleotide sequence is operably linked to a nucleotide sequence selected from the group consisting of Contigs NW 006874047.1, NW 006884592.1, NW 006881296.1, NW 003616412.1, NW 003615063.1, NW_006882936.1, and NW_003615411.1 and sequences at least 50% homologous thereto. In certain embodiments, the nucleotide sequence operably linked to the exogenous nucleotide sequence is at least about 50%, at least about 60%, at least about 70%, at least about 80%, at least about 90%, at least about 95%, at least about 99%, or at least about 99.9% homologous to a sequence selected from Contigs NW 006874047.1, NW_006884592.1, NW_006881296.1, NW_003616412.1, NW_003615063.1, NW_006882936.1, and NW_003615411.1.

In certain embodiments, the nucleic acid encoding a product of interest can be integrated into a host cell genome using transposase-based integration. Transposase-based integration techniques are disclosed, for example, in Trubitsyna et al., Nucleic Acids Res. 45(10):e89 (2017), Li et al., PNAS 110(25):E2279-E2287 (2013) and WO 2004/009792, which are incorporated by reference herein in their entireties.

In certain embodiments, the nucleic acid encoding a recombinant product of interest can be randomly integrated into a host cell genome (“Random Integration” or “RI”). In certain embodiments, the random integration can be mediated by any method or systems known in the art. In certain embodiments, the nucleic acid sequence encoding the recombinant product of interest is integrated into the cellular genome of a mammalian cell by means of a transposase-mediated gene integration (using, for e.g., Lonza's GS piggyBac transposase system, ATUM's Leap-In transposase system, or Probiogen's DirectedLuck transposase with epigenetic targeting). In certain embodiments, the random integration is mediated by MaxCyte STX® electroporation system.

In certain embodiments, targeted integration can be combined with random integration. In certain embodiments, the targeted integration can be followed by random integration. In certain embodiments, random integration can be followed by targeted integration. For example, but not by way of limitation, a nucleic acid encoding a recombinant product of interest can be randomly integrated into the genome of a cell that has been modulated to have reduced or eliminated expression of one or more particular endogenous product, e.g., GAG; BAX; BAK; ICAM-1; PERK; SIRT-1; MYC; GGTA1; CMAH; LPL; LIPA; LPLA2; BCKDHA; BCKDHB; and/or PPT1, and a nucleic acid encoding the same recombinant product of interest can be integrated in the genome of the cell in a targeted manner.

In certain embodiments, the host cells disclosed herein comprise one or more altered genes. In certain embodiments, the alteration to the gene reduces or eliminates the expression of an endogenous product. In certain embodiments, the host cells disclosed herein comprise one or more altered GAG genes and/or BAX; BAK; ICAM-1; SIRT-1; MYC; GGTA1; CMAH; LPL; LIPA; LPLA2; BCKDHA; BCKDHB; PPT1; and/or PERK genes. In certain embodiments, the subsequent transcript of an altered GAG gene and/or BAX; BAK; ICAM-1; SIRT-1; MYC; GGTA1; CMAH; LPL; LIPA; LPLA2; BCKDHA; BCKDHB; PPT1; and/or PERK gene codes for a endogenous product having reduced or eliminated expression. In certain embodiments, the one or more altered genes are altered by disruption of a coding region. In certain embodiments, the genes alteration comprises a biallelic alteration. In certain embodiments, the genes alteration comprises a deletion of 1 or more base pairs, 2 or more base pairs, 3 or more base pairs, 4 or more base pairs, 5 or more base pairs, 6 or more base pairs, 7 or more base pairs, 8 or more base pairs, 9 or more base pairs, 10 or more base pairs, 11 or more base pairs, 12 or more base pairs, 13 or more base pairs, 14 or more base pairs, 15 or more base pairs, 16 or more base pairs, 17 or more base pairs, 18 or more base pairs, 19 or more base pairs, or 20 or more base pairs.

In certain embodiments, the present disclosure relates to modified cells or compositions comprising one or more modified cells, where the modified cells or compositions comprising one or more modified cells exhibit one or more of the following features: 1) the modified cell exhibits improved cell culture performance relative to similar cells lacking the modification; 2) the modified cell exhibits improved product quality attributes relative to similar cells lacking the modification; 3) the modified cells exhibit improved drug product stability attributes relative to similar cells lacking the modification; and 4) the modified cells exhibit improved purification performance attributes relative to similar cells lacking the modification.

In certain embodiments, the present disclosure relates to cells or compositions comprising one or more cells having all of the following features: 1) the modified cell exhibits improved cell culture performance relative to similar cells lacking the modification; 2) the modified cell exhibits improved product quality attributes relative to similar cells lacking the modification; 3) the modified cells exhibit improved drug product stability attributes relative to similar cells lacking the modification; and 4) the modified cells exhibit improved purification performance attributes relative to similar cells lacking the modification.

In certain embodiments, the modified cells of the present disclosure exhibit improved cell culture performance relative to similar cells lacking the modification. In certain embodiments, the modified cells of the present disclosure exhibit improved cell culture performance due to: i) increased/extended viability and healthier mitochondria for metabolism; ii) decreased cell clumping/aggregation; and/or iii) higher productivity and higher titers. In certain embodiments, the modified cells of the present disclosure exhibit increased/extended viability and healthier mitochondria for metabolism, due to reduced or eliminated expression of BAX and/or BAK. In certain embodiments, the modified cells of the present disclosure exhibit increased/extended viability and healthier mitochondria for metabolism, due to reduced or eliminated expression of BAX. In certain embodiments, the modified cells of the present disclosure exhibit increased/extended viability and healthier mitochondria for metabolism, due to reduced or eliminated expression of BAK. In certain embodiments, the modified cells of the present disclosure exhibit decreased cell clumping/aggregation due to reduced or eliminated expression of ICAM-1. In certain embodiments, the modified cells of the present disclosure exhibit higher productivity and higher titers, due to reduced or eliminated expression of PERK. In certain embodiments, the modified cells of the present disclosure exhibit higher productivity and higher titers due to reduced or eliminated expression of SIRT-1. In certain embodiments, the modified cells of the present disclosure exhibit increased/extended viability and healthier mitochondria for metabolism, due to reduced or eliminated expression of MYC. In certain embodiments, the modified cells of the present disclosure exhibit improved cell culture performance due to reduced or eliminated expression of BAX; BAK, ICAM-1; SIRT-1 and/or MYC. In certain embodiments, the modified cells of the present disclosure exhibit improved cell culture performance due to reduced or eliminated expression of BAX; BAK, ICAM-1; SIRT-1; MYC and/or PERK.

In certain embodiments, the modified cells of the present disclosure exhibit improved product quality due to elimination of undesired types of glycosylation. In certain embodiments, the modified cells of the present disclosure exhibit improved product quality due to reduced or eliminated expression of GGTA1 and/or CMAH. In certain embodiments, the modified cells of the present disclosure exhibit improved product quality due to reduced or eliminated expression of GGTA1. In certain embodiments, the modified cells of the present disclosure exhibit improved product quality due to reduced or eliminated expression of CMAH.

In certain embodiments, the modified cells of the present disclosure exhibit improved product stability due to decreased risk of polysorbate degradation. In certain embodiments, the modified cells of the present disclosure exhibit improved product stability due to decreased levels of residual hydrolytic enzymes in the product. In certain embodiments, the decreased risk of polysorbate degradation in the modified cells of the present disclosure can be achieved by the reduced or eliminated expression of LPL, LIPA, LPLA2 and/or PPT1. In certain embodiments, the decreased risk of polysorbate degradation in the modified cells of the present disclosure can be achieved by the reduced or eliminated expression of LPL. In certain embodiments, the decreased risk of polysorbate degradation in the modified cells of the present disclosure can be achieved by the reduced or eliminated expression of LPLA2. In certain embodiments, the decreased risk of polysorbate degradation in the modified cells of the present disclosure can be achieved by the reduced or eliminated expression of PPT1.

In certain embodiments, the modified cells of the present disclosure exhibit improved purification performance. In certain embodiments, the modified cells of the present disclosure exhibit improved purification performance due to elimination of a variety of endogenous host cell products. In certain embodiments, the eliminated endogenous host cell products are virus-like particles (e.g., RVLP). In certain embodiments, the eliminated endogenous host cell products are proteins related to polysorbate degradation. In certain embodiments, the modified cells of the present disclosure exhibit improved purification performance due to reduced or eliminated expression of GAG, BAX; BAK; ICAM-1; PERK; SIRT-1; MYC; GGTA1; CMAH; LPL; LIPA; BCKDHA; BCKDHB; LPLA2; and/or PPT1. In certain embodiments, the modified cells of the present disclosure exhibit improved purification performance due to cleaner harvest with less cellular debris. In certain embodiments, the modified cells of the present disclosure exhibit cleaner harvest with less cellular debris due to reduced or eliminated expression of BAX; BAK; ICAM-1; SIRT-1 and MYC.

In certain embodiments, the present disclosure relates to modified cells or compositions comprising one or more TI cells exhibiting improved cell culture performance. In certain embodiments, the TI cells of the present disclosure exhibit reduced or eliminated expression of BAX; BAK; ICAM-1; PERK; SIRT-1 and MYC. In certain embodiments, the TI cells of the present disclosure exhibit reduced or eliminated expression of BAX. In certain embodiments, the TI cells of the present disclosure exhibit reduced or eliminated expression of BAK. In certain embodiments, the TI cells of the present disclosure exhibit reduced or eliminated expression of ICAM-1. In certain embodiments, the TI cells of the present disclosure exhibit reduced or eliminated expression of SIRT-1. In certain embodiments, the TI cells of the present disclosure exhibit reduced or eliminated expression of MYC. In certain embodiments, the TI cells of the present disclosure exhibit reduced or eliminated expression of PERK. In certain embodiments, the TI cells of the present disclosure exhibit reduced or eliminated expression of one or more of BAX; BAK; ICAM-1; PERK; SIRT-1 and/or MYC.

In certain embodiments, the present disclosure relates to TI cells or compositions comprising one or more TI cells exhibiting improved product quality due to elimination of undesired types of glycosylation. In certain embodiments, the TI cells of the present disclosure exhibit reduced or eliminated expression of GGTA1 and CMAH. In certain embodiments, the TI cells of the present disclosure exhibit reduced or eliminated expression of GGTA1. In certain embodiments, the TI cells of the present disclosure exhibit reduced or eliminated expression of CMAH. In certain embodiments, the TI cells of the present disclosure exhibit reduced or eliminated expression of GGTA1 and/or CMAH.

In certain embodiments, the present disclosure relates to TI cells or compositions comprising one or more TI cells exhibiting improved product stability due to decreased risk of polysorbate degradation. In certain embodiments, the TI cells of the present disclosure exhibit reduced or eliminated expression of LPL, LIPA, LPLA2 and PPT1. In certain embodiments, the TI cells of the present disclosure exhibit reduced or eliminated expression of LPL. In certain embodiments, the TI cells of the present disclosure exhibit reduced or eliminated expression of LPLA2. In certain embodiments, the TI cells of the present disclosure exhibit reduced or eliminated expression of PPT1. In certain embodiments, the TI cells of the present disclosure exhibit reduced or eliminated expression of LPL and LPLA2. In certain embodiments, the TI cells of the present disclosure exhibit reduced or eliminated expression of LPL and PPT1. In certain embodiments, the TI cells of the present disclosure exhibit reduced or eliminated expression of LPLA2 and PPT1. In certain embodiments, the TI cells of the present disclosure exhibit reduced or eliminated expression of one or more of LPL, LPLA2 and/or PPT1.

In certain embodiments, the present disclosure relates to TI cells or compositions comprising one or more TI cells exhibiting improved purification performance. In certain embodiments, the TI cells of the present disclosure exhibit reduced or eliminated expression of GAG; BAX; BAK; ICAM-1; PERK; SIRT-1; MYC; GGTA1; CMAH; LPL; LIPA; BCKDHA; BCKDHB; LPLA2; and/or PPT1. In certain embodiments, the cells of the present disclosure exhibit improved purification performance due to cleaner harvest with less cellular debris. In certain embodiments, the cells of the present disclosure exhibit cleaner harvest with less cellular debris due to reduced or eliminated expression of BAX; BAK; ICAM-1; SIRT-1 and MYC. In certain embodiments, the cells of the present disclosure exhibit cleaner harvest with less cellular debris due to reduced or eliminated expression of BAX; BAK; ICAM-1; and SIRT-1. In certain embodiments, the cells of the present disclosure exhibit cleaner harvest with less cellular debris due to reduced or eliminated expression of BAX; BAK; and ICAM-1. In certain embodiments, the TI cells of the present disclosure exhibit reduced or eliminated expression of one or more of GAG; BAX; BAK; ICAM-1; SIRT-1; PERK; MYC; GGTA1; CMAH; LPL; LIPA; BCKDHA; BCKDHB; LPLA2; and/or PPT1.

In certain embodiments, the present disclosure relates to TI cells or compositions comprising one or more TI cells exhibiting improved cell culture performance and with improved product quality due to elimination of undesired types of glycosylation. In certain embodiments, the TI cells of the present disclosure exhibit reduced or eliminated expression of BAX; BAK; ICAM-1; SIRT-1; MYC; GGTA1 and CMAH. In certain embodiments, the TI cells of the present disclosure exhibit reduced or eliminated expression of BAX; BAK; ICAM-1; SIRT-1; MYC and GGTA1. In certain embodiments, the TI cells of the present disclosure exhibit reduced or eliminated expression of BAX; BAK; ICAM-1; SIRT-1; MYC; and CMAH.

In certain embodiments, the TI cells of the present disclosure exhibit reduced or eliminated expression of one or more of BAX; BAK; ICAM-1; SIRT-1; MYC; GGTA1 and/or CMAH. In certain embodiments, the present disclosure relates to TI cells or compositions comprising one or more TI cells exhibit improved cell culture performance, improved product quality due to elimination of undesired types of glycosylation, and improved product stability due to decreased risk of polysorbate degradation. In certain embodiments, the TI cells of the present disclosure exhibit reduced or eliminated expression of BAX; BAK; ICAM-1; SIRT-1; MYC; GGTA1; CMAH; LPL; LIPA; BCKDHA; BCKDHB; LPLA2 and PPT1. In certain embodiments, the TI cells of the present disclosure exhibit reduced or eliminated expression of BAX; BAK; ICAM-1; PERK; SIRT-1; MYC; GGTA1; CMAH; LPL; LIPA; BCKDHA; BCKDHB; LPLA2 and PPT1. In certain embodiments, the TI cells of the present disclosure exhibit reduced or eliminated expression of BAX; BAK; ICAM-1; SIRT-1; MYC; GGTA1; CMAH; and LPL. In certain embodiments, the TI cells of the present disclosure exhibit reduced or eliminated expression of BAX; BAK; ICAM-1; PERK; SIRT-1; MYC; GGTA1; CMAH; and LPL. In certain embodiments, the TI cells of the present disclosure exhibit reduced or eliminated expression of BAX; BAK; ICAM-1; SIRT-1; MYC; GGTA1; CMAH; and LPLA2. In certain embodiments, the TI cells of the present disclosure exhibit reduced or eliminated expression of BAX; BAK; ICAM-1; PERK; SIRT-1; MYC; GGTA1; CMAH; and LPLA2. In certain embodiments, the TI cells of the present disclosure exhibit reduced or eliminated expression of BAX; BAK; ICAM-1; SIRT-1; MYC; GGTA1; CMAH; and PPT1. In certain embodiments, the TI cells of the present disclosure exhibit reduced or eliminated expression of BAX; BAK; ICAM-1; SIRT-1; PERK; MYC; GGTA1; CMAH; and PPT1. In certain embodiments, the TI cells of the present disclosure exhibit reduced or eliminated expression of BAX; BAK; MYC; SIRT-1; and ICAM. In certain embodiments, the TI cells of the present disclosure exhibit reduced or eliminated expression of BAX; BAK; LPL; LPLA2; GGTA1; and CMAH. In certain embodiments, the TI cells of the present disclosure exhibit reduced or eliminated expression of BAX; BAK; PERK; LPL; LPLA2; GGTA1; and CMAH. In certain embodiments, the TI cells of the present disclosure exhibit reduced or eliminated expression of BAX; BAK; MYC; LPL; LPLA2; GGTA1; and CMAH. In certain embodiments, the TI cells of the present disclosure exhibit reduced or eliminated expression of BAX; BAK; MYC; PERK; LPL; LPLA2; GGTA1; and CMAH. In certain embodiments, the TI cells of the present disclosure exhibit reduced or eliminated expression of BAX; BAK; LPL; LPLA2; GGTA1; CMAH; and PPT1. In certain embodiments, the TI cells of the present disclosure exhibit reduced or eliminated expression of BAX; BAK; PERK; LPL; LPLA2; GGTA1; CMAH; and PPT1. In certain embodiments, the TI cells of the present disclosure exhibit reduced or eliminated expression of BAX; BAK; MYC; LPL; LPLA2; GGTA1; CMAH; and PPT1. In certain embodiments, the TI cells of the present disclosure exhibit reduced or eliminated expression of BAX; BAK; MYC; PERK; LPL; LPLA2; GGTA1; CMAH; and PPT1. In certain embodiments, the TI cells of the present disclosure exhibit reduced or eliminated expression of one or more of BAX; BAK; ICAM-1; SIRT-1; PERK; MYC; GGTA1; CMAH; LPL; LIPA; BCKDHA; BCKDHB; LPLA2 and/or PPT1.

In certain embodiments, the present disclosure relates to TI cells or compositions comprising one or more TI cells exhibiting improved cell culture performance, improved product quality due to elimination of undesired types of glycosylation, improved product stability due to decreased risk of polysorbate degradation, and improved purification performance. In certain embodiments, the TI cells of the present disclosure exhibit reduced or eliminated expression of GAG; BAX; BAK; ICAM-1; PERK; SIRT-1; MYC; GGTA1; CMAH; LPL; LIPA; BCKDHA; BCKDHB; LPLA2 and/or PPT1. In certain embodiments, the TI cells of the present disclosure exhibit reduced or eliminated expression of one or more of GAG; BAX; BAK; ICAM-1; PERK; SIRT-1; MYC; GGTA1; CMAH; LPL; LIPA; BCKDHA; BCKDHB; LPLA2 and/or PPT1. In certain embodiments, the TI cells of the present disclosure exhibit reduced or eliminated expression of GAG; BAX; BAK; ICAM-1; PERK; SIRT-1 and/or MYC. In certain embodiments, the TI cells of the present disclosure exhibit reduced or eliminated expression of one or more of GAG; BAX; BAK; ICAM-1; PERK and/or SIRT-1. In certain embodiments, the TI cells of the present disclosure exhibit reduced or eliminated expression of one or more of BAX; BAK; PERK and/or ICAM-1.

In certain embodiments, the present disclosure relates to modified cells or compositions one or more modified cell exhibiting improved cell culture performance and improved product quality due to elimination of undesired types of glycosylation. In certain embodiments, the modified cells of the present disclosure exhibit improved cell culture performance due to: i) increased/extended viability and healthier mitochondria for metabolism; ii) decreased cell clumping/aggregation; and/or iii) higher productivity and higher titers. In certain embodiments, the modified cells of the present disclosure exhibit reduced or eliminated expression of BAX; BAK, ICAM-1; SIRT-1; PERK; MYC; GGTA1 and/or CMAH. In certain embodiments, the modified cells of the present disclosure exhibit reduced or eliminated expression of one or more of BAX; BAK, ICAM-1; PERK; SIRT-1; MYC; GGTA1 and/or CMAH. In certain embodiments, the modified cells of the present disclosure have reduced or eliminated expression of BAX; BAK, ICAM-1; SIRT-1; PERK; MYC; and/or GGTA1. In certain embodiments, the modified cells of the present disclosure have reduced or eliminated expression of BAX; BAK, ICAM-1; PERK; SIRT-1; MYC; and/or CMAH.

In certain embodiments, the modified cells of the present disclosure exhibit improved cell culture performance and improved product stability due to decreased risk of polysorbate degradation. In certain embodiments, the modified cells of the present disclosure exhibit improved cell culture performance due to: i) increased/extended viability and healthier mitochondria for metabolism; ii) decreased cell clumping/aggregation; and/or iii) higher productivity and higher titers. In certain embodiments, the modified cells of the present disclosure exhibit improved product stability due to decreased levels of residual hydrolytic enzymes in the product. In certain embodiments, the modified cells of the present disclosure exhibit reduced or eliminated expression ofBAX; BAK, ICAM-1; PERK; SIRT-1; MYC; LPL, LIPA; BCKDHA; BCKDHB; LPLA2 and/or PPT1. In certain embodiments, the modified cells of the present disclosure exhibit reduced or eliminated expression of one or more of BAX; BAK, ICAM-1; PERK; SIRT-1; MYC; LPL, LIPA; LPLA2 and/or PPT1. In certain embodiments, the modified cells of the present disclosure exhibit reduced or eliminated expression of BAX; BAK, ICAM-1; PERK; SIRT-1; MYC; and/or LPL. In certain embodiments, the modified cells of the present disclosure exhibit reduced or eliminated expression of BAX; BAK, ICAM-1; PERK; SIRT-1; MYC; and/or LPLA2. In certain embodiments, the modified cells of the present disclosure exhibit reduced or eliminated expression of BAX; BAK, ICAM-1; PERK; SIRT-1; MYC; and/or PPT1. In certain embodiments, the modified cells of the present disclosure exhibit reduced or eliminated expression of BAX; BAK, ICAM-1; SIRT-1; PERK; MYC; LPL; LIPA and/or LPLA2. In certain embodiments, the modified cells of the present disclosure exhibit reduced or eliminated expression of BAX; BAK, ICAM-1; PERK; SIRT-1; MYC; LPL; LIPA and/or PPT1. In certain embodiments, the modified cells of the present disclosure exhibit reduced or eliminated expression of BAX; BAK, ICAM-1; SIRT-1; PERK; MYC; LIPA; LPLA2 and/or PPT1.

In certain embodiments, the modified cells of the present disclosure exhibit improved cell culture performance and improved purification performance. In certain embodiments, the modified cells of the present disclosure exhibit improved cell culture performance due to: i) increased/extended viability and healthier mitochondria for metabolism; ii) decreased cell clumping/aggregation; and/or iii) higher productivity and higher titers. In certain embodiments, the modified cells of the present disclosure exhibit improved purification performance due to elimination of a variety of endogenous host cell products (e.g., endogenous virus-like particles and/or endogenous host cell proteins). In certain embodiments, the modified cells of the present disclosure exhibit improved purification performance and improved purification performance due to reduced or eliminated expression of GAG and/or BAX; BAK; ICAM-1; PERK; SIRT-1; MYC; GGTA1; CMAH; LPL; LIPA; LPLA2; and/or PPT1. In certain embodiments, the modified cells of the present disclosure exhibit improved purification performance due to cleaner harvest with less cellular debris. In certain embodiments, the modified cells of the present disclosure exhibit cleaner harvest with less cellular debris and improved purification performance due to reduced or eliminated expression of GAG and/or BAX; BAK; ICAM-1;PERK; SIRT-1 and/or MYC. In certain embodiments, the modified cells of the present disclosure exhibit cleaner harvest with less cellular debris and improved purification performance due to reduced or eliminated expression of GAG and/or BAX; BAK; ICAM-1; PERK and/or SIRT-1. In certain embodiments, the modified cells of the present disclosure exhibit cleaner harvest with less cellular debris and improved purification performance due to reduced or eliminated expression of GAG and/or BAX; BAK; PERK and/or ICAM-1.

In certain embodiments, the modified cells of the present disclosure exhibit improved cell culture performance, improved product quality due to elimination of undesired types of glycosylation, and improved product stability due to decreased risk of polysorbate degradation and/or due to decreased levels of residual hydrolytic enzymes in the product. In certain embodiments, the modified cells of the present disclosure exhibit improved cell culture performance due to: i) increased/extended viability and healthier mitochondria for metabolism; ii) decreased cell clumping/aggregation; and/or iii) higher productivity and higher titers. In certain embodiments, the modified cells of the present disclosure exhibit reduced or eliminated expression of BAX; BAK, ICAM-1; PERK; SIRT-1; GGTA1; CMAH; MYC; LPL; LIPA; BCKDHA; BCKDHB; LPLA2; and/or PPT1. In certain embodiments, the modified cells of the present disclosure exhibit reduced or eliminated expression of one or more of BAX; BAK, ICAM-1; PERK; SIRT-1; GGTA1; CMAH; MYC; LPL; LIPA; LPLA2; and/or PPT1. In certain embodiments, the modified cells of the present disclosure exhibit reduced or eliminated expression of BAX; BAK, ICAM-1; PERK; SIRT-1; GGTA1; CMAH; MYC; and/or LPL. In certain embodiments, the modified cells of the present disclosure exhibit reduced or eliminated expression of BAX; BAK, ICAM-1; SIRT-1; PERK; GGTA1; CMAH; MYC; and/or LPLA2.

In certain embodiments, the modified cells of the present disclosure exhibit reduced or eliminated expression of BAX; BAK, ICAM-1; PERK; SIRT-1; GGTA1; CMAH; MYC; and/or PPT1. In certain embodiments, the modified cells of the present disclosure exhibit reduced or eliminated expression of BAX; BAK, ICAM-1; PERK; SIRT-1; GGTA1; CMAH; MYC; LPL; LIPA and/or LPLA2. In certain embodiments, the modified cells of the present disclosure exhibit reduced or eliminated expression of BAX; BAK, ICAM-1; SIRT-1; PERK; GGTA1; CMAH; MYC; LPL; LIPA and/or PPT1. In certain embodiments, the modified cells of the present disclosure exhibit reduced or eliminated expression of BAX; BAK, ICAM-1; PERK; SIRT-1; GGTA1; CMAH; MYC; LIPA; BCKDHA; BCKDHB; LPLA2; and/or PPT1.

In certain embodiments, the modified cells of the present disclosure exhibit improved cell culture performance, improved product quality due to elimination of undesired types of glycosylation, improved product stability due to decreased risk of polysorbate degradation, and improved purification performance. In certain embodiments, the modified cells of the present disclosure exhibit reduced or eliminated expression of GAG; BAX; BAK; ICAM-1; PERK; SIRT-1; MYC; GGTA1; CMAH; LPL; LIPA; BCKDHA; BCKDHB; LPLA2 and/or PPT1. In certain embodiments, the modified cells of the present disclosure exhibit reduced or eliminated expression of one or more of GAG; BAX; BAK; ICAM-1; PERK; SIRT-1; MYC; GGTA1; CMAH; LPL; LIPA; BCKDHA; BCKDHB; LPLA2 and/or PPT1. In certain embodiments, the modified cells of the present disclosure exhibit reduced or eliminated expression of GAG; BAX; BAK; ICAM-1; PERK; GGTA1; CMAH; LPL; LIPA; BCKDHA; BCKDHB; LPLA2 and/or PPT1. In certain embodiments, the modified cells of the present disclosure exhibit reduced or eliminated expression of one or more of GAG; BAX; BAK; ICAM-1; PERK; GGTA1; CMAH; LPL; LIPA; BCKDHA; BCKDHB; LPLA2 and/or PPT1.

In certain embodiments, the modified cells of the present disclosure exhibit improved product quality due to elimination of undesired types of glycosylation and improved product stability due to decreased risk of polysorbate degradation, and/or decreased levels of residual hydrolytic enzymes in the product. In certain embodiments, the modified cells of the present disclosure exhibit improved product quality due to reduced or eliminated expression of GGTA1; CMAH; LPL; LIPA; LPLA2 and/or PPT1. In certain embodiments, the modified cells of the present disclosure exhibit improved product quality due to reduced or eliminated expression of GGTA1; CMAH and LPL. In certain embodiments, the modified cells of the present disclosure exhibit improved product quality due to reduced or eliminated expression of GGTA1; CMAH and LPLA2. In certain embodiments, the modified cells of the present disclosure exhibit improved product quality due to reduced or eliminated expression of GGTA1; CMAH and PPT1. In certain embodiments, the modified cells of the present disclosure exhibit improved product quality due to reduced or eliminated expression of GGTA1; LPL; LIPA; LPLA2 and/or PPT1. In certain embodiments, the modified cells of the present disclosure exhibit improved product quality due to reduced or eliminated expression of CMAH; LPL; LIPA; LPLA2 and/or PPT1. In certain embodiments, the modified cells of the present disclosure exhibit improved product quality due to reduced or eliminated expression of one or more of GGTA1; CMAH; LPL; LIPA; LPLA2 and/or PPT1.

In certain embodiments, the modified cells of the present disclosure exhibit improved product quality due to elimination of undesired types of glycosylation and improved purification performance. In certain embodiments, the modified cells of the present disclosure exhibit reduced or eliminated expression of GAG; BAX; BAK; ICAM-1; SIRT-1; MYC; GGTA1; CMAH; LPL; LIPA; BCKDHA; BCKDHB; LPLA2; and PPT1. In certain embodiments, the modified cells of the present disclosure exhibit reduced or eliminated expression of one or more of GAG; BAX; BAK; ICAM-1; SIRT-1; MYC; GGTA1; CMAH; LPL; LIPA; BCKDHA; BCKDHB; LPLA2; and/or PPT1. In certain embodiments, the modified cells of the present disclosure exhibit reduced or eliminated expression of GAG; BAX; BAK; ICAM-1; SIRT-1; MYC; GGTA1 and/or CMAH. In certain embodiments, the modified cells of the present disclosure exhibit reduced or eliminated expression of GAG; BAX; BAK; ICAM-1; SIRT-1; MYC; and GGTA1. In certain embodiments, the modified cells of the present disclosure exhibit reduced or eliminated expression of GAG; BAX; BAK; ICAM-1; SIRT-1; MYC; and CMAH. In certain embodiments, the modified cells of the present disclosure exhibit reduced or eliminated expression of GAG; BAX; BAK; ICAM-1; SIRT-1; and MYC. In certain embodiments, the modified cells of the present disclosure exhibit reduced or eliminated expression of GAG; BAX; BAK; LPL; LPLA2; GGTA1; and CMAH. In certain embodiments, the modified cells of the present disclosure exhibit reduced or eliminated expression of GAG; BAX; BAK; MYC; LPL; LPLA2; GGTA1; and CMAH. In certain embodiments, the modified cells of the present disclosure exhibit reduced or eliminated expression of GAG; BAX; BAK; LPL; LPLA2; GGTA1; CMAH; and PPT1. In certain embodiments, the modified cells of the present disclosure exhibit reduced or eliminated expression of GAG; BAX; BAK; MYC; LPL; LPLA2; GGTA1; CMAH; and PPT1.

In certain embodiments, the modified cells of the present disclosure exhibit improved product quality due to elimination of undesired types of glycosylation, improved product stability due to decreased risk of polysorbate degradation and/or decreased levels of residual hydrolytic enzymes in the product, and improved purification performance. In certain embodiments, the modified cells of the present disclosure exhibit reduced or eliminated expression of GAG; BAX; BAK; ICAM-1; SIRT-1; MYC; GGTA1; CMAH; LPL; LIPA; LPLA2; and PPT1. In certain embodiments, the modified cells of the present disclosure exhibit reduced or eliminated expression of one or more of GAG; BAX; BAK; ICAM-1; SIRT-1; MYC; GGTA1; CMAH; LPL; LIPA; BCKDHA; BCKDHB; LPLA2; and/or PPT1. In certain embodiments, the modified cells of the present disclosure exhibit reduced or eliminated expression of GAG; BAX; BAK; ICAM-1; SIRT-1; MYC; GGTA1 and/or CMAH. In certain embodiments, the modified cells of the present disclosure exhibit reduced or eliminated expression of GAG; BAX; BAK; ICAM-1; SIRT-1; MYC; and GGTA1. In certain embodiments, the modified cells of the present disclosure exhibit reduced or eliminated expression of GAG; BAX; BAK; ICAM-1; SIRT-1; MYC; and CMAH. In certain embodiments, the modified cells of the present disclosure exhibit reduced or eliminated expression of GAG; BAX; BAK; ICAM-1; SIRT-1; and MYC. In certain embodiments, the modified cells of the present disclosure exhibit reduced or eliminated expression of GAG; BAX; BAK; LPL; LPLA2; GGTA1; and CMAH. In certain embodiments, the modified cells of the present disclosure exhibit reduced or eliminated expression of GAG; BAX; BAK; MYC; LPL; LPLA2; GGTA1; and CMAH. In certain embodiments, the modified cells of the present disclosure exhibit reduced or eliminated expression of GAG; BAX; BAK; LPL; LPLA2; GGTA1; CMAH; and PPT1. In certain embodiments, the modified cells of the present disclosure exhibit reduced or eliminated expression of GAG; BAX; BAK; MYC; LPL; LPLA2; GGTA1; CMAH; and PPT1.

In certain embodiments, the modified cells of the present disclosure exhibit improved product stability due to decreased risk of polysorbate degradation and/or decreased levels of residual hydrolytic enzymes in the product and improved purification performance. In certain embodiments, the modified cells of the present disclosure exhibit improved purification performance due to reduced or eliminated expression of GAG; BAX; BAK; ICAM-1; SIRT-1; MYC; GGTA1; CMAH; LPL; LIPA; LPLA2; and PPT1. In certain embodiments, the modified cells of the present disclosure exhibit improved purification performance due to reduced or eliminated expression of GAG; BAX; BAK; ICAM-1; SIRT-1; MYC; GGTA1; CMAH; and LPL. In certain embodiments, the modified cells of the present disclosure exhibit improved purification performance due to reduced or eliminated expression of GAG; BAX; BAK; ICAM-1; SIRT-1; MYC; GGTA1; CMAH; and LPLA2. In certain embodiments, the modified cells of the present disclosure exhibit improved purification performance due to reduced or eliminated expression of GAG; BAX; BAK; ICAM-1; SIRT-1; MYC; GGTA1; CMAH; and LIPA. In certain embodiments, the modified cells of the present disclosure exhibit improved purification performance due to reduced or eliminated expression of GAG; BAX; BAK; ICAM-1; SIRT-1; MYC; GGTA1; CMAH; and PPT1. In certain embodiments, the modified cells of the present disclosure exhibit improved purification performance due to reduced or eliminated expression of GAG; BAX; BAK; ICAM-1; SIRT-1; MYC; GGTA1; CMAH; LPL and LPLA2. In certain embodiments, the modified cells of the present disclosure exhibit improved purification performance due to reduced or eliminated expression of GAG; BAX; BAK; ICAM-1; SIRT-1; MYC; GGTA1; CMAH; LPL and PPT1. In certain embodiments, the modified cells of the present disclosure exhibit improved purification performance due to reduced or eliminated expression of GAG; BAX; BAK; ICAM-1; SIRT-1; MYC; GGTA1; CMAH; LPLA2; and PPT1. In certain embodiments, the modified cells of the present disclosure exhibit reduced or eliminated expression of one or more of GAG; BAX; BAK; ICAM-1; SIRT-1; MYC; GGTA1; CMAH; LPL; LIPA; LPLA2; and/or PPT1. In certain embodiments, the modified cells of the present disclosure exhibit reduced or eliminated expression of GAG; BAX; BAK; ICAM-1; SIRT-1; MYC LPL; LIPA; LPLA2; and PPT1. In certain embodiments, the modified cells of the present disclosure exhibit reduced or eliminated expression of one or more of GAG; BAX; BAK; ICAM-1; SIRT-1; MYC LPL; LIPA; BCKDHA; BCKDHB; LPLA2; and/or PPT1. In certain embodiments, the modified cells of the present disclosure exhibit reduced or eliminated expression of GAG; BAX; BAK; ICAM-1; SIRT-1; MYC; LPL; LIPA; LPLA2; and PPT1. In certain embodiments, the modified cells of the present disclosure exhibit reduced or eliminated expression of GAG; BAX; BAK; ICAM-1; SIRT-1; MYC; and LPL. In certain embodiments, the modified cells of the present disclosure exhibit reduced or eliminated expression of GAG; BAX; BAK; ICAM-1; SIRT-1; MYC; and LPLA2. In certain embodiments, the modified cells of the present disclosure exhibit reduced or eliminated expression of GAG; BAX; BAK; ICAM-1; SIRT-1; MYC; and PPT1.

In certain embodiments, the modified cells of the present disclosure exhibit reduced or eliminated expression of GAG; BAX; BAK; ICAM-1; SIRT-1; MYC; LPL; and LPLA2. In certain embodiments, the modified cells of the present disclosure exhibit reduced or eliminated expression of GAG; BAX; BAK; ICAM-1; SIRT-1; MYC; LPL and PPT1. In certain embodiments, the modified cells of the present disclosure exhibit reduced or eliminated expression of GAG; BAX; BAK; ICAM-1; SIRT-1; MYC; LPLA2; and PPT1. In certain embodiments, the modified cells of the present disclosure exhibit reduced or eliminated expression of GAG; BAX; BAK; ICAM-1; SIRT-1; and MYC. In certain embodiments, the modified cells of the present disclosure exhibit reduced or eliminated expression of GAG; BAX; BAK; LPL; LPLA2; GGTA1; and CMAH. In certain embodiments, the modified cells of the present disclosure exhibit reduced or eliminated expression of GAG; BAX; BAK; MYC; LPL; LPLA2; GGTA1; and CMAH.

In certain embodiments, a host cell is a cell line. In certain embodiments, a host cell is a cell line that has been cultured for a certain number of generations. In certain embodiments, a host cell is a primary cell.

In certain embodiments, expression of a polypeptide of interest is stable if the expression level is maintained at certain levels, increases, or decreases less than 20%, over 10, 20, 30, 50, 100, 200, or 300 generations. In certain embodiments, expression of a polypeptide of interest is stable if the culture can be maintained without any selection. In certain embodiments, expression of a polypeptide of interest is high if the polypeptide product of the gene of interest reaches about 1 g/L, about 2 g/L, about 3 g/L, about 4 g/L, about 5 g/L, about 10 g/L, about 12 g/L, about 14 g/L, or about 16 g/L.

Exogenous nucleotides of interest or vectors can be introduced into a host cell by conventional cell biology methods including, but not limited to, transfection, transduction, electroporation, or injection. In certain embodiments, exogenous nucleotides of interest or vectors are introduced into a host cell by chemical-based transfection methods comprising lipid-based transfection method, calcium phosphate-based transfection method, cationic polymer-based transfection method, or nanoparticle-based transfection. In certain embodiments, exogenous nucleotides of interest are introduced into a host cell by virus-mediated transduction including, but not limited to, lentivirus, retrovirus, adenovirus, or adeno-associated virus-mediated transduction. In certain embodiments, exogenous nucleotides of interest or vectors are introduced into a host cell via gene gun-mediated injection. In certain embodiments, both DNA and RNA molecules are introduced into a host cell using methods described herein.

### 5.4. Cell Culturing Methods

In one aspect, the present disclosure provides a method for producing a recombinant product of interest comprising culturing a modified cell disclosed herein. Suitable culture conditions for mammalian cells known in the art can be used for culturing the modified cells disclosed herein (J. Immunol. Methods (1983) 56:221-234) or can be easily determined by the skilled artisan (see, for example, Animal Cell Culture: A Practical Approach 2nd Ed., Rickwood, D. and Hames, B. D., eds. Oxford University Press, New York (1992)).

Mammalian cell culture can be prepared in a medium suitable for the particular cell being cultured. Commercially available media such as Ham's F10 (Sigma), Minimal Essential Medium (MEM, Sigma), RPMI-1640 (Sigma) and Dulbecco's Modified Eagle's Medium (DMEM, Sigma) are exemplary nutrient solutions. In addition, any of the media described in Ham and Wallace, (1979) Meth. Enz., 58:44; Barnes and Sato, (1980) Anal. Biochem., 102:255; U.S. Pat. Nos. 4,767,704; 4,657,866; 4,927,762; 5,122,469 or U.S. Pat. No. 4,560,655; International Publication Nos. WO 90/03430; and WO 87/00195; the disclosures of all of which are incorporated herein by reference, can be used as culture media. Any of these media can be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as gentamycin (gentamicin), trace elements (defined as inorganic compounds usually present at final concentrations in the micromolar range) lipids (such as linoleic or other fatty acids) and their suitable carriers, and glucose or an equivalent energy source. Any other necessary supplements can also be included at appropriate concentrations that would be known to those skilled in the art.

In certain embodiments, the mammalian cell that has been modified to reduce and/or eliminate the activity of a particular endogenous product is a CHO cell. Any suitable medium can be used to culture the CHO cell of the present disclosure. In certain embodiments, a suitable medium for culturing the CHO cell can contain a basal medium component such as a DMEM/HAM F-12 based formulation (for composition of DMEM and HAM F12 media, see culture media formulations in American Type Culture Collection Catalogue of Cell Lines and Hybridomas, Sixth Edition, 1988, pages 346-349) (the formulation of medium as described in U.S. Pat. No. 5,122,469 are particularly appropriate) with modified concentrations of some components such as amino acids, salts, sugar, and vitamins, and optionally containing glycine, hypoxanthine, and thymidine; recombinant human insulin, hydrolyzed peptone, such as Primatone H S or Primatone R L (Sheffield, England), or the equivalent; a cell protective agent, such as Pluronic F68 or the equivalent pluronic polyol; gentamycin; and trace elements.

In certain embodiments, the mammalian cell that has been modified to reduce and/or eliminate the expression of a particular endogenous product, e.g., GAG and/or a BAX; BAK; ICAM-1; SIRT-1; MYC; GGTA1; CMAH; LPL; LIPA; LPLA2; BCKDHA; BCKDHB; PPT1; and/or PERK polypeptide, is a cell that expresses a recombinant product. The recombinant product can be produced by growing cells which express the recombinant product of interest under a variety of cell culture conditions. For instance, cell culture procedures for the large or small-scale production of recombinant products are potentially useful within the context of the present disclosure. Procedures including, but not limited to, a fluidized bed bioreactor, hollow fiber bioreactor, roller bottle culture, shake flask culture, or stirred tank bioreactor system can be used, in the latter two systems, with or without microcarriers, and operated alternatively in a batch, fed-batch, or continuous mode.

In certain embodiments, the cell culture of the present disclosure is performed in a stirred tank bioreactor system and a fed batch culture procedure is employed. In the fed batch culture, the mammalian host cells and culture medium are supplied to a culturing vessel initially and additional culture nutrients are fed, continuously or in discrete increments, to the culture during culturing, with or without periodic cell and/or product harvest before termination of culture. The fed batch culture can include, for example, a semi-continuous fed batch culture, wherein periodically whole culture (including cells and medium) is removed and replaced by fresh medium. Fed batch culture is distinguished from simple batch culture in which all components for cell culturing (including the cells and all culture nutrients) are supplied to the culturing vessel at the start of the culturing process. Fed batch culture can be further distinguished from perfusion culturing insofar as the supernatant is not removed from the culturing vessel during the process (in perfusion culturing, the cells are restrained in the culture by, e.g., filtration, encapsulation, anchoring to microcarriers etc. and the culture medium is continuously or intermittently introduced and removed from the culturing vessel).

In certain embodiments, the cells of the culture can be propagated according to any scheme or routine that can be suitable for the specific host cell and the specific production plan contemplated. Therefore, the present disclosure contemplates a single step or multiple step culture procedure. In a single step culture, the host cells are inoculated into a culture environment and the processes of the instant disclosure are employed during a single production phase of the cell culture. Alternatively, a multi-stage culture is envisioned. In the multi-stage culture cells can be cultivated in a number of steps or phases. For instance, cells can be grown in a first step or growth phase culture wherein cells, possibly removed from storage, are inoculated into a medium suitable for promoting growth and high viability. The cells can be maintained in the growth phase for a suitable period of time by the addition of fresh medium to the host cell culture.

In certain embodiments, fed batch or continuous cell culture conditions are devised to enhance growth of the mammalian cells in the growth phase of the cell culture. In the growth phase cells are grown under conditions and for a period of time that is maximized for growth. Culture conditions, such as temperature, pH, dissolved oxygen (d02) and the like, are those used with the particular host and will be apparent to the ordinarily skilled artisan. Generally, the pH is adjusted to a level between about 6.5 and 7.5 using either an acid (e.g., CO2) or a base (e.g., Na2CO3 or NaOH). A suitable temperature range for culturing mammalian cells such as CHO cells is between about 30° C. to 38° C. and a suitable d02 is between 5-90% of air saturation.

At a particular stage the cells can be used to inoculate a production phase or step of the cell culture. Alternatively, as described above the production phase or step can be continuous with the inoculation or growth phase or step.

In certain embodiments, the culturing methods described in the present disclosure can further include harvesting the recombinant product from the cell culture, e.g., from the production phase of the cell culture. In certain embodiments, the recombinant product produced by the cell culture methods of the present disclosure can be harvested from the third bioreactor, e.g., production bioreactor. For example, but not by way of limitation, the disclosed methods can include harvesting the recombinant product at the completion of the production phase of the cell culture. Alternatively or additionally, the recombinant product can be harvested prior to the completion of the production phase. In certain embodiments, the recombinant product can be harvested from the cell culture once a particular cell density has been achieved. For example, but not by way of limitation, the cell density can be from about 2.0×10⁷ cells/mL to about 5.0×10⁷ cells/mL prior to harvesting.

In certain embodiments, harvesting the product from the cell culture can include one or more of centrifugation, filtration, acoustic wave separation, flocculation and cell removal technologies.

In certain embodiments, the recombinant product of interest can be secreted from the host cells or can be a membrane-bound, cytosolic or nuclear protein. In certain embodiments, soluble forms of the recombinant product can be purified from the conditioned cell culture media and membrane-bound forms of the recombinant product can be purified by preparing a total membrane fraction from the expressing cells and extracting the membranes with a nonionic detergent such as TRITON® X-100 (EMD Biosciences, San Diego, Calif.). In certain embodiments, cytosolic or nuclear proteins can be prepared by lysing the host cells (e.g., by mechanical force, sonication and/or detergent), removing the cell membrane fraction by centrifugation and retaining the supernatant.

### 5.5 Production of a Recombinant Product of Interest

The cells and/or methods of the present disclosure can be used to produce any recombinant product of interest that can be expressed by the cells disclosed herein.

### 5.5.1 Viral Particle and Viral Vector Products

In certain embodiments, the cells and/or methods of the present disclosure can be used for the production of viral particles or viral vectors. In certain embodiments, the methods of the present disclosure can be used for the production of viral particles. In certain embodiments, the methods of the present disclosure can be used for the production of viral vectors. In certain embodiments, the methods of the present disclosure can be used for the expression of virus polypeptides. Non-limiting examples of such polypeptides include virus proteins, virus structural (Cap) proteins, virus packaging (Rep) proteins, AAV capsid proteins and virus helper proteins. In certain embodiments, the virus polypeptide is an AAV virus polypeptide.

In certain embodiments, the cells useful in connection with the production of viral particles or viral vectors include, but are not limited to: human embryonic kidney line (e.g., HEK 293 or HEK 293 cells subcloned for growth in suspension culture), human cervical carcinoma cells (e.g., HELA, ATCC CCL 2), human lung cells (e.g., W138, ATCC CCL 75), human liver cells (e.g., Hep G2, HB 8065), human hepatoma line (e.g., Hep G2), myeloma cell lines (e.g., Y0, NSO and Sp2/0), monkey kidney CV1 line transformed by SV40 (e.g., COS-7 ATCC CRL-1651), baby hamster kidney cells (e.g., BHK, ATCC CCL 10), mouse sertoli cells (e.g. TM4), monkey kidney cells (e.g., CV1 ATCC CCL 70), African green monkey kidney cells (e.g., VERO-76, ATCC CRL-1587), canine kidney cells (e.g., MDCK, ATCC CCL 34), buffalo rat liver cells (e.g., BRL 3A, ATCC CRL 1442), mouse mammary tumor (e.g., MMT 060562, ATCC CCL51), TRI cells, MRC 5 cells, and FS4 cells. In certain embodiments, the cells are CHO cells. Additional non-limiting examples of CHO host cells include CHO KlSV cells, CHO DG44 cells, a CHO DUKXB-11 cells, CHOKIS cells and CHO KIM cells

In certain embodiments, examples of genes of interest that can be carried by the viral particles produced by the methods describe herein include mammalian polypeptides, such as, e.g., renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; leptin; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hematopoietic growth factor; tumor necrosis factor-alpha and -beta; a tumor necrosis factor receptor such as death receptor 5 and CD120; TNF-related apoptosis-inducing ligand (TRAIL); B-cell maturation antigen (BCMA); B-lymphocyte stimulator (BLyS); a proliferation-inducing ligand (APRIL); enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; platelet-derived endothelial cell growth factor (PD-ECGF); a vascular endothelial growth factor family protein (e.g., VEGF-A, VEGF-B, VEGF-C, VEGF-D, and PTGF); a platelet-derived growth factor (PDGF) family protein (e.g., PDGF-A, PDGF-B, PDGF-C, PDGF-D, and dimers thereof); fibroblast growth factor (FGF) family such as aFGF, bFGF, FGF4, and FGF9; epidermal growth factor (EGF); receptors for hormones or growth factors such as a VEGF receptor(s) (e.g., VEGFR1, VEGFR2, and VEGFR3), epidermal growth factor (EGF) receptor(s) (e.g., ErbBI, ErbB2, ErbB3, and ErbB4 receptor), platelet-derived growth factor (PDGF) receptor(s) (e.g., PDGFR-α and PDGFR-0), and fibroblast growth factor receptor(s); TIE ligands (Angiopoietins, ANGPT1, ANGPT2); Angiopoietin receptor such as TIE1 and TIE2; protein A or D; rheumatoid factors; a neurotrophic factor such as bone-derived neurotrophic factor (BDNF), neurotrophin-3, −4, −5, or −6 (NT-3, NT-4, NT-5, or NT-6), or a nerve growth factor such as NGF-b; transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins (IGFBPs); CD proteins such as CD3, CD4, CD8, CD19 and CD20; erythropoietin; osteoinductive factors; immunotoxins; a bone morphogenetic protein (BMP); a chemokine such as CXCL12 and CXCR4; an interferon such as interferon-alpha, -beta, and -gamma; colony stimulating factors (CSFs), e.g., M-CSF, GM-CSF, and G-CSF; a cytokine such as interleukins (ILs), e.g., IL-I to IL-10; midkine; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; ephrins; Bv8; Delta-like ligand 4 (DLL4); Del-1; BMP9; BMPIO; Follistatin; Hepatocyte growth factor (HGF)/scatter factor (SF); Alki; Robo4; ESMI; Perlecan; EGF-like domain, multiple 7 (EGFL7); CTGF and members of its family; thrombospondins such as thrombospondini and thrombospondin2; collagens such as collagen IV and collagen XVIII; neuropilins such as NRP1 and NRP2; Pleiotrophin (PTN); Progranulin; Proliferin; Notch proteins such as Notchi and Notch4; semaphorins such as Sema3A, Sema3C, and Sema3F; a tumor associated antigen such as CA125 (ovarian cancer antigen); immunoadhesins; and fragments and/or variants of any of the above-listed polypeptides as well as antibodies, including antibody fragments, binding to one or more protein, including, for example, any of the above-listed proteins.

In some embodiments, the gene of interest carried by the viral particles produced by the mammalian cells of the present disclosure may encode proteins that bind to, or interact with, any protein, including, without limitation, cytokines, cytokine-related proteins, and cytokine receptors selected from the group consisting of 8MPI, 8MP2, 8MP38 (GDFIO), 8MP4, 8MP6, 8MP8, CSFI (M-CSF), CSF2 (GM-CSF), CSF3 (G-CSF), EPO, FGF1 (αFGF), FGF2 (βFGF), FGF3 (int-2), FGF4 (HST), FGF5, FGF6 (HST-2), FGF7 (KGF), FGF9, FGF1 0, FGF11, FGF12, FGF12B, FGF14, FGF16, FGF17, FGF19, FGF20, FGF21, FGF23, IGF1, IGF2, IFNA1, IFNA2, IFNA4, IFNA5, IFNA6, IFNA7, IFN81, IFNG, IFNWI, FEL1, FEL1 (EPSELON), FEL1 (ZETA), IL 1A, IL 1B, IL2, IL3, IL4, IL5, IL6, IL7, IL8, IL9, IL1 0, IL 11, IL 12A, IL 12B, IL 13, IL 14, IL 15, IL 16, IL 17, IL 17B, IL 18, IL 19, IL20, IL22, IL23, IL24, IL25, IL26, IL27, IL28A, IL28B, IL29, IL30, PDGFA, PDGFB, TGFA, TGFB1, TGFB2, TGFBb3, LTA (TNF-0), LTB, TNF (TNF-u), TNFSF4 (OX40 ligand), TNFSF5 (CD40 ligand), TNFSF6 (FasL), TNFSF7 (CD27 ligand), TNFSF8 (CD30 ligand), TNFSF9 (4-1 BB ligand), TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, HGF (VEGFD), VEGF, VEGFB, VEGFC, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL 11RA, IL12RB1, IL12RB2, IL13RA1, IL13RA2, IL15RA, IL17R, IL18R1, IL20RA, IL21R, IL22R, IL1HYl, ILIRAP, ILIRAPLI, IL1RAPL2, IL1RN, IL6ST, IL18BP, IL18RAP, IL22RA2, AIF1, HGF, LEP (leptin), PTN, and THPO.k.

In some embodiments, the gene of interest carried by the viral particles produced by the mammalian cells of the present disclosure may encode proteins that bind to, or interact with, a chemokine, chemokine receptor, or a chemokine-related protein selected from the group consisting of CCLI (1-309), CCL2 (MCP-1/MCAF), CCL3 (MIP-Iα), CCL4 (MIP-1I), CCL5 (RANTES), CCL7 (MCP-3), CCL8 (mcp-2), CCL11 (eotaxin), CCL 13 (MCP-4), CCL 15 (MIP-Iδ), CCL 16 (HCC-4), CCL 17 (TARC), CCL 18 (PARC), CCL 19 (MDP-3b), CCL20 (MIP-3α), CCL21 (SLC/exodus-2), CCL22 (MDC/STC-1), CCL23 (MPIF-1), CCL24 (MPIF-2/eotaxin-2), CCL25 (TECK), CCL26 (eotaxin-3), CCL27 (CTACK/ILC), CCL28, CXCLI (GROI), CXCL2 (GR02), CXCL3 (GR03), CXCL5 (ENA-78), CXCL6 (GCP-2), CXCL9 (MIG), CXCL 10 (IP 10), CXCL 11 (1-TAC), CXCL 12 (SDFI), CXCL 13, CXCL 14, CXCL 16, PF4 (CXCL4), PPBP (CXCL7), CX3CL 1 (SCYDI), SCYEI, XCLI (lymphotactin), XCL2 (SCM-1I), BLRI (MDR15), CCBP2 (D6/JAB61), CCRI (CKRI/HM145), CCR2 (mcp-IRB IRA), CCR3 (CKR3/CMKBR3), CCR4, CCR5 (CMKBR5/ChemR13), CCR6 (CMKBR6/CKR-L3/STRL22/DRY6), CCR7 (CKR7/EBII), CCR8 (CMKBR8/TERI/CKR-L1), CCR9 (GPR-9-6), CCRL1 (VSHK1), CCRL2 (L-CCR), XCR1 (GPR5/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Rα), IL8RB (IL8Rβ), LTB4R (GPR16), TCP10, CKLFSF2, CKLFSF3, CKLFSF4, CKLFSF5, CKLFSF6, CKLFSF7, CKLFSF8, BDNF, C5, C5R1, CSF3, GRCC10 (C10), EPO, FY (DARC), GDF5, HDF1, HDF1α, DL8, PRL, RGS3, RGS13, SDF2, SLIT2, TLR2, TLR4, TREM1, TREM2, and VHL. In some embodiments, the polypeptide expressed by the mammalian cells of the present disclosure may bind to, or interact with, 0772P (CA125, MUC16) (i.e., ovarian cancer antigen), ABCF1; ACVR1; ACVR1B; ACVR2; ACVR2B; ACVRL1; ADORA2A; Aggrecan; AGR2; AICDA; AIF1; AIGI; AKAP1; AKAP2; AMH; AMHR2; amyloid beta; ANGPTL; ANGPT2; ANGPTL3; ANGPTL4; ANPEP; APC; APOC1; AR; ASLG659; ASPHD1 (aspartate beta-hydroxylase domain containing 1; LOC253982); AZGP1 (zinc-a-glycoprotein); B7.1; B7.2; BAD; BAFF-R (B cell-activating factor receptor, BLyS receptor 3, BR3; BAGI; BAIl; BCL2; BCL6; BDNF; BLNK; BLRI (MDR15); BMP1; BMP2; BMP3B (GDF10); BMP4; BMP6; BMP8; BMPR1A; BMPR1B (bone morphogenic protein receptor-type IB); BMPR2; BPAG1 (plectin); BRCA1; Brevican; C190rfl0 (IL27w); C3; C4A; C5; C5R1; CANTI; CASPI; CASP4; CAVI; CCBP2 (D6/JAB61); CCL1 (1-309); CCL11 (eotaxin); CCL13 (MCP-4); CCL15 (MIP16); CCL16 (HCC-4); CCL17 (TARC); CCL18 (PARC); CCL19 (MIP-30); CCL2 (MCP-1); MCAF; CCL20 (MIP-3α); CCL21 (MTP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MTP-Iu); CCL4 (MDP-I0); CCL5(RANTES); CCL7 (MCP-3); CCL8 (mcp-2); CCNA1; CCNA2; CCND1; CCNE1; CCNE2; CCR1 (CKRI/HM145); CCR2 (mcp-IRP/RA);CCR3 (CKR/CMKBR3); CCR4; CCR5 (CMKBR5/ChemR13); CCR6 (CMKBR6/CKR-L3/STRL22/DRY6); CCR7 (CKBR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD22 (B-cell receptor CD22-B isoform); CD24; CD28; CD3; CD37; CD38; CD3E; CD3G; CD3Z; CD4; CD40; CD40L; CD44; CD45RB; CD52; CD69; CD72; CD74; CD79A (CD79a, immunoglobulin-associated alpha, a B cell-specific protein); CD79B; CDS; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21/WAFI/Cip1); CDKN1B (p27/Kipl); CDKN1C; CDKN2A (P16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3;CLDN7 (claudin-7); CLL-1 (CLEC12A, MICL, and DCAL2); CLN3; CLU (clusterin); CMKLR1; CMKOR1 (RDC1); CNR1; COL 18A1; COL1A1; COL4A3; COL6A1; complement factor D; CR2; CRP; CRIPTO (CR, CR1, CRGF, CRIPTO, TDGF1, teratocarcinoma-derived growth factor); CSFI (M-CSF); CSF2 (GM-CSF); CSF3 (GCSF); CTLA4; CTNNB1 (b-catenin); CTSB (cathepsin B); CX3CL1 (SCYDI); CX3CR1 (V28); CXCL1 (GRO1); CXCL10 (IP-10); CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR5 (Burkitt's lymphoma receptor 1, a G protein-coupled receptor); CXCR6 (TYMSTR/STRL33/Bonzo); CYBS; CYC1; CYSLTR1; DAB2IP; DES; DKFZp451J0118; DNCLI; DPP4; E16 (LAT1, SLC7A5); E2F1; ECGF1; EDGI; EFNA1; EFNA3; EFNB2; EGF; EGFR; ELAC2; ENG; ENOl; ENO2; ENO3; EPHB4; EphB2R; EPO; ERBB2 (Her-2); EREG; ERK8; ESRI; ESR2; ETBR (Endothelin type B receptor); F3 (TF); FADD; FasL; FASN; FCER1A; FCER2; FCGR3A; FcRH1 (Fc receptor-like protein 1); FcRH2 (IFGP4, IRTA4, SPAP1A (SH2 domain containing phosphatase anchor protein 1a), SPAPIB, SPAPIC); FGF; FGF1 (αFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR; FGFR3; FIGF (VEGFD); FEL1 (EPSILON); FILl (ZETA); FLJ12584; FLJ25530; FLRTI (fibronectin); FLT1; FOS; FOSL1 (FRA-1); FY (DARC); GABRP (GABAa); GAGEBI; GAGECI; GALNAC4S-6ST; GATA3; GDF5; GDNF-Ral (GDNF family receptor alpha 1; GFRA1; GDNFR; GDNFRA; RETL1; TRNR1; RET1L; GDNFR-alphal; GFR-ALPHA-1); GEDA; GFI1; GGT1; GM-CSF; GNASI; GNRHI; GPR2 (CCR10); GPR19 (G protein-coupled receptor 19; Mm.4787); GPR31; GPR44; GPR54 (KISS1 receptor; KISSIR; GPR54; HOT7T175; AXOR12); GPR81 (FKSG80); GPR172A (G protein-coupled receptor 172A; GPCR41; FLJ11856; D15Ertd747e);GRCCIO (C10); GRP; GSN (Gelsolin); GSTP1; HAVCR2; HDAC4; HDAC5; HDAC7A; HDAC9; HGF; HIF1A; HOPI; histamine and histamine receptors; HLA-A; HLA-DOB (Beta subunit of MHC class II molecule (Ia antigen); HLA-DRA; HM74; HMOXI; HUMCYT2A; ICEBERG; ICOSL; 1D2; IFN-α; IFNA1; IFNA2; IFNA4; IFNA5; IFNA6; IFNA7; IFNB1; IFNgamma; DFNW1; IGBP1; IGF1; IGF1R; IGF2; IGFBP2; IGFBP3; IGFBP6; IL-l; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; ILlA; IL1B; ILIF10; IL1F5; IL1F6; IL1F7; IL1F8; IL1F9; IL1HYl; IL1R1; IL1R2; ILIRAP; ILIRAPLI; IL1RAPL2; IL1RL1; IL1RL2, ILIRN; IL2; IL20; IL20Ru; IL21R; IL22; IL-22c; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); influenza A; influenza B; EL7; EL7R; EL8; IL8RA; DL8RB; IL8RB; DL9; DL9R; DLK; INHA; INHBA; INSL3; INSL4; IRAKI; IRTA2 (Immunoglobulin superfamily receptor translocation associated 2); ERAK2; ITGA1; ITGA2; ITGA3; ITGA6 (a6 integrin); ITGAV; ITGB3; ITGB4 (b4 integrin); u407 and uE07 integrin heterodimers; JAGI; JAKI; JAK3; JUN; K6HF; KAIl; KDR; KITLG; KLF5 (GC Box BP); KLF6; KLKIO; KLK12; KLK13; KLK14; KLK15; KLK3; KLK4; KLK5; KLK6; KLK9; KRT1; KRT19 (Keratin 19); KRT2A; KHTHB6 (hair-specific type H keratin); LAMAS; LEP (leptin); LGR5 (leucine-rich repeat-containing G protein-coupled receptor 5; GPR49, GPR67); Lingo-p75; Lingo-Troy; LPS; LTA (TNF-b); LTB; LTB4R (GPR16); LTB4R2; LTBR; LY64 (Lymphocyte antigen 64 (RP105), type I membrane protein of the leucine rich repeat (LRR) family); Ly6E (lymphocyte antigen 6 complex, locus E; Ly67,RIG-E,SCA-2,TSA-1); Ly6G6D (lymphocyte antigen 6 complex, locus G6D; Ly6-D, MEGT1); LY6K (lymphocyte antigen 6 complex, locus K; LY6K; HSJ001348; FLJ35226); MACMARCKS; MAG or OMgp; MAP2K7 (c-Jun); MDK; MDP; MIB1; midkine; MEF; MIP-2; MK167; (K1-67); MMP2; MMP9; MPF (MPF, MSLN, SMR, megakaryocyte potentiating factor, mesothelin); MS4A1; MSG783 (RNF124, hypothetical protein FLJ20315);MSMB; MT3 (metallothionectin-111); MTSS1; MUC1 (mucin); MYC; MY088; Napi3b (also known as NaPi2b) (NAPI-3B, NPTIIb, SLC34A2, solute carrier family 34 (sodium phosphate), member 2, type II sodium-dependent phosphate transporter 3b); NCA; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NROB1; NROB2; NR1D1; NR1D2; NR1H2; NR1H3; NR1H4; NR112; NR113; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NTSE; NTN4; ODZI; OPRD1; OX40; P2RX7; P2X5 (Purinergic receptor P2X ligand-gated ion channel 5); PAP; PART1; PATE; PAWR; PCA3; PCNA; PD-Li; PD-L2; PD-1; POGFA; POGFB; PECAMI; PF4 (CXCL4); PGF; PGR; phosphacan; PIAS2; PIK3CG; PLAU (uPA); PLG; PLXDCI; PMEL17 (silver homolog; SILV; D12S53E; PMEL17; SI; SIL); PPBP (CXCL7); PPID; PRI; PRKCQ; PRKDI; PRL; PROC; PROK2; PSAP; PSCA hlg (2700050C12Rik, C530008016Rik, RIKEN cDNA 2700050C12, RIKEN cDNA 2700050C12 gene); PTAFR; PTEN; PTGS2 (COX-2); PTN; RAC2 (p21 Rac2); RARB; RET (ret proto-oncogene; MEN2A; HSCR1; MEN2B; MTC1; PTC; CDHF12; Hs.168114; RET51; RET-ELE1); RGSI; RGS13; RGS3; RNF110 (ZNF144); ROBO2; S100A2; SCGB1D2 (lipophilin B); SCGB2A1 (mammaglobin2); SCGB2A2 (mammaglobin 1); SCYEI (endothelial Monocyte-activating cytokine); SDF2; Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain™ and short cytoplasmic domain, (semaphorin) 5B); SERPINA1; SERPINA3; SERP1NB5 (maspin); SERPINE1(PAI-1); SERPDMF1; SHBG; SLA2; SLC2A2; SLC33A1; SLC43A1; SLIT2; SPPI; SPRR1B (Sprl); ST6GAL1; STABI; STAT6; STEAP (six transmembrane epithelial antigen of prostate); STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein); TB4R2; TBX21; TCPIO; TOGFI; TEK; TENB2 (putative transmembrane proteoglycan); TGFA; TGFBI; TGFB1II; TGFB2; TGFB3; TGFBI; TGFBRI; TGFBR2; TGFBR3; THIL; THBSI (thrombospondin-1); THBS2; THBS4; THPO; TIE (Tie-1); TMP3; tissue factor; TLR1; TLR2; TLR3; TLR4; TLR5; TLR6; TLR7; TLR8; TLR9; TLR10; TMEFF1 (transmembrane protein with EGF-like and two follistatin-like domains 1; Tomoregulin-1); TMEM46 (shisa homolog 2); TNF; TNF-a; TNFAEP2 (B94); TNFAIP3; TNFRSFIIA; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSFS (CD30 ligand); TNFSF9 (4-1 BB ligand); TOLLIP; Toll-like receptors; TOP2A (topoisomerase Ea); TP53; TPM1; TPM2; TRADD; TMEM118 (ring finger protein, transmembrane 2; RNFT2; FLJ14627); TRAF1; TRAF2; TRAF3; TRAF4; TRAF5; TRAF6; TREM1; TREM2; TrpM4 (BR22450, FLJ20041, TRPM4, TRPM4B, transient receptor potential cation channel, subfamily M, member 4); TRPC6; TSLP; TWEAK; Tyrosinase (TYR; OCAIA; OCA1A; tyrosinase; SHEP3);VEGF; VEGFB; VEGFC; versican; VHL C5; VLA-4; XCL1 (lymphotactin); XCL2 (SCM-Tb); XCRI(GPR5/CCXCRI); YY1; and/or ZFPM2.

Many other virus components and/or other genes of interest may be packaged by the mammalian cells in accordance with the present disclosure, and the above lists are not meant to be limiting.

### 5.5.2 Recombinant Protein Products

In certain embodiments, the cells and/or methods of the present disclosure can be used for the production of recombinant proteins, e.g., recombinant mammalian proteins. Non-limiting examples of such recombinant proteins include hormones, receptors, fusion proteins, regulatory factors, growth factors, complement system factors, enzymes, clotting factors, anti-clotting factors, kinases, cytokines, CD proteins, interleukins, therapeutic proteins, diagnostic proteins and antibodies. The cells and/or methods of the present disclosure are not specific to the molecule, e.g., antibody, that is being produced.

In certain embodiments, the methods of the present disclosure can be used for the production of antibodies, including therapeutic and diagnostic antibodies or antigen-binding fragments thereof. In certain embodiments, the antibody produced by cell and methods of the present disclosure can be, but are not limited to, monospecific antibodies (e.g., antibodies consisting of a single heavy chain sequence and a single light chain sequence, including multimers of such pairings), multispecific antibodies and antigen-binding fragments thereof. For example, but not by way of limitation, the multispecific antibody can be a bispecific antibody, a biepitopic antibody, a T-cell-dependent bispecific antibody (TDB), a Dual Acting FAb (DAF) or antigen-binding fragments thereof.

### 5.5.2.1 Multispecific Antibodies

In certain aspects, an antibody produced by cells and methods provided herein is a multispecific antibody, e.g., a bispecific antibody. “Multispecific antibodies” are monoclonal antibodies that have binding specificities for at least two different sites, i.e., different epitopes on different antigens (i.e., bispecific) or different epitopes on the same antigen (i.e., biepitopic). In certain aspects, the multispecific antibody has three or more binding specificities. Multispecific antibodies can be prepared as full length antibodies or antibody fragments as described herein.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multispecific antibodies can also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other non-limiting examples of multispecific antibodies with three or more antigen binding sites can be found in WO 2010/115589, WO 2010/112193, WO 2010/136172, WO 2010/145792 and WO 2013/026831. The bispecific antibody or antigen binding fragment thereof also includes a “Dual Acting FAb” or “DAF” (see, e.g., US 2008/0069820 and WO 2015/095539).

Multispecific antibodies can also be provided in an asymmetric form with a domain crossover in one or more binding arms of the same antigen specificity, i.e., by exchanging the VH/VL domains (see, e.g., WO 2009/080252 and WO 2015/150447), the CH1/CL domains (see, e.g., WO 2009/080253) or the complete Fab arms (see, e.g., WO 2009/080251, WO 2016/016299, also see Schaefer et al, PNAS, 10⁸ (2011) 1187-1191, and Klein at al., MAbs 8 (2016) 1010-20). In certain embodiments, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CHI), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See, e.g., WO 2016/172485.

Various further molecular formats for multispecific antibodies are known in the art and are included herein (see, e.g., Spiess et al., Mol. Immunol. 67 (2015) 95-106).

In certain embodiments, particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells.

Additional non-limiting examples of bispecific antibody formats that can be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bduerle, Exp Cell Res 317, 1255-1260 (2011)); diabodies (Holliger et al., Prot. Eng. 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat. Rev. 36, 458-467 (2010)). Particular T cell bispecific antibody formats included herein are described in WO 2013/026833, WO 2013/026839, WO 2016/020309; Bacac et al., Oncoimmunology 5(8) (2016) e1203498.

### 5.5.2.2 Antibody Fragments

In certain aspects, an antibody produced by the cells and methods provided herein is an antibody fragment. For example, but not by way of limitation, the antibody fragment is a Fab, Fab′, Fab′-SH or F(ab′)2 fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CHI). The term “Fab fragment” thus refers to an antibody fragment comprising a light chain comprising a VL domain and a CL domain, and a heavy chain fragment comprising a VH domain and a CHI domain. “Fab′ fragments” differ from Fab fragments by the addition of residues at the carboxy terminus of the CHI domain including one or more cysteines from the antibody hinge region. Fab′-SH are Fab′ fragments in which the cysteine residue(s) of the constant domains bear a free thiol group. Pepsin treatment yields an F(ab′)2 fragment that has two antigen-binding sites (two Fab fragments) and a part of the Fc region. For discussion of Fab and F(ab′)2 fragments comprising salvage receptor binding epitope residues and having increased in vivo half-life, see U.S. Pat. No. 5,869,046.

In certain embodiments, the antibody fragment is a diabody, a triabody or a tetrabody. “Diabodies” are antibody fragments with two antigen-binding sites that can be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plackthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody.

### 5.5.2.3 Chimeric and Humanized Antibodies

In certain aspects, an antibody produced by the cells and methods provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In certain embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osboum et al., Methods 36:61-68 (2005) and Klimka et al., Br. J. Cancer, 83:252-260 (2000) (describing the “guided selection” approach to FR shuffling).

Human framework regions that can be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 5.5.2.4 Human Antibodies

In certain aspects, an antibody produced by the cells and methods provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies can be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describingXENOMOUSETMtechnology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals can be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boemer et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

### 5.5.2.5 Target Molecules

Non-limiting examples of molecules that can be targeted by an antibody produced by the cells and methods disclosed herein include soluble serum proteins and their receptors and other membrane bound proteins (e.g., adhesins). In certain embodiments, an antibody produced by the cells and methods disclosed herein is capable of binding to one, two or more cytokines, cytokine-related proteins, and cytokine receptors selected from the group consisting of 8MPI, 8MP2, 8MP38 (GDFIO), 8MP4, 8MP6, 8MP8, CSFI (M-CSF), CSF2 (GM-CSF), CSF3 (G-CSF), EPO, FGF1 (aFGF), FGF2 (OFGF), FGF3 (int-2), FGF4 (HST), FGF5, FGF6 (HST-2), FGF7 (KGF), FGF9, FGF1 0, FGF11, FGF12, FGF12B, FGF14, FGF16, FGF17, FGF19, FGF20, FGF21, FGF23, IGF1, IGF2, IFNA1, IFNA2, IFNA4, IFNA5, IFNA6, IFNA7, IFN81, IFNG, IFNWI, FEL1, FEL1 (EPSELON), FEL1 (ZETA), IL TA, IL 1B, IL2, IL3, IL4, IL5, IL6, IL7, IL8, IL9, IL 10, IL 11, IL 12A, IL 12B, IL 13, IL 14, IL 15, IL 16, IL 17, IL 17B, IL 18, IL 19, IL20, IL22, IL23, IL24, IL25, IL26, IL27, IL28A, IL28B, IL29, IL30, PDGFA, PDGFB, TGFA, TGFB1, TGFB2, TGFBb3, LTA (TNF-0), LTB, TNF (TNF-α), TNFSF4 (OX40 ligand), TNFSF5 (CD40 ligand), TNFSF6 (FasL), TNFSF7 (CD27 ligand), TNFSF8 (CD30 ligand), TNFSF9 (4-1 BB ligand), TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, HGF (VEGFD), VEGF, VEGFB, VEGFC, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL 11RA, IL12RB1, IL12RB2, IL13RA1, IL13RA2, IL15RA, IL17R, IL18R1, IL20RA, IL21R, IL22R, IL1HYl, IL1RAP, IL1RAPL1, IL1RAPL2, IL1RN, IL6ST, IL18BP, IL18RAP, IL22RA2, AIF1, HGF, LEP (leptin), PTN, and THPO.k

In certain embodiments, an antibody produced by cells and methods disclosed herein is capable of binding to a chemokine, chemokine receptor, or a chemokine-related protein selected from the group consisting of CCLI (1-309), CCL2 (MCP-1/MCAF), CCL3 (MIP-Iα), CCL4 (MIP-1I), CCL5 (RANTES), CCL7 (MCP-3), CCL8 (mcp-2), CCL11 (eotaxin), CCL 13 (MCP-4), CCL 15 (MIP-Iδ), CCL 16 (HCC-4), CCL 17 (TARC), CCL 18 (PARC), CCL 19 (MDP-3b), CCL20 (MIP-3α), CCL21 (SLC/exodus-2), CCL22 (MDC/STC-1), CCL23 (MPIF-1), CCL24 (MPIF-2/eotaxin-2), CCL25 (TECK), CCL26 (eotaxin-3), CCL27 (CTACK/ILC), CCL28, CXCLI (GROI), CXCL2 (GR02), CXCL3 (GR03), CXCL5 (ENA-78), CXCL6 (GCP-2), CXCL9 (MIG), CXCL 10 (IP 10), CXCL 11 (1-TAC), CXCL 12 (SDFI), CXCL 13, CXCL 14, CXCL 16, PF4 (CXCL4), PPBP (CXCL7), CX3CL 1 (SCYDI), SCYEI, XCLI (lymphotactin), XCL2 (SCM-1I), BLRI (MDR15), CCBP2 (D6/JAB61), CCRI (CKRI/HM145), CCR2 (mcp-IRB IRA), CCR3 (CKR3/CMKBR3), CCR4, CCR5 (CMKBR5/ChemR13), CCR6 (CMKBR6/CKR-L3/STRL22/DRY6), CCR7 (CKR7/EBII), CCR8 (CMKBR8/TER1/CKR-L1), CCR9 (GPR-9-6), CCRL1 (VSHK1), CCRL2 (L-CCR), XCR1 (GPR5/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Rα), IL8RB (IL8Rβ), LTB4R (GPR16), TCP10, CKLFSF2, CKLFSF3, CKLFSF4, CKLFSF5, CKLFSF6, CKLFSF7, CKLFSF8, BDNF, C5, C5R1, CSF3, GRCC10 (C10), EPO, FY (DARC), GDF5, HDF1, HDF1α, DL8, PRL, RGS3, RGS13, SDF2, SLIT2, TLR2, TLR4, TREM1, TREM2, and VHL.

In certain embodiments, an antibody produced by methods disclosed herein (e.g., a multispecific antibody such as a bispecific antibody) is capable of binding to one or more target molecules selected from the following: 0772P (CA125, MUC16) (i.e., ovarian cancer antigen), ABCF1; ACVR1; ACVR1B; ACVR2; ACVR2B; ACVRL1; ADORA2A; Aggrecan; AGR2; AICDA; AIF1; AIGI; AKAP1; AKAP2; AMH; AMHR2; amyloid beta; ANGPTL; ANGPT2; ANGPTL3; ANGPTL4; ANPEP; APC; APOC1; AR; ASLG659; ASPHD1 (aspartate beta-hydroxylase domain containing 1; LOC253982); AZGP1 (zinc-a-glycoprotein); B7.1; B7.2; BAD; BAFF-R (B cell-activating factor receptor, BLyS receptor 3, BR3; BAGI; BAIl; BCL2; BCL6; BDNF; BLNK; BLRI (MDR15); BMP1; BMP2; BMP3B (GDF10); BMP4; BMP6; BMP8; BMPR1A; BMPR1B (bone morphogenic protein receptor-type IB); BMPR2; BPAG1 (plectin); BRCA1; Brevican; C19orfl0 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP16); CCL16 (HCC-4); CCL17 (TARC); CCL18 (PARC); CCL19 (MIP-30); CCL2 (MCP-1); MCAF; CCL20 (MIP-3α); CCL21 (MTP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MTP-Iu); CCL4 (MDP-I0); CCL5(RANTES); CCL7 (MCP-3); CCL8 (mcp-2); CCNA1; CCNA2; CCND1; CCNE1; CCNE2; CCR1 (CKRI/HM145); CCR2 (mcp-IRP/RA);CCR3 (CKR/CMKBR3); CCR4; CCR5 (CMKBR5/ChemR13); CCR6 (CMKBR6/CKR-L3/STRL22/DRY6); CCR7 (CKBR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD22 (B-cell receptor CD22-B isoform); CD24; CD28; CD3; CD37; CD38; CD3E; CD3G; CD3Z; CD4; CD40; CD40L; CD44; CD45RB; CD52; CD69; CD72; CD74; CD79A (CD79a, immunoglobulin-associated alpha, a B cell-specific protein); CD79B; CDS; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21/WAF1/Cip1); CDKN1B (p27/Kipl); CDKN1C; CDKN2A (P16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3;CLDN7 (claudin-7); CLL-1 (CLEC12A, MICL, and DCAL2); CLN3; CLU (clusterin); CMKLR1; CMKOR1 (RDC1); CNR1; COL 18A1; COL1A1; COL4A3; COL6A1; complement factor D; CR2; CRP; CRIPTO (CR, CR1, CRGF, CRIPTO, TDGF1, teratocarcinoma-derived growth factor); CSFI (M-CSF); CSF2 (GM-CSF); CSF3 (GCSF); CTLA4; CTNNB1 (b-catenin); CTSB (cathepsin B); CX3CL1 (SCYDI); CX3CR1 (V28); CXCL1 (GRO1); CXCL10 (IP-10); CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR5 (Burkitt's lymphoma receptor 1, a G protein-coupled receptor); CXCR6 (TYMSTR/STRL33/Bonzo); CYB5; CYC1; CYSLTR1; DAB2IP; DES; DKFZp451J0118; DNCLI; DPP4; E16 (LAT1, SLC7A5); E2F1; ECGF1; EDGI; EFNA1; EFNA3; EFNB2; EGF; EGFR; ELAC2; ENG; ENOl; ENO2; ENO3; EPHB4; EphB2R; EPO; ERBB2 (Her-2); EREG; ERK8; ESRI; ESR2; ETBR (Endothelin type B receptor); F3 (TF); FADD; FasL; FASN; FCER1A; FCER2; FCGR3A; FcRH1 (Fc receptor-like protein 1); FcRH2 (IFGP4, IRTA4, SPAP1A (SH2 domain containing phosphatase anchor protein 1a), SPAPIB, SPAPIC); FGF; FGF1 (αFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR; FGFR3; FIGF (VEGFD); FEL1 (EPSILON); FILl (ZETA); FLJ12584; FLJ25530; FLRTI (fibronectin); FLT1; FOS; FOSL1 (FRA-1); FY (DARC); GABRP (GABAa); GAGEB1; GAGEC1; GALNAC4S-6ST; GATA3; GDF5; GDNF-Ral (GDNF family receptor alpha 1; GFRA1; GDNFR; GDNFRA; RETL1; TRNR1; RET1L; GDNFR-alphal; GFR-ALPHA-1); GEDA; GFI1; GGT1; GM-CSF; GNASI; GNRHI; GPR2 (CCR10); GPR19 (G protein-coupled receptor 19; Mm.4787); GPR31; GPR44; GPR54 (KISS1 receptor; KISS1R; GPR54; HOT7T175; AXOR12); GPR81 (FKSG80); GPR172A (G protein-coupled receptor 172A; GPCR41; FLJ11856; D15Ertd747e);GRCCIO (C10); GRP; GSN (Gelsolin); GSTP1; HAVCR2; HDAC4; HDAC5; HDAC7A; HDAC9; HGF; HIF1A; HOP1; histamine and histamine receptors; HLA-A; HLA-DOB (Beta subunit of MHC class II molecule (Ia antigen); HLA-DRA; HM74; HMOXI; HUMCYT2A; ICEBERG; ICOSL; 1D2; IFN-α; IFNA1; IFNA2; IFNA4; IFNA5; IFNA6; IFNA7; IFNB1; IFNgamma; DFNW1; IGBP1; IGF1; IGF1R; IGF2; IGFBP2; IGFBP3; IGFBP6; IL-l; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; ILT8RAP; IL19; ILlA; IL1B; ILIF10; IL1F5; IL1F6; IL1F7; IL1F8; IL1F9; IL1HYl; IL1R1; IL1R2; IL1RAP; IL1RAPL1; ILTRAPL2; IL1RL1; IL1RL2, ILIRN; IL2; IL20; IL20Ra; IL21R; IL22; IL-22c; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); influenza A; influenza B; EL7; EL7R; EL8; IL8RA; DL8RB; IL8RB; DL9; DL9R; DLK; INHA; INHBA; INSL3; INSL4; IRAKI; IRTA2 (Immunoglobulin superfamily receptor translocation associated 2); ERAK2; ITGA1; ITGA2; ITGA3; ITGA6 (a6 integrin); ITGAV; ITGB3; ITGB4 (b4 integrin); a407 and aE07 integrin heterodimers; JAG1; JAK1; JAK3; JUN; K6HF; KAIl; KDR; KITLG; KLF5 (GC Box BP); KLF6; KLKIO; KLK12; KLK13; KLK14; KLK15; KLK3; KLK4; KLK5; KLK6; KLK9; KRT1; KRT19 (Keratin 19); KRT2A; KHTHB6 (hair-specific type H keratin); LAMAS; LEP (leptin); LGR5 (leucine-rich repeat-containing G protein-coupled receptor 5; GPR49, GPR67); Lingo-p75; Lingo-Troy; LPS; LTA (TNF-b); LTB; LTB4R (GPR16); LTB4R2; LTBR; LY64 (Lymphocyte antigen 64 (RP105), type I membrane protein of the leucine rich repeat (LRR) family); Ly6E (lymphocyte antigen 6 complex, locus E; Ly67,RIG-E,SCA-2,TSA-1); Ly6G6D (lymphocyte antigen 6 complex, locus G6D; Ly6-D, MEGT1); LY6K (lymphocyte antigen 6 complex, locus K; LY6K; HSJ001348; FLJ35226); MACMARCKS; MAG or OMgp; MAP2K7 (c-Jun); MDK; MDP; MIB1; midkine; MEF; MIP-2; MK167; (Ki-67); MMP2; MMP9; MPF (MPF, MSLN, SMR, megakaryocyte potentiating factor, mesothelin); MS4A1; MSG783 (RNF124, hypothetical protein FLJ20315);MSMB; MT3 (metallothionectin-111); MTSS1; MUC1 (mucin); MYC; MY088; Napi3b (also known as NaPi2b) (NAPI-3B, NPTIIb, SLC34A2, solute carrier family 34 (sodium phosphate), member 2, type II sodium-dependent phosphate transporter 3b); NCA; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NROB1; NROB2; NR1D1; NR1D2; NR1H2; NR1H3; NR1H4; NR112; NR113; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZI; OPRD1; OX40; P2RX7; P2X5 (Purinergic receptor P2X ligand-gated ion channel 5); PAP; PART1; PATE; PAWR; PCA3; PCNA; PD-Li; PD-L2; PD-1; POGFA; POGFB; PECAMI; PF4 (CXCL4); PGF; PGR; phosphacan; PIAS2; PIK3CG; PLAU (uPA); PLG; PLXDC1; PMEL17 (silver homolog; SILV; D12S53E; PMEL17; SI; SIL); PPBP (CXCL7); PPID; PRI; PRKCQ; PRKDI; PRL; PROC; PROK2; PSAP; PSCA hlg (2700050C12Rik, C530008016Rik, RIKEN cDNA 2700050C12, RIKEN cDNA 2700050C12 gene); PTAFR; PTEN; PTGS2 (COX-2); PTN; RAC2 (p21 Rac2); RARB; RET (ret proto-oncogene; MEN2A; HSCR1; MEN2B; MTC1; PTC; CDHF12; Hs.168114; RET51; RET-ELE1); RGSI; RGS13; RGS3; RNF110 (ZNF144); ROBO2; S100A2; SCGB1D2 (lipophilin B); SCGB2A1 (mammaglobin2); SCGB2A2 (mammaglobin 1); SCYEI (endothelial Monocyte-activating cytokine); SDF2; Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain™ and short cytoplasmic domain, (semaphorin) 5B); SERPINA1; SERPINA3; SERP1NB5 (maspin); SERPINE1(PAI-1); SERPDMFl; SHBG; SLA2; SLC2A2; SLC33A1; SLC43A1; SLIT2; SPPI; SPRR1B (Sprl); ST6GAL1; STABI; STAT6; STEAP (six transmembrane epithelial antigen of prostate); STEAP2 (HGNC_8639, IPCA-1, PCANAP1, STAMPI, STEAP2, STMP, prostate cancer associated gene 1, prostate cancer associated protein 1, six transmembrane epithelial antigen of prostate 2, six transmembrane prostate protein); TB4R2; TBX21; TCPIO; TOGFI; TEK; TENB2 (putative transmembrane proteoglycan); TGFA; TGFBI; TGFBIII; TGFB2; TGFB3; TGFBI; TGFBRI; TGFBR2; TGFBR3; THIL; THBSI (thrombospondin-1); THBS2; THBS4; THPO; TIE (Tie-1); TMP3; tissue factor; TLRI; TLR2; TLR3; TLR4; TLR5; TLR6; TLR7; TLR8; TLR9; TLR10; TMEFF1 (transmembrane protein with EGF-like and two follistatin-like domains 1; Tomoregulin-1); TMEM46 (shisa homolog 2); TNF; TNF-a; TNFAEP2 (B94); TNFAIP3; TNFRSFIIA; TNFRSFlA; TNFRSFlB; TNFRSF21; TNFRSF5; TNFRSF6 (Fas); TNFRSF7; TNFRSF8; TNFRSF9; TNFSF1O (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSFS (CD30 ligand); TNFSF9 (4-1 BB ligand); TOLLIP; Toll-like receptors; TOP2A (topoisomerase Ea); TP53; TPM1; TPM2; TRADD; TMEM118 (ring finger protein, transmembrane 2; RNFT2; FLJ14627); TRAF1; TRAF2; TRAF3; TRAF4; TRAF5; TRAF6; TREM1; TREM2; TrpM4 (BR22450, FLJ20041, TRPM4, TRPM4B, transient receptor potential cation channel, subfamily M, member 4); TRPC6; TSLP; TWEAK; Tyrosinase (TYR; OCAIA; OCA1A; tyrosinase; SHEP3);VEGF; VEGFB; VEGFC; versican; VHL C5; VLA-4; XCL1 (lymphotactin); XCL2 (SCM-lb); XCRI(GPR5/CCXCRI); YY1; and ZFPM2.

In certain embodiments, an antibody produced by the cells and methods disclosed herein is capable of binding to CD proteins such as CD3, CD4, CD5, CD16, CD19, CD20, CD21 (CR2 (Complement receptor 2) or C3DR (C3d/Epstein Barr virus receptor) or Hs.73792); CD33; CD34; CD64; CD72 (B-cell differentiation antigen CD72, Lyb-2); CD79b (CD79B, CD790, IGb (immunoglobulin-associated beta), B29); CD200 members of the ErbB receptor family such as the EGF receptor, HER2, HER3, or HER4 receptor; cell adhesion molecules such as LFA-1, Maci, p150.95, VLA-4, ICAM-1, VCAM, alpha4/beta7 integrin, and alphav/beta3 integrin including either alpha or beta subunits thereof (e.g., anti-CD11a, anti-CD18, or anti-CD11b antibodies); growth factors such as VEGF-A, VEGF-C; tissue factor (TF); alpha interferon (alphaIFN); TNFalpha, an interleukin, such as IL-1 beta, IL-3, IL-4, IL-5, IL-6, IL-8, IL-9, IL-13, IL 17 AF, IL-iS, IL-13R alphal, IL13R alpha2, IL-4R, IL-5R, IL-9R, IgE; blood group antigens; flk2/flt3 receptor; obesity (OB) receptor; mpl receptor; CTLA-4; RANKL, RANK, RSV F protein, protein C etc.

In certain embodiments, the cells and methods provided herein can be used to produce an antibody (or a multispecific antibody, such as a bispecific antibody) that specifically binds to complement protein C5 (e.g., an anti-C5 agonist antibody that specifically binds to human C5). In certain embodiments, the anti-C5 antibody comprises 1, 2, 3, 4, 5 or 6 CDRs selected from (a) a heavy chain variable region CDR1 comprising the amino acid sequence of SSYYMA (SEQ ID NO:1); (b) a heavy chain variable region CDR2 comprising the amino acid sequence of AIFTGSGAEYKAEWAKG (SEQ ID NO:26); (c) a heavy chain variable region CDR3 comprising the amino acid sequence of DAGYDYPTHAMHY (SEQ ID NO: 27); (d) a light chain variable region CDR1 comprising the amino acid sequence of RASQGISSSLA (SEQ ID NO: 28); (e) a light chain variable region CDR2 comprising the amino acid sequence of GASETES (SEQ ID NO: 29); and (f) a light chain variable region CDR3 comprising the amino acid sequence of QNTKVGSSYGNT (SEQ ID NO: 30). For example, in certain embodiments, the anti-C5 antibody comprises a heavy chain variable domain (VH) sequence comprising one, two or three CDRs selected from: (a) a heavy chain variable region CDR1 comprising the amino acid sequence of (SSYYMA (SEQ ID NO: 1); (b) a heavy chain variable region CDR2 comprising the amino acid sequence of AIFTGSGAEYKAEWAKG (SEQ ID NO: 26); (c) a heavy chain variable region CDR3 comprising the amino acid sequence of DAGYDYPTHAMHY (SEQ ID NO: 27); and/or a light chain variable domain (VL) sequence comprising one, two or three CDRs selected from (d) a light chain variable region CDR1 comprising the amino acid sequence of RASQGISSSLA (SEQ ID NO: 28); (e) a light chain variable region CDR2 comprising the amino acid sequence of GASETES (SEQ ID NO: 29); and (f) a light chain variable region CDR3 comprising the amino acid sequence of QNTKVGSSYGNT (SEQ ID NO: 30). The sequences ofCDR1, CDR2 and CDR3 oftheheavy chain variable region and CDR1, CDR2 and CDR3 of the light chain variable region above are disclosed in US 2016/0176954 as SEQ ID NO: 117, SEQ ID NO: 118, SEQ ID NO: 121, SEQ ID NO: 122, SEQ ID NO: 123, and SEQ ID NO: 125, respectively. (See Tables 7 and 8 in US 2016/0176954.)
In certain embodiments, the anti-C5 antibody comprises the VH and VL sequences QVQLVESGGG LVQPGRSLRL SCAASGFTVH SSYYMAWVRQ APGKGLEWVG AIFTGSGAEY KAEWAKGRVT ISKDTSKNQV VLTMTNMDPV DTATYYCASD AGYDYPTHAM HYWGQGTLVT VSS (SEQ ID NO: 31) and
DIQMTQSPSS LSASVGDRVT ITCRASQGIS SSLAWYQQKP GKAPKLLIYG ASETESGVPS RFSGSGSGTD FTLTISSLQP EDFATYYCQN TKVGSSYGNT FGGGTKVEIK (SEQ ID NO: 32), respectively, including post-translational modifications of those sequences. The VH and VL sequences above are disclosed in US 2016/0176954 as SEQ ID NO: 106 and SEQ ID NO: 111, respectively. (See Tables 7 and 8 in US 2016/0176954.) In certain embodiments, the anti-C5 antibody is 305L015 (see US 2016/0176954).

In certain embodiments, an antibody produced by methods disclosed herein is capable of binding to OX40 (e.g., an anti-OX40 agonist antibody that specifically binds to human OX40). In certain embodiments, the anti-OX40 antibody comprises 1, 2, 3, 4, 5 or 6 CDRs selected from (a) a heavy chain variable region CDR1 comprising the amino acid sequence of DSYMS (SEQ ID NO: 2); (b) a heavy chain variable region CDR2 comprising the amino acid sequence of DMYPDNGDSSYNQKFRE (SEQ ID NO: 3); (c) a heavy chain variable region CDR3 comprising the amino acid sequence of APRWYFSV (SEQ ID NO: 4); (d) a light chain variable region CDR1 comprising the amino acid sequence of RASQDISNYLN (SEQ ID NO: 5); (e) a light chain variable region CDR2 comprising the amino acid sequence of YTSRLRS (SEQ ID NO: 6); and (f) a light chain variable region CDR3 comprising the amino acid sequence of QQGHTLPPT (SEQ ID NO: 7). For example, in certain embodiments, the anti-OX40 antibody comprises a heavy chain variable domain (VH) sequence comprising one, two or three CDRs selected from: (a) a heavy chain variable region CDR1 comprising the amino acid sequence of DSYMS (SEQ ID NO: 2); (b) a heavy chain variable region CDR2 comprising the amino acid sequence of DMYPDNGDSSYNQKFRE (SEQ ID NO: 3); and (c) a heavy chain variable region CDR3 comprising the amino acid sequence of APRWYFSV (SEQ ID NO: 4) and/or a light chain variable domain (VL) sequence comprising one, two or three CDRs selected from (a) a light chain variable region CDR1 comprising the amino acid sequence of RASQDISNYLN (SEQ ID NO: 5); (b) a light chain variable region CDR2 comprising the amino acid sequence ofYTSRLRS (SEQ ID NO: 6); and (c) a light chain variable region CDR3 comprising the amino acid sequence of QQGHTLPPT (SEQ ID NO: 7). In certain embodiments, the anti-OX40 antibody comprises the VH and VL sequences.
EVQLVQSGAE VKKPGASVKV SCKASGYTFT DSYMSWVRQA PGQGLEWIGD MYPDNGDSSY NQKFRERVTI TRDTSTSTAY LELSSLRSED TAVYYCVLAP RWYFSVWGQG TLVTVSS (SEQ ID NO: 8) and
DIQMTQSPSS LSASVGDRVT ITCRASQDIS NYLNWYQQKP GKAPKLLIYY TSRLRSGVPS RFSGSGSGTD FTLTISSLQP EDFATYYCQQ GHTLPPTFGQ GTKVEIK (SEQ ID NO: 9), respectively, including post-translational modifications of those sequences.

In certain embodiments, the anti-OX40 antibody comprises 1, 2, 3, 4, 5 or 6 CDRs selected from (a) a heavy chain variable region CDR1 comprising the amino acid sequence of NYLIE (SEQ ID NO: 10); (b) a heavy chain variable region CDR2 comprising the amino acid sequence of VINPGSGDTYYSEKFKG (SEQ ID NO: 11); (c) a heavy chain variable region CDR3 comprising the amino acid sequence of DRLDY (SEQ ID NO: 12); (d) a light chain variable region CDR1 comprising the amino acid sequence of HASQDISSYIV (SEQ ID NO: 13); (e) alight chain variable region CDR2 comprising the amino acid sequence of HGTNLED (SEQ ID NO: 14); and (f) a light chain variable region CDR3 comprising the amino acid sequence of VHYAQFPYT (SEQ ID NO: 15). For example, in certain embodiments, the anti-OX40 antibody comprises a heavy chain variable domain (VH) sequence comprising one, two or three CDRs selected from: (a) a heavy chain variable region CDR1 comprising the amino acid sequence ofNYLIE (SEQ ID NO: 10); (b) aheavy chain variable region CDR2 comprising the amino acid sequence of VINPGSGDTYYSEKFKG (SEQ ID NO: 11); and (c) a heavy chain variable region CDR3 comprising the amino acid sequence of DRLDY (SEQ ID NO: 12) and/or a light chain variable domain (VL) sequence comprising one, two or three CDRs selected from (a) a light chain variable region CDR1 comprising the amino acid sequence of HASQDISSYIV (SEQ ID NO: 13); (b) a light chain variable region CDR2 comprising the amino acid sequence of HGTNLED (SEQ ID NO: 14); and (c) a light chain variable region CDR3 comprising the amino acid sequence of VHYAQFPYT (SEQ ID NO: 15). In certain embodiments, the anti-OX40 antibody comprises the VH and VL sequences
EVQLVQSGAE VKKPGASVKV SCKASGYAFT NYLIEWVRQA PGQGLEWIGV INPGSGDTYY SEKFKGRVTI TRDTSTSTAY LELSSLRSED TAVYYCARDR LDYWGQGTLV TVSS (SEQ ID NO: 16) and
DIQMTQSPSS LSASVGDRVT ITCHASQDIS SYIVWYQQKP GKAPKLLIYH GTNLEDGVPS RFSGSGSGTD FTLTISSLQP EDFATYYCVH YAQFPYTFGQ GTKVEIK (SEQ ID NO: 17), respectively, including post-translational modifications of those sequences.

Further details regarding anti-OX40 antibodies are provided in WO 2015/153513, which is incorporated herein by reference in its entirety.

In certain embodiments, an antibody produced by the cells and methods disclosed herein is capable of binding to influenza virus B hemagglutinin, i.e., “fluB” (e.g., an antibody that binds hemagglutinin from the Yamagata lineage of influenza B viruses, binds hemagglutinin from the Victoria lineage of influenza B viruses, binds hemagglutinin from ancestral lineages of influenza B virus, or binds hemagglutinin from the Yamagata lineage, the Victoria lineage, and ancestral lineages of influenza B virus, in vitro and/or in vivo). Further details regarding anti-FluB antibodies are described in WO 2015/148806, which is incorporated herein by reference in its entirety.

In certain embodiments, an antibody produced by the cells and methods disclosed herein is capable of binding to low density lipoprotein receptor-related protein (LRP)-1 or LRP-8 or transferrin receptor, and at least one target selected from the group consisting of beta-secretase (BACE1 or BACE2), alpha-secretase, gamma-secretase, tau-secretase, amyloid precursor protein (APP), death receptor 6 (DR6), amyloid beta peptide, alpha-synuclein, Parkin, Huntingtin, p75 NTR, CD40 and caspase-6.

In certain embodiments, an antibody produced by the cells and methods disclosed herein is a human IgG2 antibody against CD40. In certain embodiments, the anti-CD40 antibody is RG7876.

In certain embodiments, the cells and methods of the present disclosure can be used to product a polypeptide. For example, but not by way of limitation, the polypeptide is a targeted immunocytokine. In certain embodiments, the targeted immunocytokine is a CEA-IL2v immunocytokine. In certain embodiments, the CEA-IL2v immunocytokine is RG7813. In certain embodiments, the targeted immunocytokine is a FAP-IL2v immunocytokine. In certain embodiments, the FAP-IL2v immunocytokine is RG7461.

In certain embodiments, the multispecific antibody (such as a bispecific antibody) produced by the cells or methods provided herein is capable of binding to CEA and at least one additional target molecule. In certain embodiments, the multispecific antibody (such as a bispecific antibody) produced according to methods provided herein is capable of binding to a tumor targeted cytokine and at least one additional target molecule. In certain embodiments, the multispecific antibody (such as a bispecific antibody) produced according to methods provided herein is fused to IL2v (i.e., an interleukin 2 variant) and binds an IL1-based immunocytokine and at least one additional target molecule. In certain embodiments, the multispecific antibody (such as a bispecific antibody) produced according to methods provided herein is a T-cell bispecific antibody (i.e., a bispecific T-cell engager or BiTE).

In certain embodiments, the multispecific antibody (such as a bispecific antibody) produced according to methods provided herein is capable of binding to at least two target molecules selected from: IL-1 alpha and IL-1 beta, IL-12 and IL-iS; IL-13 and IL-9; IL-13 and IL-4; IL-13 and IL-5; IL-5 and IL-4; IL-13 and IL-Ibeta; IL-13 and IL-25; IL-13 and TARC; IL-13 and MDC; IL-13 and MEF; IL-13 and TGF-˜; IL-13 and LHR agonist; IL-12 and TWEAK, IL-13 and CL25; IL-13 and SPRR2a; IL-13 and SPRR2b; IL-13 and ADAMS, IL-13 and PED2, IL17A and IL17F, CEA and CD3, CD3 and CD19, CD138 and CD20; CD138 and CD40; CD19 and CD20; CD20 and CD3; CD3S and CD13S; CD3S and CD20; CD3S and CD40; CD40 and CD20; CD-S and IL-6; CD20 and BR3, TNF alpha and TGF-beta, TNF alpha and IL-1 beta; TNF alpha and IL-2, TNF alpha and IL-3, TNF alpha and IL-4, TNF alpha and IL-5, TNF alpha and IL6, TNF alpha and IL8, TNF alpha and IL-9, TNF alpha and IL-10, TNF alpha and IL-11, TNF alpha and IL-12, TNF alpha and IL-13, TNF alpha and IL-14, TNF alpha and IL-15, TNF alpha and IL-16, TNF alpha and IL-17, TNF alpha and IL-18, TNF alpha and IL-19, TNF alpha and IL-20, TNF alpha and IL-23, TNF alpha and IFN alpha, TNF alpha and CD4, TNF alpha and VEGF, TNF alpha and MIF, TNF alpha and ICAM-1, TNF alpha and PGE4, TNF alpha and PEG2, TNF alpha and RANK ligand, TNF alpha and Te38, TNF alpha and BAFF,TNF alpha and CD22, TNF alpha and CTLA-4, TNF alpha and GP130, TNF a and IL-12p40, VEGF and Angiopoietin, VEGF and HER2, VEGF-A and HER2, VEGF-A and PDGF, HER1 and HER2, VEGFA and ANG2,VEGF-A and VEGF-C, VEGF-C and VEGF-D, HER2 and DR5,VEGF and IL-8, VEGF and MET, VEGFR and MET receptor, EGFR and MET, VEGFR and EGFR, HER2 and CD64, HER2 and CD3, HER2 and CD16, HER2 and HER3; EGFR (HER1) and HER2, EGFR and HER3, EGFR and HER4, IL-14 and IL-13, IL-13 and CD40L, IL4 and CD40L, TNFR1 and IL-1R, TNFR1 and IL-6R and TNFR1 and IL-18R, EpCAM and CD3, MAPG and CD28, EGFR and CD64, CSPGs and RGM A; CTLA-4 and BTN02; IGF1 and IGF2; IGF½ and Erb2B; MAG and RGM A; NgR and RGM A; NogoA and RGM A; OMGp and RGM A; POL-l and CTLA-4; and RGM A and RGM B.

In certain embodiments, the multispecific antibody (such as a bispecific antibody) produced according to methods provided herein is an anti-CEA/anti-CD3 bispecific antibody. In certain embodiments, the anti-CEA/anti-CD3 bispecific antibody is RG7802. In certain embodiments, the anti-CEA/anti-CD3 bispecific antibody comprises the amino acid sequences set forth in SEQ ID NOs: 18-21 are provided below:

(SEQ ID NO: 18)
DIQMTQSPSS LSASVGDRVT ITCKASAAVG TYVAWYQQKP
GKAPKLLIYS ASYRKRGVPS RFSGSGSGTD FTLTISSLQP
EDFATYYCHQ YYTYPLFTFG QGTKLEIKRT VAAPSVFIFP
PSDEQLKSGT ASVVCLLNNF YPREAKVQWK VDNALQSGNS
QESVTEQDSK DSTYSLSSTL TLSKADYEKH KVYACEVTHQ
GLSSPVTKSF NRGEC
(SEQ ID NO: 19)
QAVVTQEPSL TVSPGGTVTL TCGSSTGAVT TSNYANWVQE
KPGQAFRGLI GGTNKRAPGT PARFSGSLLG GKAALTLSGA
QPEDEAEYYC ALWYSNLWVF GGGTKLTVLS SASTKGPSVE
PLAPSSKSTS GGTAALGCLV KDYFPEPVTV SWNSGALTSG
VHTFPAVLQS SGLYSLSSVV TVPSSSLGTQ TYICNVNHKP
SNTKVDKKVE PKSC
(SEQ ID NO: 20)
QVQLVQSGAE VKKPGASVKV SCKASGYTFT EFGMNWVRQA
PGQGLEWMGW INTKTGEATY VEEFKGRVTF TTDTSTSTAY
MELRSLRSDD TAVYYCARWD FAYYVEAMDY WGQGTTVTVS
SASTKGPSVF PLAPSSKSTS GGTAALGCLV KDYFPEPVTV
SWNSGALTSG VHTFPAVLQS SGLYSLSSVV TVPSSSLGTQ
TYICNVNHKP SNTKVDKKVE PKSCDGGGGS GGGGSEVQLL
ESGGGLVQPG GSLRLSCAAS GFTFSTYAMN WVRQAPGKGL
EWVSRIRSKY NNYATYYADS VKGRFTISRD DSKNTLYLQM
NSLRAEDTAV YYCVRHGNFG NSYVSWFAYW GQGTLVTVSS
ASVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGECDKT HTCPPCPAPE
AAGGPSVFLF PPKPKDTLMI SRTPEVTCVV VDVSHEDPEV
KFNWYVDGVE VHNAKTKPRE EQYNSTYRVV SVLTVLHQDW
LNGKEYKCKV SNKALGAPIE KTISKAKGQP REPQVYTLPP
CRDELTKNQV SLWCLVKGFY PSDIAVEWES NGQPENNYKT
TPPVLDSDGS FFLYSKLTVD KSRWQQGNVF SCSVMHEALH
NHYTQKSLSL SPGK
(SEQ ID NO: 21)
QVQLVQSGAE VKKPGASVKV SCKASGYTFT EFGMNWVRQA
PGQGLEWMG WINTKTGEATY VEEFKGRVTF TTDTSTSTAY
MELRSLRSDD TAVYYCARWD FAYYVEAMD YWGQGTTVTVS
SASTKGPSVF PLAPSSKSTS GGTAALGCLV KDYFPEPVTV
SWNSGALTS GVHTFPAVLQS SGLYSLSSVV TVPSSSLGTQ
TYICNVNHKP SNTKVDKKVE PKSCDKTHT CPPCPAPEAAG
GPSVFLFPPK PKDTLMISRT PEVTCVVVDV SHEDPEVKFN
WYVDGVEVH NAKTKPREEQY NSTYRVVSVL TVLHQDWLNG
KEYKCKVSNK ALGAPIEKTI SKAKGQPRE PQVCTLPPSRD
ELTKNQVSLS CAVKGFYPSD IAVEWESNGQ PENNYKTTPP
VLDSDGSFF LVSKLTVDKSR WQQGNVFSCS VMHEALHNHY
TQKSLSLSPG K

Further details regarding anti-CEA/anti-CD3 bispecific antibodies are provided in WO 2014/121712, which is incorporated herein by reference in its entirety.

In certain embodiments, a multispecific antibody (such as a bispecific antibody) produced by the cells and methods disclosed herein is an anti-VEGF/anti-angiopoietin bispecific antibody. In certain embodiments, the anti-VEGF/anti-angiopoietin bispecific antibody bispecific antibody is a Crossmab. In certain embodiments, the anti-VEGF/anti-angiopoietin bispecific antibody is RG7716. In certain embodiments, the anti-CEA/anti-CD3 bispecific antibody comprises the amino acid sequences set forth in SEQ ID NOs: 22-25 are provided below:

(SEQ ID NO: 22)
EVQLVESGGG LVQPGGSLRL SCAASGYDFT HYGMNWVRQA
PGKGLEWVGW INTYTGEPTY AADFKRRFTF SLDTSKSTAY
LQMNSLRAED TAVYYCAKYP YYYGTSHWYF DVWGQGTLVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKK VEPKSCDKTH TCPPCPAPEA
AGGPSVFLFP PKPKDTLMAS RTPEVTCVVV DVSHEDPEVK
FNWYVDGVEV HNAKTKPREE QYNSTYRVVS VLTVLAQDWL
NGKEYKCKVS NKALGAPIEK TISKAKGQPR EPQVYTLPPC
RDELTKNQVS LWCLVKGFYP SDIAVEWESN GQPENNYKTT
PPVLDSDGSF FLYSKLTVDK SRWQQGNVFS CSVMHEALHN
AYTQKSLSLS PGK
(SEQ ID NO: 23)
QVQLVQSGAE VKKPGASVKV SCKASGYTFT GYYMHWVRQA
PGQGLEWMGW INPNSGGTNY AQKFQGRVTM TRDTSISTAY
MELSRLRSDD TAVYYCARSP NPYYYDSSGY YYPGAFDIWG
QGTMVTVSSA SVAAPSVFIF PPSDEQLKSG TASVVCLLNN
FYPREAKVQW KVDNALQSGN SQESVTEQDS KDSTYSLSST
LTLSKADYEK HKVYACEVTH QGLSSPVTKS FNRGECDKTH
TCPPCPAPEA AGGPSVFLFP PKPKDTLMAS RTPEVTCVVV
DVSHEDPEVK FNWYVDGVEV HNAKTKPREE QYNSTYRVVS
VLTVLAQDWL NGKEYKCKVS NKALGAPIEK TISKAKGQPR
EPQVCTLPPS RDELTKNQVS LSCAVKGFYP SDIAVEWESN
GQPENNYKTT PPVLDSDGSF FLVSKLTVDK SRWQQGNVFS
CSVMHEALHN AYTQKSLSLS PGK
(SEQ ID NO: 24)
DIQLTQSPSS LSASVGDRVT ITCSASQDIS NYLNWYQQKP
GKAPKVLIYF TSSLHSGVPS RFSGSGSGTD FTLTISSLQP
EDFATYYCQQ YSTVPWTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC
(SEQ ID NO: 25)
SYVLTQPPSV SVAPGQTARI TCGGNNIGSK SVHWYQQKPG
QAPVLVVYDD SDRPSGIPER FSGSNSGNTA TLTISRVEAG
DEADYYCQVW DSSSDHWVFG GGTKLTVLSS ASTKGPSVFP
LAPSSKSTSG GTAALGCLVK DYFPEPVTVS WNSGALTSGV
HTFPAVLQSS GLYSLSSVVT VPSSSLGTQT YICNVNHKPS
NTKVDKKVEP KSC

In certain embodiments, the multispecific antibody (such as a bispecific antibody) produced by methods disclosed herein is an anti-Ang2/anti-VEGF bispecific antibody. In certain embodiments, the anti-Ang2/anti-VEGF bispecific antibody is RG7221. In certain embodiments, the anti-Ang2/anti-VEGF bispecific antibody is CAS Number 1448221-05-3.

Soluble antigens or fragments thereof, optionally conjugated to other molecules, can be used as immunogens for generating antibodies. For transmembrane molecules, such as receptors, fragments of these (e.g., the extracellular domain of a receptor) can be used as the immunogen. Alternatively, cells expressing the transmembrane molecule can be used as the immunogen. Such cells can be derived from a natural source (e.g., cancer cell lines) or can be cells which have been transformed by recombinant techniques to express the transmembrane molecule. Other antigens and forms thereof useful for preparing antibodies will be apparent to those in the art.

In certain embodiments, the polypeptide (e.g., antibodies) produced by the cells and methods disclosed herein is capable of binding to can be further conjugated to a chemical molecule such as a dye or cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). An immunoconjugate comprising an antibody or bispecific antibody produced using the methods described herein can contain the cytotoxic agent conjugated to a constant region of only one of the heavy chains or only one of the light chains.

### 5.5.2.6 Antibody Variants

In certain aspects, amino acid sequence variants of the antibodies provided herein are contemplated, e.g., the antibodies provided in Section 5.5.5. For example, it can be desirable to alter the binding affinity and/or other biological properties of the antibody. Amino acid sequence variants of an antibody can be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### 5.5.2.6.1 Substitution, Insertion, and Deletion Variants

Substitute Specification-Clean Copy Attorney Docket No.: 00B3206.1375 In certain aspects, antibody variants having one or more amino acid substitutions are provided. Sites of interest for substitutional mutagenesis include the CDRs and FRs. Conservative substitutions are shown in Table 1 undertheheading of“preferred substitutions”. More substantial changes are provided in Table 1 under the heading of “exemplary substitutions”, and as further described below in reference to amino acid side chain classes. Amino acid substitutions can be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids can be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which can be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) can be made in CDRs, e.g., to improve antibody affinity. Such alterations can be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding can be specifically identified, e.g., using alanine scanning mutagenesis or modeling.

CDR-H3 and CDR-L3 in particular are often targeted.

In certain aspects, substitutions, insertions, or deletions can occur within one or more CDRs so long as such alterations do not substantially reduce the ability of the antibody to bind antigen. For example, conservative alterations (e.g., conservative substitutions as provided herein) that do not substantially reduce binding affinity can be made in the CDRs. Such alterations can, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that can be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by aneutral or negatively charged amino acid (e.g., alanine or polyalanine) to determine whether the interaction of the antibody with antigen is affected. Further substitutions can be introduced at the amino acid locations demonstrating functional sensitivity to the initial substitutions. Alternatively, or additionally, a crystal structure of an antigen-antibody complex can be used to identify contact points between the antibody and antigen. Such contact residues and neighboring residues can be targeted or eliminated as candidates for substitution. Variants can be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### 5.5.2.6.2 Glycosylation Variants

In certain aspects, an antibody provided herein is altered to increase or decrease the extent to which the antibody is glycosylated. Addition or deletion of glycosylation sites to an antibody can be conveniently accomplished by altering the amino acid sequence such that one or more glycosylation sites is created or removed.

Where the antibody comprises an Fc region, the oligosaccharide attached thereto can be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide can include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the disclosure can be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides can be at least about 20%, at least about 40%, at least about 60%, at least about 80%, or even about 100% (i.e., no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 can also be located about ±3 amino acids upstream or downstream of position 297, i.e., between positions 294 and 300, due to minor sequence variations in antibodies. Such antibodies having an increased proportion of non-fucosylated oligosaccharides in the Fc region can have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants can have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants can have improved CDC function. Such antibody variants are described, e.g., in WO 1997/30087; WO 1998/58964; and WO 1999/22764.

### 5.5.2.6.3 Fc Region Variants

In certain aspects, one or more amino acid modifications can be introduced into the Fc region of an antibody provided herein, thereby generating an Fc region variant. The Fc region variant can comprise a human Fc region sequence (e.g., a human IgG1, IgG2, IgG3 or IgG4 Fc region) comprising an amino acid modification (e.g., a substitution) at one or more amino acid positions.

In certain aspects, the present disclosure contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods can be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest can be assessed in vivo, e.g., in a animal model such as that disclosed in Clynes et al. Proc. Nat'l Acad. Sci. USA 95:652-656 (1998). C1q binding assays can also be carried out to confirm that the antibody is unable to bind C1q and hence lacks CDC activity. See, e.g., C1q and C3c binding ELISA in WO 2006/029879 and WO 2005/100402. To assess complement activation, a CDC assay can be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).) In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG1 Fc region. In one aspect, the substitutions are L234A, L235A and P329G (LALA-PG) in an Fc region derived from a human IgG1 Fc region. (See, e.g., WO 2012/130831). In another aspect, the substitutions are L234A, L235A and D265A (LALA-DA) in an Fc region derived from a human IgG1 Fc region.

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g., Dall'Acqua, W. F., et al. J. Immunol 169 (2002) 5171-5180). Residues 1253, H310, H433, N434, and H435 (EU index numbering) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues 1253, H310, and H435 were found to be critical for the interaction of human Fc with murine FcRn (Kim, J. K., et al., Eur. J. Immunol. 29 (1999) 2819). Studies of the human Fc-human FcRn complex have shown that residues 1253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are 1253A, H310A and H435A in an Fc region derived from a human IgG1 Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 A1).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG1 Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### 5.5.2.6.4 Cysteine Engineered Antibody Variants

In certain aspects, it can be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and can be used to conjugate the antibody to other moieties, such as drug moieties or linker-drug moieties, to create an immunoconjugate, as described further herein. Cysteine engineered antibodies can be generated as described, e.g., in U.S. Pat. Nos. 7,521,541, 8,30,930, 7,855,275, 9,000,130, or WO 2016040856.

### 5.5.2.6.5 Antibody Derivatives

In certain aspects, an antibody provided herein can be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde can have advantages in manufacturing due to its stability in water. The polymer can be of any molecular weight, and can be branched or unbranched. The number of polymers attached to the antibody can vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### 5.5.2.7 Immunoconjugates

The present disclosure also provides immunoconjugates comprising an antibody disclosed herein conjugated (chemically bonded) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In another aspect, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica Charantia inhibitor, curcin, crotin, sapaonaria Officinalis inhibitor, gelonin, mitogellin, restrictocin, phenomycin, enomycin, and the tricothecenes.

In another aspect, an immunoconjugate comprises an antibody as described herein conjugated to a radioactive atom to form a radioconjugate. A variety of radioactive isotopes are available for the production of radioconjugates. Examples include At211, 1131, 1125, Y90, Re186, Re188, Sm153, Bi212, P32, Pb212 and radioactive isotopes of Lu. When the radioconjugate is used for detection, it can comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent can be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker can be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) can be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### 6. EXAMPLES

The following examples are merely illustrative of the presently disclosed subject matter and should not be considered as limitations in any way.

### Example 1—Materials and Methods

### Cell Culture

Parental and KO host CHO cell lines were maintained as previously described (Domingos et al., Biotechnology Progress. Published online 2021:e3140). Briefly, CHO cells were cultured in a proprietary DMEM/F12-based medium in 125 mL shake flask vessels maintained at 150 rpm agitation, 37° C., and 5% CO₂. Cells were passaged at a seeding density of 4×10⁵ cells/mL every 3-4 days.

### Synthetic gRNA target design and screening

The gene targets used are listed in Tables 2-6. gRNA sequences were designed using the CRISPR Guide RNA Design software (Benchling) and manufactured by Integrated DNA Technologies (IDT). gRNA sequences were selected based on the software's on and off-target scoring, and at least three gRNAs targeting an early exon were screened for each gene target.

The following reagents were used from IDT: Alt-R® CRISPR-Cas9 sgRNA (sgRNA) and Alt-R® S.p. Cas9 Nuclease V3. Ribonucleoprotein (RNP)-based transfection of Cas9 protein was used. RNPs were formed by combining 20 pmol sgRNA with 20pmol of Cas9 protein at a 1:1 ratio for each target gene. Twelve million CHO cells were transfected using the RNPs with a Neon™ Transfection System and Neon™ Transfection System 100 μL Kit (Thermo Fisher Scientific). Transfection parameters were set to 1610 V, 10 ms pulse width, and 3 pulses.

TABLE 2
10x KO Target Knockout Gene Specifications

| Repre- | Protein name | | | |
|---|---|---|---|---|
| sented | (HUGO/NCBI | | Pro- | |
| gene | gene | Gene | tein | *gRNA |
| name | database) | symbol | symbol | sequence |
| Gene | BCL2 | Bax | BAX | GGGTCGGGGGAG |
| A | associated | | | CAGCTCGG |
| | X, | | | |
| | apoptosis | | | |
| | regulator | | | |
| Gene | BCL2 | Bak1 | BAK1 | ATGGCGTCTGGA |
| B | antagonist/ | | | CAAGGACC |
| | killer 1 | | | |
| Gene | Sirtuin 1 | Sirt1 | SIRT1 | GCTCTAGTGACT |
| C | | | | GGACTCCA |
| Gene | MYC proto- | Myc | MYC | CACCATCTCCAG |
| D | oncogene, | | | CTGATCCG |
| | bHLH trans- | | | |
| | cription | | | |
| | factor | | | |
| Gene | Intercel- | Icam | ICAM1 | ACCTGCATGGAT |
| E | lular | | | GCACCCCG |
| | adhesion | | | |
| | molecule 1 | | | |
| Gene | Lipoprotein- | PLA2G7 | Lp- | ATCCAGCAGTCA |
| F | associated | | PLA₂ | ATGATAAC |
| | phospho- | | | |
| | lipase A2 | | | |
| Gene | Lipoprotein | Lpl | LPL | CAGAGTTTGACC |
| G | lipase | | | GCCTCCCA |
| Gene | Palmitoyl- | Ppt1 | PPT1 | CTGCTGTAACCC |
| H | protein | | | CATAAGCA |
| | thioester- | | | |
| | ase 1 | | | |
| Gene | Cytidine | Cmah | CMAH | ACATTGAGGATT |
| I | monophospho- | | | TAGACGGA |
| | N-acetyl- | | | |
| | neuraminic | | | |
| | acid | | | |
| | hydroxylase | | | |
| Gene | Glycoprotein | Ggta1 | GGTA1 | TCACCGTCAAAA |
| J | alpha- | | | CCTCTGGG |
| | galactosyl- | | | |
| | transferase | | | |
| | 1 | | | |

*5′ to 3′ strand with underlined PAM site

TABLE 3
6X KO Target Knockout Gene Specifications

| Repre- | Protein name | | | |
|---|---|---|---|---|
| sented | (HUGO/NCBI | | Pro- | |
| gene | gene | Gene | tein | *gRNA |
| name | database) | symbol | symbol | sequence |
| Gene | BCL2 | Bax | BAX | GGGTCGGGGGAG |
| A | associated | | | CAGCTCGG |
| | X, | | | |
| | apoptosis | | | |
| | regulator | | | |
| Gene | BCL2 | Bak1 | BAK1 | ATGGCGTCTGGA |
| B | antagonist/ | | | CAAGGACC |
| | killer 1 | | | |
| Gene | Lipoprotein- | PLA2G7 | Lp- | ATCCAGCAGTCA |
| F | associated | | PLA₂ | ATGATAAC |
| | phospho- | | | |
| | lipase A2 | | | |
| Gene | Lipoprotein | Lpl | LPL | CAGAGTTTGACC |
| G | lipase | | | GCCTCCCA |
| Gene | Cytidine | Cmah | CMAH | ACATTGAGGATT |
| I | monophospho- | | | TAGACGGA |
| | N-acetyl- | | | |
| | neuraminic | | | |
| | acid | | | |
| | hydroxylase | | | |
| Gene | Glycoprotein | Ggta1 | GGTA1 | TCACCGTCAAAA |
| J | alpha- | | | CCTCTGGG |
| | galactosyl- | | | |
| | transferase | | | |
| | 1 | | | |

*5′ to 3′ strand with underlined PAM site

TABLE 4
8x KO Target Knockout Gene Specifications

| Repre- | Protein name | | | |
|---|---|---|---|---|
| sented | (HUGO/NCBI | | Pro- | |
| gene | gene | Gene | tein | *gRNA |
| name | database) | symbol | symbol | sequence |
| Gene | BCL2 | Bax | BAX | GGGTCGGGGGAG |
| A | associated | | | CAGCTCGG |
| | X, | | | |
| | apoptosis | | | |
| | regulator | | | |
| Gene | BCL2 | Bak1 | BAK1 | ATGGCGTCTGGA |
| B | antagonist/ | | | CAAGGACC |
| | killer 1 | | | |
| Gene | Lipoprotein- | PLA2G7 | LPLA₂ | ATCCAGCAGTCA |
| F | associated | | | ATGATAAC |
| | phospho- | | | |
| | lipase A2 | | | |
| Gene | Lipoprotein | Lpl | LPL | CAGAGTTTGACC |
| G | lipase | | | GCCTCCCA |
| Gene | Cytidine | Cmah | CMAH | ACATTGAGGATT |
| I | monophospho- | | | TAGACGGA |
| | N-acetyl- | | | |
| | neuraminic | | | |
| | acid | | | |
| | hydroxylase | | | |
| Gene | Glycoprotein | Ggta1 | GGTA1 | TCACCGTCAAAA |
| J | alpha- | | | CCTCTGGG |
| | galactosyl- | | | |
| | transferase | | | |
| | 1 | | | |
| Gene | Branched | BCKDHA | BCKDHA | GGTCCATGACCC |
| K | chain keto | | | GGTAGATG |
| | acid | | | |
| | dehydro- | | | |
| | genase | | | |
| | E1 alpha | | | |
| | subunit | | | |
| Gene | Branched- | BCKDHB | BCKDHB | CGCGGGCGGCCG |
| L | chain alpha- | | | GGATTCTG |
| | keto acid | | | |
| | dehydro- | | | |
| | genase | | | |
| | E1 beta | | | |
| | subunit | | | |

TABLE 5
9X KO Target Knockout Gene Specifications

| Repre- | Protein name | | | |
|---|---|---|---|---|
| sented | (HUGO/NCBI | | Pro- | |
| gene | gene | Gene | tein | *gRNA |
| name | database) | symbol | symbol | sequence |
| Gene | BCL2 | Bax | BAX | GGGTCGGGGGAG |
| A | associated | | | CAGCTCGG |
| | X, | | | |
| | apoptosis | | | |
| | regulator | | | |
| Gene | BCL2 | Bak1 | BAK1 | ATGGCGTCTGGA |
| B | antagonist/ | | | CAAGGACC |
| | killer 1 | | | |
| Gene | Sirtuin 1 | Sirt1 | SIRT1 | GCTCTAGTGACT |
| C | | | | GGACTCCA |
| Gene | Intercel- | Icam | ICAM1 | ACCTGCATGGAT |
| E | lular | | | GCACCCCG |
| | adhesion | | | |
| | molecule 1 | | | |
| Gene | Lipoprotein- | PLA2G7 | Lp- | ATCCAGCAGTCA |
| F | associated | | PLA₂ | ATGATAAC |
| | phospho- | | | |
| | lipase A2 | | | |
| Gene | Lipoprotein | Lpl | LPL | CAGAGTTTGACC |
| G | lipase | | | GCCTCCCA |
| Gene | Palmitoyl- | Ppt1 | PPT1 | CTGCTGTAACCC |
| H | protein | | | CATAAGCA |
| | thioesterase | | | |
| | 1 | | | |
| Gene | Cytidine | Cmah | CMAH | ACATTGAGGATT |
| I | monophospho- | | | TAGACGGA |
| | N-acetyl- | | | |
| | neuraminic | | | |
| | acid | | | |
| | hydroxylase | | | |
| Gene | Glycoprotein | Ggta1 | GGTA1 | TCACCGTCAAAA |
| J | alpha- | | | CCTCTGGG |
| | galactosyl- | | | |
| | transferase | | | |
| | 1 | | | |

TABLE 6
Penta (5x) KO Target Knockout Gene
Specifications

| Repre- | Protein name | | | |
|---|---|---|---|---|
| sented | (HUGO/NCBI | | Pro- | |
| gene | gene | Gene | tein | *gRNA |
| name | database) | symbol | symbol | sequence |
| Gene | BCL2 | Bax | BAX | GGGTCGGGGGAG |
| A | associated | | | CAGCTCGG |
| | X, | | | |
| | apoptosis | | | |
| | regulator | | | |
| Gene | BCL2 | Bak1 | BAK1 | ATGGCGTCTGGA |
| B | antagonist/ | | | CAAGGACC |
| | killer 1 | | | |
| Gene | Sirtuin 1 | Sirt1 | SIRT1 | GCTCTAGTGACT |
| C | | | | GGACTCCA |
| Gene | MYC proto- | Myc | MYC | CACCATCTCCAG |
| D | oncogene, | | | CTGATCCG |
| | bHLH | | | |
| | transcrip- | | | |
| | tion | | | |
| | factor | | | |
| Gene | Intercel- | Icam | ICAM1 | ACCTGCATGGATG |
| E | lular | | | CACCCCG |
| | adhesion | | | |
| | molecule 1 | | | |

Genomic DNA PCR and gRNA Indel Analysis

At 48-72 hours post-transfection, DNA from RNP-transfected cells was extracted using the DNeasy Blood and Tissue Kit (Qiagen). A 400-500 bp region of DNA centered on each gRNA cut site was PCR amplified. Amplicons were purified using the QIAquick PCR Purification Kit (Qiagen) and sequenced using Sanger sequencing. The Sanger sequencing traces for each test sample and its corresponding control sample were uploaded to the Inference of CRISPR Edits (ICE) software tool and analyzed according to the developer's instructions. ICE analysis reports “indel percentage” and “knockout score.” The “Indel percentage” represents the editing efficiency of the edited trace against the control trace, regardless of whether the indel results in a frameshift; the “knockout score” represents the proportion of cells that have either a frameshift indel or a fragment deletion, which likely results in a functional knockout.

Multiplex CRISPR editing and generation of CHO KO cell pools and single cell clones For the 6× CHO KO pools and cell lines (BAX, BAK, LPLA2, LPL, CMAH, and GGTA1), 8× CHO KO pools and cell lines (BAX, BAK, LPLA2, LPL, CMAH, GGTA1, BCKDHA, and BCKDHB), 9× CHO KO pools and cell lines (genes BAX, BAK, SIRT-1, ICAM-1, LPLA2, LPL, PPT1, CMAH, and GGTA1), and the 10× CHO KO pools and cell lines (genes BAX, BAK, SIRT-1, MYC, ICAM-1, LPLA2, LPL, PPT1, CMAH, and GGTA1), only one gRNA was used for each gene target. The most efficient guide for each target gene was identified and used to generate the 6×, 8×, 9×, and 10× CHO KO pools. A parental CHO host with the Bax and Bak genes previously knocked out, was used to generate the 6×, 8×, 9×, and 10× CHO KO pools and cell lines. Therefore, an additional four genes, six genes, seven genes or eight genes were targeted to generate the 6×, 8×, 9×, and 10× CHO KO pools and cell lines, respectively. The strategy for the Penta (5×) KO is described below in Example 8.

Four sgRNAS, six sgRNAs, seven sgRNAs or eight sgRNAs were pooled together, at a ratio of 1:1 sgRNA (20 pmol) to Cas9 protein (20 pmol), to form 20 pmols of RNP for each target gene, to generate the 6×, 8×, 9×, and 10× CHO KO pools and cell lines. Twelve million cells were transfected with the combined RNPs. Therefore, when targeting 4 genes, 6 genes, 7 genes, or 8 genes, a total of 80 pmols, 120 pmols, 140 pmols, or 160 pmols of RNP was used, respectively. Three sequential transfections of the 1:1 ratio of sgRNA and Cas9 protein were performed to improve the knockout efficiency for each target gene. The editing efficiency was measured after each transfection.

The 6×, 8×, 9×, and 10× cell KO pools were single-cell cloned by single cell printing (SCP) into 384-well plates with a target seeding density of 1 cell/well. Plates were cultured for 2 weeks at 37° C., 5% CO2, and 80% humidity. This step was followed by automated confluency-based hit-picking of wells with a targeted occupancy of 1 cell/well and subsequent expansion to 96-well plates using Microlab STAR (Hamilton).

### DNA Sequencing and ICE Analysis of Knockout Cell Pools and Single Cell Clones

Genomic DNA was extracted from transfected pools and single cell clones using the MagNA Pure 96 Instrument (Roche Life Science), followed by PCR to amplify the genomic region around each gRNA cut site as described previously. PCR products were then purified using a QIAquick 96 PCR purification kit (Qiagen) or a ZR-96 DNA Clean-Up Kit (Zymo Research) according to the manufacturer's instructions, followed by Sanger sequencing and ICE indel analysis.

### Fed-Batch Production Cultures

A 12-day production culture assay was performed with the 9× KO (genes BAX, BAK, SIRT-1, ICAM-1, LPLA2, LPL, PPT1, CMAH, and GGTA1) and 1Ox KO (genes BAX, BAK, SIRT-1, MYC, ICAM-1, LPLA2, LPL, PPT1, CMAH, and GGTA1) CHO pools in an ambr15 microbioreactor system (Sartorius Stedim Biotech). Parameters such as growth, viability, and titer were assessed. Cells were seeded at 40×10⁶ cells/mL at day 0 of production in a proprietary serum free production medium, followed by a temperature shift to 33° C. on day 2. The production cultures were maintained in an environment with pH and dissolved oxygen control. The production cultures received a proprietary feed on days 1, 4, and 8. On day 12, harvested cell culture fluid (HCCF) was collected and analyzed. Day 12 titers were determined using protein A affinity chromatography with UV detection. Percent viability and viable cell counts were monitored using the FLEX2 Automated Cell Culture Analyzer (Nova Biomedical). Integrated viable cell count (IVCC) for each production culture was calculated using viable cell count measurements; IVCC represents the integral of the area under the growth curve for viable cells over the culture duration.

### Vector Constructs, Cell Culture Conditions and Production

Expression of both the heavy chain (HC) and light chain (LC), as two separate units, was directed by their respective cytomegalovirus (CMV) promoter and regulator elements. Plasmids encoded dihydrofolate reductase (DHFR) or puromycin as selection markers directed by the Simian Virus (SV) 40 early promoter and enhancer elements. The SV40 late polyadenylation (poly A) signal sequences were used in the 3′ region of the HC DNA and LC DNA. Cells were cultured in a proprietary serum-free DMEM/F12-based medium in 50-mL tube spin vessels shaking at 150 rpm, 37° C. and 5% CO₂ and were passaged at a seeding density of 4×10⁵ cells/mL every 3-4 days (Hu, et al., 2013).

Fed-batch production cultures were performed as disclosed here and below in Example 2, with proprietary chemically defined medium using different vessels (e.g., tube-spin and AMBR15) along with bolus feeds on Days 3, 7 and 10 as previously mentioned (Hsu, Aulakh, Traul, & Yuk, 2012). Anti-cell aggregation agent was used in all cultures during production assay to prevent cell aggregation due to the release of DNA from dying cells. Cells were seeded at low (1-2×10⁶ cells/mL) or high (10×10⁶ cells/mL) seeding densities using lean or rich production media. Cultures were temperature shifted from 37° C. to 35° C. on Day 3. Titers were determined using Protein-A affinity chromatography with UV detection. Percent viability and viable cell count were determined using a Vi-Cell XR instrument (Beckman Coulter Item #383721).

CRISPR/Cas9-mediated disruption of PERK (EIF2AK3)

sgRNA primer sequences were as follows:

PERK sgRNA 1:
5′AGTCACGGCGGGCACTCGCG
PERK sgRNA 2:
5′TACGGCCGAAGTGACCGTGG
PERK sgRNA 3:
5′GCGTGACTCATGTTCGCCAG
Luciferase sgRNA:
5′ATCCTGTCCCTAGTGGCCC

Five million cells were washed and suspended in buffer R (Neon 100 uL kit cat: MPK10025 Invitrogen). Five micrograms of Cas9:sgRNA RNP complex were added to the cell culture mixture. Cells were electroporated using 3×10 ms pulses at 1,620 V. The transfected cells were cultured for 3 days followed by single cell cloning via limiting dilution. Pool and single cell clones were screened for PERK knockout by western blot analysis.

### RT-PCR Analysis to Detect IRE1 Alpha RNase Activity

CHO-XBP1s Forward Primer:
5′CCTTGTAATTGAGAACCAGG
CHO-XBP1s Reverse Primer:
5′CCAAAAGGATATCAGACTCGG

Power SYBR Green RNA-to CT-1 Step Kit and protocol used from Applied Biosystems (#4389986).

### Immunoblotting and Reagents

1.5 million cells were lysed in 1× NP40 buffer (10 mM Tris, pH 8.0, 0.5% NP40, 150 mM NaCl, 10 mM DTT and 5 mM MgCl₂) containing protease inhibitor cocktails (Roche EDTA free mini-tablets cocktail) for 20 min on ice. Lysates were subjected to sodium dodecyl sulfate-polyacrylamide gel electrophoresis (SDS-PAGE) (4-12% Tris glycine) and transferred to nitrocellulose membrane. After blocking with 5% milk in tris-buffered saline (TBS)-0.1% Tween buffer, the membranes were blotted with respective antibodies. Blots were visualized using HRP-conjugated anti-rabbit anti-body and SuperSignal West Dura Extended Duration Substrate. The following inhibitors were used: ATF6i (10 μM Ceapin-A7 (Gallagher, et al., 2016)), PERKi (10 μM Compound 39 (Axten, et al., 2012)), IRE1i6 (10 μM 4u8c (Cross, et al., 2012)), IRE1i9 (10 μM in-house/Genentech), PDGFRi (5-20 μM Abcam, AG-1296). The following antibodies were used: anti-PDGFRa (Cell Signaling Technology (CST), D1E1E), rabbit anti-BiP (C50B12, Cell Signaling Technology, 3177), rabbit anti-PERK (CST, C33E10), mouse anti-o-actin-HRP (AC-15) (Abcam, ab49900), rabbit anti-phospho-Akt (Ser473) (CST, D9E), rabbit anti-Akt (CST, 5G3), rabbit cleaved caspase3 (CST, asp175), goat anti-human IgG-HRP (MP Biomedicals, 0855252), rabbit IRE1a (CST, 14C10), mouse anti-phospho-IRE1, mouse anti-XBP1, rabbit anti-Bax (Abcam, ab32503), rabbit anti-Bak (CST, D4E4), donkey anti-rabbit HRP (Jackson ImmunoResearch Laboratories, Inc., 711-035-152), rabbit anti-sod2 (CST, D3×8F)

### Example 2: Multiplex CRISRP/Cas9 KO Workflows Using Ribonucleoproteins (RNPs)

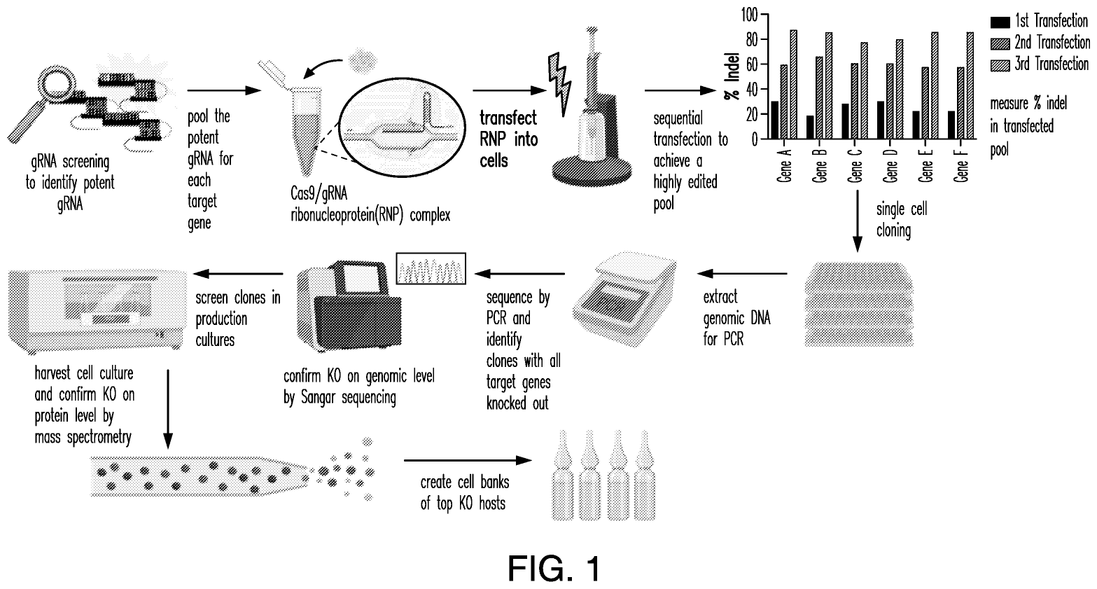

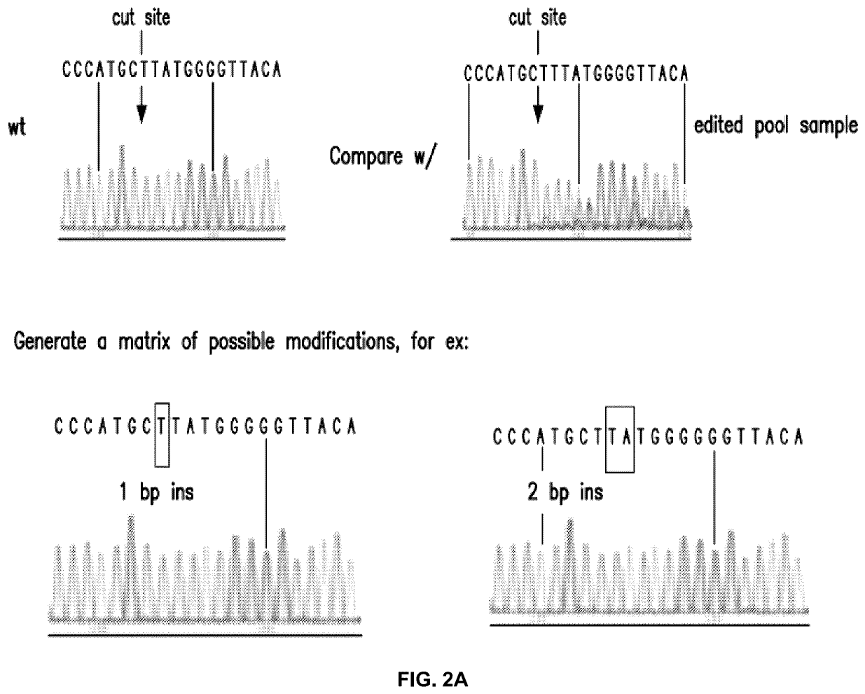

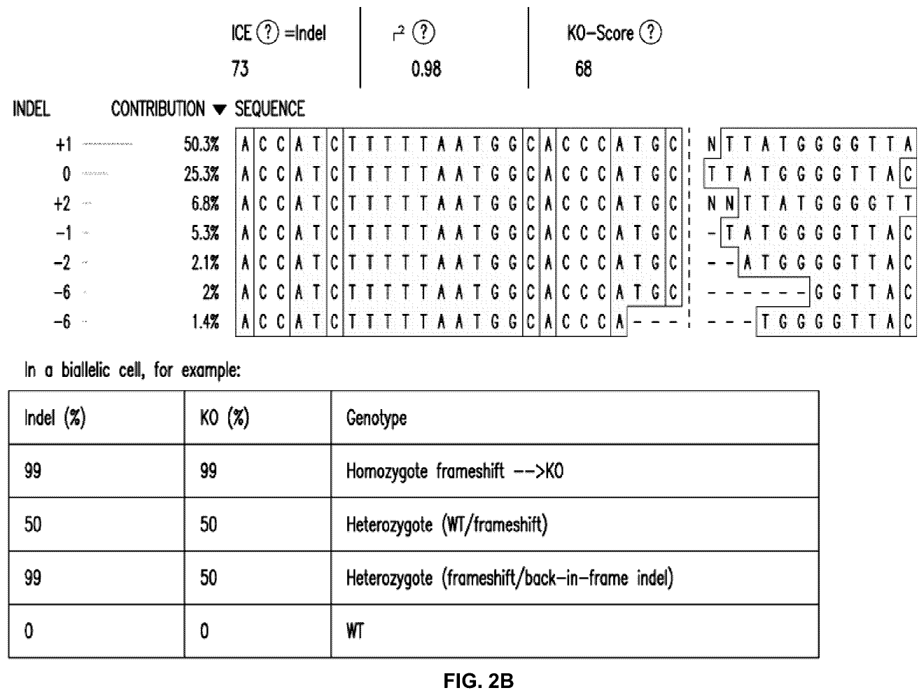

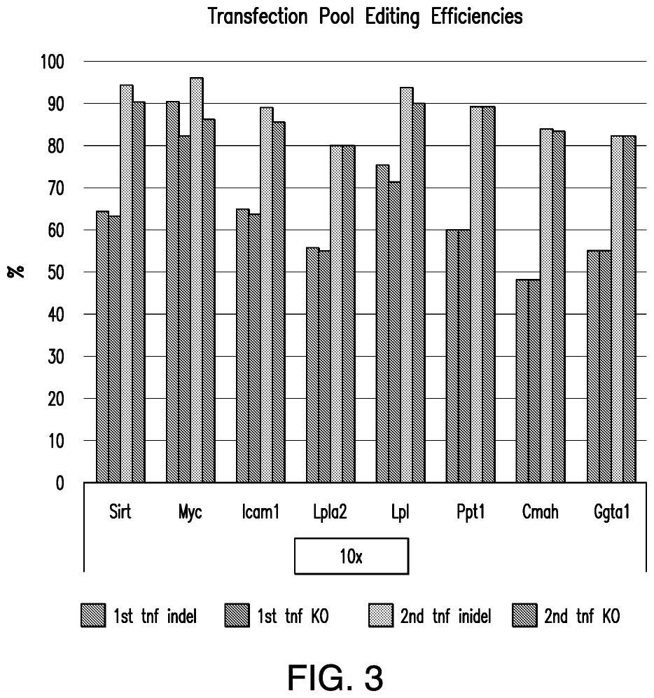

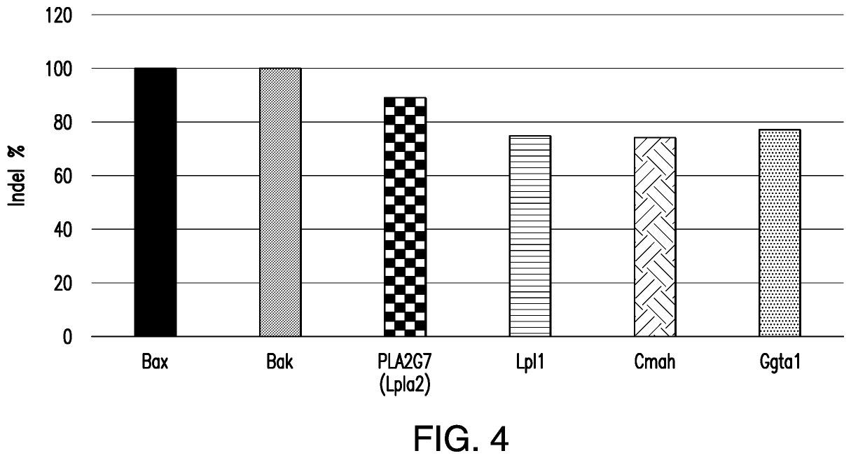

FIG. 1 illustrates an exemplary workflow to generate single cell clones from a pool in which multiple genes are knocked out (e.g., ten genes (BAX; BAK; ICAM-1; SIRT-1; MYC; GGTA1; CMAH; LPL; LPLA2; and PPT1), the “10×” KO pool, or eight genes (BAX; BAK; ICAM-1; GGTA1; CMAH; LPL; LPLA2; and PPT1), the “8×” KO pool). To identify a potent gRNA for each target gene, transfections of purified Cas9 protein bound to synthetic gRNA in an RNP complex to simultaneously screen several gRNAs for a given gene were performed (FIGS. 2A-2B). For quantification of editing efficiencies, Inference of CRISPR Edits (ICE) was used, an online software for analyzing Sanger sequencing data (How To Use ICE: A Detailed Guide for Analyzing CRISPR Editing Results; www.synthego.com/guide/how-to-use-crispr/ice-analysis-guide), which has been extensively validated for targeted NGS (Hsiau T, et al. Inference of CRISPR edits from Sanger trace data. BioRxiv. Published online 2018:251082.), to identify the type and quantitatively infer the abundance of Cas9 induced edits (Brinkman E K, et al., Easy quantitative assessment of genome editing by sequence trace decomposition. Nucleic acids research. 2014;42(22):e168-e168). The proposed workflow for transfecting cells with RNP, extracting DNA from the transfected cells, amplifying the region surrounding the gRNA cut sites, and analyzing the sequenced amplicon can be accomplished in only four days (FIGS. 2A-2B). For these gRNAs, crRNA-XT versions were used to transfect cells four times sequentially. For the highly potent gRNA targeting gene SMPD1E only one round of transfection (in the last round) was performed. The efficiency for the sequential transfection of the 10× pools as an example is shown in FIG. 3. The KO efficiencies are at least 70% for all genes after sequential transfections. The BAX/BAK double KO host was used for sequentially knocking out 8 genes to generate the 10× KO cells. FIG. 4 provides the indel knockout efficiency for each gene in the 6× CHO KO hosts. The percentage of KO measured in targeted pool by ICE. The indel percentage for the Bax and Bak1 genes were determined by Western blot analysis at 100%. The indel percentage of the remaining genes were determined by genomic DNA sequencing analysis.

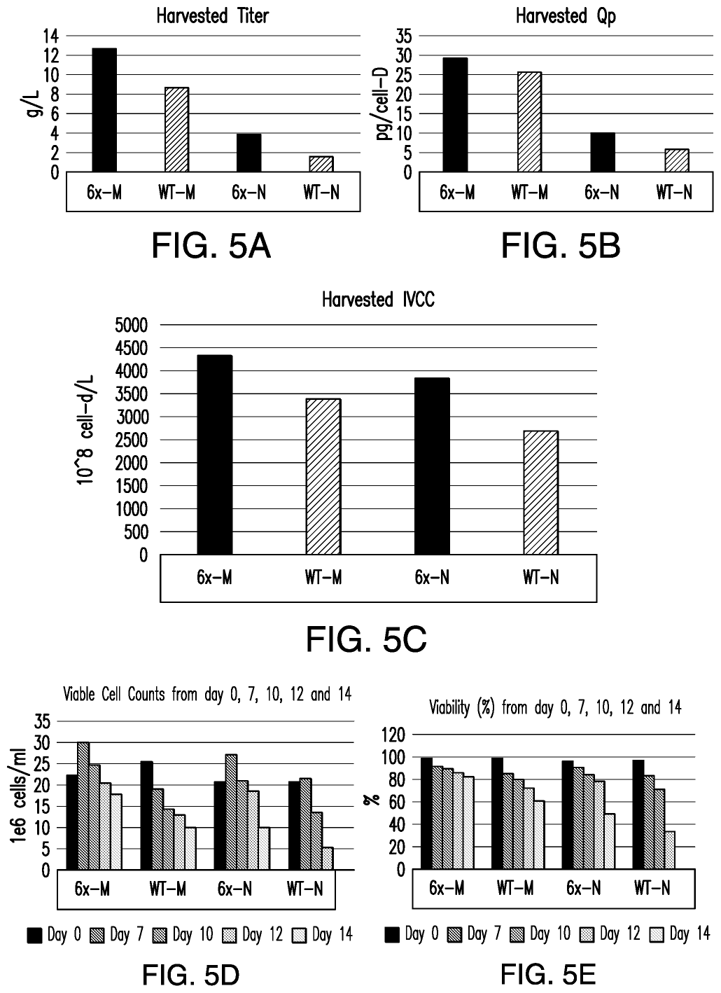

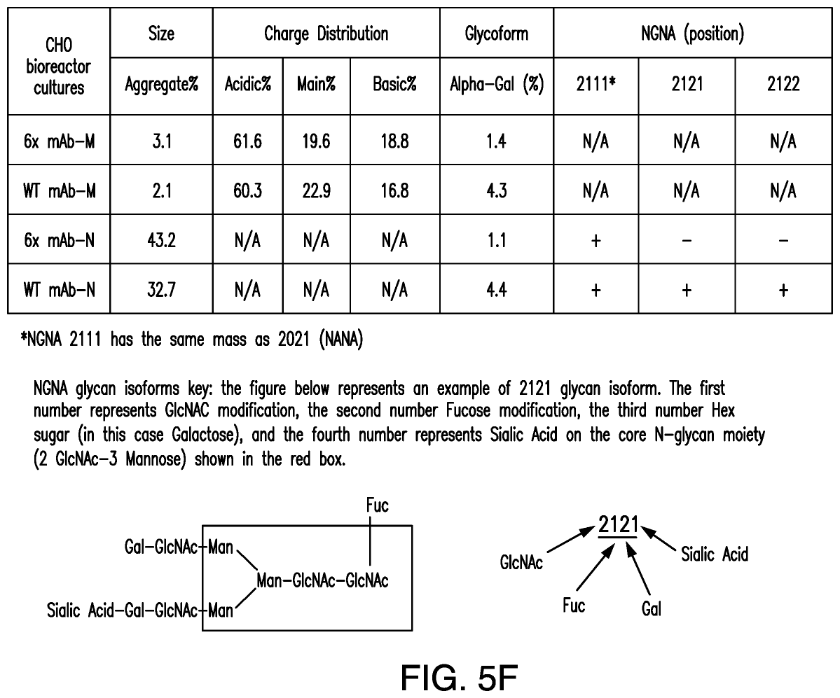

As illustrated in FIGS. 5A-5F, wild-type (WT) control and 6× KO pooled CHO cells were transfected with vectors expressing mAb-M or mAb-N and recovered pools were used to set up bioreactor production cultures in 2L vessels. The WT and 6× KO cultures were assessed for (5A) titer, (5B) cell-specific productivity (Qp), (5C) integrated viable cell count (IVCC), (5D) viable cell counts, (5E) viability. The cell-specific productivity (Qp) is also referred to as specific productivity and is calculated from dividing the product titer (for the mAb product) by the integrated viable cell count (IVCC). The IVCC represents the cumulative viable cell count over the duration of the bioreactor production culture, and it is calculated as the area under the viable cell count growth curve. The cultures were also assessed for impact on (5F) product quality in terms of % aggregates (which gives an indication of higher molecular weight forms of the mAb product), charge distribution (in terms of acidic, main and basic species), and glycoforms for alpha-Gal, and NGNA (N-glycolylneuraminic acid). Alpha-Gal and NGNA represent non-human glycosylation patterns present in CHO-derived recombinant proteins, and the CMAH and GGT1 gene knockouts were implemented in the 6× KO cells to minimize the expression of these non-human glycoforms in the recombinant mAb products. The WT CHO pool is the parental host with no gene knockouts. The WT-N production run was stopped on day 12 instead of day 14 because of low viability by day 12. NGNA Method: The levels of N-glycolylneuraminic acid (NGNA) containing glycans were determined by hydrophilic interaction liquid chromatography-mass spectrometry (HILIC-MS). In this analysis, glycans were enzymatically released from the protein by treatment with PNGase F and then fluorescently labeled with a procaine-based IPC fluorophore (InstantPC, Agilent Technologies) prior to their separation by hydrophilic interaction liquid chromatography. Relative quantification of the labeled glycans was accomplished by integration of the glycan fluorescence signals and identification of the separated glycans was determined by mass spectrometry. Alpha-Gal Method: The levels of alpha-Gal containing glycans were determined by hydrophilic interaction liquid chromatography-mass spectrometry (HILIC-MS) analysis of sialidase treated glycans. In this analysis, glycans were first treated with sialidase to remove sialic acids and then enzymatically released from the protein by treatment with PNGase F. The release glycans were subsequently labeled with a procaine-based IPC fluorophore (InstantPC, Agilent Technologies) prior to their separation by hydrophilic interaction liquid chromatography. Relative quantification of the labeled glycans was accomplished by integration of the glycan fluorescence signals and identification of the separated glycans was determined by mass spectrometry.

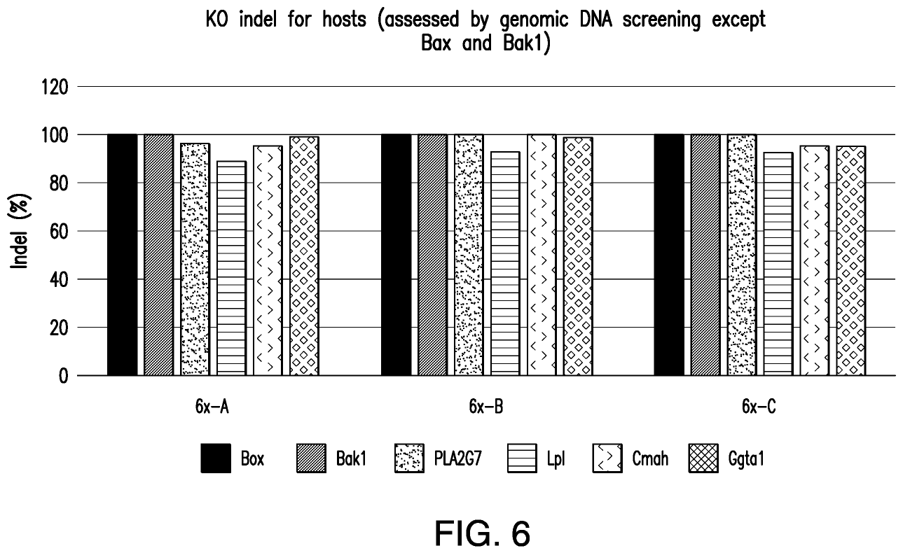

Three 6× clonal hosts were isolated as illustrated in FIG. 1 and the KO efficiency for each gene in each of the three 6× KO clonal hosts was determined (FIG. 6). The indel percentage for the Bax and Bak1 genes were determined by Western blot analysis. The indel percentage of the remaining genes were determined by genomic DNA sequencing analysis.

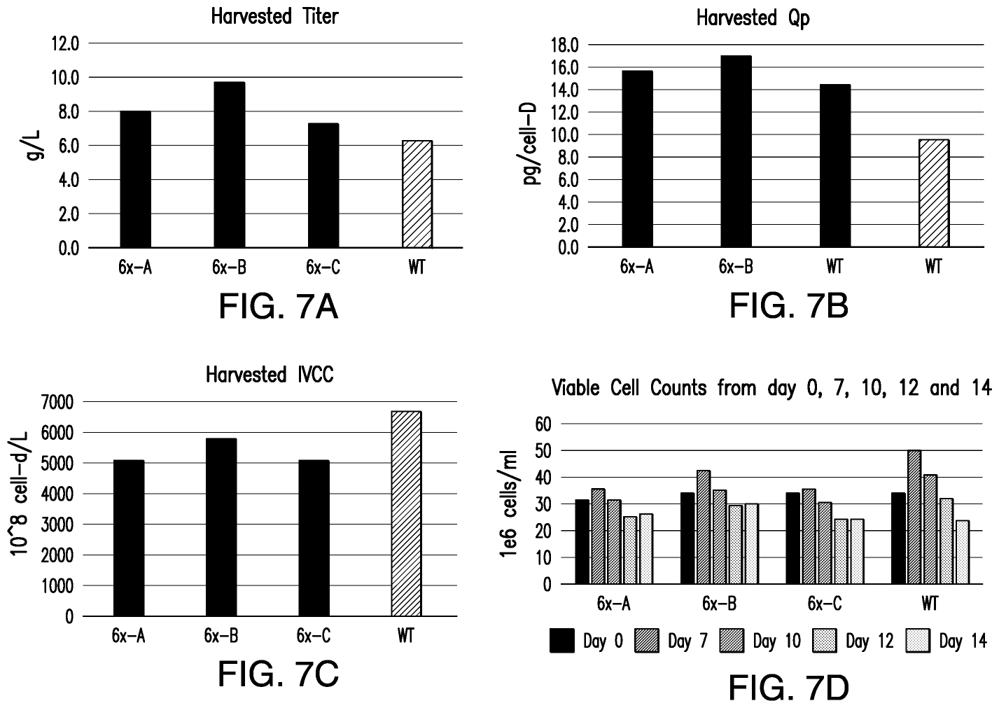

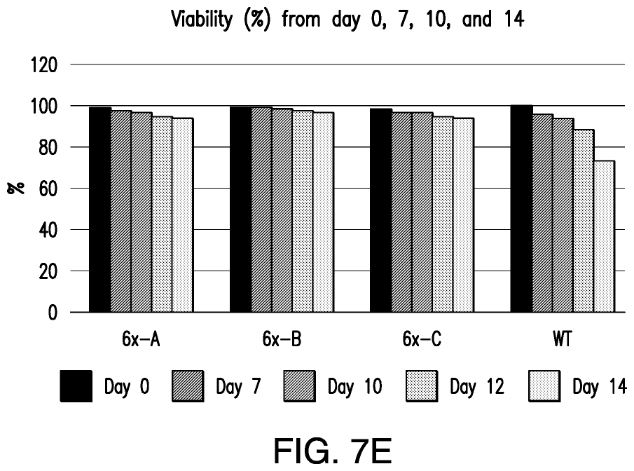

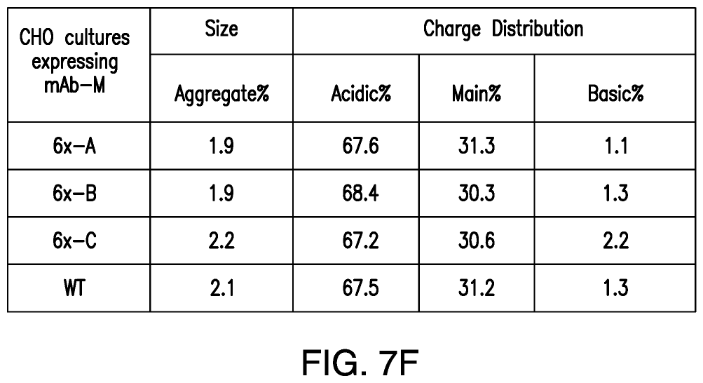

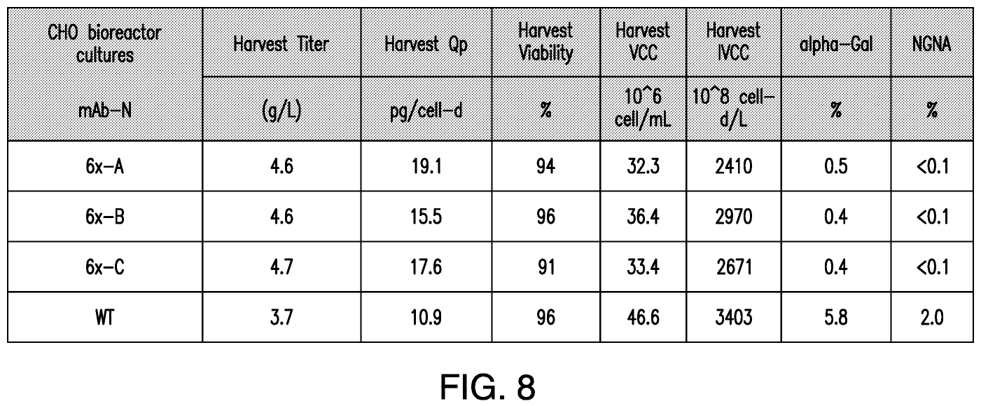

As illustrated in FIGS. 7A-7F, WT Control and 6× KO individually-developed clonal CHO hosts were transfected with vectors expressing mAb-M and the recovered pools were used to set up bioreactor production cultures in AMBR15 vessels. The WT and 6× CHO cultures were assessed for (7A) titer, (7B) cell-specific productivity (Qp), (7C) integrated viable cell count (IVCC), (7D) viable cell counts, (7E) viability for the 6X KO CHO host pools for mAb M. (7F) Product quality analysis measuring % aggregates and charge variant levels in the WT and 6X KO hosts were measured. In addition, WT Control and 6× KO individually developed clonal hosts were transfected with vectors expressing mAb-N and the recovered pools were used to set up bioreactor production runs in AMBR15 vessels. FIG. 8 shows harvest day titer, specific productivity (Qp), % viability, VCC, IVCC, and alpha-Gal and NGNA glycoform levels in the control WT and 6X KO hosts. Alpha-Gal and NGNA represent non-human glycosylation patterns. NGNA Method: The levels of N-glycolylneuraminic acid (NGNA) containing glycans were determined by hydrophilic interaction liquid chromatography-mass spectrometry (HILIC-MS). In this analysis, glycans were enzymatically released from the protein by treatment with PNGase F and then fluorescently labeled with a procaine-based IPC fluorophore (InstantPC, Agilent Technologies) prior to their separation by hydrophilic interaction liquid chromatography. Relative quantification of the labeled glycans was accomplished by integration of the glycan fluorescence signals and identification of the separated glycans was determined by mass spectrometry. Alpha-Gal Method: The levels of alpha-Gal containing glycans were determined by hydrophilic interaction liquid chromatography-mass spectrometry (HILIC-MS) analysis of sialidase treated glycans. In this analysis, glycans were first treated with sialidase to remove sialic acids and then enzymatically released from the protein by treatment with PNGase F. The release glycans were subsequently labeled with a procaine-based IPC fluorophore (InstantPC, Agilent Technologies) prior to their separation by hydrophilic interaction liquid chromatography. Relative quantification of the labeled glycans was accomplished by integration of the glycan fluorescence signals and identification of the separated glycans was determined by mass spectrometry.

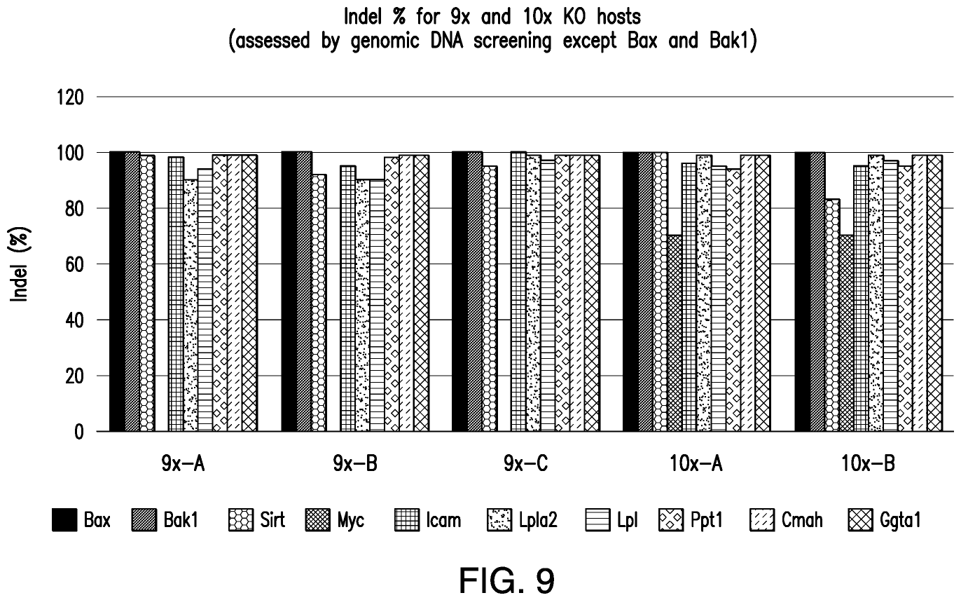

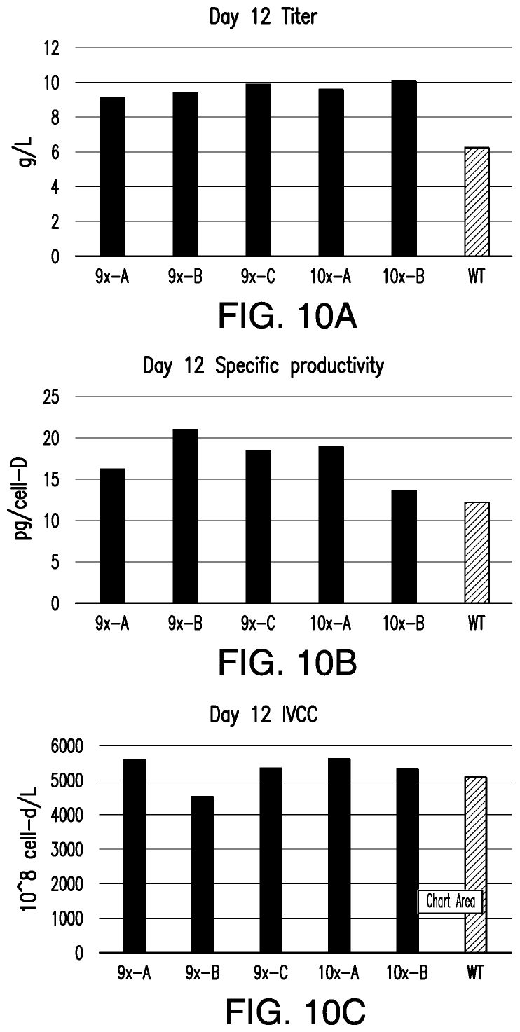

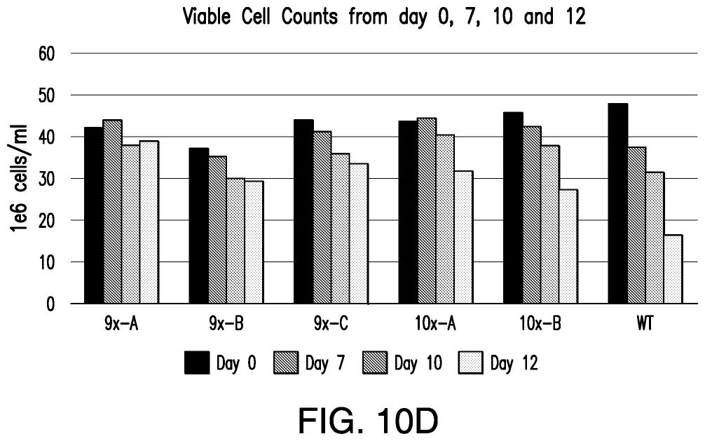

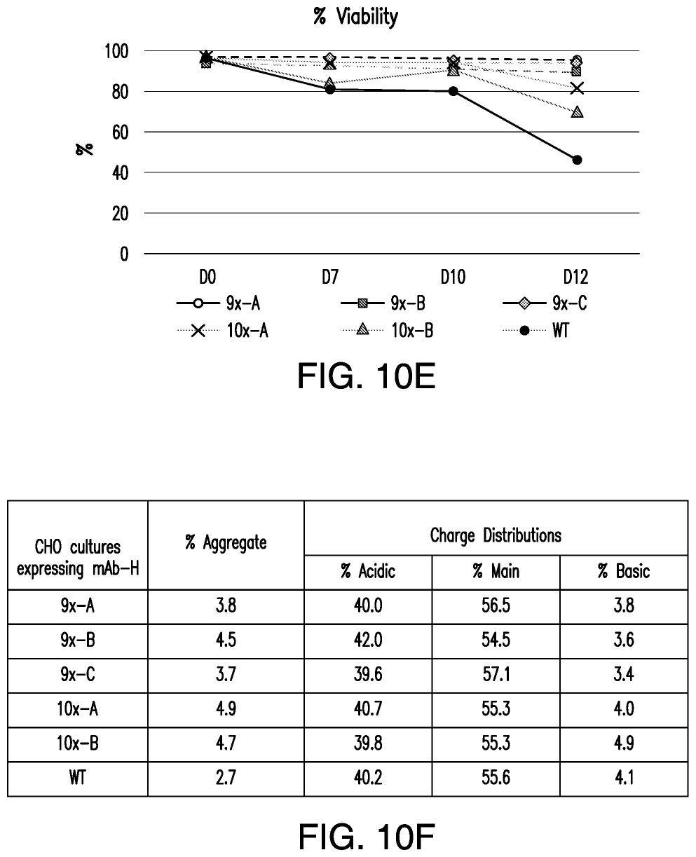

Indel knockout efficiency for each gene in the 9× (genes BAX, BAK, SIRT-1, ICAM-1, LPLA2, LPL, PPT1, CMAH, and GGTA1) and 10× KO (genes BAX, BAK, SIRT-1, MYC, ICAM-1, LPLA2, LPL, PPT1, CMAH, and GGTA1) hosts by genomic DNA analysis is shown in FIG. 9. The indel percentage for the Bax and Bak1 genes were determined by Western blot analysis. The indel percentage of the remaining genes were determined by genomic DNA sequencing analysis. Three different pools for the 9× KO CHO host and two different pools for the 1Ox KO CHO host were assessed for indel percentage. The 1Ox KO hosts differed from the 9× KO hosts in the use of Myc as a KO target. FIG. 10 depicts the titer (FIG. 10A), specific productivity (Qp) (FIG. 10B), integrated viable cell count (IVCC) (FIG. 10C) and viable cell counts for days 0, 7, 10 and 12 (FIG. 10D) for the 9× and 10× KO CHO pools for mAb-H. The WT CHO pool was the parental host with no gene knockouts. Three different pools for the 9× KO host and two different pools for the 10× KO host were assessed in fed-batch production cultures in bioreactors.

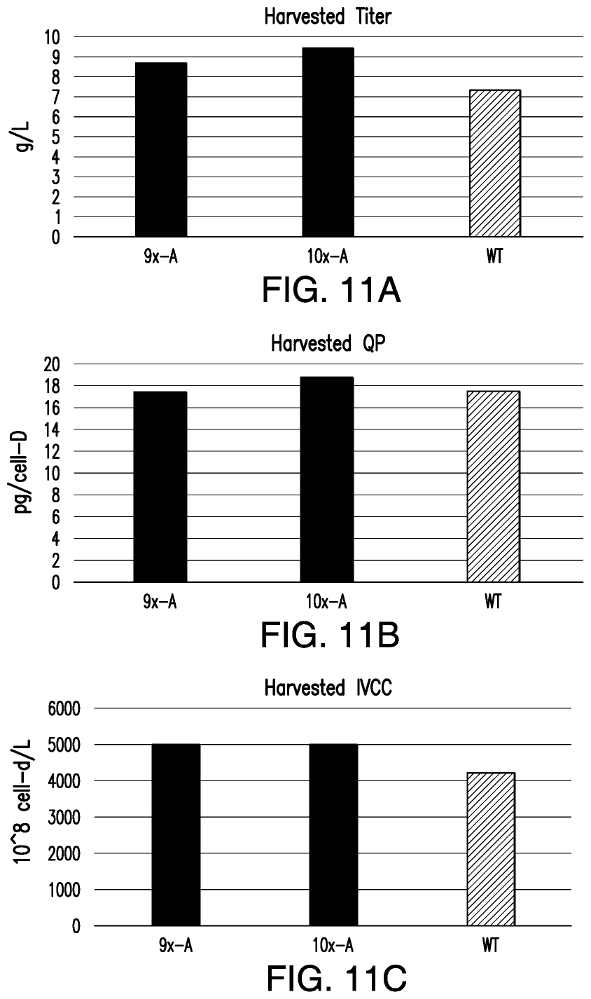

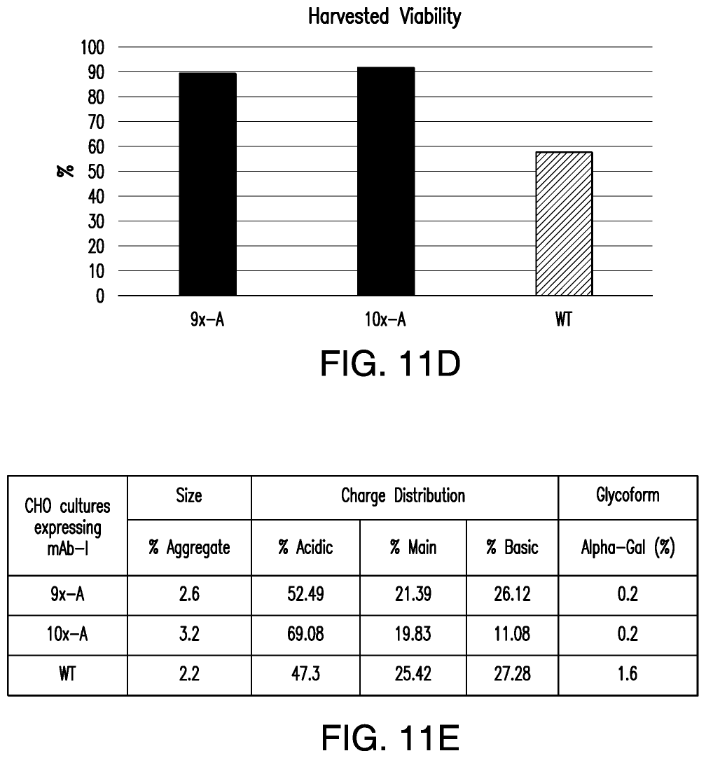

As illustrated in FIGS. 11A-11E, a 14-day production run in AMBR15 bioreactors was performed for WT, 9× KO, and 10× KO hosts expressing mAb-I. The Figures depict (11 A) titer, (11B) specific productivity (Qp), (11C) integrated viable cell count (IVCC), (11D) viable cell counts, and (11E) Product quality analysis was performed for the harvests from the bioreactors and they measured % aggregates, charge distribution, and non-human glycosylation levels (in terms of alpha-Gal).

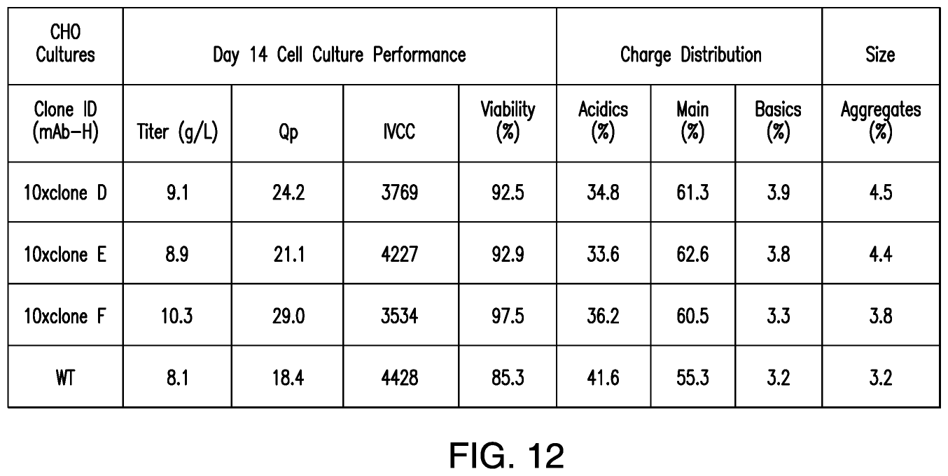

As illustrated in FIG. 12, 14-day production cultures were performed for WT and 1Ox KO (10×-A) mAb-H expressing top clones in AMBR15 bioreactors. WT and 10×-A mAb-H expressing CHO pools were single cell cloned and after screening, top clones from each arm were evaluated in 14-day production cultures in AMBR15 bioreactors. Day-14 titer, specific productivity (Qp), integrated viable cell count (IVCC), % viability, charge variant levels, and % aggregates were measured to assess the impact on cell culture performance and product quality.

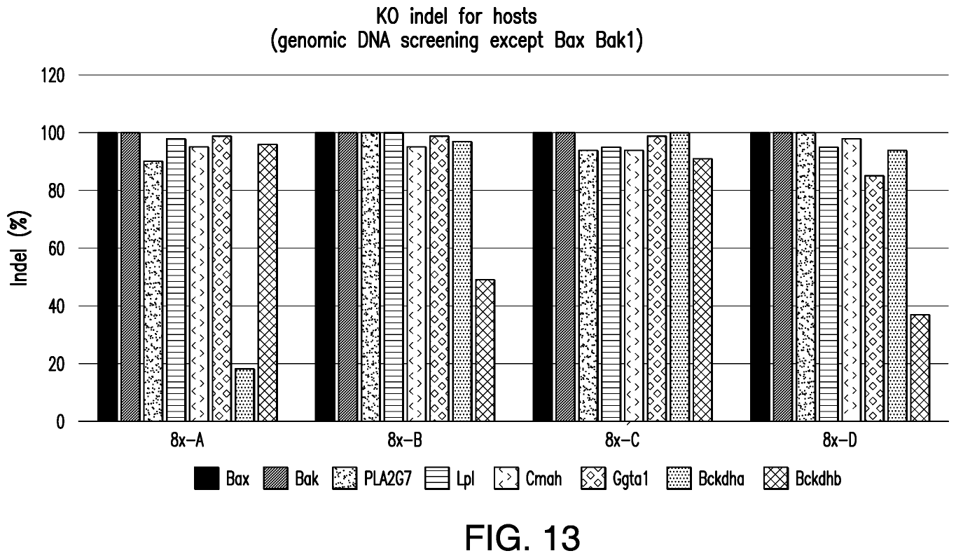

Four 8× clonal CHO hosts were isolated as illustrated in FIG. 1 and FIG. 13 illustrates a comparison of KO efficiency for each gene in each of four 8× KO clonal hosts. The indel percentage for the Bax and Bak1 genes were determined by Western blot analysis. The indel percentage of the remaining genes were determined by genomic DNA sequencing analysis.

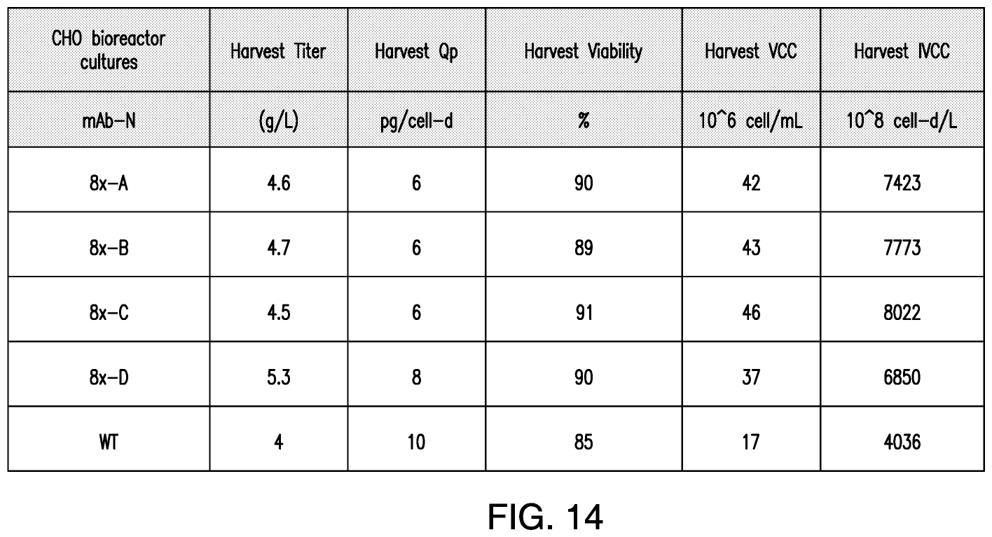

As illustrated in FIG. 14, a 14-day pool production cultures were performed for WT and four 8× KO clonal CHO hosts expressing mAb-N in AMBR15 bioreactors. WT and 8× KO clonal hosts were transfected and the recovered CHO pools were evaluated in 14-day bioreactor production cultures. FIG. 14 shows harvest day-14 titer, specific productivity (Qp), % Viability, viable cell count (VCC), and integrated viable cell count (IVCC) measured for the production cultures.

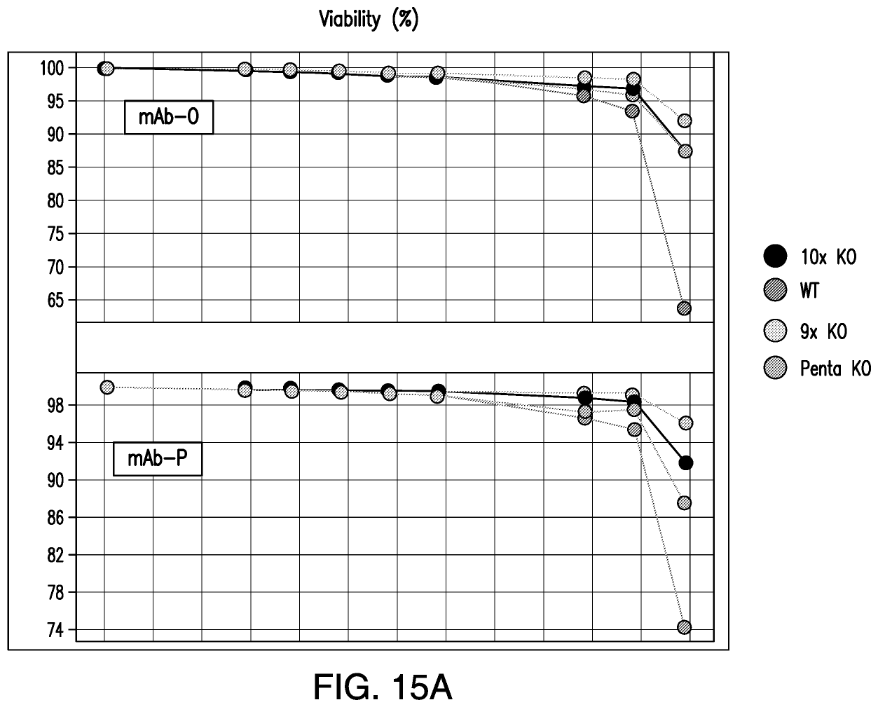

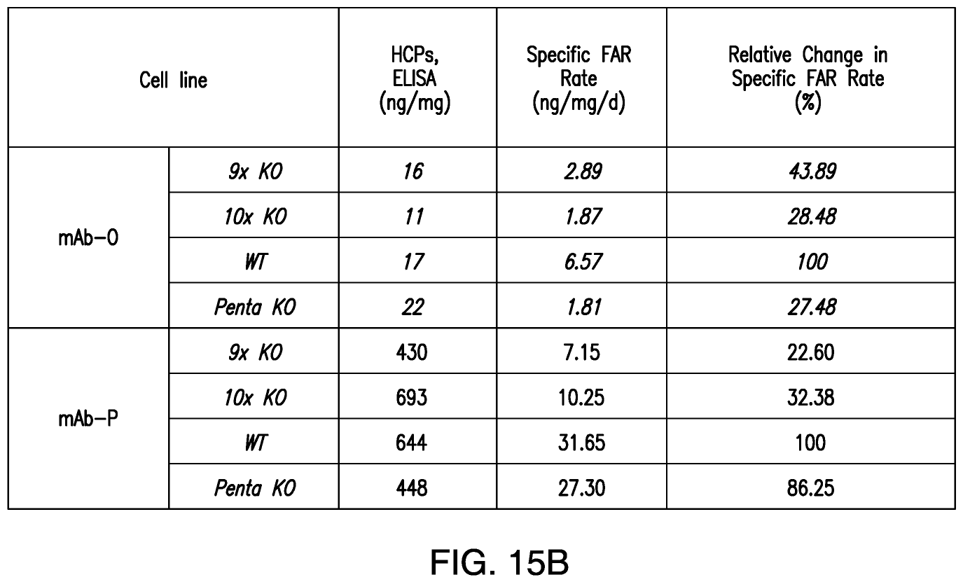

As illustrated in FIGS. 15A-15B, 12-day pool production cultures were performed for WT and Penta (5×), 9×, and 10× KO CHO pools expressing mAb-O or mAb-P. WT and KO hosts were transfected and the recovered pools were evaluated over 12 days AMBR250 bioreactors. (15A) Viability percentages for the production bioreactor cultures expressing mAb-O (top panel) and mAb-P (bottom panel) are shown. The bioreactor production cultures were harvested on day 12 and purified through affinity chromatography and two polishing chromatography steps. After undergoing three chromatography operations, which are representative of typical downstream processing for mAb products, the purified materials (mAb-O or mAb-P) were then analyzed for their levels of residual HCPs. The levels of HCPs in the purified materials were measured by our in-house platform CHP host cell protein (HCP) enzyme-linked immunosorbent assay (ELISA). The HCP levels were normalized to the amount of mAb product in the purified materials and quantified in ng/mg (i.e., ng of HCP per mg of mAb). The level of residual hydrolytic HCPs in the purified materials that can degrade polysorbate to release fatty acids was also assessed via fatty acid release (FAR) rates. The FAR rate studies were performed by incubating the purified materials with polysorbate 20, and using liquid chromatography (LC-MS) to measure the levels of fatty acids released by hydrolytic degradation of the polysorbate 20 over incubation time. The overall study procedure for incubating the purified materials with polysorbate 20 to assess the enzymatic activity towards polysorbate degradation via FAR rates has been previously described in detail (Cheng et al., 2019, Journal of Pharmaceutical Sciences, 108: 2880-2886). The LC-MS method to quantify the fatty acids released during the FAR rate studies has also been described in detail (Honenmann et al., 2019, Journal of Chromatography B, 1116:1-8). The specific FAR rate is calculated from the FAR rate by normalizing the FAR rate to the concentration of the mAb product. A higher specific FAR rate means higher hydrolysis of polysorbate, which therefore indicates higher risk of polysorbate degradation and particle formation in the drug product. Polysorbate is added as a surfactant to protect the drug product against interfacial stresses, and polysorbate degradation should be minimized over long-term storage of the drug product to ensure adequate surfactant remains to protect the product. When polysorbate degrades during long-term storage of the drug product, the resulting free fatty acids (produced as degradants) can accumulate and precipitate as particles. To maintain the quality of the drug product, it is important to minimize polysorbate degradation and risk of particle formation. Therefore, it is desirable to lower the specific FAR rates measured in the purified materials. (15B) Table shows levels of HCPs as measured by HCP ELISA and rates of polysorbate degradation as represented by specific FAR rates.

### Example 3: Disrupting Endogenous Expression of RVLPs

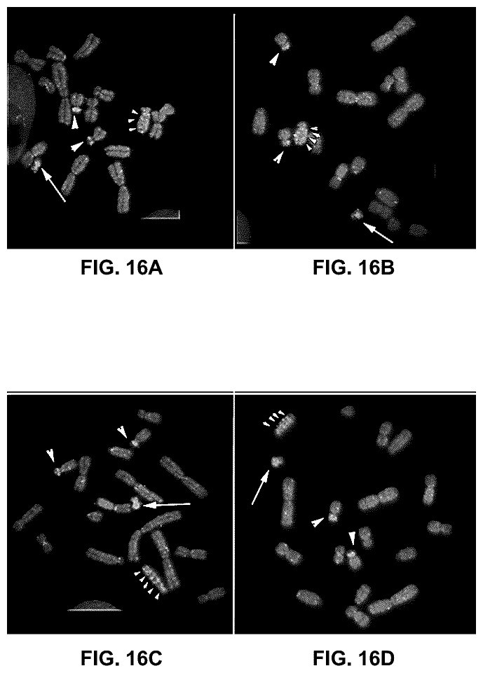

FIGS. 16A-16D illustrate fluorescence in situ hybridization (FISH) analysis of four different CHO cell lines (FIGS. 16A-16D). Two of the cell lines were CHO host cell lines (one derived from CHO-Kl and one that is a TI cell line) and two of the cell lines were CHO recombinant cell lines (generated from transfection of TI host) that produce a recombinant monoclonal antibody. Probe for RVLP was used to seek RVLP signal on CHO chromosomes. A strong RVLP signal (as indicated by arrow with line) on one chromosome and several weak signals on various other chromosomes (as indicated by arrow head without line) were observed for all four tested CHO cell lines.

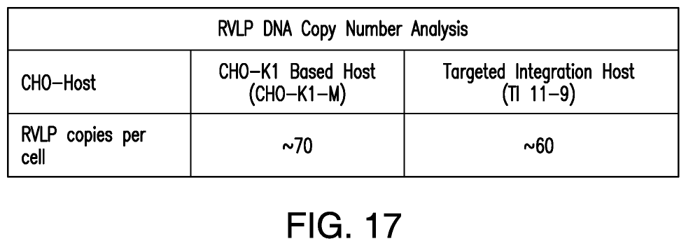

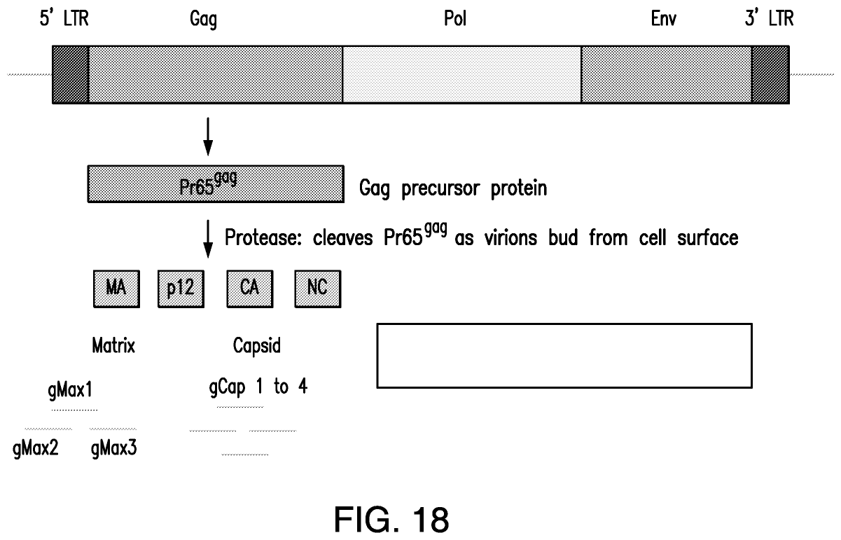

FIG. 17 provides RVLP DNA copy number analysis of two CHO host cell lines. A plasmid specific for RVLP was used as the standard (1 uL of the DNA standard was equivalent to 1.8×10⁸ copies). This plasmid used the same sequence as the RVLP probe for FISH analysis. FIG. 18 illustrates the design of guide RNA (gRNA) construct for disrupting RVLP expression in CHO cells. Different guide RNAs for matrix (gMax) and capsid (gCap) of the RVLPs were designed with the aim to disrupt the endogenous RVLP expression in CHO cells, and thereby generate a modified CHO host cell that expresses lower levels of RVLP; for e.g., by eliminating or reducing GAG expression. Such a modified mammalian host would decrease the burden on downstream processing to remove endogenous RVLPs in biomanufacturing.

### Example 4: UPR Activation Attenuates PDGFRa Transcription and Downregulates Its Expression

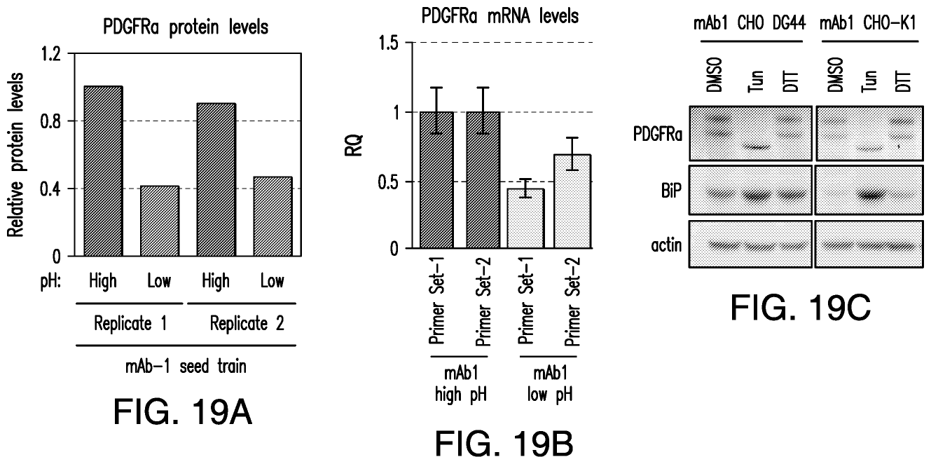

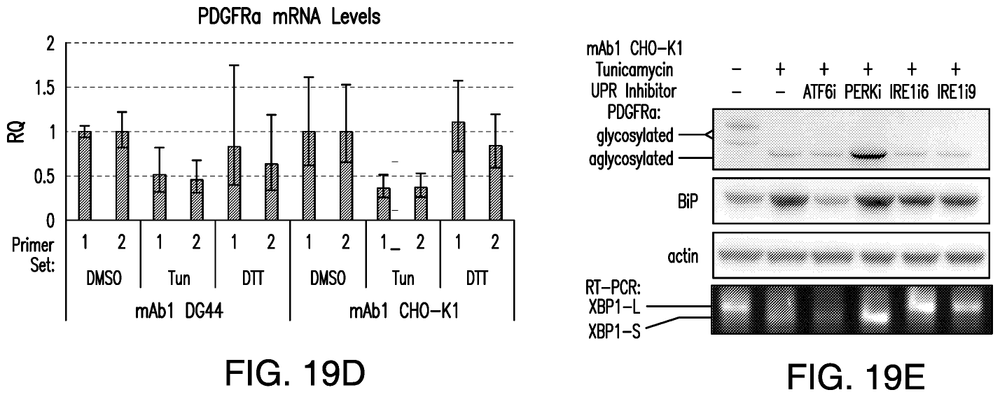

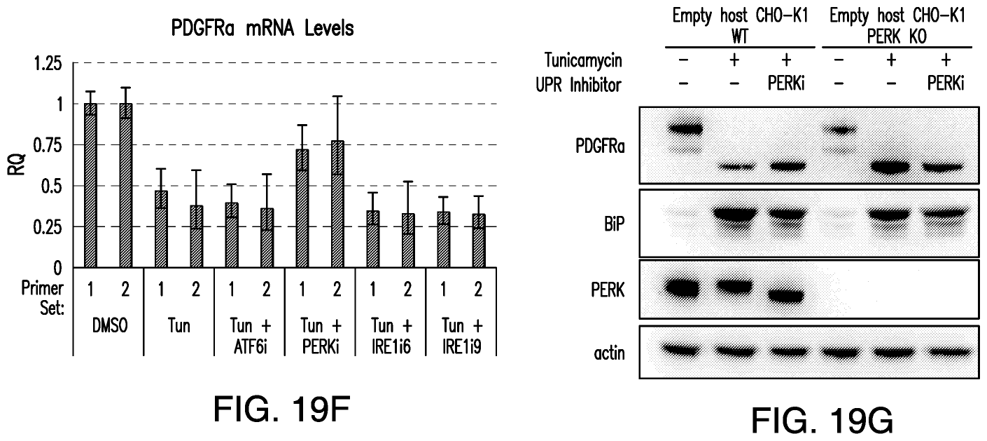

An interesting phenomenon was previously described where activation of UPR in seed train culture, triggered by lower pH conditions in a particular CHO cell line, negatively affected culture growth in production media at target pH (Tung, et al., 2018). When exposed to low pH conditions, high intracellular BiP levels were detected in this cell line that correlated to low growth profiles during production and poor bioprocess outcomes (Tung, et al., 2018). To better understand the underlying mechanism of reduced production culture growth in low vs high pH conditions, seed train cultures were maintained under high and low pH conditions and subjected to proteomic analysis by mass spectrometry. Significant reductions in expression of PDGFRa protein levels were observed under low pH conditions (FIG. 19A), which was linked to transcriptional attenuation of the PDGFRa gene (FIG. 19B). Since high intracellular BiP levels are indicative of UPR activation, it was decided to investigate the potential correlation between the UPR and reduced PDGFRa levels in CHO cells. Tunicamycin (Tun, strong UPR inducer) and DTT (weak UPR inducer) were used to chemically induce UPR in the seed train cultures of two antibody-expressing (mAb1) CHO host lines, CHO DG44 and CHO-Kl. Under optimal pH conditions and with a strong UPR inducer (Tun), fully functional PDGFRa levels were reduced at both the protein and mRNA levels in both CHO host backgrounds (FIGS. 19C and 19D). Note that BiP levels, as an indicator of UPR activation, increase accordingly in response to strong and weak UPR chemical inducers (FIG. 19C). The lower molecular weight PDGFRa protein band observed upon tunicamycin treatment represents the non-glycosylated form of this protein as tunicamycin treatment inhibits protein glycosylation (FIG. 19C).

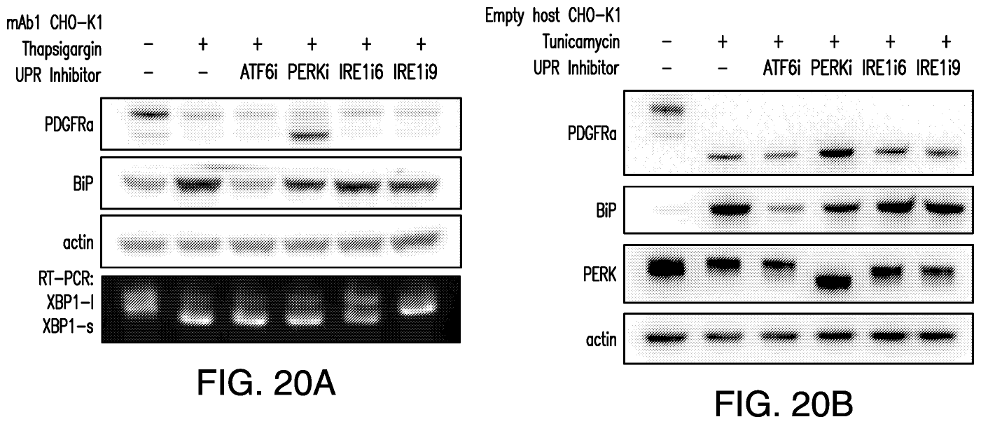

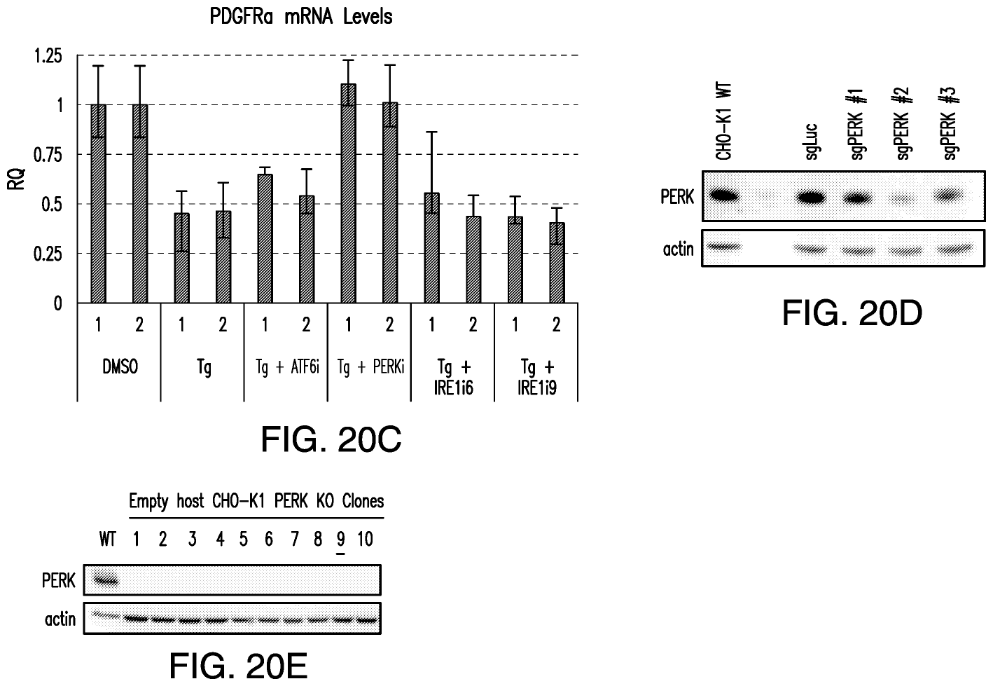

To further dissect which branch of the UPR is responsible for regulating PDGFRa levels, strong UPR inducers (tunicamycin and thapsigargin) were used to induce UPR in CHO-K1 cells treated with specific inhibitors against ATF6, PERK, or IRE1a branches of the UPR pathway (FIGS. 19E, 19F and FIGS. 20A, 20B and 20C). These data revealed that inhibition of the PERK branch of the UPR pathway rescued downregulation of PDGFRa at the protein (FIG. 19E) and mRNA levels (FIG. 19F), without affecting activation of other branches of the UPR as evident by increased levels of intracellular BiP protein and XBP-1 RNA processing in both Tunicamycin (FIG. 19E) and Thapsigargin (FIG. 20A) treated cultures. Downregulation of PDGFRa via activation of the PERK branch of the UPR pathway occurred both in antibody-expressing (FIG. 19E and FIG. 20A) and empty host cells (FIG. 20B). The slightly lower molecular weight of PERK protein observed in the presence of PERK inhibitor is likely due to covalent modifications of PERK by this specific inhibitor (FIG. 20B).

Furthermore, sgRNAs were designed and tested to knockout the PERK gene in CHO-K1 cells using CRISPR-Cas9 (FIG. 20D) and a transfected a pool with the best knockout phenotype (sgPERK #2) was single cell cloned to isolate empty CHO-Kl host cell lines that did not express PERK protein (FIG. 20E). These empty CHO-Kl PERK KO host cell lines were evaluated for growth, transfection rate, recovery in selection media, and culture performance to identify a PERK KO host cell line with comparable overall culture performance to the wild-type (WT) CHO-Kl host. Empty WT and an empty PERK KO host cell line (Clone 9, FIG. 20E) were then treated with or without tunicamycin and PERK inhibitor to evaluate PDGFRa regulation upon UPR induction (FIG. 19G). Relative to the WT control, PDGFRa expression was not downregulated upon UPR induction and addition of PERK inhibitor did not further stabilize PDGFRa expression in the PERK KO host (FIG. 19G).

This investigation into one of our antibody-expressing cell lines, mAb1 CHO DG44, revealed that transcriptional downregulation, and hence lower expression of PDGFRa protein, was the likely cause of poor growth outcomes during production when the cells were sourced from seed train cultures exposed to low pH (FIGS. 19A and 19B). It was previously shown that this poor growth outcome correlated with increased intracellular BiP levels, which is indicative of UPR activation (Tung, et al., 2018). When the UPR was chemically induced, PDGFRa protein levels also decreased due to transcriptional downregulation, a phenomenon that could be reversed by chemical inhibition of the PERK branch of the UPR pathway, suggesting that PERK activation mediates PDGFRa downregulation (FIGS. 19C, 19D, 19E, 19F, and FIGS. 20A, 20B and 20C). This was further confirmed when chemical induction of UPR in a PERK KO cell line did not result in downregulation of PDGFRa expression (FIG. 19G).

### Example 5: PDGFRa Signaling Pathway is Critical for CHO Culture Growth and Functions in Parallel to Insulin Signaling Pathway

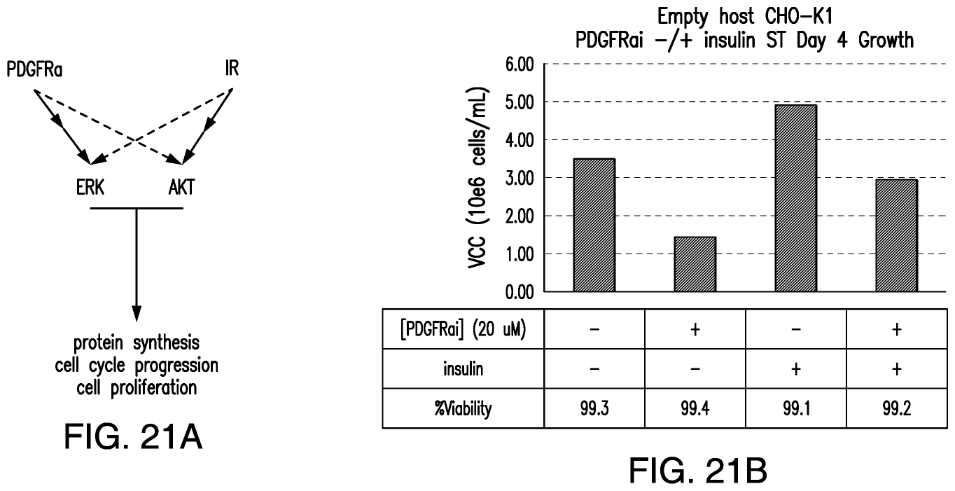

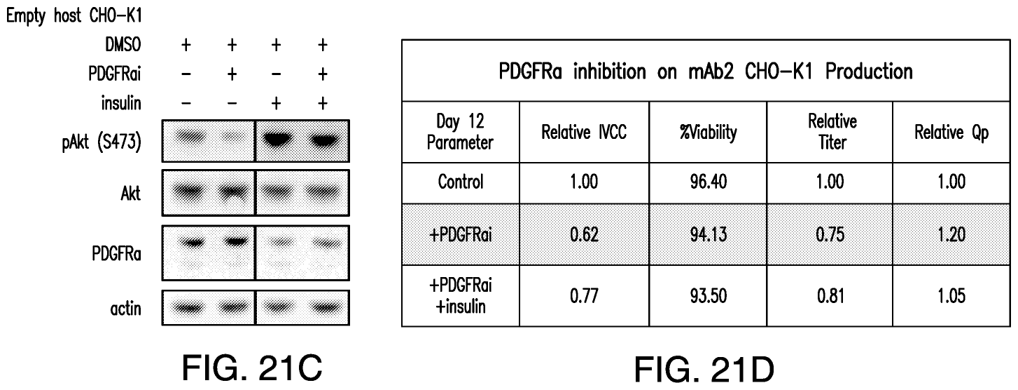

It was previously shown that UPR-induced poor growth profiles correlated with a decrease in PDGFRa levels (FIGS. 19A and 19B) (Tung, et al., 2018). PDGFRa and insulin signaling pathways have overlapping downstream targets (FIG. 21A), however insulin signaling negatively regulates PDGFRa signaling (Cirri, et al., 2005). To test the importance of PDGFRa signaling pathway in CHO cell growth, empty host CHO-Kl cells were cultured in the presence of different concentrations of PDGFRa inhibitor, which reduced cell growth by approximately 50% at 20 μM concentrations (FIG. 21B) due to attenuation of the Akt signaling pathway (FIG. 21C). Addition of insulin to the CHO cultures treated with the PDGFRa inhibitor partially rescued cellular growth (FIG. 21B) and increased Akt phosphorylation and hence its activation compared to the untreated cultures (FIG. 21C). These findings confirmed that PDGFRa and insulin signaling pathways indeed have overlapping downstream targets in CHO cells and that Akt signaling pathway remains intact in the presence of PDGFRa inhibitor (FIGS. 21B and 21C). PDGFRa signaling is also important for CHO production culture growth as its inhibition on Day 3 of a fed-batch production significantly decreased cell growth, without affecting cell viability, in an antibody-expressing (mAb2) CHO cell line (FIG. 21D). Similar to the seed train cultures (FIG. 21B), addition of insulin on Day 3 of the production culture partially rescued the observed cell growth inhibition (FIG. 21D).

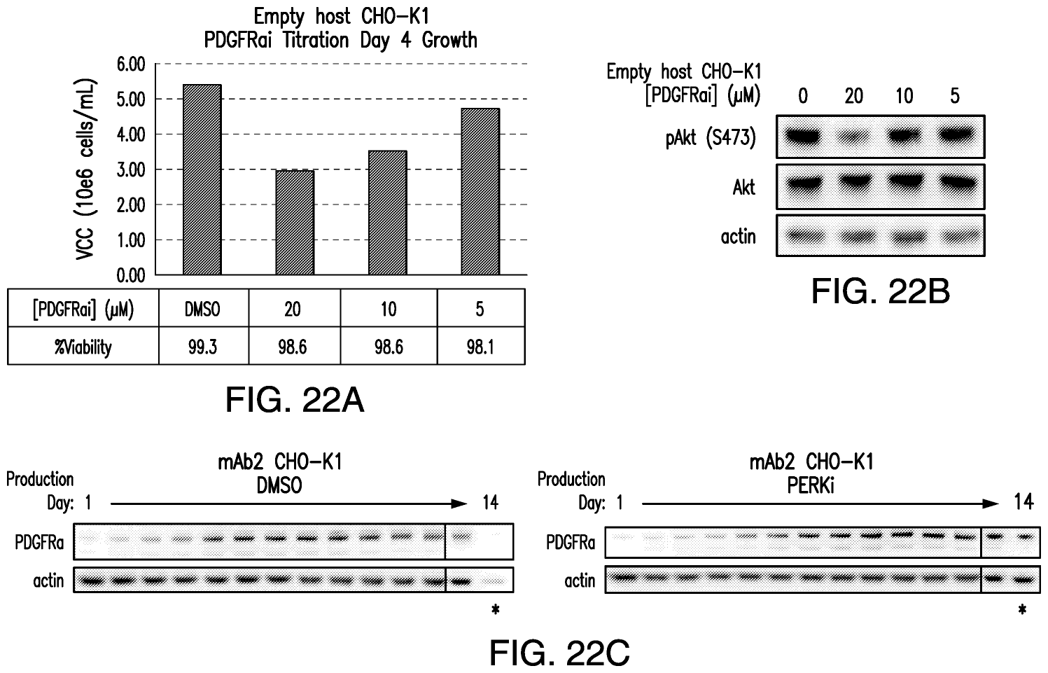

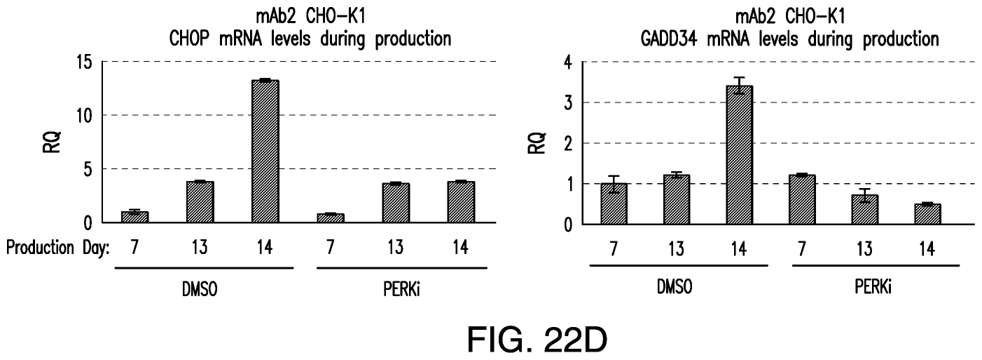

The PDGFRa signaling pathway proved to be critical for cell growth in our CHO cells, which are cultured in chemically defined media without any growth factors (FIGS. 22A and 22B), suggesting that either our CHO cells secrete a PDGFRa ligand, or PDGFRa signaling pathway is intrinsically active in these cells. Addition of insulin to the culture media partially rescued cell growth when PDGFRa signaling was inhibited, implicating that PDGFRa inhibitor is specific and does not affect downstream signaling (FIGS. 21B and 21C), as both PDGFRa and insulin receptor (IR) have partially overlapping signaling pathways (FIG. 21A).

Downregulation of PDGFRa by the PERK branch of the UPR was also observed in production culture where a decline in PDGFRa levels towards the end of the culture period, coincided with the higher levels of PERK activity as evident by a surge in mRNA levels of its downstream target proteins (FIGS. 22C and 22D). Chemical inhibition of PERK prevented transcriptional increase of its downstream targets and also stabilized PDGFRa levels during production (FIGS. 22C and 22D).

### Example 6: Activation of the PERK Branch of the UPR Attenuates PDGFRa Signaling, Reduces Specific Productivity and Promotes Culture Viability During Production

The correlation between PERK activation and downregulation of PDGFRa expression was monitored in production culture, using a mAb2-expressing CHO-Kl cell line, in the absence (control) or presence of PERK inhibitor (added on Day 3 of production). The observed downregulation of PDGFRa on days 13 and 14 of the production culture (FIG. 22C, left panel) correlated with an increase in mRNA levels of CHOP and GADD34 genes, which are downstream targets of PERK (Marciniak, et al., 2004), indicating activation of PERK signaling pathway (FIG. 22D). Addition of PERK inhibitor blocked PERK signaling (no increase in CHOP and GADD34 mRNA levels) and prevented downregulation of PDGFRa expression (FIG. 22C right panel, and 22D). Since use of PERK inhibitor is cost prohibitive and its potential off-target activity on cultured cells cannot be fully ruled out, it was decided to generate PERK KO mAb2-expressing CHO-Kl cell lines to directly investigate the role of this signaling pathway in PDGFRa downregulation and production culture performance.

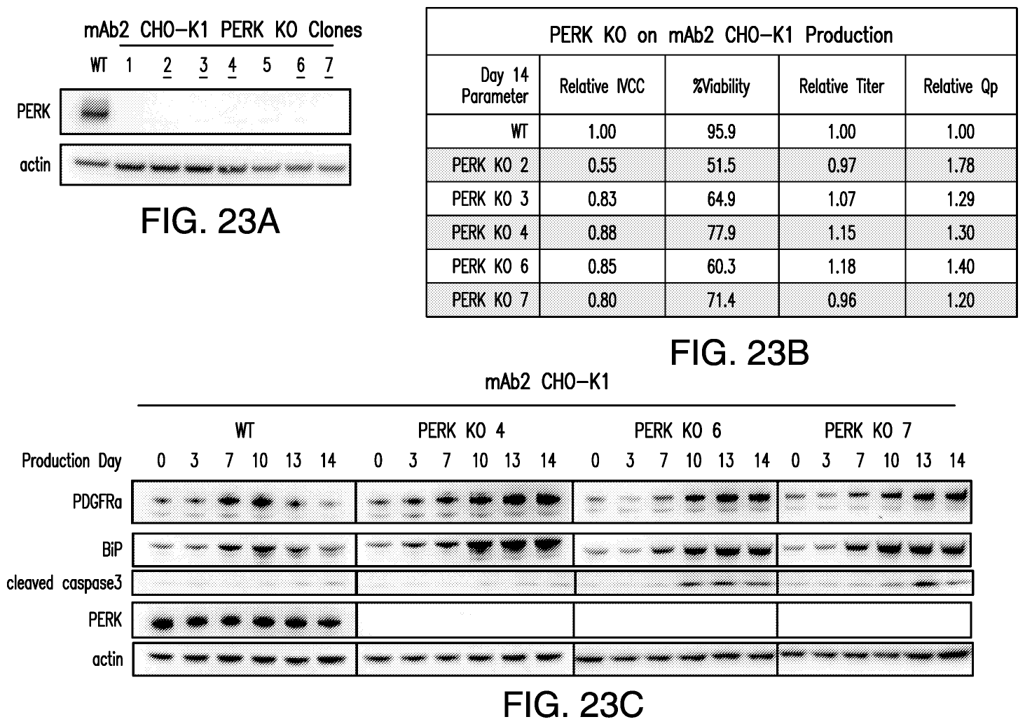

CRISPR-Cas9 technology was used to knockout the PERK gene in the mAb2-expressing CHO-Kl cell line and after single cell cloning, derived PERK KO cell lines with comparable growth profiles to the parental cell line (FIG. 23A, underlined clones) were evaluated in production culture (FIGS. 23B and 23C). PERK KO cell lines overall showed decreased growth and viability, compared to the parental cell line (FIG. 23B), however, all the PERK KO cell lines had higher specific productivities, and for most part titers, compared to the WT parental cell line (FIG. 23B). Western blot analysis of these cell lines during production confirmed that PDGFRa levels were stabilized in the PERK KO cell lines compared to the WT parental cell line, which displayed reduced levels of PDGFRa expression, towards the end of the production (FIG. 23C). Higher levels of intracellular BiP protein in the PERK KO cell lines indicated increased UPR activation (FIG. 23C), while the observed decrease in cellular growth and viability (FIG. 23B) correlated with increased caspase-3 cleavage, implying activation of the apoptotic pathway towards the end of the production culture (FIG. 23C). Antibodies expressed by WT or PERK KO cell lines had comparable product quality.

These findings confirmed that activation of the PERK branch of the UPR downregulates expression of PDGFRa in both seed train and production cultures. Interestingly, PERK KO cultures displayed lower overall viability and growth, but higher titer and specific productivity during production (FIG. 23B). Increased intracellular BiP levels and higher levels of caspase-3 cleavage in these cultures indicated activation of the UPR and apoptotic pathways, respectively, and correlated with lower culture viabilities (FIG. 23C). Likely, higher levels of specific productivity during production triggers cellular apoptosis and early PERK activation attenuates cellular apoptosis by simply reducing the specific productivity of these cells.

### Example 7: Knocking Out PERK in a Bax/Bak Double Knockout CHO Cell Line Drastically Increased Specific Productivity and Titer by Enhancing-Transgene Transcription and Attenuating Apoptotic Cell Death

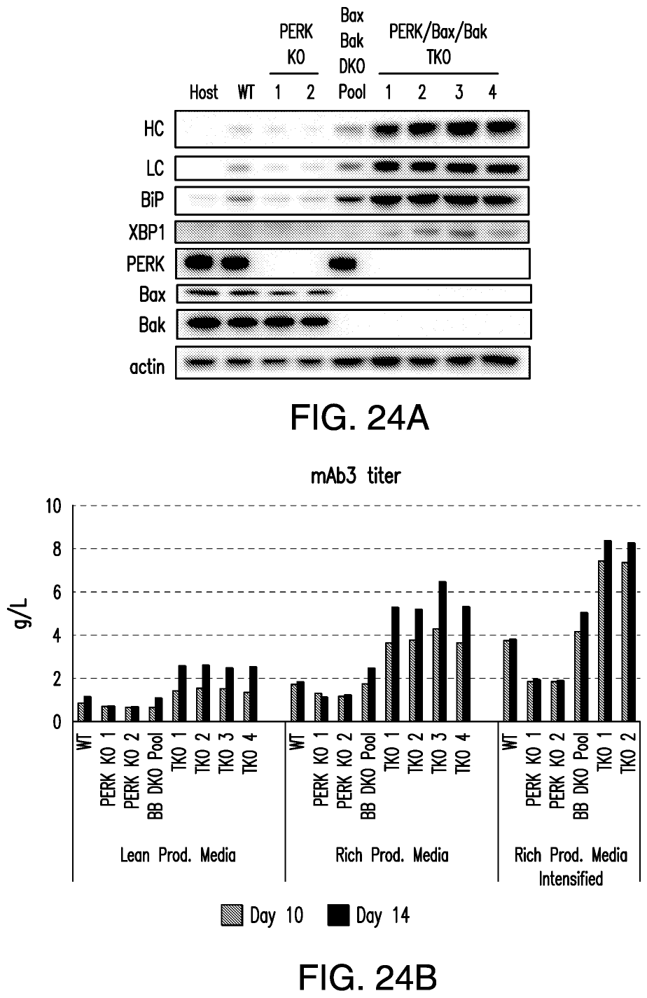

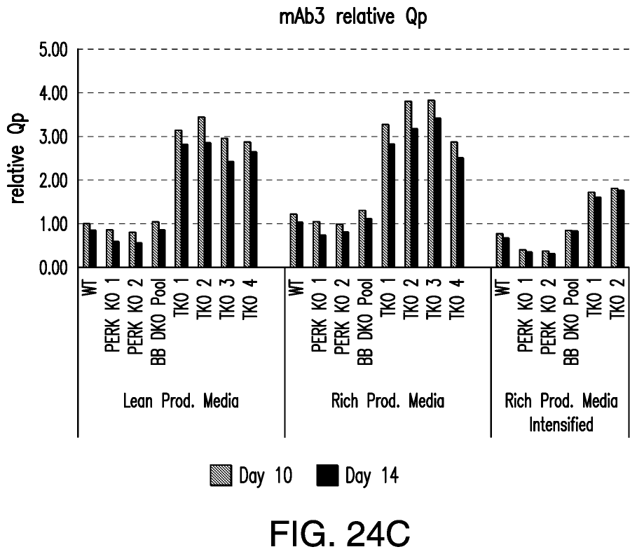

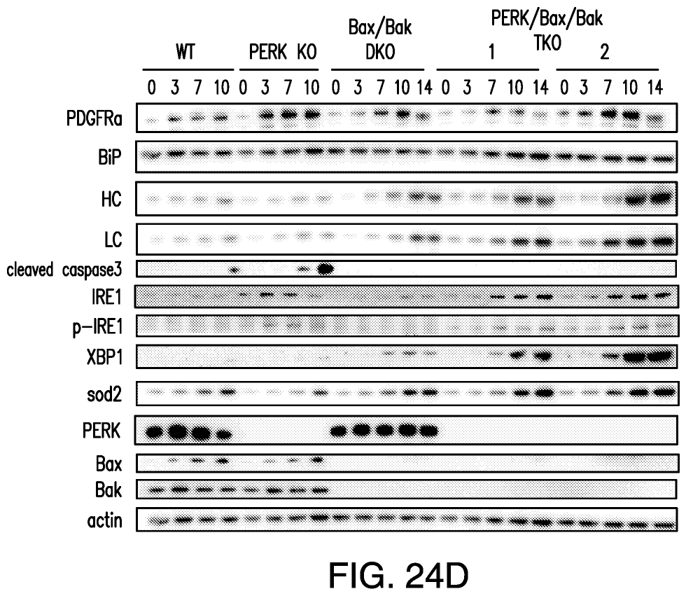

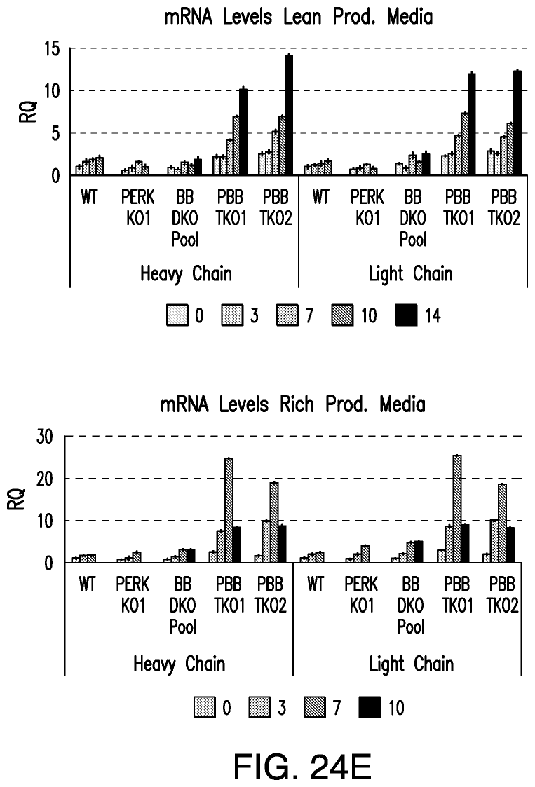

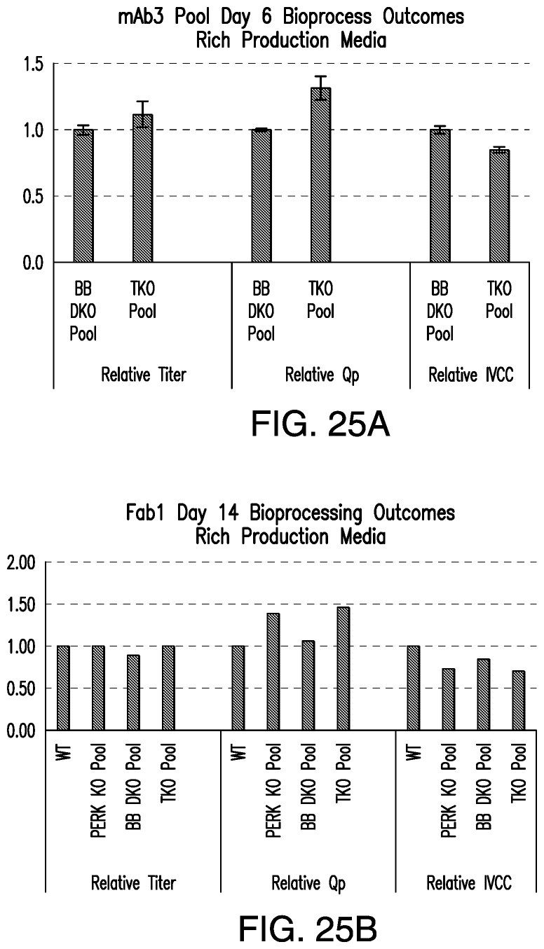

Since the PERK KO clones showed higher levels apoptosis during production (FIG. 23C), the PERK gene was knocked out in a mAb3-expressing WT cell line or a mAb3-expressing pool of Bax/Bak double knockout (DKO) cell line (FIG. 24A). Bax/Bak are proteins that act at the mitochondria to initiate apoptotic cell death (Taylor, Cullen, & Martin, 2008) and the deletion of these genes make cell lines more resistant to apoptosis and potentially improve viability and productivity during long production processes compared to WT CHO cell lines (Misaghi, Qu, Snowden, Chang, & Snedcor, 2013). After single cell sorting the PERK/Bax/Bak triple knockout (TKO) clones (FIG. 24A) were compared to controls (WT, PERK KO, and Bax/Bak DKO pool) across three different production platforms: 1) using lean production media, 2) using rich production media, and 3) using rich production media in an intensified process. The TKO clones exhibited better bioprocess outcomes showing higher titer and relative specific productivity as compared to controls (FIGS. 24B and 24C, and Table 7), while maintaining comparable product quality attributes (Table 8) across all production platforms. Similar production platforms testing PERK/Bax/Bak TKO pools and clones clearly reveal that deletion of the PERK gene results in higher specific productivity CHO cells expressing antibody (mAb3) or Fab (Fab1) (FIGS. 25A, 25B and Table 9). These data suggest that the observed increase in specific productivity of Bax/Bak/PERK TKO CHO cells is not clone or product specific but is rather a general phenomenon.

TABLE 7
Bioprocess outcomes for mAb3-expressing
CHO-K1 TKO cells across different bioprocesses.

| | | Parameter | | | | | | | |
|---|---|---|---|---|---|---|---|---|---|
| | | Titer (g/L) | | Relative Qp | | Relative IVCC | | % Viability | |
| | | Day | | | | | | | |
| | | 10 | 14 | 10 | 14 | 10 | 14 | 10 | 14 |
| Lean Prod. | WT | 0.86 | 1.16 | 1.00 | 0.84 | 1.00 | 1.60 | 98.0 | 5.4 |
| Media | PERK KO 1 | 0.71 | 0.72 | 0.85 | 0.59 | 0.97 | 1.44 | 88.5 | 1.1 |
| | PERK KO 2 | 0.66 | 0.69 | 0.80 | 0.55 | 0.96 | 1.46 | 97.8 | 1.1 |
| | BB DKO | 0.67 | 1.10 | 1.04 | 0.86 | 0.75 | 1.49 | 99.0 | 97.0 |
| | TKO 1 | 1.42 | 2.59 | 3.14 | 2.82 | 0.53 | 1.07 | 96.5 | 94.1 |
| | TKO 2 | 1.55 | 2.62 | 3.45 | 2.86 | 0.52 | 1.07 | 97.4 | 94.4 |
| | TKO 3 | 1.52 | 2.50 | 2.96 | 2.43 | 0.60 | 1.20 | 97.3 | 93.9 |
| | TKO 4 | 1.36 | 2.54 | 2.87 | 2.64 | 0.55 | 1.12 | 96.1 | 94.2 |
| Rich Prod. | WT | 1.72 | 1.85 | 1.00 | 0.85 | 1.00 | 1.27 | 97.7 | 0.0 |
| Media | PERK KO 1 | 1.31 | 1.15 | 0.86 | 0.60 | 0.89 | 1.12 | 94.2 | 0.0 |
| | PERK KO 2 | 1.18 | 1.23 | 0.80 | 0.66 | 0.85 | 1.09 | 96.6 | 0.0 |
| | BB DKO | 1.74 | 2.48 | 1.06 | 0.91 | 0.95 | 1.58 | 98.7 | 96.1 |
| | TKO 1 | 3.66 | 5.30 | 2.68 | 2.32 | 0.79 | 1.33 | 92.9 | 91.7 |
| | TKO 2 | 3.78 | 5.20 | 3.12 | 2.60 | 0.70 | 1.16 | 92.7 | 89.4 |
| | TKO 3 | 4.30 | 6.48 | 3.14 | 2.81 | 0.79 | 1.34 | 95.4 | 90.6 |
| | TKO 4 | 3.66 | 5.32 | 2.36 | 2.06 | 0.90 | 1.50 | 95.3 | 91.3 |
| Rich Prod. | WT | 3.76 | 3.82 | 1.00 | 0.87 | 1.00 | 1.17 | 94.0 | 63.7 |
| Media | PERK KO 1 | 1.86 | 1.94 | 0.53 | 0.46 | 0.94 | 1.13 | 95.0 | 77.9 |
| Intensified | PERK KO 2 | 1.84 | 1.92 | 0.49 | 0.41 | 1.01 | 1.23 | 97.0 | 85.3 |
| | BB DKO | 4.16 | 5.05 | 1.09 | 1.07 | 1.01 | 1.25 | 98.6 | 96.0 |
| | TKO 1 | 7.44 | 8.38 | 2.24 | 2.10 | 0.88 | 1.06 | 97.5 | 89.9 |
| | TKO 2 | 7.36 | 8.28 | 2.35 | 2.29 | 0.83 | 0.96 | 92.0 | 55.8 |

TABLE 8
Product quality of mAb3-expressing CHO-K1 TKO cells across different bioprocesses.

| | | Aggregate | % Charge Variants | | | | | Aggregate | % Charge Variants | | |
|---|---|---|---|---|---|---|---|---|---|---|---|
| | Parameter | % HMWS | Acidic | Main | Basic | | Parameter | % HMWS | Acidic | Main | Basic |
| Lean | WT | 4.39 | 32.12 | 61.02 | 6.86 | Rich | WT | 3.04 | 39.35 | 52.39 | 8.26 |
| Prod. | PERK KO 1 | 3.83 | 36.47 | 57.19 | 0.35 | Prod. | PERK KO 1 | 2.70 | 38.70 | 54.92 | 6.39 |
| Media | PERK KO 2 | 3.31 | 34.80 | 57.76 | 7.43 | Media | PERK KO 2 | 2.89 | 38.54 | 56.11 | 5.35 |
| | BB DKO Pool | 19.69 | 33.24 | 60.22 | 6.53 | | BB DKO Pool | 14.19 | 38.71 | 55.09 | 6.21 |
| | TKO 1 | 10.16 | 32.12 | 62.46 | 5.41 | | TKO 1 | 12.03 | 41.65 | 53.82 | 4.53 |
| | TKO 2 | 13.71 | 33.25 | 61.64 | 5.11 | | TKO 2 | 13.16 | 42.73 | 52.52 | 4.76 |
| | TKO 3 | 16.24 | 32.87 | 61.57 | 5.57 | | TKO 3 | 15.70 | 43.82 | 51.83 | 4.35 |
| | TKO 4 | 11.54 | 30.98 | 63.54 | 5.48 | | TKO 4 | 11.90 | 40.77 | 53.48 | 5.75 |

TABLE 9
Bioprocess outcomes for single cell clones of Bax/Bak DKO and PERK/Bax/Bak TKO.

| | mAb3 CHO-K1 Production - TKO Single Cell Clones | | | | | mAb3 CHO-K1 Production - DKO Single Cell Clones | | | |
|---|---|---|---|---|---|---|---|---|---|
| | Day 14 | | | | | Day 14 | | | |
| | Parameter | Rel. Titer | Rel. Qp | Rel. IVCC | | Parameter | Rel. Titer | Rel. Qp | Rel. IVCC |
| Rich Prod. | WT | 1.00 | 1.00 | 1.00 | Rich Prod. | WT Average | 1.00 | 1.00 | 1.00 |
| Media | DKO Pool | 1.34 | 1.08 | 1.24 | Media | DKO Average | 1.21 | 1.03 | 1.15 |
| | TKO Average | 3.02 | 2.89 | 1.05 | | WT1 | 0.65 | 0.54 | 1.16 |
| | TKO 1 | 2.87 | 2.74 | 1.05 | | WT2 | 1.23 | 1.34 | 0.88 |
| | TKO 2 | 2.82 | 3.08 | 0.92 | | WT3 | 1.12 | 1.12 | 0.96 |
| | TKO 3 | 3.51 | 3.32 | 1.06 | | BB DKO1 | 1.34 | 0.82 | 1.58 |
| | TKO 4 | 2.88 | 2.43 | 1.19 | | BB DKO2 | 1.04 | 0.94 | 1.06 |
| | | | | | | BB DKO3 | 1.16 | 1.21 | 0.92 |
| | | | | | | BB DKO4 | 1.01 | 0.99 | 0.97 |
| | | | | | | BB DKO5 | 1.52 | 1.20 | 1.22 |

Western blot analysis revealed that PERK/Bax/Bak TKO clones had higher intracellular levels of antibody heavy chain and light chain in the seed train (FIG. 24A) and production media (FIG. 24D) relative to the parental line. Additionally, the TKO clones displayed more stabilized PDGFRa expression and no caspase-3 cleavage, indicative of inhibition of apoptosis pathway, in production compared to the parental line (FIG. 24D). Interestingly, PERK/Bax/Bak TKO clones had higher levels of IRE1a, phosphor-IRE1a and significantly higher levels of spliced XBP-1 transcription factor, indicating that these cells are experiencing increased protein translation and proteostatic stress in production (FIG. 24D). TKO clones also displayed higher levels of Sod2 protein, implying activation of reactive oxygen species (ROS) pathway (FIG. 24D). These findings suggest that activation of PERK branch of the UPR during production cumulatively reduces proteostatic stress by reducing protein translation and attenuation of IRE1a and ROS pathways, thereby mitigating apoptotic cell death in production culture. Further investigation of these production processes linked the increase in specific productivity to increased mRNA levels of the heavy chain and light chain transcripts in the TKO clones compared to the parental cell line (FIG. 24E). This suggests that the PERK branch of the UPR either directly or indirectly, through attenuation of IRE1a or PDGFRa signaling, reduces transgene transcription from the CMV promoter during production.

As mentioned above, to prevent apoptosis due to increased specific productivity in the PERK KO cell lines, PERK was knocked out in an antibody expressing Bax/Bak double knockout cell line (FIG. 24A). Excitingly, a synergistic effect in the TKO clones during production resulted in higher overall titers (up to 8 g/L) and relative specific productivities compared to the parental line while having comparable IVCC and viability (FIGS. 24B, 24C and Table 7). This synergistic effect may be explained by increased IRE1a signaling caused by the deletion of PERK, which has been shown to attenuate IRE1a branch of UPR (Chang, et al., 2018), and in conjunction with a prolonged IRE1a activity due to attenuation of apoptosis signaling pathway because of deletion of Bax and Bak genes. It was observed increased and prolonged IRE1a signaling in our TKO clones during production indicated by more phosphorylated IRE1 a and increased presence of its downstream target, spliced XBP-1 (FIG. 24D). XBP-1 has been shown to improve bioprocess outcomes transiently (Rajendra, Hougland, Schmitt, & Barnard, 2015) and the observed increase in antibody transcription levels (FIG. 24E) suggests that either activation of PERK branch of the UPR attenuates transgene(s) transcription from the CMV promoter, or PDGFRa and/or IRE1a signaling play a role in enhancing transcription from CMV promoter either directly or through their downstream targets. The exact mechanisms and interplay between these signaling pathways, however, remain to be determined.

The findings presented in the present disclosure suggest that chronic activation of UPR in antibody-expressing CHO cells can trigger poor growth, primarily through the PERK pathway which downregulates PDGFRa levels. The UPR in these cells is largely caused by proteostatic stress in the ER, which can be triggered by many different factors ranging from cell culture parameters to the amino acid sequence and composition of expressed proteins. It is suspected that this is a way to promote adaptive growth when protein production, and hence burden on the ER, increases. Slowing down cellular proliferation and metabolism by regulating PDGFRa levels can allow more time for ER expansion, which is also regulated by the PERK pathway. Knocking out the PERK pathway might allow the cells to grow, but can also result in apoptosis as cells are unable to accommodate the additional stress imposed by high rates of specific productivity and protein synthesis. To bypass this, knocking out the PERK pathway in conjunction with the deletion of components of the apoptotic pathway (Bax/Bak genes) achieves both high rates of specific productivity and increased cell viability. Hence, it is proposed in the present disclosure that knocking out PERK in a mammalian protein expression host cell line with attenuated apoptosis pathway(s) may significantly increase specific productivity and hence culture titers.

### Example 8: Penta (5×) KO CHO Cells

### General Techniques

### l) Recombinant DNA Techniques

Standard methods were used to manipulate DNA as described in Sambrook et al., Molecular Cloning: A Laboratory Manual, Second Edition, Cold Spring Harbor Laboratory Press, Cold Spring Harbor, N.Y, (1989). The molecular biological reagents were used according to the manufacturer's instructions.

### 2) DNA Sequence Determination

DNA sequencing was performed at SequiServe GmbH (Vaterstetten, Germany) or Eurofins Genomics GmbH (Ebersberg, Germany) or Microsynth AG (Balgach, Switzerland).

### 3) DNA and Protein Sequence Analysis and Sequence Data Management

The EMBOSS (European Molecular Biology Open Software Suite) software package and Geneious prime 2021 (Auckland, New Zealand) were used for sequence creation, mapping, analysis, annotation and illustration.

### 4) Gene and Oligonucleotide Synthesis

Desired gene segments were prepared by chemical synthesis at Geneart GmbH (Regensburg, Germany) or Twist Bioscience (San Francisco, USA). The synthesized gene fragments were cloned into an E. coli plasmid for propagation/amplification. The DNA sequences of subcloned gene fragments were verified by DNA sequencing. Alternatively, short synthetic DNA fragments were assembled by annealing chemically synthesized oligonucleotides or via PCR. The respective oligonucleotides were prepared by metabion GmbH (Planegg-Martinsried, Germany).

### 5) Reagents

All commercial chemicals, antibodies and kits were used as provided according to the manufacturer's protocol if not stated otherwise.

### 6) Cultivation of TI Host Cell Line

TI CHO host cells were cultivated at 37° C. in a humidified incubator with 85% humidity and 5% CO₂. They were cultivated in a proprietary DMEM/F12-based medium containing 300 pg/ml Hygromycin B and 4 pg/ml of a second selection marker. The cells were splitted every 3 or 4 days at a concentration of 0.3×10E6 cells/ml in a total volume of 30 ml. For the cultivation 125 ml non-baffle Erlenmeyer shake flasks were used. Cells were shaken at 150 rpm with a shaking amplitude of 5 cm. The cell count was determined with Cedex HiRes Cell Counter (Roche). Cells were kept in culture until they reached an age of 60 days.

### 7) Cloning

### a) General:

Cloning with R-sites depends on DNA sequences next to the gene of interest (GOI) that are equal to sequences lying in following fragments. Like that, assembly of fragments is possible by overlap of the equal sequences and subsequent sealing of nicks in the assembled DNA by a DNA ligase. Therefore, a cloning of the single genes in particular preliminary vectors containing the right R-sites is necessary. After successful cloning of these preliminary vectors the gene of interest flanked by the R-sites is cut out via restriction digest by enzymes cutting directly next to the R-sites. The last step is the assembly of all DNA fragments in one-step. In more detail, a 5′-exonuclease removes the 5′-end of the overlapping regions (R-sites). After that, annealing of the R-sites can take place and a DNA polymerase extends the 3′-end to fill the gaps in the sequence. Finally, the DNA ligase seals the nicks in between the nucleotides. Addition of an assembly master mix containing different enzymes like exonucleases, DNA polymerases and ligases, and subsequent incubation of the reaction mix at 50° C. leads to an assembly of the single fragments to one plasmid. After that, competent E. coli cells are transformed with the plasmid.

For some vectors, a cloning strategy via restriction enzymes was used. By selection of suitable restriction enzymes, the wanted gene of interest can be cut out and afterwards inserted into a different vector by ligation. Therefore, enzymes cutting in a multiple cloning site (MCS) are preferably used and chosen in a smart manner, so that a ligation of the fragments in the correct array can be conducted. If vector and fragment are previously cut with the same restriction enzyme, the sticky ends of fragment and vector fit perfectly together and can be ligated by a DNA ligase, subsequently. After ligation, competent E. coli cells are transformed with the newly generated plasmid.

### b) Cloning Via Restriction Digestion:

For the digest of plasmids with restriction enzymes, the following components were pipetted together on ice:

TABLE 10
Restriction Digestion Reaction Mix

| component | ng (set point) | μl |
|---|---|---|
| purified DNA | tbd | tbd |
| CutSmart Buffer (10×) | | 5 |
| Restriction Enzyme | | 1 |
| PCR-grade Water | | ad 50 |
| Total | | 50 |

If more enzymes were used in one digestion, 1 μl of each enzyme was used and the volume adjusted by addition of more or less PCR-grade water. All enzymes were selected on the preconditions that they are qualified for the use with CutSmart buffer from new England Biolabs (100% activity) and have the same incubation temperature (all 37° C.).

Incubation was performed using thermomixers or thermal cyclers, allowing incubating the samples at a constant temperature (37° C.). During incubation the samples were not agitated. Incubation time was set at 60 min. Afterwards the samples were directly mixed with loading dye and loaded onto an agarose electrophoresis gel or stored at 4° C./on ice for further use.

A 1% agarose gel was prepared for gel electrophoresis. Therefor 1.5 g of multi-purpose agarose were weighed into a 125 Erlenmeyer shake flask and filled up with 150 ml TAE-buffer. The mixture was heated up in a microwave oven until the agarose was completely dissolved. 0.5 μg/ml ethidium bromide were added into the agarose solution. Thereafter the gel was cast in a mold. After the agarose was set, the mold was placed into the electrophoresis chamber and the chamber filled with TAE-buffer. Afterwards the samples were loaded. In the first pocket (from the left), an appropriate DNA molecular weight marker was loaded, followed by the samples. The gel was run for around 60 minutes at <130 V. After electrophoresis, the gel was removed from the chamber and analyzed in an UV-Imager.

The target bands were cut and transferred to 1.5 ml Eppendorf tubes. For purification of the gel, the QIAquick Gel Extraction Kit from Qiagen was used according to the manufacturer's instructions. The DNA fragments were stored at −20° C. for further use.

The fragments for the ligation were pipetted together in a molar ratio of 1:2, 1:3 or 1:5 vector to insert, depending on the length of the inserts and the vector-fragments and their correlation to each other. If the fragment, that should be inserted into the vector was short, a 1:5-ratio was used. If the insert was longer, a smaller amount of it was used in correlation to the vector. An amount of 50 ng of vector were used in each ligation and the particular amount of insert calculated with NEBioCalculator. For ligation, the T4 DNA ligation kit from NEB was used. An example for the ligation mixture is depicted in the following Table.

TABLE 11
Ligation Reaction Mix

| component | ng (set point) | conc. [ng/μl] | μl |
|---|---|---|---|
| T4 DNA Ligase Buffer | | | 2 |
| (10×) | 50 | 50 | 1 |
| Vector DNA (4000 bp) | 125 | 20 | 6.25 |
| Insert DNA (2000 bp) | | | 9.75 |
| Nuclease-free Water | | | 1 |
| T4 Ligase | | | |
| Total | | | 20 |

All components were pipetted together on ice, starting with the mixing of DNA and water, addition of buffer and finally addition of the enzyme. The reaction was gently mixed by pipetting up and down, briefly microfuged and then incubated at room temperature for 10 minutes. After incubation, the T4 ligase was heat inactivated at 65° C. for 10 minutes. The sample was chilled on ice. In a final step, 10-beta competent E. coli cells were transformed with 2 μl of the ligated plasmid (see below).

### c) Cloning Via R-Site Assembly:

For assembly, all DNA fragments with the R-sites at each end were pipetted together on ice. An equimolar ratio (0.05 ng) of all fragments was used, as recommended by the manufacturer, when more than 4 fragments are being assembled. One half of the reaction mix was embodied by NEBuilder HiFi DNA Assembly Master Mix. The total reaction volume was 40 μl and was reached by a fill-up with PCR-clean water. In the following Table, an exemplary pipetting scheme is depicted.

TABLE 12
Assembly Reaction Mix

| | | pmol | ng | conc. | |
|---|---|---|---|---|---|
| component | bp | (set point) | (set point) | [ng/μl] | μl |
| Insert 1 | 2800 | 0.05 | 88.9 | 21 | 4.23 |
| Insert 2 | 2900 | 0.05 | 90.5 | 35 | 2.59 |
| Insert 3 | 4200 | 0.05 | 131.6 | 35.5 | 3.71 |
| Insert 4 | 3600 | 0.05 | 110.7 | 23 | 4.81 |
| Vector | 4100 | 0.05 | 127.5 | 57.7 | 2.21 |
| NEBuilder HiFi | | | | | 20 |
| DNA Assembly | | | | | |
| Master Mix | | | | | |
| PCR-clean Water | | | | | 2.45 |
| Total | | | | | 40 |

After set up of the reaction mixture, the tube was incubated in a thermocycler at constantly 50° C. for 60 minutes. After successful assembly, 10-beta competent E. coli bacteria were transformed with 2 μl of the assembled plasmid DNA (see below).

d) Transformation 10-Beta Competent E. coli Cells:

For transformation, the 10-beta competent E. coli cells were thawed on ice. After that, 2 μl of plasmid DNA were pipetted directly into the cell suspension. The tube was flicked and put on ice for 30 minutes. Thereafter, the cells were placed into the 42° C.-warm thermal block and heat-shocked for exactly 30 seconds. Directly afterwards, the cells were chilled on ice for 2 minutes. 950 μl of NEB 10-beta outgrowth medium were added to the cell suspension. The cells were incubated under shaking at 37° C. for one hour. Then, 50-100 μl were pipetted onto a pre-warmed (37° C.) LB-Amp agar plate and spread with a disposable spatula. The plate was incubated overnight at 37° C. Only bacteria, which have successfully incorporated the plasmid, carrying the resistance gene against ampicillin, can grow on these plates. Single colonies were picked the next day and cultured in LB-Amp medium for subsequent plasmid preparation.

### e) Bacterial Culture:

Cultivation of E. coli was done in LB-medium, short for Luria Bertani, which was spiked with 1 ml/L 100 mg/ml ampicillin resulting in an ampicillin concentration of 0.1 mg/ml. For the different plasmid preparation quantities, the following amounts were inoculated with a single bacterial colony.

TABLE 13
E. coli cultivation volumes

| | volume LB-Amp | incubation time |
|---|---|---|
| quantity plasmid preparation | medium [ml] | [h] |
| Mini-Prep 96-well (EpMotion) | 1.5 | 23 |
| Mini-Prep 15 ml-tube | 3.6 | 23 |
| Maxi-Prep | 200 | 16 |

For Mini-Prep, a 96-well 2 ml deep-well plate was filled with 1.5 ml LB-Amp medium per well. The colonies were picked and the toothpick was tuck in the medium. When all colonies were picked, the plate closed with a sticky air porous membrane. The plate was incubated in a 37° C. incubator at a shaking rate of 200 rpm for 23 hours.

For Mini-Preps a 15 ml-tube (with a ventilated lid) was filled with 3.6 ml LB-Amp medium and equally inoculated with a bacterial colony. The toothpick was not removed but left in the tube during incubation. Like the 96-well plate, the tubes were incubated at 37° C., 200 rpm for 23 hours.

For Maxi-Prep 200 ml of LB-Amp medium were filled into an autoclaved glass 1 L Erlenmeyer flask and inoculated with 1 ml of bacterial day-culture, which was roundabout 5 hours old. The Erlenmeyer flask was closed with a paper plug and incubated at 37° C., 200 rpm for 16 hours.

### f) Plasmid Preparation:

For Mini-Prep, 50 μl of bacterial suspension were transferred into a 1 ml deep-well plate. After that, the bacterial cells were centrifuged down in the plate at 3000 rpm, 4° C. for 5 mn. The supernatant was removed and the plate with the bacteria pellets placed into an EpMotion. After approx. 90 minutes, the run was done and the eluted plasmid-DNA could be removed from the EpMotion for further use.

For Mini-Prep, the 15 ml tubes were taken out of the incubator and the 3.6 ml bacterial culture splitted into two 2 ml Eppendorf tubes. The tubes were centrifuged at 6,800×g in a tabletop microcentrifuge for 3 minutes at room temperature. After that, Mini-Prep was performed with the Qiagen QIAprep Spin Miniprep Kit according to the manufacturer's instructions. The plasmid DNA concentration was measured with Nanodrop.

Maxi-Prep was performed using the Macherey-Nagel NucleoBond® Xtra Maxi EF Kit according to the manufacturer's instructions. The DNA concentration was measured with Nanodrop.

### g) Ethanol Precipitation:

The volume of the DNA solution was mixed with the 2.5-fold volume ethanol 100%. The mixture was incubated at −20° C. for 10 min. Then the DNA was centrifuged for 30 min. at 14,000 rpm, 4° C. The supernatant was carefully removed and the pellet washed with 70% ethanol. Again, the tube was centrifuged for 5 min. at 14,000 rpm, 4° C. The supernatant was carefully removed by pipetting and the pellet dried. When the ethanol was evaporated, an appropriate amount of endotoxin-free water was added. The DNA was given time to re-dissolve in the water overnight at 4° C. A small aliquot was taken and the DNA concentration was measured with a Nanodrop device.

### Plasmid Generation

### Expression Cassette Composition

For the expression of an antibody chain, a transcription unit comprising the following functional elements were used:
the immediate early enhancer and promoter from the human cytomegalovirus including intron A,
a human heavy chain immunoglobulin 5′-untranslated region (5′UTR),
a murine immunoglobulin heavy chain signal sequence,
a nucleic acid encoding the respective antibody chain,
the bovine growth hormone polyadenylation sequence (BGH pA), and
optionally the human gastrin terminator (hGT).

Beside the expression unit/cassette including the desired gene to be expressed, the basic/standard mammalian expression plasmid contains:
an origin of replication from the vector pUC18 which allows replication of this plasmid in E. coli, and
a beta-lactamase gene which confers ampicillin resistance in E. coli.

### Front—and Back-Vector Cloning

To construct two-plasmid antibody constructs, antibody HC and LC fragments were cloned into a front vector backbone containing L3 and LoxFas sequences, and a back vector containing LoxFas and 2L sequences and a pac selectable marker. The Cre recombinase plasmid pOG231 (Wong, E. T., et al., Nucl. Acids Res. 33 (2005) e147; O'Gorman, S., et al., Proc. Natl. Acad. Sci. USA 94 (1997) 14602-14607) was used for all RMCE processes.

The cDNAs encoding the respective antibody chains were generated by gene synthesis (Geneart, Life Technologies Inc.). The gene synthesis and the backbone-vectors were digested with HindIII-HF and EcoRI-HF (NEB) at 37° C. for 1 h and separated by agarose gel electrophoresis. The DNA-fragment of the insert and backbone were cut out from the agarose gel and extracted by QIAquick Gel Extraction Kit (Qiagen). The purified insert and backbone fragment was ligated via the Rapid Ligation Kit (Roche) following the manufacturer's protocol with an Insert/Backbone ratio of 3:1. The ligation approach was then transformed in competent E. coli DH5u via heat shock for 30 sec. at 42° C. and incubated for 1 h at 37° C. before they were plated out on agar plates with ampicillin for selection. Plates were incubated at 37° C. overnight.

On the following day clones were picked and incubated overnight at 37° C. under shaking for the Mini or Maxi-Preparation, which was performed with the EpMotion® 5075 (Eppendorf) or with the QIAprep Spin Mini-Prep Kit (Qiagen)/NucleoBond Xtra Maxi EF Kit (Macherey & Nagel), respectively. All constructs were sequenced to ensure the absence of any undesirable mutations (SequiServe GmbH).

In the second cloning step, the previously cloned vectors were digested with KpnI-HF/SalI-HF and SalI-HF/MfeI-HF with the same conditions as for the first cloning. The TI backbone vector was digested with KpnI-HF and MfeI—HF. Separation and extraction was performed as described above. Ligation of the purified insert and backbone was performed using T4 DNA Ligase (NEB) following the manufacturing protocol with an Insert/Insert/Backbone ratio of 1:1:1 overnight at 4° C. and inactivated at 65° C. for 10 min. The following cloning steps were performed as described above.

The cloned plasmids were used for the TI transfection and pool generation.

Cultivation, transfection, selection and single cell cloning

TI host cells were propagated in disposable 125 ml vented shake flasks under standard humidified conditions (95% rH, 37° C., and 5% CO₂) at a constant agitation rate of 150 rpm in a proprietary DMEM/F12-based medium. Every 3-4 days the cells were seeded in chemically defined medium containing selection marker 1 and selection marker 2 in effective concentrations with a concentration of 3×10E5 cells/ml. Density and viability of the cultures were measured with a Cedex HiRes cell counter (F. Hoffmann-La Roche Ltd, Basel, Switzerland).

For stable transfection, equimolar amounts of front and back vector were mixed. Total DNA used per transfection was 30 μg with plasmid ratio 2.5:2.5:1 (front-, back-, Cre plasmid).

Two days prior to transfection TI host cells were seeded in fresh medium with a density of 4×10E5 cells/ml. Transfection was performed with the MaxCyte STX electroporation device (MaxCyte Inc., Gaithersburg) using OC-400 electroporation cassettes according to the manufacturer's protocol. 3×10E7 cells were transfected with a total of 30 μg nucleic acids, i.e. either with 30 μg plasmid (with a molar ratio of 2.5:2.5:1 of front:back:Cre plasmid)) or with 5 μg Cre mRNA and 25 μg front-and back-vector mixture. After transfection, the cells were seeded in 30 ml medium without selection agents.

On day 5 after seeding the cells were centrifuged and transferred to 80 mL chemically defined medium containing puromycin (selection agent 1) and 1-(2′-deoxy-2′-fluoro-1-beta-D-arabinofuranosyl-5-iodo)uracil (FIAU; selection agent 2) at effective concentrations at 6×10E5 cells/ml for selection of recombinant cells. The cells were incubated at 37° C., 150 rpm. 5% CO2, and 85% humidity from this day on without splitting. Cell density and viability of the culture was monitored regularly. When the viability of the culture started to increase again, the concentrations of selection agents 1 and 2 were reduced to about half the amount used before.

In more detail, to promote the recovering of the cells, the selection pressure was reduced if the viability is >40% and the viable cell density (VCD) is >0.5×10E6 cells/mL. Therefore, 4×10E5 cells/ml were centrifuged and resuspended in 40 ml selection media II (chemically defined medium, ½ selection marker 1 & 2). The cells were incubated with the same conditions as before and also not split.

Ten days after starting selection, the success of Cre mediated cassette exchange was checked by flow cytometry measuring the expression of intracellular GFP and extracellular heterologous polypeptide bound to the cell surface. An APC antibody (allophycocyanin-labeled F(ab′)2 Fragment goat anti-human IgG) against human antibody light and heavy chain was used for FACS staining. Flow cytometry was performed with a BD FACS Canto II flow cytometer (BD, Heidelberg, Germany). Ten thousand events per sample were measured. Living cells were gated in a plot of forward scatter (FSC) against side scatter (SSC). The live cell gate was defined with non-transfected TI host cells and applied to all samples by employing the FlowJo 10.8.1 EN software (TreeStar, Olten, Switzerland). Fluorescence of GFP was quantified in the FITC channel (excitation at 488 nm, detection at 530 nm). Heterologous polypeptide was measured in the APC channel (excitation at 645 nm, detection at 660 nm). Parental CHO cells, i.e., those cells used for the generation of the TI host cell, were used as a negative control with regard to GFP and heterologous polypeptide expression. Fourteen to twenty-one days after the selection had been started, the viability exceeded 90% and selection was considered as complete.

After selection, the pool of stably transfected cells can be subjected to single-cell cloning by limiting dilution. For this purpose, cells are stained with Cell Tracker Green™ (Thermo Fisher Scientific, Waltham, MA) and plated in 384-well plates with 0.6 cells/well. For single-cell cloning and all further cultivation steps, selection agent 2 is omitted from the medium. Wells containing only one cell are identified by bright field and fluorescence-based plate imaging. Only wells that contain one cell are further considered. Approximately three weeks after plating colonies are picked from confluent wells and further cultivated in 96-well plates.

### FACS Screening

FACS analysis was performed to check the transfection efficiency and the RMCE efficiency of the transfection. 4×10E5 cells of the transfected approaches were centrifuged (1200 rpm, 4 min.) and washed twice with 1 mL PBS. After the washing steps with PBS the pellet was resuspended in 400 μL PBS and transferred in FACS tubes (Falcon® Round-Bottom Tubes with cell strainer cap; Corning). The measurement was performed with a FACS Canto II and the data were analyzed by the software FlowJo.

### Fed-Batch Cultivation

Fed-batch production cultures were performed in shake flasks or Ambr 15 vessels (Sartorius Stedim) with proprietary chemically defined medium. Cells were seeded at 2×10E6 cells/ml on day 0. Cultures received proprietary feed medium on days 3, 7, and 10. Viable cell count (VCC) and percent viability of cells in culture was measured on days 0, 3, 7, 10, and 14 using a Cedex HiRes instrument (Roche Diagnostics GmbH, Mannheim, Germany). Glucose, lactate and product titer concentrations were measured on days 3, 5, 7, 10, 12 and 14 using a Cobas Analyzer (Roche Diagnostics GmbH, Mannheim, Germany). The supernatant was harvested 14 days after start of fed-batch cultivation by centrifugation (10 min, 1000 rpm and 10 min, 4000 rpm) and cleared by filtration (0.22 μm). Day 14 titers were determined using protein A affinity chromatography with UV detection. Product quality was determined by Caliper's LabChip (Caliper Life Sciences).

RNP-based CRISPR-Cas9 gene knock-outs in CHO cells

### Material/Resources:

Geneious 2021.2.2 software for guide and primer design
CHO TI host cell line; cultivation state: day 30-60
Gibco TrueCut Cas9 Protein, A45220P, Thermo Fisher
sgRNA (each sgRNA is custom designed against a target gene from the list in Table 6 of Example 1, 3 nm chemically modified sgRNA, Synthego)
medium (200 μg/ml Hygromycin B, 4 μg/ml selection agent 2)
DPBS—Dulbecco's Phosphate-Buffered Saline w/o Ca and Mg (Thermo Fisher)
Microplate 24 deep well plate (Agilent Technologies, Porvoir science) with cover (self-made)
Thin, long RNase, DNase, pyrogen free filter tips for loading OC-100 cassettes. (Biozyme)
Hera Safe Hood (Thermo Fisher)
Cedex HiRes Analyzer (Innovatis)
Liconic Incubator Storex IC
HyClone electroporation buffer
MaxCyte OC-100 cassettes
MaxCyte STX electroporation system

### CRISPR-Cas9 RNP Delivery

RNPs were preassembled by mixing 30 pmol Cas9 with 30 pmol pg gRNA mix (equal ratio of each gRNA—see below for exemplary genes-specific gRNA sequences) and incubated for 20 minutes at RT. Cells with a concentration between 2-4×10E6 cell/mL were centrifuged (3 minutes, 300 g). Afterwards the cells were resuspended in 90 μL HyClone electroporation buffer. The pre-incubated RNP mix was added to the cells and incubated for 5 minutes. The cell/RNP solution was then transferred into an OC-100 cuvette and electroporated with program “CHO2” using a MaxCyte electroporation system. Immediately after electroporation, the cell suspension was transferred into a 24 dwell and incubated at 37° C. for 30 minutes. Fresh and pre-warmed medium was added to result in a final cell concentration of 1×10E6 and incubated at 37° C. with shaking at 350 rpm for cell expansion. For genomic DNA preparation (day 6 or 8), QuickExtract kit (Lucigen) was added to the cells and served as a PCR template. Specific gene amplicons were PCR-amplified using standard Q5 Hot Start Polymerase protocol (NEB) and gene-specific primers that span the gRNA target sites (see below for examples). The respective amplicon was purified using QIAquick PCR purification kit (Qiagen) and analyzed by Sanger sequencing by Eurofins Genomics GmbH to verify gene inactivation by knock-out.

SIRT-1 guide RNAs
gRNA_SIRT1_1:
TATCATCCAACTCAGGTGGA
gRNA_SIRT1_2:
GCAGCATCTCATGATTGGCA
gRNA_SIRT1_3:
GCATTCTTGAAGTAACTTCA
SIRT-1 PCR primers
SIRT1_for:
ATGGCAGTTTTAGACACC
SIRT1_rev:
CTTGGAACTCAGACAAGG
MYC guide RNAs
gRNA_MYC_1:
CTATGACCTCGACTACGACT
gRNA_MYC_2:
GGACGCAGCGACCGTCACAT
gRNA_MYC_3:
CACCATCTCCAGCTGATCCG
MYC PCR primers
MYC_for:
CACACACACACTTGGAAG
MYC_rev:
CTTGATGAAGGTCTCGTC
ICAM-1 guide RNAs
gRNA_ICAM1_1:
ACCTGCATGGATGCACCCCG
gRNA_ICAM1_2:
GCACCGTGCCCACCTCCAGG
gRNA_ICAM1_3:
TAACCGCCAGAGAAAGATC
gRNA_ICAM1_4:
ACCTGCATGGATGCACCCCG
ICAM-1 PCR primers
ICAM1_for:
CCAAGCTAGATGATGTGAG
ICAM1_rev:
GCCCTACCCTTTTAATAC
BAK guide RNAs
gRNA_BAK_1:
TACAGCATCTTGGGTCAGGT
gRNA_BAK_2:
GTCCATCTCGGGGTTGGCAG
gRNA_BAK_3:
AATCTTGGTGAAGAGTTCGT
gRNA_BAK_4:
TCATCACAGTCCTGCCTAGG
gRNA_BAK_5:
ATGGCGTCTGGACAAGGACC
BAK PCR primer
BAK_for:
CGTATCTGAGTTCACGAAC
BAK_rev:
CCATCAGGAACAAGAGAC
BAX guide RNAs
gRNA_BAX_1:
ACAGGGGCCTTTTTGCTACA
gRNA_BAX_2:
GCTCATCTCCAATTCGCCTG
gRNA_BAX_3:
ACGAGAGGTCTTCTTCCGTG
gRNA_BAX_4:
GGGTCGGGGGAGCAGCTCGG
gRNA_BAX_5:
GGGTCCCGAAGTATGAGAGG
BAX PCR primers:
BAX_for:
ATCTTGTCTCCCTCGTAG
BAX_rev:
TCCTGGACTTCTCTAACC

### Fed-Batch Cultivation

Fed-batch production cultures were performed in Ambr 15 or Ambr 250 or 2-L bioreactors (Sartorius Stedim) with proprietary chemically defined medium. Cells were seeded at 2×10E6 cells/ml. Cultures received proprietary feed medium on days 3, 7, and 10. Viable cell count (VCC) and percent viability of cells in culture was measured on days 0, 3, 7, 10, 12 and 14 using a Cedex HiRes (Roche Diagnostics GmbH, Mannheim, Germany). Glucose concentration, lactate concentration and product titer were measured on days 3, 5, 7, 10, 12 and 14 using a Cobas analyzer (Roche Diagnostics GmbH, Mannheim, Germany). The supernatant was harvested 10, 12 or 14 days after start of fed-batch by centrifugation (10 min., 1000 rpm followed by 10 min., 4000 rpm) and cleared by filtration (0.22 μm). Harvest titers were further determined using protein A affinity chromatography with UV detection. Product quality was determined by Caliper's LabChip (Caliper Life Sciences).

### High Cell Density Fed-Batch Cultivation

Fed-batch production cultures were performed in Ambr 15 or Ambr 250 or 2-L bioreactors (Sartorius Stedim) with proprietary chemically defined medium. Cells were seeded at 15×10E6 cells/ml on day 0. Cultures received proprietary feed medium on days 1, 3, and 6. Viable cell count (VCC) and percent viability of cells in culture was measured on days 0, 3, 7, 10, 12 and 14 using a Cedex HiRes instrument (Roche Diagnostics GmbH, Mannheim, Germany). Glucose concentration, lactate concentration and product titer were measured on days 3, 5, 7, 10, 12, and 14 using a Cobas Analyzer (Roche Diagnostics GmbH, Mannheim, Germany). The supernatant was harvested 10 or 12 or 14 days after start of the cultivation by centrifugation (10 min., 1000 rpm followed by 10 min., 4000 rpm) and cleared by filtration (0.22 μm). Harvest titers were further determined using protein A affinity chromatography with UV detection. Product quality was determined by Caliper's LabChip (Caliper Life Sciences).

### Results

In a fed-batch cultivation process, a productivity increase of 40% or more for the modified cell with reduced expression of the BAK, BAX, SIRT-1, ICMA-1, and MYC genes has been observed.

This effect has been observed in cell pools or clones expressing different antibodies in different formats compared to the unmodified cell pools or clones (data presented in the following Tables for a 10-day and a 14-day fed-batch cultivation, respectively). The control cells and the modified cells have the same genotype except for the additional reduction in the transcriptional activity of the identified genes, i.e., the modification has been introduced into cells stably expressing the respective antibody.

| | day 10 harvest | |
|---|---|---|
| | titer without | day 10 harvest |
| | modification | titer with |
| | (control) | modification |
| antibody - modification | [μg/mL] | [μg/mL] |
| mAb-Q | 3463 | 5528 + 60% |
| modification of BAK, BAX, SIRT-1, | | |
| MYC | | |
| mAb-Q | 3639 | 5519 + 52% |
| modification of BAK, BAX, SIRT-1, | | |
| MYC, ICAM-1 | | |
| mAb-R | 4074 | 6089 + 49% |
| modification of BAK, BAX, SIRT-1, | | |
| MYC, ICAM-1 | | |
| | day 14 harvest | |
| | titer w/o | day 14 harvest |
| | modification | titer w/ |
| | (control) | modification |
| antibody - modification | [μg/mL] | [μg/mL] |
| mAb-Q | 5200 | 7789 + 50% |
| modification of BAK, BAX, SIRT-1, | | |
| MYC, ICAM-1 | | |
| mAb-R | 5373 | 8044 + 50% |
| modification of BAK, BAX, SIRT-1, | | |
| MYC, ICAM-1 | | |
| mAb-S | 2218 | 4414 + 99% |
| modification of BAK, BAX, SIRT-1, | | |
| MYC, ICAM-1 gene transcriptional | | |
| activity | | |
| mAb-T | 2140 | 3624 + 69% |
| modification of BAK, BAX, SIRT-1, | | |
| MYC, ICAM-1 | | |
| mAb-U | 2188 | 3076 + 41% |
| modification of BAK, BAX, SIRT-1, | | |
| MYC, ICAM-1 | | |

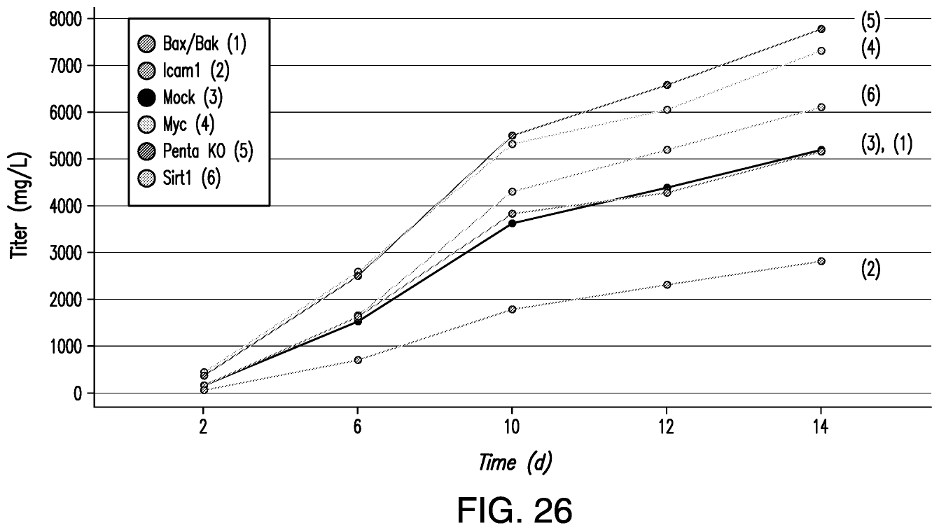

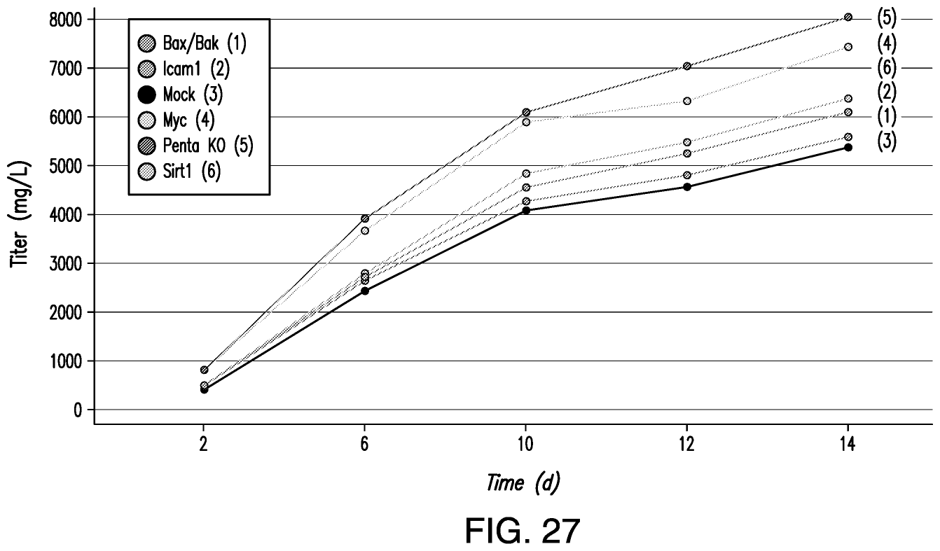

The subject matter of the current disclosure is based, at least in part, on the finding that the effect of the combination of modifications according to the subject matter of the current disclosure is more pronounced with cultivation times of more than 10 days. As shown in FIGS. 26 and 27, modified cells with reduced expression of the BAK, BAX, SIRT-1, MYC and ICAM-1 genes show no growth defect, have increased bioprocess viability, and exhibit an increased volumetric productivity.

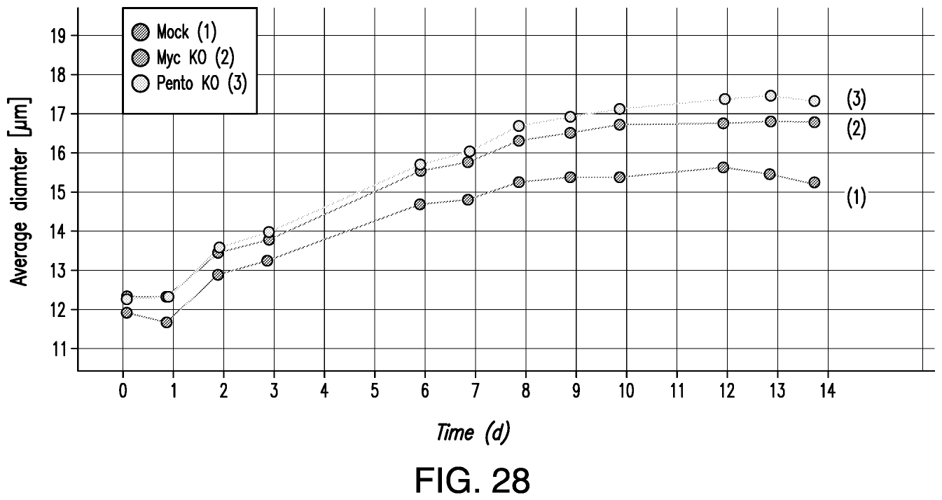

The increased volumetric productivity is based on an increase in the average cell diameter by 1-2 μm resulting in a volume increase of 15%-⁴⁵%. This is exemplarily shown in FIG. 28.

### Summary of the Cultivation Results

| | | | | | | | | Sum | Sum | |
|---|---|---|---|---|---|---|---|---|---|---|
| | | | | | Titer | | Monomer | LMW | HMW | CE-SDS |
| | | | | Harvest | Harvest | rel | (SEC) | (SEC) | (SEC) | monomer |
| Run | antibody | Description | gRNA | Day | [μg/ml] | titer | [%] | [%] | [%] | [%] |
| 8/9-1 | mAb-Q | no modification | no modification | 10 | 3395 | n/a | 79.1 | 9.9 | 10.9 | 79.3 |
| 8/9-2 | mAb-Q | no modification | no modification | 10 | 3530 | n/a | 80.1 | 8.6 | 11.3 | 80.1 |
| 8/9-1/2 | mAb-Q | no modification | no modification | 10 | 3463 | 100 | 79.6 | 9.2 | 11.1 | 79.7 |
| | | (average) | | | | | | | | |
| 8/9-3 | mAb-Q | reduction of | gRNA_MYC_1, 2, 3 | 10 | 5105 | 147 | 81.2 | 6.9 | 11.9 | 79.8 |
| | | MYC | | | | | | | | |
| 8/9-4 | mAb-Q | reduction of | gRNA_MYC_1, 2, 3 | 10 | 5141 | 148 | 74.0 | 6.7 | 19.3 | 79.2 |
| | | MYC + SIRT-1 | gRNA_SIRT1_1, 2, 3 | | | | | | | |
| 8/9-5 | mAb-Q | reduction of | gRNA_MYC_1, 2, 3 | 10 | 4283 | 124 | 82.5 | 7.0 | 10.6 | 81.2 |
| | | MYC + ICAM-1 | gRNA_ICAM1_1, 2, 3 | | | | | | | |
| 8/9-6 | mAb-Q | reduction of | gRNA_MYC_1, 2, 3 | 10 | 4690 | 135 | 82.7 | 7.5 | 9.7 | 79.5 |
| | | MYC + BAX + | gRNA_BAK_2 | | | | | | | |
| | | BAK | gRNA_BAX_3 | | | | | | | |
| 8/9-7 | mAb-Q | reduction of | gRNA_MYC_1, 2, 3 | 10 | 4814 | 139 | 81.4 | 7.4 | 11.2 | 80.3 |
| | | MYC + BAX + | gRNA_ICAM1_1, 2, 3 | | | | | | | |
| | | BAK + ICAM-1 | gRNA_BAK_2 | | | | | | | |
| | | | gRNA_BAX_3 | | | | | | | |
| 8/9-8 | mAb-Q | reduction of | gRNA_MYC_1, 2, 3 | 10 | 5528 | 160 | 82.4 | 7.4 | 10.2 | 80.4 |
| | | MYC + BAX + | gRNA_SIRT1_1, 2, 3 | | | | | | | |
| | | BAK + SIRT-1 | gRNA_BAK_2 | | | | | | | |
| | | | gRNA_BAX_3 | | | | | | | |
| 8/9-9 | mAb-Q | reduction of | gRNA_MYC_1, 2, 3 | 10 | 4220 | 122 | 83.8 | 5.5 | 10.7 | 82.4 |
| | | MYC + ICAM-1 + | gRNA_SIRT1_1, 2, 3 | | | | | | | |
| | | SIRT-1 | gRNA_ICAM1_1, 2, 3 | | | | | | | |
| 8/9-10 | mAb-Q | reduction of | gRNA_SIRT1_1, 2, 3 | 10 | 4611 | 133 | 80.6 | 7.6 | 11.8 | 78.8 |
| | | SIRT-1 + BAX + | gRNA_ICAM1_1, 2, 3 | | | | | | | |
| | | BAK + ICAM-1 | gRNA_BAK_2 | | | | | | | |
| | | | gRNA_BAX_3 | | | | | | | |
| 8/9-11 | mAb-R | no modification | no modification | 10 | 3966 | n/a | 87.1 | 7.5 | 4.7 | 87.6 |
| 8/9-12 | mAb-R | no modification | no modification | 10 | 4085 | n/a | 86.8 | 8.0 | 5.3 | 86.5 |
| 8/9-11/12 | mAb-R | no modification | no modification | 10 | 4026 | 100 | 87.0 | 7.8 | 5.0 | 87.0 |
| | | (average) | | | | | | | | |
| 8/9-13 | mAb-R | reduction of | gRNA_MYC_1, 2, 3 | 10 | 5742 | 143 | 87.1 | 7.4 | 5.6 | 86.9 |
| | | MYC | | | | | | | | |
| 8/9-14 | mAb-R | reduction of | gRNA_MYC_1, 2, 3 | 10 | 5783 | 144 | 88.6 | 6.5 | 5.0 | 87.6 |
| | | MYC + SIRT-1 | gRNA_SIRT1_1, 2, 3 | | | | | | | |
| 8/9-15 | mAb-R | reduction of | gRNA_MYC_1, 2, 3 | 10 | 5548 | 138 | 87.6 | 7.1 | 5.4 | 87.9 |
| | | MYC + ICAM-1 | gRNA_ICAM1_1, 2, 3 | | | | | | | |
| 8/9-16 | mAb-R | reduction of | gRNA_MYC_1, 2, 3 | 10 | 5805 | 144 | 86.7 | 7.6 | 5.6 | 85.8 |
| | | MYC + BAX + | gRNA_BAK_2 | | | | | | | |
| | | BAK | gRNA_BAX_3 | | | | | | | |
| 8/9-17 | mAb-R | reduction of | gRNA_MYC_1, 2, 3 | 10 | 5616 | 140 | 87.5 | 7.3 | 5.1 | 86.8 |
| | | MYC + BAX + | gRNA_ICAM1_1, 2, 3 | | | | | | | |
| | | BAK + ICAM-1 | gRNA_BAK_2 | | | | | | | |
| | | | gRNA_BAX_3 | | | | | | | |
| 8/9-18 | mAb-R | reduction of | gRNA_MYC_1, 2, 3 | 10 | 5605 | 139 | 88.2 | 6.7 | 5.0 | 87.9 |
| | | MYC + BAX + | gRNA_SIRT1_1, 2, 3 | | | | | | | |
| | | BAK + SIRT-1 | gRNA_BAK_2 | | | | | | | |
| | | | gRNA_BAX_3 | | | | | | | |
| 8/9-19 | mAb-R | reduction of | gRNA_MYC_1, 2, 3 | 10 | 5993 | 149 | 88.1 | 6.4 | 5.4 | 87.9 |
| | | MYC + ICAM-1 + | gRNA_SIRT1_1, 2, 3 | | | | | | | |
| | | SIRT-1 | gRNA_ICAM1_1, 2, 3 | | | | | | | |
| 8/9-20 | mAb-R | reductino of | gRNA_SIRT1_1, 2, 3 | 10 | 4688 | 116 | 87.7 | 7.3 | 5.0 | 88.0 |
| | | SIRT-1 + | gRNA_ICAM1_1, 2, 3 | | | | | | | |
| | | BAX + BAK + | gRNA_BAK_2 | | | | | | | |
| | | ICAM-1 | gRNA_BAX_3 | | | | | | | |
| 11-1 | anti-CD20/TfR | no modification | no modification | 10 | 585 | 100 | 29.1 | 67.8 | 3.0 | 18.9 |
| | antibody BS | | | | | | | | | |
| | format (pool) | | | | | | | | | |
| 11-2 | mAb-V | reduction of | gRNA_MYC_3 | 10 | 1088 | 186 | 30.7 | 66.1 | 3.2 | 16.8 |
| | (pool) | MYC + BAX + | gRNA_ICAM1_2 | | | | | | | |
| | | BAK + ICAM-1 | gRNA_BAK_2 | | | | | | | |
| | | | gRNA_BAX_3 | | | | | | | |
| 11-3 | mAb-V | no modification | no modification | 10 | 3150 | 100 | 85.2 | 9.4 | 5.4 | 71.8 |
| | (clone) | | | | | | | | | |
| 11-4 | mAb-V | reduction of | gRNA_MYC_3 | 10 | 4439 | 141 | 87.1 | 7.1 | 5.9 | 73.6 |
| | (clone) | MYC + BAX + | gRNA_ICAM1_2 | | | | | | | |
| | | BAK + ICAM-1 | gRNA_BAK_2 | | | | | | | |
| | | | gRNA_BAX_3 | | | | | | | |
| 11-5 | mAb-W | no | no modification | 10 | 1614 | 100 | 95.3 | 0.9 | 3.9 | 57.8 |
| | (clone) | modification | | | | | | | | |
| 11-6 | mAb-W | reduction of | gRNA_MYC_3 | 10 | 2774 | 172 | 96.4 | 0.9 | 2.6 | 56.5 |
| | (clone) | MYC + BAK + | gRNA_ICAM1_2 | | | | | | | |
| | | BAK + ICAM-1 | gRNA_BAK_2 | | | | | | | |
| | | | gRNA_BAX_3 | | | | | | | |
| 11-7 | mAb-Q | no modification | no modification | 10 | 4053 | 100 | 77.0 | 9.7 | 13.2 | 79.1 |
| 11-8 | mAb-Q | reduction of | gRNA_MYC_3 | 10 | 5982 | 148 | 75.5 | 7.9 | 16.6 | 78.3 |
| | | MYC + BAX + | gRNA_ICAM1_2 | | | | | | | |
| | | BAK + ICAM-1 | gRNA_BAK_2 | | | | | | | |
| | | | gRNA_BAX_3 | | | | | | | |
| 23/25-1 | mAb-Q | no modification | no modification | 10 | 3639 | 100 | 2.9 | 59.8 | 37.3 | 75.8 |
| 23/25-2 | mAb-Q | no modification | no modification | 12 | 4394 | 100 | 4.7 | 52.8 | 42.5 | 71.8 |
| 23/25-3 | mAb-Q | no modification | no modification | 14 | 5200 | 100 | 3.3 | 59.9 | 36.8 | 76.3 |
| 23/25-4 | mAb-Q | reduction of | gRNA_SIRT1_1, 2, 3 | 10 | 4306 | 118 | 3.4 | 79.5 | 17.2 | 76.4 |
| | | SIRT-1 | | | | | | | | |
| 23/25-5 | mAb-Q | reduction of | gRNA_SIRT1_1, 2, 3 | 12 | 5200 | 118 | 3.7 | 80.9 | 15.4 | 73.4 |
| | | SIRT-1 | | | | | | | | |
| 23/25-6 | FAP/4-1BBL | reduction of | gRNA_SIRT1_1, 2, 3 | 14 | 6129 | 118 | 3.6 | 75.5 | 20.9 | 73.9 |
| | | SIRT-1 | | | | | | | | |
| 23/25-7 | mAb-Q | reduction of | gRNA_MYC_1, 2, 3 | 10 | 5327 | 146 | 3.3 | 77.4 | 19.4 | 73.3 |
| | | MYC | | | | | | | | |
| 23/25-8 | mAb-Q | reduction of | gRNA_MYC_1, 2, 3 | 12 | 6070 | 138 | 3.0 | 67.6 | 29.4 | 70.9 |
| | | MYC | | | | | | | | |
| 23/25-9 | mAb-Q | reduction of | gRNA_MYC_1, 2, 3 | 14 | 7328 | 141 | 3.1 | 72.3 | 24.7 | 70.4 |
| | | MYC | | | | | | | | |
| 23/25-10 | mAb-Q | reduction of | gRNA_ICAM1_1, 2, 3 | 10 | 1785 | 49 | 3.3 | 63.6 | 33.1 | 82.4 |
| | | ICAM-1 | | | | | | | | |
| 23/25-11 | mAb-Q | reduction of | gRNA_ICAM1_1, 2, 3 | 12 | 2313 | 53 | 3.0 | 69.6 | 27.4 | 81.4 |
| | | ICAM-1 | | | | | | | | |
| 23/25-12 | mAb-Q | reduction of | gRNA_ICAM1_1, 2, 3 | 14 | 2829 | 54 | 3.4 | 0.0 | 25.7 | 84.0 |
| | | ICAM-1 | | | | | | | | |
| 23/25-13 | mAb-Q | reduction of | gRNA_BAK_4, 5 | 10 | 3831 | 105 | 3.9 | 80.5 | 15.6 | 75.6 |
| | | BAX + BAK | gRNA_BAX_4, 5 | | | | | | | |
| 23/25-14 | mAb-Q | reduction of | gRNA_BAK_4, 5 | 12 | 4290 | 97 | 6.7 | 78.4 | 15.0 | 74.8 |
| | | BAX + BAK | gRNA_BAX_4, 5 | | | | | | | |
| 23/25-15 | mAb-Q | reduction of | gRNA_BAK_4, 5 | 14 | 5162 | 99 | 3.4 | 78.3 | 18.3 | 74.7 |
| | | BAX + BAK | gRNA_BAX_4, 5 | | | | | | | |
| 23/25-16 | mAb-Q | reduction of | gRNA_MYC_1, 2, 3 | 10 | 5519 | 152 | 3.3 | 75.1 | 21.7 | 73.4 |
| | | MYC + BAX + | gRNA_SIRT1_1, 2, 3 | | | | | | | |
| | | BAK + ICAM-1 + | gRNA_ICAM1_1, 2, 3 | | | | | | | |
| | | SIRT-1 | gRNA_BAK_4, 5 | | | | | | | |
| | | | gRNA_BAX_4, 5 | | | | | | | |
| 23/25-17 | mAb-Q | reduction of | gRNA_MYC_1, 2, 3 | 12 | 6589 | 150 | 4.2 | 81.6 | 14.1 | 73.4 |
| | | MYC + BAX + | gRNA_SIRT1_1, 2, 3 | | | | | | | |
| | | BAK + ICAM-1 + | gRNA_ICAM1_1, 2, 3 | | | | | | | |
| | | SIRT-1 | gRNA_BAK_4, 5 | | | | | | | |
| | | | gRNA_BAX_4, 5 | | | | | | | |
| 23/25-18 | mAb-Q | reduction of | gRNA_MYC_1, 2, 3 | 14 | 7789 | 150 | 3.6 | 82.4 | 14.0 | 74.2 |
| | | MYC + BAX + | gRNA_SIRT1_1, 2, 3 | | | | | | | |
| | | BAK + ICAM-1 + | gRNA_ICAM1_1, 2, 3 | | | | | | | |
| | | SIRT-1 | gRNA_BAK_4, 5 | | | | | | | |
| | | | gRNA_BAX_4, 5 | | | | | | | |
| 23/25-19 | mAb-R | no modification | no modification | 10 | 4074 | 100 | 92.6 | 4.7 | 2.6 | 87.9 |
| 23/25-20 | mAb-R | no modification | no modification | 12 | 4565 | 100 | 92.0 | 5.0 | 2.9 | 87.3 |
| 23/25-21 | mAb-R | no modification | no modification | 14 | 5373 | 100 | 91.3 | 5.4 | 3.3 | 86.7 |
| 23/25-22 | mAb-R | reduction of | gRNA_SIRT1_1, 2, 3 | 10 | 4820 | 118 | 93.1 | 3.9 | 3.0 | 89.8 |
| | | SIRT-1 | | | | | | | | |
| 23/25-23 | mAb-R | reduction of | gRNA_SIRT1_1, 2, 3 | 12 | 5466 | 120 | 91.2 | 5.8 | 2.9 | 87.5 |
| | | SIRT-1 | | | | | | | | |
| 23/25-24 | mAb-R | reduction of | gRNA_SIRT1_1, 2, 3 | 14 | 6380 | 119 | 90.3 | 6.1 | 3.6 | 86.5 |
| | | SIRT-1 | | | | | | | | |
| 23/25-25 | mAb-R | reduction of | gRNA_MYC_1, 2, 3 | 10 | 5888 | 145 | 91.0 | 5.9 | 3.2 | 86.7 |
| | | MYC | | | | | | | | |
| 23/25-26 | mAb-R | reduction of | gRNA_MYC_1, 2, 3 | 12 | 6318 | 138 | 90.7 | 6.2 | 3.1 | 85.0 |
| | | MYC | | | | | | | | |
| 23/25-27 | mAb-R | reduction of | gRNA_MYC_1, 2, 3 | 14 | 7440 | 138 | 89.8 | 6.8 | 3.4 | 85.0 |
| | | MYC | | | | | | | | |
| 23/25-28 | mAb-R | reduction of | gRNA_ICAM1_1, 2, 3 | 10 | 4549 | 112 | 92.2 | 4.8 | 3.0 | 87.2 |
| | | ICAM-1 | | | | | | | | |
| 23/25-29 | mAb-R | reduction of | gRNA_ICAM1_1, 2, 3 | 12 | 5244 | 115 | 91.0 | 5.4 | 3.6 | 86.9 |
| | | ICAM-1 | | | | | | | | |
| 23/25-30 | mAb-R | reduction of | gRNA_ICAM1_1, 2, 3 | 14 | 6082 | 113 | 88.9 | 6.6 | 4.5 | 85.5 |
| | | ICAM-1 | | | | | | | | |
| 23/25-31 | mAb-R | reduction of | gRNA_BAK_4, 5 | 10 | 4247 | 104 | 92.0 | 5.2 | 2.8 | 87.9 |
| | | BAX + BAK | gRNA_BAX_4, 5 | | | | | | | |
| 23/25-32 | mAb-R | reduction of | gRNA_BAK_4, 5 | 12 | 4792 | 105 | 91.3 | 5.7 | 3.0 | 87.0 |
| | | BAX + BAK | gRNA_BAX_4, 5 | | | | | | | |
| 23/25-33 | mAb-R | reduction of | gRNA_BAK_4, 5 | 14 | 5580 | 104 | 91.3 | 5.4 | 3.3 | 87.9 |
| | | BAX + BAK | gRNA_BAX_4, 5 | | | | | | | |
| 23/25-34 | mAb-R | reduction of | gRNA_MYC_1, 2, 3 | 10 | 6089 | 149 | 91.3 | 5.5 | 3.2 | 86.8 |
| | | MYC + BAX + | gRNA_SIRT1_1, 2, 3 | | | | | | | |
| | | BAK + ICAM-1 + | gRNA_ICAM1_1, 2, 3 | | | | | | | |
| | | SIRT-1 | gRNA_BAK_4, 5 | | | | | | | |
| | | | gRNA_BAX_4, 5 | | | | | | | |
| 23/25-35 | mAb-R | reduction of | gRNA_MYC_1, 2, 3 | 12 | 7031 | 154 | 90.2 | 6.2 | 3.6 | 86.4 |
| | | MYC + BAX + | gRNA_SIRT1_1, 2, 3 | | | | | | | |
| | | BAK + ICAM-1 + | gRNA_ICAM1_1, 2, 3 | | | | | | | |
| | | SIRT-1 | gRNA_BAK_4, 5 | | | | | | | |
| | | | gRNA_BAX_4, 5 | | | | | | | |
| 23/25-36 | mAb-R | reduction of | gRNA_MYC_1, 2, 3 | 14 | 8044 | 150 | 90.1 | 6.2 | 3.7 | 85.9 |
| | | MYC + BAX + | gRNA_SIRT1_1, 2, 3 | | | | | | | |
| | | BAK + ICAM-1 + | gRNA_ICAM1_1, 2, 3 | | | | | | | |
| | | SIRT-1 | gRNA_BAK_4, 5 | | | | | | | |
| | | | gRNA_BAX_4, 5 | | | | | | | |
| 29-1 | mAb-S | no modification | no modification | 14 | 2218 | 100 | 85.8 | 7.6 | 6.7 | 78.7 |
| 29-2 | mAb-S | reduction of | gRNA_MYC_3 | 14 | 4414 | 199 | 79.2 | 10.6 | 10.3 | 67.1 |
| | | MYC + BAX + | gRNA_BAX_3 | | | | | | | |
| | | BAK + ICAM-1 + | gRNA_BAK_2 | | | | | | | |
| | | SIRT-1 | gRNA_ICAM1_4 | | | | | | | |
| | | | gRNA_SIRT1_2 | | | | | | | |
| 29-3 | mAb-T | no modification | no modification | 14 | 2140 | 100 | 85.5 | 3.9 | 10.6 | 79.3 |
| 29-4 | mAb-T | reduction of | gRNA_MYC_3 | 14 | 3624 | 169 | 75.8 | 13.7 | 10.4 | 55.5 |
| | | MYC + BAX + | gRNA_BAX_3 | | | | | | | |
| | | BAK + ICAM-1 + | gRNA_BAK_2 | | | | | | | |
| | | SIRT-1 | gRNA_ICAM1_4 | | | | | | | |
| | | | gRNA_SIRT1_2 | | | | | | | |
| 29-5 | mAb-R | no modification | no modification | 14 | 2991 | 100 | 84.5 | 5.7 | 9.8 | 95.4 |
| 29-6 | mAb-R | reduction of | gRNA_MYC_3 | 14 | 4674 | 156 | 82.8 | 4.3 | 12.9 | 96.5 |
| | | MYC + BAX + | gRNA_BAX_3 | | | | | | | |
| | | BAK + ICAM-1 + | gRNA_BAK_2 | | | | | | | |
| | | SIRT-1 | gRNA_ICAM1_4 | | | | | | | |
| | | | gRNA_SIRT1_2 | | | | | | | |
| 29-7 | mAb-U | no modification | no modification | 14 | 2188 | 100 | 83.1 | 14.6 | 2.4 | 95.7 |
| 29-8 | mAb-U | reduction of | gRNA_MYC_3 | 14 | 3076 | 141 | 85.3 | 11.2 | 3.5 | 91.9 |
| | | MYC + BAX + | gRNA_BAX_3 | | | | | | | |
| | | BAK + ICAM-1 + | gRNA_BAK_2 | | | | | | | |
| | | SIRT-1 | gRNA_ICAM1_4 | | | | | | | |
| | | | gRNA_SIRT1_2 | | | | | | | |

The contents of all figures and all references, patents and published patent applications and Accession numbers cited throughout this application are expressly incorporated herein by reference.

What is claimed is:

1. A modified mammalian cell, wherein the cell is modified to reduce or eliminate the expression of one or more endogenous products relative to the expression of the endogenous products in an unmodified cell, wherein the one or more endogenous products:
(a) promotes apoptosis of the modified cell during cell culture;
(b) promotes clumping and/or aggregation of the modified cell during cell culture;
(c) is not essential for the growth, survival, and/or productivity of the modified cell during cell culture;
(d) promotes non-human glycosylation patterns in a recombinant protein product produced by the modified cell during cell culture;
(e) can co-purify with the product of interest produced by the modified cell during cell culture;
(f) promotes branched chain amino acid catabolism; and/or
(g) requires removal by purification for product quality and/or safety reasons.

2. The modified mammalian cell of claim 1, wherein the cell is modified to reduce or eliminate the expression of one or more endogenous products relative to the expression of the endogenous products in an unmodified cell, wherein the one or more endogenous products is selected from endogenous virus-like particles such as retrovirus-like particles (RVLPs) and/or the endogenous protein group consisting of: BCL2 Associated X, Apoptosis Regulator (BAX); BCL2 Antagonist/Killer 1 (BAK); Intercellular Adhesion Molecule 1 (ICAM-1); Protein Kinase R-like ER Kinase (PERK); Sirtuin 1 (SIRT-1); MYC Proto-Oncogene, BHLH Transcription Factor (MYC); Glycoprotein Alpha-Galactosyltransferase 1 (GGTA1); Cytidine Monophosphate-N-Acetylneuraminic Acid Hydroxylase (CMAH); Branched Chain Keto Acid Dehydrogenase El alpha subunit (BCKDHA); Branched Chain Keto Acid Dehydrogenase El beta subunit (BCKDHB); Lipoprotein lipase (LPL); Phospholipase A2 group XV (LPLA2); Palmitoyl-protein thioesterase 1 (PPT1); and Lipase A (Lysosomal acid lipase/cholesteryl ester hydrolase, Lipase) (LIPA).

3. The modified mammalian cell of claim 2, wherein the expression of RVLP is reduced or eliminated by reducing or eliminating the expression of RVLP group antigen (GAG).

4. The modified mammalian cell of claim 2, wherein the expression of:
a) BAX; BAK; ICAM-1; GGTA1; CMAH; LPL; LPLA2; and PPT1;
b) BAX; BAK; ICAM-1; SIRT-1; GGTA1; CMAH; LPL, LPLA2; and PPT1;
c) BAX; BAK; ICAM-1; SIRT-1; MYC; GGTA1; CMAH; LPL; LPLA2; and PPT1;
d) BAX; BAK; ICAM-1; SIRT-1; MYC; GGTA1; CMAH; LPL, LPLA2; PPT1; and LIPA;
e) BAX; BAK; ICAM-1; GGTA1; CMAH; LPLA2; PPT1; and LIPA;
f) BAX; BAK; ICAM-1; SIRT-1; GGTA1; CMAH; LPLA2; PPT1; and LIPA;
g) BAX; BAK; ICAM-1; SIRT-1; MYC; GGTA1; CMAH; LPLA2; PPT1; and LIPA;
h) BAX; BAK; ICAM-1; LPL; LPLA2; and PPT1;
i) BAX; BAK; ICAM-1; SIRT-1; LPL, LPLA2; and PPT1;
j) BAX; BAK; ICAM-1; SIRT-1; MYC; LPL; LPLA2; and PPT1;
k) BAX; BAK; ICAM-1; SIRT-1; MYC; LPL; LPLA2; PPT1; and LIPA;
l) BAX; BAK; ICAM-1; SIRT-1; and MYC;
m) BAX; BAK; ICAM-1; PERK; SIRT-1; and MYC;
n) BAX; BAK; ICAM-1; SIRT-1; PERK; MYC; GGTA1; CMAH; LPL, LPLA2; PPT1; and LIPA;
o) BAX; BAK; LPL; LPLA2; GGTA1; and CMAH;
p) BAX; BAK; PERK; LPL; LPLA2; GGTA1; and CMAH;
q) BAX; BAK; MYC; LPL; LPLA2; GGTA1; and CMAH;
r) BAX; BAK; MYC; PERK; LPL; LPLA2; GGTA1; and CMAH;
s) BAX; BAK; LPL; LPLA2; GGTA1; CMAH; and PPT1;
t) BAX; BAK; PERK; LPL; LPLA2; GGTA1; CMAH; and PPT1;
u) BAX; BAK; MYC; LPL; LPLA2; GGTA1; CMAH; and PPT1;
v) BAX; BAK; MYC; PERK; LPL; LPLA2; GGTA1; CMAH; and PPT1;
w) BAX; BAK; ICAM-1; and SIRT-1;
x) BAX; BAK; and ICAM-1;
y) BAX; BAK; BCKDHA; ICAM-1; GGTA1; CMAH; LPL; LPLA2; and PPT1;
z) BAX; BAK; BCKDHA; ICAM-1; SIRT-1; GGTA1; CMAH; LPL; LPLA2; and PPT1;
aa) BAX; BAK; BCKDHA; ICAM-1; SIRT-1; MYC; GGTA1; CMAH; LPL; LPLA2; and PPT1;
bb) BAX; BAK; BCKDHA; ICAM-1; SIRT-1; MYC; GGTA1; CMAH; LPL, LPLA2; PPT1; and LIPA;
cc) BAX; BAK; BCKDHA; ICAM-1; GGTA1; CMAH; LPLA2; PPT1; and
LIPA;
dd) BAX; BAK; BCKDHA; ICAM-1; SIRT-1; GGTA1; CMAH; LPLA2; PPT1; and LIPA;
ee) BAX; BAK; BCKDHA; ICAM-1; SIRT-1; MYC; GGTA1; CMAH; LPLA2; PPT1; and LIPA;
ff) BAX; BAK; BCKDHA; ICAM-1; LPL; LPLA2; and PPT1;
gg) BAX; BAK; BCKDHA; ICAM-1; SIRT-1; LPL, LPLA2; and PPT1;
hh) BAX; BAK; BCKDHA; ICAM-1; SIRT-1; MYC; LPL; LPLA2; and PPT1;
ii) BAX; BAK; BCKDHA; ICAM-1; SIRT-1; MYC; LPL; LPLA2; PPT1; and LIPA;
jj) BAX; BAK; BCKDHA; ICAM-1; SIRT-1; and MYC;
kk) BAX; BAK; BCKDHA; ICAM-1; PERK; SIRT-1; and MYC;
ll) BAX; BAK; BCKDHA; ICAM-1; PERK; SIRT-1; MYC; GGTA1; CMAH; LPL, LPLA2; PPT1; and LIPA;
mm) BAX; BAK; BCKDHA; LPL; LPLA2; GGTA1; and CMAH;
nn) BAX; BAK; BCKDHA; PERK; LPL; LPLA2; GGTA1; and CMAH;
oo) BAX; BAK; BCKDHA; MYC; LPL; LPLA2; GGTA1; and CMAH;
pp) BAX; BAK; BCKDHA; MYC; PERK; LPL; LPLA2; GGTA1; and CMAH;
qq) BAX; BAK; BCKDHA; LPL; LPLA2; GGTA1; CMAH; and PPT1;
rr) BAX; BAK; BCKDHA; PERK; LPL; LPLA2; GGTA1; CMAH; and PPT1;
ss) BAX; BAK; BCKDHA; MYC; LPL; LPLA2; GGTA1; CMAH; and PPT1;
tt) BAX; BAK; BCKDHA; MYC; PERK; LPL; LPLA2; GGTA1; CMAH; and PPT1;
uu) BAX; BAK; BCKDHA; ICAM-1; and SIRT-1;
vv) BAX; BAK; BCKDHA; and ICAM-1;
ww) BAX; BAK; BCKDHB; ICAM-1; GGTA1; CMAH; LPL; LPLA2; and PPT1;
xx) BAX; BAK; BCKDHB; ICAM-1; SIRT-1; GGTA1; CMAH; LPL; LPLA2; and PPT1;
yy) BAX; BAK; BCKDHB; ICAM-1; SIRT-1; MYC; GGTA1; CMAH; LPL; LPLA2; and PPT1;
zz) BAX; BAK; BCKDHB; ICAM-1; SIRT-1; MYC; GGTA1; CMAH; LPL, LPLA2; PPT1; and LIPA;
aaa) BAX; BAK; BCKDHB; ICAM-1; GGTA1; CMAH; LPLA2; PPT1; and LIPA;
bbb) BAX; BAK; BCKDHB; ICAM-1; SIRT-1; GGTA1; CMAH; LPLA2; PPT1; and LIPA;
ccc) BAX; BAK; BCKDHB; ICAM-1; SIRT-1; MYC; GGTA1; CMAH; LPLA2; PPT1; and LIPA;
ddd) BAX; BAK; BCKDHB; ICAM-1; LPL; LPLA2; and PPT1;
eee) BAX; BAK; BCKDHB; ICAM-1; SIRT-1; LPL, LPLA2; and PPT1;
fff) BAX; BAK; BCKDHB; ICAM-1; SIRT-1; MYC; LPL; LPLA2; and PPT1;
ggg) BAX; BAK; BCKDHB; ICAM-1; SIRT-1; MYC; LPL; LPLA2; PPT1; and
LIPA;
hhh) BAX; BAK; BCKDHB; ICAM-1; SIRT-1; and MYC;
iii) BAX; BAK; BCKDHB; ICAM-1; PERK; SIRT-1; and MYC;
jjj) BAX; BAK; BCKDHB; ICAM-1; PERK; SIRT-1; MYC; GGTA1; CMAH;
LPL, LPLA2; PPT1; and LIPA;
kkk) BAX; BAK; BCKDHB; LPL; LPLA2; GGTA1; and CMAH;
lll) BAX; BAK; BCKDHB; PERK; LPL; LPLA2; GGTA1; and CMAH;
mmm) BAX; BAK; BCKDHB; MYC; LPL; LPLA2; GGTA1; and CMAH;
nnn) BAX; BAK; BCKDHB; MYC; PERK; LPL; LPLA2; GGTA1; and CMAH;
ooo) BAX; BAK; BCKDHB; LPL; LPLA2; GGTA1; CMAH; and PPT1;
ppp) BAX; BAK; BCKDHB; PERK; LPL; LPLA2; GGTA1; CMAH; and PPT1;
qqq) BAX; BAK; BCKDHB; MYC; LPL; LPLA2; GGTA1; CMAH; and PPT1;
rrr) BAX; BAK; BCKDHB; MYC; PERK; LPL; LPLA2; GGTA1; CMAH; and PPT1;
sss) BAX; BAK; BCKDHB; ICAM-1; and SIRT-1;
ttt) BAX; BAK; BCKDHB; and ICAM-1;
uuu) BAX; BAK; BCKDHA; BCKDHB; ICAM-1; GGTA1; CMAH; LPL; LPLA2; and PPT1;
vvv) BAX; BAK; BCKDHA; BCKDHB; ICAM-1; SIRT-1; GGTA1; CMAH; LPL; LPLA2; and PPT1;
www) BAX; BAK; BCKDHA; BCKDHB; ICAM-1; SIRT-1; MYC; GGTA1; CMAH; LPL; LPLA2; and PPT1;
xxx) BAX; BAK; BCKDHA; BCKDHB; ICAM-1; SIRT-1; MYC; GGTA1; CMAH; LPL, LPLA2; PPT1; and LIPA;
yyy) BAX; BAK; BCKDHA; BCKDHB; ICAM-1; GGTA1; CMAH; LPLA2; PPT1; and LIPA;
zzz) BAX; BAK; BCKDHA; BCKDHB; ICAM-1; SIRT-1; GGTA1; CMAH; LPLA2; PPT1; and LIPA;
aaaa) BAX; BAK; BCKDHA; BCKDHB; ICAM-1; SIRT-1; MYC; GGTA1; CMAH; LPLA2; PPT1; and LIPA;
bbbb) BAX; BAK; BCKDHA; BCKDHB; ICAM-1; LPL; LPLA2; and PPT1;
cccc) BAX; BAK; BCKDHA; BCKDHB; ICAM-1; SIRT-1; LPL, LPLA2; and PPT1;
dddd) BAX; BAK; BCKDHA; BCKDHB; ICAM-1; SIRT-1; MYC; LPL; LPLA2; and PPT1;
eeee) BAX; BAK; BCKDHA; BCKDHB; ICAM-1; SIRT-1; MYC; LPL; LPLA2; PPT1; and LIPA;
ffff) BAX; BAK; BCKDHA; BCKDHB; ICAM-1; SIRT-1; and MYC;
gggg) BAX; BAK; BCKDHA; BCKDHB; ICAM-1; PERK; SIRT-1; and MYC;
hhhh) BAX; BAK; BCKDHA; BCKDHB; ICAM-1; PERK; SIRT-1; MYC; GGTA1; CMAH; LPL, LPLA2; PPT1; and LIPA;
iiii) BAX; BAK; BCKDHA; BCKDHB; LPL; LPLA2; GGTA1; and CMAH;
jjjj) BAX; BAK; BCKDHA; BCKDHB; PERK; LPL; LPLA2; GGTA1; and CMAH;
kkkk) BAX; BAK; BCKDHA; BCKDHB; MYC; LPL; LPLA2; GGTA1; and CMAH;
llll) BAX; BAK; BCKDHA; BCKDHB; MYC; PERK; LPL; LPLA2; GGTA1; and CMAH;
mmmm) BAX; BAK; BCKDHA; BCKDHB; LPL; LPLA2; GGTA1; CMAH; and PPT1;
nnnn) BAX; BAK; BCKDHA; BCKDHB; PERK; LPL; LPLA2; GGTA1; CMAH; and PPT1;
oooo) BAX; BAK; BCKDHA; BCKDHB; MYC; LPL; LPLA2; GGTA1; CMAH; and PPT1;
pppp) BAX; BAK; BCKDHA; BCKDHB; MYC; PERK; LPL; LPLA2; GGTA1; CMAH; and PPT1;
qqqq) BAX; BAK; BCKDHA; BCKDHB; ICAM-1; and SIRT-1; or
rrrr) BAX; BAK; BCKDHA; BCKDHB; and ICAM-1 is reduced or eliminated.

5. The modified mammalian cell of claim 1, wherein the expression of:
a) GAG; BAX; BAK; ICAM-1; GGTA1; CMAH; LPL; LPLA2; and PPT1;
b) GAG; BAX; BAK; ICAM-1; SIRT-1; GGTA1; CMAH; LPL, LPLA2; and PPT1;
c) GAG; BAX; BAK; ICAM-1; SIRT-1; MYC; GGTA1; CMAH; LPL; LPLA2; and PPT1;
d) GAG; BAX; BAK; ICAM-1; SIRT-1; MYC; GGTA1; CMAH; LPL, LPLA2; PPT1; and LIPA;
e) GAG; BAX; BAK; ICAM-1; GGTA1; CMAH; LPLA2; PPT1; and LIPA;
f) GAG; BAX; BAK; ICAM-1; SIRT-1; GGTA1; CMAH; LPLA2; PPT1; and LIPA;
g) GAG; BAX; BAK; ICAM-1; SIRT-1; MYC; GGTA1; CMAH; LPLA2; PPT1; and LIPA;
h) GAG; BAX; BAK; ICAM-1; LPL; LPLA2; and PPT1;
i) GAG; BAX; BAK; ICAM-1; SIRT-1; LPL, LPLA2; and PPT1;
j) GAG; BAX; BAK; ICAM-1; SIRT-1; MYC; LPL; LPLA2; and PPT1;
k) GAG; BAX; BAK; ICAM-1; SIRT-1; MYC; LPL; LPLA2; PPT1; and LIPA;
l) GAG; BAX; BAK; ICAM-1; SIRT-1; and MYC;
m) GAG; BAX; BAK; ICAM-1; PERK; SIRT-1; and MYC;
n) GAG; BAX; BAK; ICAM-1; SIRT-1; PERK; MYC; GGTA1; CMAH; LPL, LPLA2; PPT1; and LIPA;
o) GAG; BAX; BAK; LPL; LPLA2; GGTA1; and CMAH;
p) GAG; BAX; BAK; PERK; LPL; LPLA2; GGTA1; and CMAH;
q) GAG; BAX; BAK; MYC; LPL; LPLA2; GGTA1; and CMAH;
r) GAG; BAX; BAK; MYC; PERK; LPL; LPLA2; GGTA1; and CMAH;
s) GAG; BAX; BAK; LPL; LPLA2; GGTA1; CMAH; and PPT1;
t) GAG; BAX; BAK; PERK; LPL; LPLA2; GGTA1; CMAH; and PPT1;
u) GAG; BAX; BAK; MYC; LPL; LPLA2; GGTA1; CMAH; and PPT1;
v) GAG; BAX; BAK; MYC; PERK; LPL; LPLA2; GGTA1; CMAH; and PPT1;
w) GAG; BAX; BAK; ICAM-1; and SIRT-1;
x) GAG; BAX; BAK; and ICAM-1;
y) BAX; BAK; BCKDHA; ICAM-1; GGTA1; CMAH; LPL; LPLA2; and PPT1;
z) BAX; BAK; BCKDHA; ICAM-1; SIRT-1; GGTA1; CMAH; LPL; LPLA2; and PPT1;
aa) BAX; BAK; BCKDHA; ICAM-1; SIRT-1; MYC; GGTA1; CMAH; LPL; LPLA2; and PPT1;
bb) BAX; BAK; BCKDHA; ICAM-1; SIRT-1; MYC; GGTA1; CMAH; LPL, LPLA2; PPT1; and LIPA;
cc) BAX; BAK; BCKDHA; ICAM-1; GGTA1; CMAH; LPLA2; PPT1; and LIPA;
dd) BAX; BAK; BCKDHA; ICAM-1; SIRT-1; GGTA1; CMAH; LPLA2; PPT1; and LIPA;
ee) BAX; BAK; BCKDHA; ICAM-1; SIRT-1; MYC; GGTA1; CMAH; LPLA2; PPT1; and LIPA;
ff) BAX; BAK; BCKDHA; ICAM-1; LPL; LPLA2; and PPT1;
gg) BAX; BAK; BCKDHA; ICAM-1; SIRT-1; LPL, LPLA2; and PPT1;
hh) BAX; BAK; BCKDHA; ICAM-1; SIRT-1; MYC; LPL; LPLA2; and PPT1;
ii) BAX; BAK; BCKDHA; ICAM-1; SIRT-1; MYC; LPL; LPLA2; PPT1; and LIPA;
jj) BAX; BAK; BCKDHA; ICAM-1; SIRT-1; and MYC;
kk) BAX; BAK; BCKDHA; ICAM-1; PERK; SIRT-1; and MYC;
ll) BAX; BAK; BCKDHA; ICAM-1; PERK; SIRT-1; MYC; GGTA1; CMAH;
LPL, LPLA2; PPT1; and LIPA;
mm) BAX; BAK; BCKDHA; LPL; LPLA2; GGTA1; and CMAH;
nn) BAX; BAK; BCKDHA; PERK; LPL; LPLA2; GGTA1; and CMAH;
oo) BAX; BAK; BCKDHA; MYC; LPL; LPLA2; GGTA1; and CMAH;
pp) BAX; BAK; BCKDHA; MYC; PERK; LPL; LPLA2; GGTA1; and CMAH;
qq) BAX; BAK; BCKDHA; LPL; LPLA2; GGTA1; CMAH; and PPT1;
rr) BAX; BAK; BCKDHA; PERK; LPL; LPLA2; GGTA1; CMAH; and PPT1;
ss) BAX; BAK; BCKDHA; MYC; LPL; LPLA2; GGTA1; CMAH; and PPT1;
tt) BAX; BAK; BCKDHA; MYC; PERK; LPL; LPLA2; GGTA1; CMAH; and PPT1;
uu) BAX; BAK; BCKDHA; ICAM-1; and SIRT-1;
vv) BAX; BAK; BCKDHA; and ICAM-1;
ww) BAX; BAK; BCKDHB; ICAM-1; GGTA1; CMAH; LPL; LPLA2; and PPT1;
xx) BAX; BAK; BCKDHB; ICAM-1; SIRT-1; GGTA1; CMAH; LPL; LPLA2; and PPT1;
yy) BAX; BAK; BCKDHB; ICAM-1; SIRT-1; MYC; GGTA1; CMAH; LPL; LPLA2; and PPT1;
zz) BAX; BAK; BCKDHB; ICAM-1; SIRT-1; MYC; GGTA1; CMAH; LPL, LPLA2; PPT1; and LIPA;
aaa) BAX; BAK; BCKDHB; ICAM-1; GGTA1; CMAH; LPLA2; PPT1; and LIPA;
bbb) BAX; BAK; BCKDHB; ICAM-1; SIRT-1; GGTA1; CMAH; LPLA2; PPT1; and LIPA;
ccc) BAX; BAK; BCKDHB; ICAM-1; SIRT-1; MYC; GGTA1; CMAH; LPLA2; PPT1; and LIPA;
ddd) BAX; BAK; BCKDHB; ICAM-1; LPL; LPLA2; and PPT1;
eee) BAX; BAK; BCKDHB; ICAM-1; SIRT-1; LPL, LPLA2; and PPT1;
fff) BAX; BAK; BCKDHB; ICAM-1; SIRT-1; MYC; LPL; LPLA2; and PPT1;
ggg) BAX; BAK; BCKDHB; ICAM-1; SIRT-1; MYC; LPL; LPLA2; PPT1; and LIPA;
hhh) BAX; BAK; BCKDHB; ICAM-1; SIRT-1; and MYC;
iii) BAX; BAK; BCKDHB; ICAM-1; PERK; SIRT-1; and MYC;
jjj) BAX; BAK; BCKDHB; ICAM-1; PERK; SIRT-1; MYC; GGTA1; CMAH; LPL, LPLA2; PPT1; and LIPA;
kkk) BAX; BAK; BCKDHB; LPL; LPLA2; GGTA1; and CMAH;
lll) BAX; BAK; BCKDHB; PERK; LPL; LPLA2; GGTA1; and CMAH;
mmm) BAX; BAK; BCKDHB; MYC; LPL; LPLA2; GGTA1; and CMAH;
nnn) BAX; BAK; BCKDHB; MYC; PERK; LPL; LPLA2; GGTA1; and CMAH;
ooo) BAX; BAK; BCKDHB; LPL; LPLA2; GGTA1; CMAH; and PPT1;
ppp) BAX; BAK; BCKDHB; PERK; LPL; LPLA2; GGTA1; CMAH; and PPT1;
qqq) BAX; BAK; BCKDHB; MYC; LPL; LPLA2; GGTA1; CMAH; and PPT1;
rrr) BAX; BAK; BCKDHB; MYC; PERK; LPL; LPLA2; GGTA1; CMAH; and PPT1;
sss) BAX; BAK; BCKDHB; ICAM-1; and SIRT-1;
ttt) BAX; BAK; BCKDHB; and ICAM-1;
uuu) BAX; BAK; BCKDHA; BCKDHB; ICAM-1; GGTA1; CMAH; LPL; LPLA2; and PPT1;
vvv) BAX; BAK; BCKDHA; BCKDHB; ICAM-1; SIRT-1; GGTA1; CMAH; LPL; LPLA2; and PPT1;
www) BAX; BAK; BCKDHA; BCKDHB; ICAM-1; SIRT-1; MYC; GGTA1; CMAH; LPL; LPLA2; and PPT1;
xxx) BAX; BAK; BCKDHA; BCKDHB; ICAM-1; SIRT-1; MYC; GGTA1; CMAH; LPL, LPLA2; PPT1; and LIPA;
yyy) BAX; BAK; BCKDHA; BCKDHB; ICAM-1; GGTA1; CMAH; LPLA2; PPT1; and LIPA;
zzz) BAX; BAK; BCKDHA; BCKDHB; ICAM-1; SIRT-1; GGTA1; CMAH; LPLA2; PPT1; and LIPA;
aaaa) BAX; BAK; BCKDHA; BCKDHB; ICAM-1; SIRT-1; MYC; GGTA1; CMAH; LPLA2; PPT1; and LIPA;
bbbb) BAX; BAK; BCKDHA; BCKDHB; ICAM-1; LPL; LPLA2; and PPT1;
cccc) BAX; BAK; BCKDHA; BCKDHB; ICAM-1; SIRT-1; LPL, LPLA2; and PPT1;
dddd) BAX; BAK; BCKDHA; BCKDHB; ICAM-1; SIRT-1; MYC; LPL; LPLA2; and PPT1;
eeee) BAX; BAK; BCKDHA; BCKDHB; ICAM-1; SIRT-1; MYC; LPL; LPLA2; PPT1; and LIPA;
ffff) BAX; BAK; BCKDHA; BCKDHB; ICAM-1; SIRT-1; and MYC;
gggg) BAX; BAK; BCKDHA; BCKDHB; ICAM-1; PERK; SIRT-1; and MYC;
hhhh) BAX; BAK; BCKDHA; BCKDHB; ICAM-1; PERK; SIRT-1; MYC;
GGTA1; CMAH; LPL, LPLA2; PPT1; and LIPA;
iiii) BAX; BAK; BCKDHA; BCKDHB; LPL; LPLA2; GGTA1; and CMAH;
jjjj) BAX; BAK; BCKDHA; BCKDHB; PERK; LPL; LPLA2; GGTAT; and CMAH;
kkkk) BAX; BAK; BCKDHA; BCKDHB; MYC; LPL; LPLA2; GGTA1; and CMAH;
llll) BAX; BAK; BCKDHA; BCKDHB; MYC; PERK; LPL; LPLA2; GGTA1; and CMAH;
mmmm) BAX; BAK; BCKDHA; BCKDHB; LPL; LPLA2; GGTA1; CMAH; and PPT1;
nnnn) BAX; BAK; BCKDHA; BCKDHB; PERK; LPL; LPLA2; GGTA1; CMAH; and PPT1;
oooo) BAX; BAK; BCKDHA; BCKDHB; MYC; LPL; LPLA2; GGTAT; CMAH; and PPT1;
pppp) BAX; BAK; BCKDHA; BCKDHB; MYC; PERK; LPL; LPLA2; GGTA1; CMAH; and PPT1;
qqqq) BAX; BAK; BCKDHA; BCKDHB; ICAM-1; and SIRT-1; or
rrrr) BAX; BAK; BCKDHA; BCKDHB; and ICAM-1 is reduced or eliminated.

6. The modified mammalian cell of claim 1, wherein:
(a) the modified cell is transfected to express a recombinant product of interest; or
(b) the modified cell is generated from a recombinant cell that expresses a recombinant product of interest.

7. The modified mammalian cell of claim 6, wherein the one or more endogenous products have no detectable expression.

8. The modified mammalian cell of claim 6, wherein the recombinant product of interest comprises a viral vector, or a viral particle.

9. The modified mammalian cell of claim 6, wherein the recombinant product of interest comprises a recombinant protein.

10. The modified mammalian cell of claim 9, wherein the recombinant protein is antibody or an antigen-binding fragment thereof.

11. The modified mammalian cell of claim 6, wherein the exogenous nucleic acid sequence is integrated in the cellular genome of the mammalian cell at one or more targeted locations.

12. The modified mammalian cell of claim 1, wherein the modified cell does not express detectable BAX; BAK; ICAM-1; SIRT-1; PERK; MYC; GGTA1; CMAH; LPLA2; PPT1; BCKDHA; BCKDHB; LPL; and/or LIPA.

13. The modified mammalian cell of claim 1, wherein the modified cell expresses decreased levels of GAG; BAX; BAK; ICAM-1; SIRT-1; PERK; MYC; GGTA1; CMAH; LPLA2; PPT1; BCKDHA; BCKDHB; LPL; and/or LIPA.

14. The modified mammalian cell of claim 1, wherein the modified mammalian cell is a modified CHO cell.

15. A composition comprising the modified mammalian cell of claim 1.

16. A method of producing a recombinant product of interest comprising:
i) culturing a modified mammalian cell of claim 1;
ii) recovering the recombinant product of interest from the cultivation medium or the modified mammalian cells,
wherein the modified cells expressing the recombinant product of interest exhibit reduced or eliminated expression of one or more of the following GAG; BAX; BAK; ICAM-1; SIRT-1; PERK; MYC; GGTA1; CMAH; LPLA2; PPT1; BCKDHA; BCKDHB; LPL; and/or LIPA.

17. The method of claim 16, wherein the reduction of gene expression is mediated by RNA silencing.

18. The method of claim 16, wherein the modified cells expressing the recombinant product of interest exhibit reduced or eliminated expression of:
a) BAX; BAK; ICAM-1; GGTA1; CMAH; LPL; LPLA2; and PPT1;
b) BAX; BAK; ICAM-1; SIRT-1; GGTA1; CMAH; LPL; LPLA2; and PPT1;
c) BAX; BAK; ICAM-1; SIRT-1; MYC; GGTA1; CMAH; LPL; LPLA2; and PPT1;
d) BAX; BAK; ICAM-1; SIRT-1; MYC; GGTA1; CMAH; LPL, LPLA2; PPT1; and LIPA;
e) BAX; BAK; ICAM-1; GGTA1; CMAH; LPLA2; PPT1; and LIPA;
f) BAX; BAK; ICAM-1; SIRT-1; GGTA1; CMAH; LPLA2; PPT1; and LIPA;
g) BAX; BAK; ICAM-1; SIRT-1; MYC; GGTA1; CMAH; LPLA2; PPT1; and
LIPA;
h) BAX; BAK; ICAM-1; LPL; LPLA2; and PPT1;
i) BAX; BAK; ICAM-1; SIRT-1; LPL, LPLA2; and PPT1;
j) BAX; BAK; ICAM-1; SIRT-1; MYC; LPL; LPLA2; and PPT1;
k) BAX; BAK; ICAM-1; SIRT-1; MYC; LPL; LPLA2; PPT1; and LIPA;
l) BAX; BAK; ICAM-1; SIRT-1; and MYC;
m) BAX; BAK; ICAM-1; PERK; SIRT-1; and MYC;
n) BAX; BAK; ICAM-1; SIRT-1; PERK; MYC; GGTA1; CMAH; LPL, LPLA2; PPT1; and LIPA;
o) BAX; BAK; LPL; LPLA2; GGTA1; and CMAH;
p) BAX; BAK; PERK; LPL; LPLA2; GGTA1; and CMAH;
q) BAX; BAK; MYC; LPL; LPLA2; GGTA1; and CMAH;
r) BAX; BAK; MYC; PERK; LPL; LPLA2; GGTA1; and CMAH;
s) BAX; BAK; LPL; LPLA2; GGTA1; CMAH; and PPT1;
t) BAX; BAK; PERK; LPL; LPLA2; GGTA1; CMAH; and PPT1;
u) BAX; BAK; MYC; LPL; LPLA2; GGTA1; CMAH; and PPT1;
v) BAX; BAK; MYC; PERK; LPL; LPLA2; GGTA1; CMAH; and PPT1;
w) BAX; BAK; ICAM-1; and SIRT-1;
x) BAX; BAK; and ICAM-1;
y) BAX; BAK; BCKDHA; ICAM-1; GGTA1; CMAH; LPL; LPLA2; and PPT1;
z) BAX; BAK; BCKDHA; ICAM-1; SIRT-1; GGTA1; CMAH; LPL; LPLA2; and PPT1;
aa) BAX; BAK; BCKDHA; ICAM-1; SIRT-1; MYC; GGTA1; CMAH; LPL; LPLA2; and PPT1;
bb) BAX; BAK; BCKDHA; ICAM-1; SIRT-1; MYC; GGTA1; CMAH; LPL, LPLA2; PPT1; and LIPA;
cc) BAX; BAK; BCKDHA; ICAM-1; GGTA1; CMAH; LPLA2; PPT1; and
LIPA;
dd) BAX; BAK; BCKDHA; ICAM-1; SIRT-1; GGTA1; CMAH; LPLA2; PPT1; and LIPA;
ee) BAX; BAK; BCKDHA; ICAM-1; SIRT-1; MYC; GGTA1; CMAH; LPLA2; PPT1; and LIPA;
ff) BAX; BAK; BCKDHA; ICAM-1; LPL; LPLA2; and PPT1;
gg) BAX; BAK; BCKDHA; ICAM-1; SIRT-1; LPL, LPLA2; and PPT1;
hh) BAX; BAK; BCKDHA; ICAM-1; SIRT-1; MYC; LPL; LPLA2; and PPT1;
ii) BAX; BAK; BCKDHA; ICAM-1; SIRT-1; MYC; LPL; LPLA2; PPT1; and
LIPA;
jj) BAX; BAK; BCKDHA; ICAM-1; SIRT-1; and MYC;
kk) BAX; BAK; BCKDHA; ICAM-1; PERK; SIRT-1; and MYC;
ll) BAX; BAK; BCKDHA; ICAM-1; PERK; SIRT-1; MYC; GGTA1; CMAH;
LPL, LPLA2; PPT1; and LIPA;
mm) BAX; BAK; BCKDHA; LPL; LPLA2; GGTA1; and CMAH;
nn) BAX; BAK; BCKDHA; PERK; LPL; LPLA2; GGTA1; and CMAH;
oo) BAX; BAK; BCKDHA; MYC; LPL; LPLA2; GGTA1; and CMAH;
pp) BAX; BAK; BCKDHA; MYC; PERK; LPL; LPLA2; GGTA1; and CMAH;
qq) BAX; BAK; BCKDHA; LPL; LPLA2; GGTA1; CMAH; and PPT1;
rr) BAX; BAK; BCKDHA; PERK; LPL; LPLA2; GGTA1; CMAH; and PPT1;
ss) BAX; BAK; BCKDHA; MYC; LPL; LPLA2; GGTA1; CMAH; and PPT1;
tt) BAX; BAK; BCKDHA; MYC; PERK; LPL; LPLA2; GGTA1; CMAH; and PPT1;
uu) BAX; BAK; BCKDHA; ICAM-1; and SIRT-1;
vv) BAX; BAK; BCKDHA; and ICAM-1;
ww) BAX; BAK; BCKDHB; ICAM-1; GGTA1; CMAH; LPL; LPLA2; and PPT1;
xx) BAX; BAK; BCKDHB; ICAM-1; SIRT-1; GGTA1; CMAH; LPL; LPLA2; and PPT1;
yy) BAX; BAK; BCKDHB; ICAM-1; SIRT-1; MYC; GGTA1; CMAH; LPL; LPLA2; and PPT1;
zz) BAX; BAK; BCKDHB; ICAM-1; SIRT-1; MYC; GGTA1; CMAH; LPL, LPLA2; PPT1; and LIPA;
aaa) BAX; BAK; BCKDHB; ICAM-1; GGTA1; CMAH; LPLA2; PPT1; and
LIPA;
bbb) BAX; BAK; BCKDHB; ICAM-1; SIRT-1; GGTA1; CMAH; LPLA2; PPT1; and LIPA;
ccc) BAX; BAK; BCKDHB; ICAM-1; SIRT-1; MYC; GGTA1; CMAH; LPLA2; PPT1; and LIPA;
ddd) BAX; BAK; BCKDHB; ICAM-1; LPL; LPLA2; and PPT1;
eee) BAX; BAK; BCKDHB; ICAM-1; SIRT-1; LPL, LPLA2; and PPT1;
fff) BAX; BAK; BCKDHB; ICAM-1; SIRT-1; MYC; LPL; LPLA2; and PPT1;
ggg) BAX; BAK; BCKDHB; ICAM-1; SIRT-1; MYC; LPL; LPLA2; PPT1; and
LIPA;
hhh) BAX; BAK; BCKDHB; ICAM-1; SIRT-1; and MYC;
iii) BAX; BAK; BCKDHB; ICAM-1; PERK; SIRT-1; and MYC;
jjj) BAX; BAK; BCKDHB; ICAM-1; PERK; SIRT-1; MYC; GGTA1; CMAH;
LPL, LPLA2; PPT1; and LIPA;
kkk) BAX; BAK; BCKDHB; LPL; LPLA2; GGTA1; and CMAH;
lll) BAX; BAK; BCKDHB; PERK; LPL; LPLA2; GGTA1; and CMAH;
mmm) BAX; BAK; BCKDHB; MYC; LPL; LPLA2; GGTA1; and CMAH;
nnn) BAX; BAK; BCKDHB; MYC; PERK; LPL; LPLA2; GGTA1; and CMAH;
ooo) BAX; BAK; BCKDHB; LPL; LPLA2; GGTA1; CMAH; and PPT1;
ppp) BAX; BAK; BCKDHB; PERK; LPL; LPLA2; GGTA1; CMAH; and PPT1;
qqq) BAX; BAK; BCKDHB; MYC; LPL; LPLA2; GGTA1; CMAH; and PPT1;
rrr) BAX; BAK; BCKDHB; MYC; PERK; LPL; LPLA2; GGTA1; CMAH; and PPT1;
sss) BAX; BAK; BCKDHB; ICAM-1; and SIRT-1;
ttt) BAX; BAK; BCKDHB; and ICAM-1;
uuu) BAX; BAK; BCKDHA; BCKDHB; ICAM-1; GGTA1; CMAH; LPL; LPLA2; and PPT1;
vvv) BAX; BAK; BCKDHA; BCKDHB; ICAM-1; SIRT-1; GGTA1; CMAH;
LPL; LPLA2; and PPT1;
www) BAX; BAK; BCKDHA; BCKDHB; ICAM-1; SIRT-1; MYC; GGTA1; CMAH; LPL; LPLA2; and PPT1;
xxx) BAX; BAK; BCKDHA; BCKDHB; ICAM-1; SIRT-1; MYC; GGTA1; CMAH; LPL, LPLA2; PPT1; and LIPA;
yyy) BAX; BAK; BCKDHA; BCKDHB; ICAM-1; GGTA1; CMAH; LPLA2; PPT1; and LIPA;
zzz) BAX; BAK; BCKDHA; BCKDHB; ICAM-1; SIRT-1; GGTA1; CMAH; LPLA2; PPT1; and LIPA;
aaaa) BAX; BAK; BCKDHA; BCKDHB; ICAM-1; SIRT-1; MYC; GGTA1; CMAH; LPLA2; PPT1; and LIPA;
bbbb) BAX; BAK; BCKDHA; BCKDHB; ICAM-1; LPL; LPLA2; and PPT1;
cccc) BAX; BAK; BCKDHA; BCKDHB; ICAM-1; SIRT-1; LPL, LPLA2; and PPT1;
dddd) BAX; BAK; BCKDHA; BCKDHB; ICAM-1; SIRT-1; MYC; LPL; LPLA2; and PPT1;
eeee) BAX; BAK; BCKDHA; BCKDHB; ICAM-1; SIRT-1; MYC; LPL; LPLA2; PPT1; and LIPA;
ffff) BAX; BAK; BCKDHA; BCKDHB; ICAM-1; SIRT-1; and MYC;
gggg) BAX; BAK; BCKDHA; BCKDHB; ICAM-1; PERK; SIRT-1; and MYC;
hhhh) BAX; BAK; BCKDHA; BCKDHB; ICAM-1; PERK; SIRT-1; MYC;
GGTA1; CMAH; LPL, LPLA2; PPT1; and LIPA;
iiii) BAX; BAK; BCKDHA; BCKDHB; LPL; LPLA2; GGTA1; and CMAH;
jjjj) BAX; BAK; BCKDHA; BCKDHB; PERK; LPL; LPLA2; GGTA1; and CMAH;
kkkk) BAX; BAK; BCKDHA; BCKDHB; MYC; LPL; LPLA2; GGTA1; and CMAH;
llll) BAX; BAK; BCKDHA; BCKDHB; MYC; PERK; LPL; LPLA2; GGTA1; and CMAH;
mmmm) BAX; BAK; BCKDHA; BCKDHB; LPL; LPLA2; GGTA1; CMAH; and PPT1;
nnnn) BAX; BAK; BCKDHA; BCKDHB; PERK; LPL; LPLA2; GGTA1; CMAH; and PPT1;
oooo) BAX; BAK; BCKDHA; BCKDHB; MYC; LPL; LPLA2; GGTA1; CMAH; and PPT1;
pppp) BAX; BAK; BCKDHA; BCKDHB; MYC; PERK; LPL; LPLA2; GGTA1; CMAH; and PPT1;
qqqq) BAX; BAK; BCKDHA; BCKDHB; ICAM-1; and SIRT-1; or
rrrr) BAX; BAK; BCKDHA; BCKDHB; and ICAM-1.

19. The method of claim 16, wherein the modified cells expressing the recombinant product of interest exhibit reduced or eliminated expression of:
a) GAG; BAX; BAK; ICAM-1; GGTA1; CMAH; LPL; LPLA2; and PPT1;
b) GAG; BAX; BAK; ICAM-1; SIRT-1; GGTA1; CMAH; LPL; LPLA2; and PPT1;
c) GAG; BAX; BAK; ICAM-1; SIRT-1; MYC; GGTA1; CMAH; LPL; LPLA2; and PPT1;
d) GAG; BAX; BAK; ICAM-1; SIRT-1; MYC; GGTA1; CMAH; LPL, LPLA2; PPT1; and LIPA;
e) GAG; BAX; BAK; ICAM-1; GGTA1; CMAH; LPLA2; PPT1; and LIPA;
f) GAG; BAX; BAK; ICAM-1; SIRT-1; GGTA1; CMAH; LPLA2; PPT1; and
LIPA;
g) GAG; BAX; BAK; ICAM-1; SIRT-1; MYC; GGTA1; CMAH; LPLA2; PPT1; and LIPA;
h) GAG; BAX; BAK; ICAM-1; LPL; LPLA2; and PPT1;
i) GAG; BAX; BAK; ICAM-1; SIRT-1; LPL, LPLA2; and PPT1;
j) GAG; BAX; BAK; ICAM-1; SIRT-1; MYC; LPL; LPLA2; and PPT1;
k) GAG; BAX; BAK; ICAM-1; SIRT-1; MYC; LPL; LPLA2; PPT1; and
LIPA;
l) GAG; BAX; BAK; ICAM-1; SIRT-1; and MYC;
m) GAG; BAX; BAK; ICAM-1; PERK; SIRT-1; and MYC;
n) GAG; BAX; BAK; ICAM-1; SIRT-1; PERK; MYC; GGTA1; CMAH; LPL, LPLA2; PPT1; and LIPA;
o) GAG; BAX; BAK; LPL; LPLA2; GGTA1; and CMAH;
p) GAG; BAX; BAK; PERK; LPL; LPLA2; GGTA1; and CMAH;
q) GAG; BAX; BAK; MYC; LPL; LPLA2; GGTA1; and CMAH;
r) GAG; BAX; BAK; MYC; PERK; LPL; LPLA2; GGTA1; and CMAH;
s) GAG; BAX; BAK; LPL; LPLA2; GGTA1; CMAH; and PPT1;
t) GAG; BAX; BAK; PERK; LPL; LPLA2; GGTA1; CMAH; and PPT1;
u) GAG; BAX; BAK; MYC; LPL; LPLA2; GGTA1; CMAH; and PPT1;
v) GAG; BAX; BAK; MYC; PERK; LPL; LPLA2; GGTA1; CMAH; and PPT1;
w) GAG; BAX; BAK; ICAM-1; and SIRT-1;
x) GAG; BAX; BAK; and ICAM-1
y) BAX; BAK; BCKDHA; ICAM-1; GGTA1; CMAH; LPL; LPLA2; and PPT1;
z) BAX; BAK; BCKDHA; ICAM-1; SIRT-1; GGTA1; CMAH; LPL; LPLA2; and PPT1;
aa) BAX; BAK; BCKDHA; ICAM-1; SIRT-1; MYC; GGTA1; CMAH; LPL; LPLA2; and PPT1;
bb) BAX; BAK; BCKDHA; ICAM-1; SIRT-1; MYC; GGTA1; CMAH; LPL, LPLA2; PPT1; and LIPA;
cc) BAX; BAK; BCKDHA; ICAM-1; GGTA1; CMAH; LPLA2; PPT1; and
LIPA;
dd) BAX; BAK; BCKDHA; ICAM-1; SIRT-1; GGTA1; CMAH; LPLA2; PPT1; and LIPA;
ee) BAX; BAK; BCKDHA; ICAM-1; SIRT-1; MYC; GGTA1; CMAH; LPLA2; PPT1; and LIPA;
ff) BAX; BAK; BCKDHA; ICAM-1; LPL; LPLA2; and PPT1;
gg) BAX; BAK; BCKDHA; ICAM-1; SIRT-1; LPL, LPLA2; and PPT1;
hh) BAX; BAK; BCKDHA; ICAM-1; SIRT-1; MYC; LPL; LPLA2; and PPT1;
ii) BAX; BAK; BCKDHA; ICAM-1; SIRT-1; MYC; LPL; LPLA2; PPT1; and
LIPA;
jj) BAX; BAK; BCKDHA; ICAM-1; SIRT-1; and MYC;
kk) BAX; BAK; BCKDHA; ICAM-1; PERK; SIRT-1; and MYC;
ll) BAX; BAK; BCKDHA; ICAM-1; PERK; SIRT-1; MYC; GGTA1; CMAH;
LPL, LPLA2; PPT1; and LIPA;
mm) BAX; BAK; BCKDHA; LPL; LPLA2; GGTA1; and CMAH;
nn) BAX; BAK; BCKDHA; PERK; LPL; LPLA2; GGTA1; and CMAH;
oo) BAX; BAK; BCKDHA; MYC; LPL; LPLA2; GGTA1; and CMAH;
pp) BAX; BAK; BCKDHA; MYC; PERK; LPL; LPLA2; GGTA1; and CMAH;
qq) BAX; BAK; BCKDHA; LPL; LPLA2; GGTA1; CMAH; and PPT1;
rr) BAX; BAK; BCKDHA; PERK; LPL; LPLA2; GGTA1; CMAH; and PPT1;
ss) BAX; BAK; BCKDHA; MYC; LPL; LPLA2; GGTA1; CMAH; and PPT1;
tt) BAX; BAK; BCKDHA; MYC; PERK; LPL; LPLA2; GGTA1; CMAH; and PPT1;
uu) BAX; BAK; BCKDHA; ICAM-1; and SIRT-1;
vv) BAX; BAK; BCKDHA; and ICAM-1;
ww) BAX; BAK; BCKDHB; ICAM-1; GGTA1; CMAH; LPL; LPLA2; and PPT1;
xx) BAX; BAK; BCKDHB; ICAM-1; SIRT-1; GGTA1; CMAH; LPL; LPLA2; and PPT1;
yy) BAX; BAK; BCKDHB; ICAM-1; SIRT-1; MYC; GGTA1; CMAH; LPL; LPLA2; and PPT1;
zz) BAX; BAK; BCKDHB; ICAM-1; SIRT-1; MYC; GGTA1; CMAH; LPL, LPLA2; PPT1; and LIPA;
aaa) BAX; BAK; BCKDHB; ICAM-1; GGTA1; CMAH; LPLA2; PPT1; and
LIPA;
bbb) BAX; BAK; BCKDHB; ICAM-1; SIRT-1; GGTA1; CMAH; LPLA2; PPT1; and LIPA;
ccc) BAX; BAK; BCKDHB; ICAM-1; SIRT-1; MYC; GGTA1; CMAH; LPLA2; PPT1; and LIPA;
ddd) BAX; BAK; BCKDHB; ICAM-1; LPL; LPLA2; and PPT1;
eee) BAX; BAK; BCKDHB; ICAM-1; SIRT-1; LPL, LPLA2; and PPT1;
fff) BAX; BAK; BCKDHB; ICAM-1; SIRT-1; MYC; LPL; LPLA2; and PPT1;
ggg) BAX; BAK; BCKDHB; ICAM-1; SIRT-1; MYC; LPL; LPLA2; PPT1; and
LIPA;
hhh) BAX; BAK; BCKDHB; ICAM-1; SIRT-1; and MYC;
iii) BAX; BAK; BCKDHB; ICAM-1; PERK; SIRT-1; and MYC;
jjj) BAX; BAK; BCKDHB; ICAM-1; PERK; SIRT-1; MYC; GGTA1; CMAH;
LPL, LPLA2; PPT1; and LIPA;
kkk) BAX; BAK; BCKDHB; LPL; LPLA2; GGTA1; and CMAH;
lll) BAX; BAK; BCKDHB; PERK; LPL; LPLA2; GGTA1; and CMAH;
mmm) BAX; BAK; BCKDHB; MYC; LPL; LPLA2; GGTA1; and CMAH;
nnn) BAX; BAK; BCKDHB; MYC; PERK; LPL; LPLA2; GGTA1; and CMAH;
ooo) BAX; BAK; BCKDHB; LPL; LPLA2; GGTA1; CMAH; and PPT1;
ppp) BAX; BAK; BCKDHB; PERK; LPL; LPLA2; GGTA1; CMAH; and PPT1;
qqq) BAX; BAK; BCKDHB; MYC; LPL; LPLA2; GGTA1; CMAH; and PPT1;
rrr) BAX; BAK; BCKDHB; MYC; PERK; LPL; LPLA2; GGTA1; CMAH; and PPT1;
sss) BAX; BAK; BCKDHB; ICAM-1; and SIRT-1;
ttt) BAX; BAK; BCKDHB; and ICAM-1;
uuu) BAX; BAK; BCKDHA; BCKDHB; ICAM-1; GGTA1; CMAH; LPL; LPLA2; and PPT1;
vvv) BAX; BAK; BCKDHA; BCKDHB; ICAM-1; SIRT-1; GGTA1; CMAH;
LPL; LPLA2; and PPT1;
www) BAX; BAK; BCKDHA; BCKDHB; ICAM-1; SIRT-1; MYC; GGTA1; CMAH; LPL; LPLA2; and PPT1;
xxx) BAX; BAK; BCKDHA; BCKDHB; ICAM-1; SIRT-1; MYC; GGTA1; CMAH; LPL, LPLA2; PPT1; and LIPA;
yyy) BAX; BAK; BCKDHA; BCKDHB; ICAM-1; GGTA1; CMAH; LPLA2; PPT1; and LIPA;
zzz) BAX; BAK; BCKDHA; BCKDHB; ICAM-1; SIRT-1; GGTA1; CMAH; LPLA2; PPT1; and LIPA;
aaaa) BAX; BAK; BCKDHA; BCKDHB; ICAM-1; SIRT-1; MYC; GGTA1; CMAH; LPLA2; PPT1; and LIPA;
bbbb) BAX; BAK; BCKDHA; BCKDHB; ICAM-1; LPL; LPLA2; and PPT1;
cccc) BAX; BAK; BCKDHA; BCKDHB; ICAM-1; SIRT-1; LPL, LPLA2; and PPT1;
dddd) BAX; BAK; BCKDHA; BCKDHB; ICAM-1; SIRT-1; MYC; LPL; LPLA2; and PPT1;
eeee) BAX; BAK; BCKDHA; BCKDHB; ICAM-1; SIRT-1; MYC; LPL; LPLA2; PPT1; and LIPA;
ffff) BAX; BAK; BCKDHA; BCKDHB; ICAM-1; SIRT-1; and MYC;
gggg) BAX; BAK; BCKDHA; BCKDHB; ICAM-1; PERK; SIRT-1; and MYC;
hhhh) BAX; BAK; BCKDHA; BCKDHB; ICAM-1; PERK; SIRT-1; MYC;
GGTA1; CMAH; LPL, LPLA2; PPT1; and LIPA;
iiii) BAX; BAK; BCKDHA; BCKDHB; LPL; LPLA2; GGTA1; and CMAH;
jjjj) BAX; BAK; BCKDHA; BCKDHB; PERK; LPL; LPLA2; GGTA1; and CMAH;
kkkk) BAX; BAK; BCKDHA; BCKDHB; MYC; LPL; LPLA2; GGTA1; and CMAH;
llll) BAX; BAK; BCKDHA; BCKDHB; MYC; PERK; LPL; LPLA2; GGTA1; and CMAH;
mmmm) BAX; BAK; BCKDHA; BCKDHB; LPL; LPLA2; GGTA1; CMAH; and PPT1;
nnnn) BAX; BAK; BCKDHA; BCKDHB; PERK; LPL; LPLA2; GGTA1; CMAH; and PPT1;
oooo) BAX; BAK; BCKDHA; BCKDHB; MYC; LPL; LPLA2; GGTA1; CMAH; and PPT1;
pppp) BAX; BAK; BCKDHA; BCKDHB; MYC; PERK; LPL; LPLA2; GGTA1; CMAH; and PPT1;
qqqq) BAX; BAK; BCKDHA; BCKDHB; ICAM-1; and SIRT-1; or
rrrr) BAX; BAK; BCKDHA; BCKDHB; and ICAM-1.

20. The method of claim 16, wherein the recombinant product of interest is encoded by a nucleic acid sequence.

21. The method of claim 20, wherein the nucleic acid sequence is integrated in the cellular genome of the modified cells at one or more targeted locations.

22. The method of claim 20, wherein the nucleic acid sequence is randomly integrated in the cellular genome of the mammalian cells.

23. The method of claim 16, wherein the recombinant product of interest comprises a viral vector, or a viral particle.

24. The method of claim 16, wherein the recombinant product of interest comprises a recombinant protein.

25. The method of claim 24, wherein the recombinant protein is an antibody or an antigen-binding fragment thereof.

26. The method of claim 16, comprising purifying the product of interest, harvesting the product of interest, and/or formulating the product of interest.

27. The method of claim 16, wherein the modified mammalian cell is a modified CHO cell.

28. A method for producing a modified mammalian cell, comprising:
(1) applying a nuclease-assisted and/or nucleic acid targeting at least one endogenous gene selected from the group of genes consisting of GAG; BAX; BAK; ICAM-1; PERK; SIRT-1; MYC; GGTA1; CMAH; LPLA2; PPT1; BCKDHA; BCKDHB; LPL; and LIPA, in the mammalian cell to reduce or eliminate the expression of said endogenous gene, and
(2) selecting the modified mammalian cell wherein the expression of said endogenous gene has been reduced or eliminated as compared to an unmodified mammalian cell.

29. The method of claim 28, wherein the modification is performed before the introduction of the exogenous nucleic acid encoding the recombinant product of interest, or after the introduction of the exogenous nucleic acid encoding the recombinant product of interest.
